SEQUENCE LISTING (62 sequences)

SEQ ID NO: 1 - DNA, 5034 bp, Synthetic construct
  source 1..5034: mol_type = other DNA
ggagctgtggggacgactcttctggaggaagcagcgcgggcttgaccggcgtcggcccgccgcctccgctgccgcttcgccccaatccggtccctctggcccggcctgacccggtctggcttgttcgggctcagcggccgcgaggccgcagctcccgatggaaatcatattatgtagaatacttgggtgacatctgcctgagagatctccaagaattacagattgagtctcgctctgttgcccaggctggagtgcagtggtgcaatctctgctcactgcaacctccgtctcccgagttcaagcagttctcctgcctcagcctcctgagtagctgggattacagaagacaaaaatactaatgcatttgagaaagcggtagttttggggggagggggaaaaagcaactgctttcctgatctgcaacttggctggatgctaagatgtcagtggacatgaatagccaggggtctgacagcaatgaagaggactatgacccaaattgtgaggaagaggaagaagaagaagaagacgaccctggggacatagaggactattacgtgggagtagccagcgatgtggagcagcagggggctgatgcctttgatcccgaggagtaccagttcacttgcttgacctacaaggaatctgagggtgccctcaatgagcacatgaccagcttagcttctgtcctaaaggtatctcattcagttgctaaacttatattagttaatttccactggcaagtttcagagatattggacagatacaagtccaattctgctcaactgcttgttgaggctcgagttcagcctaatccatcaaaacatgttcccacatcccatccccctcaccactgtgcagtgtgtatgcagtttgtgcgaaaggaaaacctactctctctggcctgtcagcaccagttttgccgcagctgctgggagcagcactgctcagttctcgtcaaggacggcgtgggcgtgggagtctcttgcatggctcaggactgtccactccgtacaccagaggactttgtgtttccattgcttcccaatgaagaattgagagagaaatacaggcgctacctcttcagggactatgtggagagtcattaccagctccagctgtgccctggtgcagactgccccatggttattcgggtacaggagcctagagctcgccgagtacagtgcaatcggtgcaacgaggtcttctgtttcaagtgtcgtcagatgtatcacgcacccacagactgtgccacaatccggaaatggctcacgaagtgtgcagacgactctgaaacagccaactacattagtgctcacactaaagactgtcccaagtgcaacatctgcattgagaagaatggaggctgcaatcacatgcaatgctccaaatgtaaacacgacttctgctggatgtgtctaggagattggaagactcatggcagtgaatactatgagtgcagtcgttacaaggagaatcctgacatcgtgaaccagagccaacaagcccaggcgagggaagccctcaagaagtacttattctactttgagaggtgggaaaaccacaataaaagcttgcagctagaggcacagacataccagcggattcacgagaagattcaggagagggtcatgaacaatctggggacatggatcgactggcagtacctacagaatgctgccaagctcttggccaagtgtcgatacaccctgcaatacacctacccatatgcatattacatggagtccggacccaggaagaagctgtttgaataccagcaggctcagctggaggctgagatcgaaaacctctcatggaaagtggagcgtgcagacagctatgacagaggggacttggagaaccagatgcatatagcggagcagcggaggagaaccctgctgaaagatttccatgacacctaagttgggatgtggatgtgccggggtgaggaagatgtggctgcaaggtctcccggctgccatactgcatgctgcaggctctgcctttcatgaccccaggcaacagccagggccccactcctgagagacactggcaacacctcttagttgatttctgttttcttctcttttcactttttgtttctaccagggtagaggccatgttgaactggcctcttttcaggacttttatttccccctggatggttgttgggagggagggaaagtgttttctgaatggctattaatagtattagatcattacaacttatgtaactttcaaaggttgtacaattatacaaaaaaaaaaaaaaggcaaactataggataacacagagccctttttgaaaataaattggcattggagtgttttaccctctagctgttttacttagaatgtaacatatgctgcctacccacctcaaaatgtctgtactgcaagagggccctgggcctctgctttccatattcacgtttggccagagttgtagtcccaaagaagagcatgggtggcagatggtagggaattgaactggcctgtgcaatgggcatggagcacaaggggtcacagcatgcctcctgccttaccgtggcagtacggagacagtccagaacatggtcttcttgccacggggtgttgttgtctctggtggtgctgcatgtctgtggctcacctttattcttgaaactgaggtttacctggatctggctactgaggctagagcccacagcagaatggggttgggcctgtggccccccaaactagggggtgtgggttcatcacagtgttgccttttgtctcctaaagatagggatctacttttgaagggaattgttcctcccaaataaatttgctttaccttggtcctttcttttgtgccagtattcaagtggtatagctctgagcagggtcacatttggccaaacctgacactgtcttgctgcattctcctttggcaaacatcagggtcagaattcaggatagcccttcctagggcactggactttctggcatgggggctgtgtttgcacaagttattttcatgttacctggagagtgtccagaggctgctctgaggctgaggtgtgttcccccttgcctggttccagctgtcagagggataccatcctagggtctgggaatccaaggccacgagactccttggtttgtggtccgagatcctgtactaaggagggtctggccagaggaacagaccagcttttgcacaatgaagcgcaagggaacaagtggtttgcctggtgtcctacctgtcctgaacctggtcctgtgggccattgaaaagttagatctgtgatctctggggtttttgtggctttgttcaatgcttccactctagggcaggcagagcagtctatactctcccaagcctgcttgacctccaagtagagctgatacagagatctgtgaatattgtgatagaaattctttggtattcatacatttcagctgcaagtcagcaatttcccaggtaccatgtaagctataaaacagtcattcttaaagacagaggatagctgtgactcatgggatcatgaggtccatggctggttgcaggttccctttttccttcctcaggttttgtctcttcctgtgttgtccccagcaagggagagactgtggggtggattgggagaacagattaggagtatagcaaatgaacccagaatggaacagtggggagctaactgtgaatgaggagagtacctgctgcaggacctggaggtcaggtgtgaatgctgtattggcacagggaataaatatcctggcgtctggagccttcacctctccgtcaagtccttcctgtgatactgccatggcacaggatctgagttgcagctctgcaccctaaatcacaccctgggcattgtctgggctgcagggctgccaggttctgtacttgtgtccagctgtggccctggatgctggagctggagggttttctgtgctcagactgtagcctgtagctcttggcctgtgtagagccccctcctgtgccctcagtggctgtcgtttgttaacatcatcaggaagatgggaaaggtcaggcagaatttttctgccctacaaagggtggaagagaaaggacacagtattttcatgaatttaccatatatctttgtttttcttcaacgaaaaagttaattgaggcaatgtcatctgctcaaagttgagtggtttattcacaataaactgtaagtttctgattataaaaatgtgtctagagtctttcttcatggggtctaggtagtcttctgagcccacaggggtctggccagtgaacaccagaggagtgccttcctatcagtcagccccttcttccagagggctgtggaggccccagtggccaaactcggctctgttttccctttctggctgttgggggagagggcacctgtaagtgatccctgtggaatgaagtagaaaatcctgccagttttccgctctcctctccctcagtacaggctgaatcattccccaccacagatagatcccagggttgtggtagagtactgatataaatgtcccaacccccccattctgagacctccggacccctccaaagcagacatgggaatgtagggctgtggaatatggcagggtgaactccagcttcacgggagccctaagggttatgaagagacaacttggggcagaaacaagtgtggagtctaatctcaagggtgaaggacaggaaatgaggtttggattgattaagggggtccacatttgtggtgtcagaagtggcagtagtgtcagtgataagtagctcaggggtagagtggaggctccatagctgtgggcaacatgcacagggcacctgtggctagaagaggcctcatacattgccagtctgaactccgcagctcttcctctggacctcaggtgtgccaagccccacgcttggcacctgggcatacagtggtatgcaagtgattcacgtgctctagcttccaggagcttccaatccatggggaacatagaaggaattgatcactagtgaatgtttgttttttgttttgagacagtctcgctctatcgcccagactggagtac

SEQ ID NO: 2 - DNA, 3975 bp, Synthetic construct
  source 1..3975: mol_type = other DNA
acgcgcggcgcagaacacttccggagctgcgggggacggctctgccggaggaagctgcgcgggcctgagccgcgtccgccggctgcctccgctgccgctacgcctccgtcagcctggtttggtctggcttgacctggtccggccggcctgggctctgcggccgcgaggtcgcggctcccgatggaaatcctatgacagacagtttgggtgatatctgcaggaaagatctttaaacattacagaagacaatacacgctaatgcatttgagaaaacgataaaaattgggggtagggaaaaaagcaactgtttctgcaactttgctggatgttaagatgtctgtggacatgaacagccaagggtcagatagcaatgaagaggactatgacccaaactgtgaggaagaggaagaagaggaagaggaccctggggacatagaggactactatgtgggagtagccagtgatgtggagcagcagggggctgatgcttttgatcctgaagagtatcagttcacttgcttgacctataaggagtctgagggcgccctccatgaacatatgaccagcttagcctctgtcttaaaggtatctcattcagttgcaaaacttatattagttaatttccactggcaagtctcagagattctggacaggtacaggtctaattctgctcagctgcttgttgaggctcgagttcaacctaatccatccaaacatgtccccacagcccatccccctcaccactgtgcagtgtgtatgcaatttgtgcggaaggaaaacctactctctctggcctgccaacatcagttttgccgaagctgttgggaacagcattgctcagtgcttgtcaaagatggtgttggtgtaggaatctcatgtatggctcaggattgtccacttcgaacaccagaggactttgtatttcccttgcttcccaatgaagaattgagagataaatacaggcgctacctctttagggactatgtggagagccatttccagctccagctgtgccccggtgcagactgccccatggttatccgggtacaggagcctagagcccgccgagtgcagtgcaatcggtgcagcgaggtcttctgtttcaagtgtcgacagatgtatcacgcccccacagactgcgccacaatccggaaatggctcaccaagtgtgcagacgactccgaaacggctaactacattagtgctcacactaaagactgtcccaagtgcaacatctgcatcgagaagaatggaggctgtaatcacatgcaatgctccaagtgtaagcacgacttctgctggatgtgtctaggagactggaagacgcatggtagtgagtactatgaatgtagtcggtacaaggagaatcctgacattgtcaaccagagccagcaagcacaggccagggaggccctcaagaagtacttgttctactttgagaggtgggaaaatcacaacaaaagcttgcagctggaggcacaaacatatgagcggattcacgagaagattcaggagagggttatgaacaacctgggaacttggattgactggcagtacctacagaatgctgccaagctcttagccaagtgtcgatataccctgcagtacacctatccatatgcatactacatggaatccggtcccaggaagaagctgtttgaataccagcaggctcagctggaggctgagatcgaaaacctttcatggaaagttgagcgtgcagacagctacgacagaggggacttagaaaaccagatgcacatagcagagcagcggaggagaaccctgctaaaggattttcacgacacctaaactgggctgtggctgtgccggggtgaggaagatgtagctgtgaggccgcccggctgccctcctgcaggctgtaggctctgcctttcaccacccaggcaacagccagggccccactcctgaaagacactggcaacacacctcttagccgatttgttttctcctcatctttttgatttttatttctaccagggtagaggccgcgttgagctggcctctaatgaggactttacatttcccctggatggttgttgggcgggagggaacttttttttcctgaatggctattaatagtattagatcattacaacttatgtaattttcaaaggttgtacgattataaaggaaagaggcaaagcatagataacacagagcccgattaaaataaattggcattggagtgcttcaccctttagctatttgacttagaatgtaacttgatactgcccagccactgagcctcagcgtgtctgcactgccgggggccctgggacggcccctgctttccagcatcatgtttgactggagccggagtcctggggaaggtgcacgggtggcagagagtaggaaactgagttggcctgcataataggatggagcacacagttacttacctggccttagctggacagccagggcacagagcagagcaaggtcgcactgcaggcttgcttttagtcttgaatgtgaggtttttcgctaagtcaggccatccagaccagcaccacagcagaggtgggctaccacacctctggacttggagactggttcagcatggtgcttccttttgtttcttaaagaaaagaaatccacctttgagaggagttgttccccagatgagtttctttttccttggttgttccttttgatccagtattcaagtggtgtactctgacctgggtcactgtcttgtgttcttatgaacagagttcatgaggtcagagttcagcatggctcttagaccaagggcatgggtcttgtggctcaagagctaggtttgcacaagttctttttctgttcacctagagtgtggccagaagctgctccagggtggggtgtgtttctctttgcctagttgcagctgccagaagtccccatctctatggtctgggagtctaaggccaggagaatccctggtttgtggtctcagttcctgtactagaaggggctggccagaagaaaggaccagttcttgcacagtcaagtgcaagggaacaggcagcttgcctggtgtccttcctatcttgaacctggtcctgtgggcctttgaaaagttagatctgtgatctctggggtttccatggctttgttcagtgcctcccctctgagactggcagagctgtctatacttgacaggacctccttgaactcctaggagagcctatagagaaatctgtgaatattatgatgcagattctttgatattcatacattttcacctccagtcagcaatttcctaggtaccatgtaaagctgtaaaattgaattattcttgaagccaggtgacagctgtgacgcctggggtacatggagctgcacgtcctctcccacctcctcaggtgttttgtctcttcatgtcatccctgacaagtgaggttgggaggtggattgggagtaatagtatagtagagtatggtgagtgaaccctaacaccaatgggagctgattgtgacccaggagtgtccctgctgcaggcccagggtggctgtgatgctctgttagcacacagacctttccatcaagtcctgccagtggcactgccgtgacacagcaccagagttgcagctctgcagcctaactcccccttggtactttctgaactgcacagctgccatcctccactcctcagctgtgaccctgaccattggcactgcaggaggctctgccaacctgtagctggtggcgtctcatctgtggaaacctttgtttttgcaccctcagtggttgtccttgttaacatcagaacatctgaaaggtcaggcagaggtagggtttggcttggtgtttttaccctgcaaagggtagagaaaggtgtttccatgacctgaccagattcttgtctttcttcaaagaaaagttgaggtaatgtcatctcgaaaagttgagtgatttattcaaaataaactcattttctgattataaaaaaaaaaaaaaaaaaaaaaaaaa

SEQ ID NO: 3 - DNA, 6171 bp, Synthetic construct
  source 1..6171: mol_type = other DNA
gcgcgtccccgccctcgcgtcacgtgacgtggccgcggaacctgagctgcggggcctaagccgagctaaattcgttgcaggtggccgcggcgggtgcaaccacaaaggctaatccgaaggagtcggggaggctcgtggagtcgatgcttcctcttccaagtcaggtcggctcccgttaccttctcagcattcgccgttccggtcttcctgagcgcgtgcatgaggtctttcgcgtggggaagctccggtgaccatgtaggggagaagagtgaggaagctcctggtgcttgggacgaggtcagcgctgtcggcgcgctgctccagcgcccaccacaccctggtgcgggccgacgggccctgggccctggtgggagctccggcctccggtcaaggcctggccgggagcgccacgaattctcgcgtcgtctcgcgagagtccaagttaaagaacatggcgacgtctaatctgttaaagaataaaggttctcttcagtttgaagacaaatgggattttatgcgcccgattgttttgaagcttttacgccaggaatctgttacaaaacagcagtggtttgatctgttttcggatgtgcatgcagtctgtctttgggatgataaaggcccagcaaaaattcatcaggctttaaaagaagatattcttgagtttattaagcaagcacaggcacgagtactgagccatcaagatgatacggctttgctaaaagcatatattgttgaatggcgaaagttctttacacaatgtgatattttaccaaaacctttttgtcaactagagattactttaatgggtaaacagggcagcaataaaaaatcaaatgtggaagacagtattgttcgaaagcttatgcttgatacatggaatgagtcaatcttttcaaacataaaaaacagactccaagatagtgcaatgaagctggtacatgctgagagattgggagaagcttttgattctcagctggttattggagtaagagaatcctatgttaacctttgttctaatcctgaggataaacttcaaatttatagggacaattttgagaaggcatacttggattcaacagagagattttatagaacacaagcaccctcgtatttacaacaaaatggtgtacagaattatatgaaatatgcagatgctaaattaaaagaagaagaaaaacgagcactacgttatttagaaacaagacgagaatgtaactccgttgaagcactcatggaatgctgtgtaaatgccctggtgacatcatttaaagagactatcttagctgagtgccaaggcatgatcaagagaaatgaaactgaaaaattacatttaatgttttcattgatggacaaagttcctaatggtatagagccaatgttgaaagacttggaggaacatatcattagtgctggcctggcagatatggtagcagctgctgaaactattactactgactctgagaaatacgttgagcagttacttacactatttaatagatttagtaaactcgtcaaagaagcttttcaagatgatccacgatttcttactgcaagagataaggcgtataaagcagttgttaatgatgctaccatatttaaacttgaattacctttgaagcagaagggggtgggattaaaaactcagcctgaatcaaaatgccctgagctgcttgccaattactgtgacatgttgctaagaaaaacaccattaagcaaaaaactaacctctgaagagattgaagcaaagcttaaagaagtgctcttggtacttaagtatgtacagaacaaagatgtttttatgaggtatcataaagctcatttgacacgacgtcttatattagacatctctgccgatagtgaaattgaagaaaacatggtagagtggctaagagaagttggtatgccagcggattatgtaaacaagcttgctagaatgtttcaggacataaaagtatctgaagatttgaaccaagcttttaaggaaatgcacaaaaataataaattggcattaccagctgattcagttaatataaaaattctgaatgctggcgcctggtcaagaagttctgagaaagtctttgtctcacttcctactgaactggaggacttgataccggaagtagaagaattctacaaaaaaaatcatagtggtagaaaattacattggcatcatctcatgtcaaatggaattataacatttaagaatgaagttggtcaatatgatttggaggtaaccacgtttcagctcgctgtattgtttgcatggaaccaaagacccagagagaaaatcagctttgaaaatcttaagcttgcaactgaactccctgatgctgaacttaggaggactttatggtctttagtagctttcccaaaactcaaacggcaagttttgttgtatgaacctcaagtcaactcacccaaagactttacagaaggtaccctcttctcagtgaaccaggagttcagtttaataaaaaatgcaaaggttcagaaaaggggtaaaatcaacttgattggacgtttgcagctcactacagaaaggatgagagaagaagagaatgaaggaatagttcaactacgaatactaagaacccaggaagctatcatacaaataatgaaaatgagaaagaaaattagtaatgctcagctgcagactgaattagtagaaattttgaaaaacatgttcttgccacaaaagaaaatgataaaagagcaaatagagtggctaatagagcacaaatacatcagaagagatgaatctgatatcaacactttcatatatatggcataattttgaatatcatggacaatatttagaacccaaattttggagtgcttgggcagaaagttgtaaagtttgtgctggagaaaggtttatttggactttgattacataaatattaaaatctctgccttaccttacaaaaacaactatattttgccaatcacattagttagcatgatggcattcccttcatgttgcacactctttgacagcatgctgttttgtggagaaacttgcattcatgaagagcccattatcaacttttaaaagtgaatttgatttgtacccaccaggagaaatacagttgggaagggtgagggtggggttcttgttgcttttttctcttttttcctctcttaaaaaatgtacttgcacaaggaaggatcttcagatattcatgcactgaaaaatgctgttatcttttgtttttaaaaaatgcaattaaaactagaaatagcactcttggtttcttttgatgaaaagagtgagctggtcaatgcaacattgggaaacggcagatgtaggtgagaagttaagtatttaatagtatcaaattgttcaaacattgctcagtataataactggattttaatggtgacctaaaaatggtattaaatattttcaactttcaaatgttggttttctttaagcaggagactgctattttagttgttgttaaatgatgaggagtttttttttttttaaatatggctggaaagcttgttttgaaaaatgaagcttatattaattgttgcttcaatagtttaaaaaatttatggagataggtaggtcattatgattgcagagccaaaatagcaaaacaaacatattaagtgtgtttactaaacatattgtgtgtttactaacacacttaatgtttatatacctaatatttattatgccatatctgaatttattaaaaacaacacagaaatctcctgctctgatatagttatgtaactgatctcttaatctgtagcatagaagtattttggtattttaaggtctcatgattagggatttgtagcctcaggtttgaatcagtcacctaggggtaagagaaaaaccacaacataagacctgaaaaacagaagcaaaattgttgcactcttaatacgagcagtaaacttgccaaatatatgtacatatatatttatatattttaaaaataaacattttttaaatcgtcagaaggcaacatttaccatgttcctttcagtctatccaaagtagcaacttaactagttgctggcaactgaatacccagagtagtggaaattttaggacttgaggacgtgaacaacttgaaagagaaaattgattccccttgaggaaagaatggctcaacgtggatttttattcattgtggtgcacgtttcaaattttcttgcaaattattttatatcttatttgatgttaagtaatatttttaaagtatggtttttgaagatagtaggaaatggagtacagaaaggcatacagtattatgttacagtggagtgctttcaagagcatttcgtaggatctgtccccatccgaagaggctttgtgaactgcctgctgaatggaaggaaagcaactttaaaacatttagagttttgctgagtatagccaaatgcctgttctgaaacttataggaaattggaattttgtttaaaattttacactttaaggtagcaaatgtcaattattagaccgagcatattgtgaagaattaaactaaaattaatacgtacagttttctctaattaggtgactgttcataatgggtatatttggaatttagaagaaataattttgtagtaattgtggcccttatgtttaacagataattcagcattggcgtatttgcttgtcccaatacaagaatgccaaaggaggaaacaggaaaaattgccgtccattttaaaaatttaggtttgttgttttaaaccataattgttctcagaacttttttggaacttctaattttgggtcatttaatttaagaatgagagtcatgatgttttgatttatgaaggtgaatttaatgcaaaagtgggtaacactacattcatagcaaagttttttattaaattttataaatttttaatagccaattatgtcctaattgtattttggggaatgaacagcagcaaatactgtaaatgtacagttttcttttttttttttttttggttatgtatacaaaagttttaactactttttggtgtttatgaaaatcatttagttgacaaggtgcctatgccattgttaaattttctttaagtaatttaacaacagatttgctaatttaaaaaaaaatgaaaaaaaaaatcttaccagcagtttgtaaaggtctggaatcacagttggcatcctttattcaggtctcttctctccagcgtacacttaaaattggtgccgagcagggatataacctgcagttaagtgaaaagaaaatccagcctccccctccaaaaaaaaaaaaaaaatttaatttttaaaaattagtggtatggcaataagacacttcagaggctatcttaacctctgaatacccatcttctagtttaaagacagagacatcccatctggaaaatgttaacttgtgttgccatctcgttgccggagtaagtagacataagacagagtttaagaagtaaaaatatagaaaaattttgatggtcacaatgagataaatattagaatattactattccaatgattaaatgaggatcttgaaataaattctgaagtcttccaatttttacatttattggaggggtccctgagttctgtcaacttttttatttaagtctcttgctcttattttgtgcataaatgttaaaccttccaaaaatgaaatgttagctttctttcttttactttttattaaatttaatagaaaatatgacctgagtagttaaaaagtattttgcattatttgcagtaagatgtctctagcactgctcaaagggcaaattttaaaacttcagtctgggtgaaagatttgctagttttacagaaagatttgctatcttaaactcaagctggtttttctgttctcatgtaagtgactgggatgctgtcttatgaattcttccaaggtcatgtttgtgaaataaacattacatgagagctttcctgtcatctacactatatgttgtctggagtgttgaacaaatttattttagtttctaagttgtaatctatcctcatatggtctatacgattttgaatgtgtgccactacatactgagatgataatgctgtacaattttaagtggtagcagtttctgtatgcagtaggctgaaatattttgatgaactgcttaatttttggattttattttttaagttgtataatttattttcttgcaaaataaaaatgtaatataaaa

SEQ ID NO: 4 - DNA, 6024 bp, Synthetic construct
  source 1..6024: mol_type = other DNA
acgcccccgcccgccgcgtcacgtgacgccgccacggaccctgaggtgcgggccctaagccgagataaagtcgttgccggcggcccaagcgggtgcaagcccagcggcagaagcgaaggcgagctggggaggcccgaggaagtggctactacctcttcccggtctggtcggctcccggtcccttcccaccattcgccgctcgcgtctcctcaagcgtttgcatgcgctctctcgcgtgggcaggccggggtgaccatgtagctggaaagcccgaggaagcacggctgcccgggacgagctcggcgctgacggcacgccgctccggcgtcccccgcatcccccgccgcggcctgcggggtcctgctgggaaccccggcctctcgaggaggcctggccccgagcgccgccaagtctcgccccgtctcgcgagagtccaagttgagaacatggcgacgtctaatctgttaaagaataaaggttctctccagtttgaagacaagtgggacttcatgcatccaattgttttgaagcttttacgccaggaatctgtaacaaaacagcagtggtttgatctattttcggatgtacatgctgtctgtctctgggatgataaaggctcatcaaaaattcatcaggctttaaaagaagatattcttgagtttattaagcaagcacaggctcgtgtactgagccatcaagatgacacagctttgctgaaggcatatattgttgaatggcggaaattcttcacacagtgtgatattttaccaaaacctttttgtcaattagaggtgactctattgggtaaacaaagcagcaataaaaaatcaaatatggaagacagtattgttcgaaagctcatgcttgatacgtggaatgagtcgattttttcaaatataaagaacagactccaggacagtgcaatgaagctggtgcatgctgagagattaggggaagcttttgattcccagctggtcatcggggtgcgagagtcctatgttaatctttgctccaaccccgaggacaagcttcagatctatagggataattttgagaaggcatacttggactcaacagagaggttttatagaacacaggcaccctcatatttacagcaaaatggtgtgcagaattacatgaaatatgcagatgctaaattaaaagaagaagaaaaaagagcactccgatacttagaaacacgacgagagtgtaactctgtggaagcactcatggaatgctgtgtaaatgcgctggtgacctcctttaaagagactattttagcagaatgccaaggcatgatcaagcgaaatgaaactgaaaagttacatttgatgttttccttgatggacaaagttcctaatgggatagagccgatgttgaaggacttggaggagcatattataagtgcgggcctagcagacatggtggccgcagctgaaaccatcactactgactctgagaagtatgtggagcaattacttacactgtttaatagattcagtaaactggtcaaagaagcttttcaggatgatcctcgtttccttactgcaagagataaggcatataaagcagttgttaatgatgctactatatttaaacttgaattgcctttgaagcaaaaaggagtggggttgaaaactcagcctgagtcaaaatgccccgagttgcttgccaattactgtgacatgttgttaaggaaaacgccattaagcaaaaaactaacatctgaggagattgaagcaaagcttaaagaagtgctcttggtacttaaatatgtacaaaacaaagatgtttttatgaggtatcacaaagctcatcttacccgacgtctcatattggacatctctgctgatagtgagattgaagaaaacatggtagagtggctaagagaagttggtatgccagcagattatgtgaacaagcttgctagaatgtttcaggacataaaagtatctgaagacttgaaccaagcttttaaggaaatgcacaaaaataataagttggcattaccagctgattccgttaatataaagattttgaatgctggtgcttggtctagaagctccgagaaagtctttgtctcacttcctactgaactggaggatttgatacctgaagtagaagaattttacaaaaaaaatcacagtggtagaaaattacactggcaccatctcatgtcaaatggaattataacatttaaaaatgaagtaggtcagtatgatttggaagtaaccacgtttcagttggctgtgttgtttgcatggaaccaaaggcctagagagaaaatcagctttgaaaatctaaaacttgcaacggaactcccagatgctgaacttagaaggactttatggtctttagtagcttttcccaagctcaaacggcaagttttgttgtatgaccctcaagtcaactcacccaaagattttacagaaggcaccctcttctcagtgaaccaggacttcagtctcataaaaaatgcaaaagtacagaaaagggggaaaatcaatttgattggacgcttgcagctcactacagaacgaatgagagaagaagaaaatgaagggatagtccaactaagaatattaagaacccaggaagccatcatacaaataatgaaaatgagaaagaaaattagcaatgcccagctgcagactgaattagtagaaattctgaaaaacatgttcctgcctcagaagaagatgataaaggagcagatggagtggctgattgaacacaggtacatccggagggacgaggccgacatcaacaccttcatctacatggcctagccgggccgctgctgccgcacacacgcccctgaaggcctgggcagaggctgtcccagccccagctggaggaagctttatttggactttgattacataaatattaaactctgccttaccttacaaaacgactctattttgccagtcacattagttagcatgatggcattcccttcatgttgcacactctttaacagcatgctgttttgtggagaaaattgcattcatgaagagcccattgtgaactttcaaagtcaattcaatttttacccacctagagaaataacatgtcggaagggtgagggtggggttctttttgcttcttttatccccttttcttcttcaaagaaatatacttgcacaaggaaggattttcagatattcatgcactgaaaaatgctggggatttttgtttgtttgtttgggttttttaaattctttttttttttttttttttatgaaattaaagctagacgtagcactaagctacttctgatgcaaagaatgaatcgtcaacactgtgctgggcaagggcaggcacacttgagaagacacgtgtgccagtgtcaagttgtttgtcaaacactgctttcactttagtgactggatcttaatggtaacctgaaatggtattaaatatttctaccttataaatcctgattttcaatgagcaggagagttctgtttagttatttgttgttaaatgataaagatttgggatttttcttttaaaccttgtacggctggagagcttgttttgaaaacatgaagtttataatgaatgttgcttcagttaaaaaatgtgtgggtatacgtacattattgattgcataatcagaatcttgaagcaaaaattaagtgtgttactaatacactcaattctcatacgtctaatatttattatactgtacctgaatttgttgaaaacaatgcagaaatattctgatgcagtgcagtgagagaattgctttcttaacctgtagcatagaattatttggtgattgaaagtgttgtgattagggatttaccacctcagttttctggaaagacattctctatttaaaagtaataattacctgccacttagcaggaagaaaggtgagattacaacctgcaacctgaaaagcactcaaagttgttagtcttaacatgagcagtaaccttgccaaatatgtacatatatttatatattttaaaaataaacatttttaaaatgttcagagggcaacacctaccttgctcctctcagtgatccagagcagtgactcaggactttacagagcagtggaaacttagagattgaggactcaagcaactgaagagaattattccccagcaggaaaaatggctccctgtggattttactcattgtggtgcacgtttcgagttttcttgcaaattttagaacttttgatgttaagtagtattttgaagtattggttctgaagctagcagaagatggagtgttgagaagcatacagtattatctatcctattaccacagagggctgtggatctgccctgctgctcagcagctggggtctccttgctggaaagaagtaaagtatatgttagatgtttaaggctttgataattatagtcatatatctgttgtgaaactcataggaagttggaagtgcatgtgtgcttgctgtgccctgcagctctgaggtgatggctaaagcaggtgtgcagcgcaaactcagtccctgctgctctaccacgtggaggatggaacatccggaatttagagggatagttttatcatgactgttcaatagcttggtgttggcaaattttcctgtcccaataccagaatgccaaaggaggaaacaatcaagggaaaaattttaggccatttaaaatgacaaaaaagatttgtcattttaaaccatcattttctttagaacatctttgaaatttctaatcgctttggtcatttaagaatgagagctgtgatgttttgatttataaaggcaaacttaatgcaaagtgggtaacactaaatccatagcaaagttttttattaaatttttataaattttttaatagttaattatgtcctaattgtattttggggaatgagcagcagtaaataccacagacttaacagtttcatccttttttaaaaatatatatataagagtgtaactactttttggtgtttatcaaaactatttagttgacaaggtgcctataccattgttgggattttctttaaatgttttatgaatagatttgataatttatctgaagatccagaaccactgacagcgcccctctctctccctgcagacttcatagtcttgctatctgagcagggccagacatgcacagggagggaaggagaggccagctcccactcacactgggaggcattgttaagtgaacggtaccgcaggctcagatttgagcacttctgtctacctaaggtggcgccatctgtaaatgctaacctgtcaccaactcacggccagagtaaggagacagggaacagatgcatgcagtggaaaggggagactcctggacgcgcagttagacaaatgtgaggccgtcatcagtctaataatttataaggatcctcaatcaaattctgaagtctccgactttcacagttcttggaagggatccctgagtttaccagcgtgctcactgaaagctctcactctttgtgcagaaatattaaagtctccgaaaaggtgaactgttaatcattcttattttttttaactcttcattaaattgaatatgatatcattagctctgctccaagggcaaattttcaagtttaatctgggtgaaatatttgctagtttacagaaagatttgctatcatatcaatagctggctcttctgtttttgtgtgaatgactgggatgctgacacaagttgtcccaaggtcacagttatgagagaaacactgttggagagcgttcctgtcatctgcactatgtgtggtctggagttcctaaggtgtagcctctcatcgtgacctgtacagttttgaatgtgcaccactacatacccggatggcactgtacagtttcccacggtagcagtctgtatgcagtaggctgaaatattttgatgaacgcttaatttttggattttattttttaagttgtataatttattttcttgcaaaataaaagtgtaatataaaacatttcatctatccagaaaatcttgatgttctaccataaaaatttttgcaacagtaaaaaattttggcaagcca

SEQ ID NO: 5 - DNA, 6136 bp, Synthetic construct
  source 1..6136: mol_type = other DNA
agatgaagaggaggcggtggcagtggtggaagaagaggcggcggcggcgggggtagggagcctggaaacgcgagcggggatggtaggtggtttggacccgccgggccgccgtcgtttccagaaagggtttgactggaggaacctctggagcagctgttggctggctcctctggctgatggcatgttgaggtacatgggccagcggcagcgagggcatccaatccagaggggtccactctagaggccaggccaccagcaccatgggccagtgtgtcaccaagtgtaagaatccctcatcgaccctgggcagcaaaaatggagaccgtgagcccagcaacaagtcacatagcaggaggggtgcaggccaccgtgaggagcaggtaccaccctgtggcaagccaggtggagatatcctcgtcaacgggaccaagaaggccgaggctgccactgaggcctgccagctgccaacgtcctcgggagatgctgggagggagtccaagtccaatgccgaggagtcttccttgcaaagattggaagaactgttcaggcgctacaaggatgagcgggaagatgcaattttggaggaaggcatggagcgcttttgcaatgacctgtgtgttgaccccacagaatttcgagtgctgctcttggcttggaagttccaggctgcaaccatgtgcaaattcaccaggaaggagttttttgatggctgcaaagcaataagtgcagacagcattgacggaatctgtgcacggttccctagcctcttaacagaagccaaacaagaggataaattcaaggatctctaccggtttacatttcagtttggcctggactctgaagaagggcagcggtcactgcatcgggaaatagccattgccctgtggaaactagtctttacccagaacaatcctccggtattggaccaatggctaaacttcctaacagagaacccctcggggatcaagggcatctcccgggacacttggaacatgttccttaacttcactcaggtgattggccctgacctcagcaactacagtgaagatgaggcctggccaagtctctttgacacctttgtggagtgggaaatggagcgaaggaaaagagaaggggaagggagaggtgcactcagctcagggcctgagggcttgtgtcccgaggagcagacttagtggctctgtcccaggagcagcagcaaggatctgccagctgccctgcagccaactgaggaattggaccattttggaaattactgaagatccggatattttctactttacacctttctctgccttgtatctgaaagggctctaaaatgctgtatcatgttttaggcactttcttcatttttttggttattttggttatttcctttttggggggatctcccagaatatttgaacctggttacatgttgtgtatctttttttgaagccttcagatagaataagcctgccatttcttgcacaaatttaggttttttttttgtttttttttgtttttttttttttttttttggtaggggagggcatagagcagggcggggggatgggactgttaggttgaattaacattacaaaatgatacagtgccagatctcagtttcgcatattgtttttcagggcaggtctgtactgtgtgtagtgctgtttacatagatgaatttaggttgtaataattatttttaaagatttacacagatttgaatagcagtgttaactgttaaccacattgcattaattcccaggcgatttagagctcttggagagccaaggccagccaagagcatttgtagtctggtgacaacccccttttaagctaatttatccagaaccctgatttccctcacttcttgctcattccttctttgacctattgcatttcatgttgagtttttccatcaacatgctgcacctgtcagtcaagtgagcattttttaagaacacattgtactgagaaccacttaagcattgaatgcggagaaagcagtgctacctcagttttgctggaagtagacttctttgatagttttctttctttgatgaagtttctgtattttcatgttgtaagtggaaatacttttttttgtttgtttgtttcatttgccttggagccaaagtttctgttcctggtggtcgggaaactgcctgccggccaactgacttgaaggaaaactgtggtatggagctctgcttgaatttttttttttttaatatttttatttttttctttgaatatcatcagcttacttgtctggcaagggcagaagcctggggttggcctgaactctgccaaacaaatatcaaagtgtatttaatagttaaatttgtgccctttcccttcttgctgcacccatgttgtcacttaacccccaggagttatttattatctttttgttaaagtcaggctcatttggggtaatgtgatgactgtttaggtttacatgaccctcctctcctttccctacccccaaatatgtatatatacatatataaaatatgtatatattttacctatataaaatatatatatatacacatatatgtatctatattcctttgtttctttgcctgcttatactggccataaaagagggagctgccttcaatgtataaagtataagaagagtgccagggaatgccataatggaggcttttggatctgaatttggaccatttcactaaagagaacatgagtttgctcagccctttcctcacaagagggagggccccggttccccagacttctccacgcgctggctccataaaggccagctttggccaggctgccacaggggcctgaggagctcactctgggcctacctggtttcagttagagggtcctcctgttatttttccatttaaaaagtatgtcctcagaaaactgtactggaaggatgggtggcaggaacttgtatagttcagcttccaacactttggaacagattaaaaagggaatcttttaaataaaaacgtataaaaatatttatactcttgaggtaatgagactttgtctgttaaacatttggaggtttctcccttaacctaataccccgctgcgtcccaggtatgtttctcagcctgggcaggaattgcatgttttgagttccattgtgagcagccttacagctctgggctcccatcttaaggattattaaagagttttggcagttggtatttttctaaaactgatattgaccatagggaacatacttggaatttgtttttttttttaaatcctaaacctgacagttcctccttcagaggggcaaatgggaaactttaagatcacccacggtatagtactgctagaaagctgaagggggtcttgggccagatgcctggcttctgttctagctaaataagactcttgaataagtctccaggggtcccagcaggttctgtgggatcagttgtagctgggtacccagactgaggttttaaaaagactgctttttcattaagccagtttgcaactcattttccccggttctggattgggtagtggttgactgagggaaaagccaggctgctcttggccagataaggccctttccatcccagatcagagtgtatcagagaaagctgcacaaaagaggcaggcagcccaggaccctgagtactctggagaaactaggttccttccccaccctttaagaagacattccgtgcattagatgtactagagtggatgtattttgttgttttttaaattaactatttagcctcctcatcccccaccaaaaaagccatttagttattttttggttatattgatccatttgcaaatgagaagccagaaaagggagcagtcagggagggacttacaagttcctttcaagtttgagtacttgatgctcagcaaagatttcaagcttctgcagtagctctgggccaatgcttgactctttcatgaccacaagaaatgcagtttttctgcaaagggatccaaggtgaggtgtgtgtagggtttgaagttatacttctgggaagtgaaaagtcttgttcctttcaacctagaaataggtttgccacttaatgagtgagcagtaagtctgtgtaagaggctgaatgcatgcccctcagataagccagtacactccttgcttagcaacagaacatcagggtgatgtggagaggggcaggatgtggacgccactttggaaatcggcaacatctgagggcaacaacaaacaagtgtgttgggaaataagaaataactcagttttgacaactggctttgtcagcttttgtgatgtttctttagcagtttattggaaagatggtatgagatgacgtgctgcttcattgaattgctctttcccccatctttgccaaatctcaatgtatcgttcttaaccccacctcctgtaaggggctttgctatgcttcagctggttgtctcagcagctgaagtgctgcccacctgtgtgagttgggtccaggaaaccatgtctgcccttctgataagggaagatgaatctagagctgggtgaagatctaaattttaaccaaacccctgggcccaggaaaataacaattgaaaatgtacaaggcagtgttttcaatattaaacttccccaaggaaagcacaaactagtctttttggaaagggagaaaggattaagccacacagtattagtctttgaagcagtactggtctctaggggctggtgccaaaatggagtcccatagtagttacactcgatggcctcatgtactatatactgtgccaaattgtattaaacagtggtggggagttactgggataagaacttgtctaaaagtttacaaaccaaaacagatctgttaggttggtgcaaaagtaagtttttgccatacttaatgtattgccattaagtatggcaaaaaccacaattacttttacaccaacctatgtatttaagaatgtttggtttgccagattccaaatgaggtcttcagtgcagcaaagcccaaaaggtgtagactcagttatgcaattataaggttaaggcgtagaagaaagctgctgctaggtttttgttgcattttacttgactgctctgctgtttttcttgtctctcatgtttggttagctatgacttgagcatcttggtaactgacaaaggtcttccttgggggacttgaacatcttggtaaatgacaggtcttcttggaggactccagcagtatcttgtttaaacgactgaaaggactattaaggttgttgaattgtgttaattgggactcattgaggaaatgcgacattgatcctcctcttattccacagtgtgttttctgatcatataaagaaggttccgaaccatccatccccctcagagtttattcccctggtaagctgtaattgcatatccagtttaaactggactgggactgcatgttggtgaggatcggcaggggttttcccccttttcgaaagatgaaatagattcttgagcactggttgcagaagccaaaatagttcaaatagctttgcataaccattgggttctgcttctgattcaggtgctgggcatcatgtcctccctattcttctcttcttggaaacccagcctatctcataaatacctacttccgccacccatccaacctccctgctcctttcaacacaaatcttggatattgccaaaggaagccattcagcagctgctggggtttttcatccccctgacatgcatacatttgctctgggagaagtgtcttccctctgaccctggtccccagctccgtctgtgcttaattgctcttacctgttgcactaatgaatatgactaggttttaaagggggaatgtgaagcaataggcacatggggcttggatgaattggtcccacagatataccctgccttaagccgctgaggtgatgagtccactgctcatgtgaccctccacctttgtggatccctcttggtttgtgaccagtgtgtctgtttgttgaggttgtacaaacttgacaaaagttaatacttttgtttgtattttctgcactgttgcactctccaaatggccccttgagtatttttattgacttgttacacacatttttgtctttgatgtctacattttttcctttaatgttttttatttggaaggttacctgctgttggatttaataaatttgtttacttgaa

SEQ ID NO: 6 - DNA, 5637 bp, Synthetic construct
  source 1..5637: mol_type = other DNA
gaggagtgcatatcagcgccttgtctggactggtccctctggaagaagaggctgtgaggggagcctgtggcttgtcaagcgcaaggctccgaaaacttacctactgtccctgcctgtccatcgccagcacccctcaatcccgggcctggggacctggggctgtagatccccggggagggtctgcagactctgagctgaaagtccagggttgcctggctcctctaactgatggcatgttgaagtacatgggccagtggcagcgagggcatttgatccagaggggtccactctagaggccagggtactaccaccatgggccagtgtgtcaccaagtgcaagaatccttcatcaaccttgggcagcaagaatggagaccgggaccccagcaacaagtcacacagcaggcggggggctagccaccgtgaggaacaggtgccaccttgtggcaaaccagctggggatattcttgtcaatgggaccaagaaagcagaggctgccactgaggcctgccagctgccaacttcttctggagatgctgggagggagtccaagaccaatgctgaggagtcttctttgcagaggttggaagaactattcaggcgctacaaggatgagcgggaggatgcaattttggaggaaggcatggagcgcttttgcaatgacctatgtgttgatcctacagaatttcgagtgctgctcttggcctggaagttccaggctgctaccatgtgcaaattcaccaggaaggagttttttgatggctgtaaagcaataagtgcagacagcattgatgggatctgtgcacggttccctagcctcttaacagaagccaaacaagaagataaattcaaggatctctaccggtttacatttcagtttggcctggactctgaagaagggcagcggtcactgcatcgtgaaatagccattgccctatggaaactagtatttacacagaacaatcctccagtattggaccagtggctaaatttcctaacagagaacccttcgggaatcaagggcatctcccgggacacttggaacatgtttcttaacttcactcaggtgattggccccgacctcagcaactacagtgaagatgaggcctggccgagtctctttgatacctttgtggagtgggaaatggagagaaggaaaagagaagtggaagggagaggtacactcagctcagggcaagagggcttgtgtccagaagagcagacttagccgctctgttccaggagcagcactgaggatctgcctgctgccctgcagccaaatgaggaattggacctttctggaaattactgaagatccggatattttctactttacacctttctctgccttgtatttgaaagggctctaaaatgctgtatcatgttttaggcactttcttcatttttttttttttttgttattttggttatttcttttttgggggtgggaaatcccccaaaatatttgaacctggctacatgttgtgtacctttttttgaagccttcagatagaataagcctgccatttcttgcacaacttgataggatttgttttgtgggggaggggagggtgtagcccgggttgggagtgggttgttaggttgaattaacattacaaaatgatgcagtgccagacctcagttttgcatattatttttcagggcaggtctgtactgtgtgtagtgctgtttacatggttgaatttaggttgtaataattattttaaaagatttacacagatatgaatatagcagtgttaactgttagccacattgcattaatttccagacagtttggagctcttggagagccaaggccaaggccagtcaagagcatttgcagtctgacaccccaccccgtccctttaagctaatttatccaaagccctgtctccctcacttcttgctcattccttctttgacctattgcatttcatatttaacttacccatcatcatgctgtacctgtcagtcaagtgagcagttttgttttgttttgttagaacacattgtactgaaaaccacttaagcattgaatgcagagaaagcagtgctacctcagttttgctggaagtagacttctttgatagttttctttgatgtttttgtattttcatgtcataagtggaaatacttctttttctccccttttcatttgccttggagccaaagtttccgttctgggtggtgagaaaagtgtgcctgccagccgacctgaaagaaagcggtggcatggagctctgcttcaaactgttttttctctagttttgttgagtatcattagcttacttgtctggcaagtgcagaagcctgaggctggcctgaactctgccaaacaaattatccacgtgtattgaatcattaaattgtgtgccctctccctctttgctgcgcccacgttgtcacttaacccctaggaattatttattgtctttttgttaatgtcaagctcggttggggtaacgtggtgactgtttaggtttacatgaccctcttctctttcccttaccccagacatgtatatatacatatataagatatgtatatattttacctttataaaatatatatatatacacatatatgtatctatatccctttgtttgtctgctaaaactggccatgaaagagggagctgccttcaatctgtagtttaagaagatgccagggagcatcataaacggaggctattgtatattaatttagactttttcactaaaaagaacatgaatgaatttgcttgccgaaggaggaaccagttctctagactccatgctcactctacaaggccagctttggtcaagctgccactggggcctgaagagctgactctgtcctaccttattccactaaagtaccatcttgataatttccccttaaaaaatattctcaaaaatgtactggaaagtgggtggcaggagcttgtacagttcagcttccaacatttcggaacagatttaaaagggaatcttttaataaataaaagttacaaaaatatttatactcttgatacaatgaaaaagtgtctattaaatatttggaatttcctcccttgattagtctaccccaaatcctgggtacatgtttctcaacctgggtaagaattgcatgctttgagttcttcatgagtcttatagctctaggcacttatctttataaaaatgattatttaaggagatgacagtcatatttcttaaagtcggtattgacacagggaacatttttgtaagcctcacaattgtccttccttgagggatgagatgatgaatatcagccacagacttacgatagtgctggaaggcccaagtggctgttgggccagatgtaccccttttttttctagccagaataagatgaaatccttgagtaagaatccaggagcccagtagtttctgagggcacagctacattataacatttggttaagatctcatctagccagttagttgttcctaattcaggattaggtagaaatctaatgagggaagaggtggagttaggtaagactttctgtcccagataagaaagggtgtatgtggggaaagtgccaagagggagtttgccaagggccccatggactcaaggaactagatcttttcctcattgggtagaggtttttttaactgctcagtttctccaatcccaatgcctgcaccccaaccccagccatttagaaattgtgattatatttttaatcatctgttgttgagaaaccagagaaaggaacagtattcagggagggactttgggttttctgtgtagttgctttgctttgccaagagattctcagtgaggtgtaaggtttgcagttaacacttctgggaaacaaaagtttctgtttcactcaccctcagataggcttgctgcttattgggtggatgagcagtggctgggagccaaattgctcctcaggttagctagctttgtggcttcaggtgatttgggaaggggtgatatcagggtgccattttggaattctgcaatgtctaagcattactacacagaccgttggaaattgcatactttgggcaaatgactttttgagtttccatggtaattctttagtaacttattagcaagctgatatgagataaaaagttactttattgaattctttcccttagtcttccttgccaaacttgaatgtatcattctcattgacgctttgctgtacttctgggggttgcttcagcagctctctcttcattgtggcagatgcgccaacatgtctgttgggtccagaagtctgtatgtacctatctgataaagaagttctcagagctgactgaaaatccaaatttcagccaacactttgggccaagggaaagataacagaaaatgtgcaaagcagtactgttcatattagaagggcagtgaagaattaccatgcacacagaattagcttgaaacagtactgtctcctagggtgtggtctccaggccagatcaggttatgggatcccatagattatgtacgtagactgcacattggtgggaagttgctaagataaagacttgtccaaaaactaacaaagcaaaagatgtgtttattggaatgtatagtctgctgtatttcaagtggggtcttcagtgcagcaggtagacctagttatacagttatgtaagggatagagaactactgttaaaaggtttttatcccctctcaccacttaatctcctatgtatgttaggatggctagtgatgacctgaacatcttggtaacagacagaggtcttacctggaggactaaagcagtacttagcttaaatacttgaaaggacaaagttatcaaaatacgttgattaggattatttaaggtcatgtgacactgatcttttcttctgggtcacgtagaggataggttcttgccacactcttattcagagtttccttggtaagctgtaattgcagcccataccagtttgacctggcccaggactacatgctggggatgctcaccaggagtcgcccctggtctcagcccctctcctctttatttcagagaaggatgcttgagcattgggcacagatgctgaggtggtttgattagctgaaaaaccaagttctgattcaggtgttggggcatcatgttctgtttctcttggaaacccagcctgctctcatagatacctacttccatcacatatcctgagcattgccaaaagaagccatttagcagctgctgtttttctctcagcacaaatatatttgctacagaagaattctcttttctctggtcctggtccccagacctcagaatctgttaactgctcttccctgctgcactgaatatggttctaggttttgaagagggaatttgaagcaataggcaatcagggcttggttgaattggtcttgcatatatcctgtcttaatcctctgaggtgatgagacactgctcatgtgaccttctacctttgtggatccctcttggtttgtgaccttcgtaccttccctgaggttgtacaaacttgacaaaagttaatacttttgtattctctgcactgttgcactcttcaaatagccccctgaatattaacttgatatacacatctttctcttttatgtctacagtattttttccttttttttttttgaaggttatctgctgttgaattcaataaatttgtttacctaa

SEQ ID NO: 7 - DNA, 2669 bp, Synthetic construct
  source 1..2669: mol_type = other DNA
cttccccaagccaacgtctccgccgtcggctccgcggcgccgccatggccgacgtggaagacggagaggaaacctgcgccctggcctctcactccgggagctcaggctccaagtcgggaggcgacaagatgttctccctcaagaagtggaacgcggtggccatgtggagctgggacgtggagtgcgatacgtgcgccatctgcagggtccaggtgatggatgcctgtcttagatgtcaagctgaaaacaaacaagaggactgtgttgtggtctggggagaatgtaatcattccttccacaactgctgcatgtccctgtgggtgaaacagaacaatcgctgccctctctgccagcaggactgggtggtccaaagaatcggcaaatgagagtggttagaaggcttcttagcgcagttgttcagagccctggtggatcttgtaatccagtgccctacaaaggctagaacactacaggggatgaattcttcaaataggagccgatggatctgtggtcctttgggactcatcaaagccttggtttagcattttgtcagttttatcttcagaaattctctgcgattaagaagataatttattaaaggtggtccttcctacctctgtggtgtgtgtcgcgcacacagcttagaagtgctataaaaaaggaaagagctccaaattgaatcacctttataatttacccatttctatacaacaggcagtggaagcagtttcagagaactttttgcatgcttatggttgatcagttaaaaaagaatgttacagtaacaaataaagtgcagtttaaaacccaactcttactcttaatttgttcctaatacgtatttttggcagggagagggaacggtccatgaaatctttatgtgatataaggattttaagtttgggccagtgaacagggtaaataaaatttaacttttgagcatatggaattttgattgcctttaaagttactattctgtatcattgattcatcaagaaaacctagatctgcactcacctcactgttcaatcattgagtggtaaaggacagaagtattttctagttctggtcagatgatataattttttttttttttttttgtgggggtgaagtcttgctctgccacccaggctggagtgcagtggcatgatgtcaactcactgcaacctccgcctcccgggttcaagtgattctcctgcctcagcctcccgagtagctgggattacaggtgtgtgccaccacacccggctaatttttgtatttttagtagagacgggatttccccatgttggccaggctggtctcaaactcctgacctcaggtgattcgcccacctcagcctcccaaagtgctgggattacaggtgtgggccactgcacctggcccagatgatataatttaactgtgttttaggtaacatgttaatgagggaaaatgtttaccagtgtagcattggatcaaaaagtttatcacacagtgttttaataaatggtttattctgcttaccttatttgaattgtcattataattttatatgcaaccttaatttgtcaagtaaatactttcatttaatttgaaagaattcatattcatttgctctttcatttgacaataataaaatttaaggtttattacctggaaagagtgcaagaaaatactaaacaactttatcaaactgattcacactgaggggaggttattttgtaacaaataatggaccacaagttcattttgtattcttctgtagtagaggctactgatatgaatttaagaaaaaaccttagaatcttaacttttcacatctgcagatgcagtgtgccattctgaatttttcaatctaagggatgtggatacatagaatgtttaaaaagataacaattagaatgaaaaattaaaattatgtatgcttttatatgggttactttttattgatcacatttctctggaaatttttctttatattttttcacagttgtctttctagctactgaaaaaattatacaaatatctttggccatttaaatgtttatcattgtgcagtgaactgttttagtaactttctgtttgacctttttagaaggttgcatccttaaaatgtgaaccagatacatggctttgcattgaagaggaaagcagtagaatgtcaggttactttaggcagtgtgacagtatataaatgaaatgtggaggatggtggtaacaatttgattttccacacagcaaaaccacagtcccgccacctaacaaagccctgtttatgccacacaccagcatctgagggaagaggaggcactgtgacaaagcagcgctgatgctgctcagcctgcgatgattacagctcatctgcacagccatctgaaagtttaatacatactcatcagagggaacatgccgggacaattttgggttccaatacaaaagttaagcatcaaacaggtttttctttccatttgattaaatgtgctgattttattttactgttgcattagaaacaaacaagttatctttcgctatttaattttccctctatgcaaattgtgttattacagctgaccgctatcagtaaatcttaattctgtcagtcccattgcttccttttttataaaaatactgtgtccccctttactgtcatgaagtgaaattcataaatatatttcctacacatataa

SEQ ID NO: 8 - DNA, 1232 bp, Synthetic construct
  source 1..1232: mol_type = other DNA
gggaggctccgtggcgtgtgttgtggccccgctgagccaccgtacctcccgcctccgcgcccaacgtccgcgtcgccgttctgcgccgccgccatggccgacgtggaggacggcgaggaaccctgcgtcctttcttcgcactccgggagcgcaggctccaagtcgggaggcgacaagatgttctctctcaagaagtggaacgcggtagccatgtggagctgggacgttgagtgcgatacctgtgccatctgcagggtccaggtgatggatgcctgccttcgatgtcaagctgaaaacaagcaagaggactgtgttgtggtctggggagagtgtaaccattccttccacaactgctgcatgtccctgtgggtgaaacagaacaatcgctgccctctgtgccagcaggactgggtagtccaaagaatcggcaaatgagaggtggcccaggcgctcctggtgtggttgctgaccctggacaaagactaaacactgcaggggattcatccttgagagagagaggatgctgtgcgcctttgagactcaccaaaggcttgctttattaatttgtctgtttagttttgggaaattctctacaattaagataatttgttaaaaatggcctttcctacctctggtgtgtgtgtgtgatacgaatgcatagaagagcgagaacaccagaaaatgatctttgtttatctgtacccacgactgtgggaacattgtgttcacagaagaacattgtttgtgtttatgcttgagggttaaaaaatagataaacgaatgttacagtaacaaataaaatgcattgaaaagccgactcctcctaatcctttttgtgttgggagagaggcaagcgaggccaccctgctgtcttcatttgctgtgaatgaggattttaacctgcactcagtgaagaggcgtaactgtcgggtaaactgtaatatggcgtaactgtcgggtaaacggctttgtctcctgacttctccatctttgacttggccaggaagcctggattgttcaaccacttagttctaaagaactgttttctgtttttgcgaaggttgtattgtatgttttagtcaaaatattagtaggaaaatgcttactagtataacactgaagttcattatgcaatgttttaataaaatattgtgctttgagttattaaagtttgatatatactcttaaaatcattaaactaattcatcaattaaataatttgaaaaaattaa

SEQ ID NO: 9 - DNA, 2137 bp, Synthetic construct
  source 1..2137: mol_type = other DNA
acttccggtcccgccgccgggagccggtgcggctgtgaggggccgcgtctcgcagcagccgcccggaccgggcatggtgttgggcgccgggcccgcctcgcctgtctcggggagcccagggtaaaggcagcagtaatgctaacgctagcaagtaaactgaagcgtgacgatggtctcaaagggtcccggacggcagccacagcgtccgactcgactcggagggtttctgtgagagacaaattgcttgttaaagaggttgcagaacttgaagctaatttaccttgtacatgtaaagtgcattttcctgatccaaacaagcttcattgttttcagctaacagtaaccccagatgagggttactaccagggtggaaaatttcagtttgaaactgaagttcccgatgcgtacaacatggtgcctcccaaagtgaaatgcctgaccaagatctggcaccccaacatcacagagacaggggaaatatgtctgagtttattgagagaacattcaattgatggcactggctgggctcccacaagaacattaaaggatgtcgtttggggattaaactctttgtttactgatcttttgaattttgatgatccactgaatattgaagctgcagaacatcatttgcgggacaaggaggacttccggaataaagtggatgactacatcaaacgttatgccagatgataaaaggggacgattgcaggcccatggactgtgttacagtttgtctctaacatgaaacagcaagaggtagccccctctcccgtcctcatgctccctctcagtcccctggattgccccagtcctgtgaccatgttgccctgaagaagaccatcttcatgactgctcattgtagatggagaattcaacataaatacagcaagaaaatgtgtttgggcttctgaagagttgtctgcttaccttaacatgtttacttttttgaacttgtactgtataggctgttggtgaaattcttaagaagttgtaatgaactcaaaattgaggccagagcttgctttcccttttcccaaacaaaattggttttctgcacaagcgatgctaatgatgtgttcagtgtaactcgcagattggcaataagatacccgctacaaactgtgattggatgcaaaatctcttagcttctttcacgaatgttggccctgcctagatgttgtgaagcctcccagaatgcatagagtcattcactgtagatctcttattgaaatgcgtattttatttaatgtaagtatattttggaacagatttgtaatttgtacaattcaatgctttaattattttttctattctcatttagtttgtattttcattgtatagagcagacagaaagatgttgggtcaagcaactattgaagagaaatacaaagaaaatatgaaaggcacattattcattttgtccaaatgcaatgagaatctcactcttaaaaatcagctcttgctttcgggtccggatgtggtgagcacattttggagccctttgaagctagatttggatgatcaaaacaaaaaggcagggagcccattctaacatgctgcccagaggaaactggctggagcctggaccagctggggttgatgcttttgcagtggtcatgtgattgtgacctggtagctacttatcagagagccagaccctgctgtcctgggagacaggagcgatgcctcaggaatcagcccaatgtctgatgtcactgagactgtacctgtggccttcttctgagtttgctatggctccaggccctgccggtggggtgagcctcctaggccttggaggaccaggagtcaacagtggcatatgccatcctcggccaggttaatatactgcagaggaaaagccctgaagagaggcaagtggatttactccagcatgtagacatttgaaccagtgaaatcaaacacaaaataaatgtctgtctagtttcatttgctgcctgcctaacactctcaaacttaactgccagcttccttagacctctggattcccccatctgccatctactgggtttagtttgtttttcttaatttatagaatctctgattgtgagatcagtctgtcacggagacccagcaggtgaggaatgtaggccttgcttcctctttgcacccatta

SEQ ID NO: 10 - DNA, 1892 bp, Synthetic construct
  source 1..1892: mol_type = other DNA
agtccccgccccgccacttccggtctcgccgccgggagccggtgcggctgtgaaggccagcgcgactcagtgccgctgctccgccgggcatggttttgggtgccggctcgcctcgcctgactcgcggtgctcagaagaaaggtggcaggaatgctaacgctggcaagcaagttgaagcgggatgatggtctcaaaggatcccggacatcagcctccacatctgactctacccggagggtttctgtgagagacaagttgcttgttaaagaggttgcagaacttgaagctaatttaccttgtacatgtaaagtacattttcctgatccaaacaagcttcattgctttcagctgactgtaagcccagatgagggttactaccagggtggaaaatttcagtttgaaactgaagttcccgatgcctacaacatggtgcctcccaaggtgaaatgcttgactaaaatctggcaccccaacatcacagaaacgggggaaatatgtctaagtttactaagagaacattcaattgatggcactggctgggctcccactagaacattaaaggatgttgtttggggattaaactctttatttaccgatctcttgaattttgatgatccactgaatattgaagctgcagaacatcatttgcgggacaaggaggattttcgggacaaagtggatgaatacatcaaacgctatgccagatgataagaggagagatcgcaggtctgaggactttgtcacagttgtctctgatgtgaaacagctcgaggtagccctcctccccatcctcatactccctgtcagccccttgggattgtcccagtcctgcgaccatgctgtcctgaagaagaccctcctgactgcccactataggtggcgaacaacataatacagcaagaaagtgtgtctgggcctcaagagagttgtctgcttcccttaacatgtttactgaaactactgtctaggctgtctggtggaatccctctgaagttgtaatgaactcaccactgaggatatagcgcttgcttcctttcccccaacaatcggtgtcctgcacaagtggtgtaatgacatgttccatgtgactggcagattggcaataagaaacccgctataaactgtgattggatgcacactctcttagcttcttccacgaatgttgaccctacctagatgtgaagcttcccagaatgcatagtcattcactgtagatcttactgaaatgcgtattttatttaatgtaagtatattttggaacagatttgtaatttgtacaatttgatgctttaattattttttctattcttatttactttgtattcattgtatagagcaaacacaaagacattgagtcaagaaactactgaagagaaatacaaagagaccatgctaggcacataattcagtttctccagacgcagcagtagtctggctcctagacagcagctcttccttcaggtctacatgtggatgtcttagagccttagaacctacacctggacgatcagaaccaaaggcagggaggcttccctcgattgctacccagacacagcaggctagggctctctaagcaaggctggtaggtgattatgccatggtcatagcatgagccagggctccttaacagactctgtcattgccatcctgcctggggagtcagcccagtctaagactctgccacaactccattatgactgtcttccaatttgccagtggttccaagagggggagtttcatagctgcagcatacgccataatcagggtagtcacttcagggtcagccctggagagacaggtgggcttattctggctttagacatctgaaccagtgaaatctaatgcaaaataaatgcatgtctggttttactcacaaaaaaaaaaaaaaaaaa

SEQ ID NO: 11 - DNA, 2176 bp, Synthetic construct
  source 1..2176: mol_type = other DNA
agtccctgtcctgcgcccgcgcgccccgggagcctacccagcacgcgctccgcgcccactggttccctccagccgccgccgtccagccgagtccccactccggagtcgccgctgccgcggggacatggtcctctgcgttcaggggtgagcacccccttgtaagctcagggctactgttgggtgtcagggaacaaagttttagactgctgcgctccaaagcgggcacacacatgtacctagaacacaccagccactgtccccaccatgatgatgacacagccatggacacacccctgcccagacctcgtcctttgctggctgtggagcggactgggcagcggcccctgtgggccccgtccctggaactgcccaagccagtcatgcagcccttgcctgctggggccttcctcgaggaggtggcagagggtaccccagcccagacagagagtgagccaaaggtgctggacccagaggaggatctgctgtgcatagccaagaccttctcctaccttcgggaatctggctggtattggggttccattacggccagcgaggcccgacaacacctgcagaagatgccagaaggcacgttcttagtacgtgacagcacgcaccccagctacctgttcacgctgtcagtgaaaaccactcgtggccccaccaatgtacgcattgagtatgccgactccagcttccgtctggactccaactgcttgtccaggccacgcatcctggcctttccggatgtggtcagccttgtgcagcactatgtggcctcctgcactgctgatacccgaagcgacagccccgatcctgctcccaccccggccctgcctatgcctaaggaggatgcgcctagtgacccagcactgcctgctcctccaccagccactgctgtacacctaaaactggtgcagccctttgtacgcagaagcagtgcccgcagcctgcaacacctgtgccgccttgtcatcaaccgtctggtggccgacgtggactgcctgccactgccccggcgcatggccgactacctccgacagtaccccttccagctctgactgtacggggcaatctgcccaccctcacccagtcgcaccctggaggggacatcagccccagctggacttgggcccccactgtccctcctccaggcatcctggtgcctgcatacctctggcagctggcccaggaagagccagcaagagcaaggcatgggagaggggaggtgtcacacaacttggaggtaaatgcccccaggccgcatgtggcttcattatactgagccatgtgtcagaggatggggagacaggcaggaccttgtctcacctgtgggctgggcccagacctccactcgcttgcctgccctggccacctgaactgtatgggcactctcagccctggtttttcaatccccagggtcgggtaggacccctactggcagccagcctctgtttctgggaggatgacatgcagaggaactgagatcgacagtgactagtgaccccttgttgaggggtaagccaggctaggggactgcacaattatacactatttatttatttattctccttggggttggtgtcaggggcgagccaaccccacctctatgccctgagccctggtagtccagagaccccaactctgccctggcttctctggttcttccctgtggaaagcccatcctgagacatcttgctggaaccaaggcaatcctggatgtcctggtactgacccacccgtctgtgaatgtgtccactctcttctgcccccagccatatttggggaggatggacaactacaataggtaagaaaatgcagccggagcctcagtccccagcagagcctgtgtctcaccccctcacaggacagagctgtatctgcatagagctggtctcactgtggcgcaggccccggggggagtgcctgtgctgtcaggaagagggggtgctggtttgagggccgccactgcagttctgctaggtctgcttcctgcccaggaaggtgcctgcacatgagaggagagaaatacacgtctgataagacttcatgaaataataattatagcaaagaacagtttggtggtcttttctcttccactgatttttctgtaatgacattatacctttattacctctttattttattacctctataataaaatgatacctttcatgta

SEQ ID NO: 12 - DNA, 3014 bp, Synthetic construct
  source 1..3014: mol_type = other DNA
ttcctggaaagttcttggaaatctgtcaaaggtgtttcctttctcggtccaaagcactagacgcctgcacccccgttcccctccgggccgccgcaaagcccgcggttctaggaagatgaggcttccgggaagggctgggacgcagcggacaaaagattaggaggcgcctggccccgccccaccggcccagcccgtcccccctcgtccttccaagctgttcgcaccacagcctttcagtccctgctcgccgcccgtgtgccccgggaccctgaccttcgcacccctggcacccattggctcctttctccttccatcccgccgaactccgactctcgagccgccgttgtctctgggacatggtcctttgcgtacagggatcttgtcctttgctggctgtggagcaaattgggcggcggcctctgtgggcccagtccctggagctgcccgggccagccatgcagcccttacccactggggcattcccagaggaagtgacagaggagacccctgtccaggcagagaatgaaccgaaggtgctagaccctgagggggatctgctgtgcatagccaagacgttctcctaccttcgggaatctgggtggtactggggttctattacagccagcgaggcccggcagcacctacagaagatgccggagggtacattcctagttcgagacagcacccaccccagctacctgttcacactgtcagtcaaaaccacccgtggccccaccaacgtgcggatcgagtacgccgattctagcttccggctggactctaactgcttgtcaagacctcgaatcctggccttcccagatgtggtcagccttgtgcagcactatgtggcctcctgtgcagctgacacccggagcgacagcccggatcctgctcccaccccagccctgcctatgtctaagcaagatgcacctagtgactcggtgctgcctatccccgtggctactgcagtgcacctgaaactggtgcagccctttgtgcgcaggagcagcgcccgcagcttacaacatctgtgtcggctagtcatcaaccgtctggtggccgacgtggactgcctacccctgccccggcgtatggccgactacctccgacagtaccccttccaactctgactgagccaggcaccctgctctgcctcacacagtcacatcctggagggaacacagtccccagctggacttggggttctgctgtcctttcttcagtcatcctggtgcctgcatgcatgtgacagctggaccagagaatgccagcaagaacaaggcaggtggaggagggattgtcacacaactctgaggtcaacgcctctaggtacaatatggctctttgtggtgagccatgtatcagagcgagacaggcaggacctcgtctctccacagaggctggacctaggtctccactcacttgcctgcccttgccacctgaactgtgtctattctcccagccctggtttctcagtctgctgagtagggcaggccccctacccatgtatagaatagcgagcctgtttctgggagaatatcagccagaggttgatcatgccaaggccccttacggggacgcagactgggctaggggactacacagttatacagtatttatttatttattctccttgcaggggttgggggtggaatgatggcgtgagccatcccacttctctgccctgtgctctgggtggtccagagacccccaggtctggttcttccctgtggagacccccatcccaaaacattgttgggcccaaagtagtcctgaatgtcctgggcccatccacctgtgtatggatgtgcccacttttttctcccaagcctcttttgggaggctgggtggccagacagacaggagccagaaacacaagggctcccactcttctcctcacagggcagcaccatggcttcatagagctggcttctctatgttgtgccccacctcacccccctgccgaggggcgtgtgctgggtcgggaagtggatgcttatccaagggccgcagatgtagctcccttgtgtccgtttcctgcctaggaagttgcctgcacgtgagagagggagaaatacatacacacctaacaagactttagaaaacaagtgttagaacacaagaaccagtttgggagtttttcttccactgatttttttctgtaatgataataaaagtatgccttccacttatgaagccttcagtggggtgtctggagtttcgcgtggtttgacctctttgattctcgtgtctcgatccacgtcacatctggaaaagccaaaacttcaatgactccataggctttgccagagggctgcggggcgggggaggggccttcgtcctgttccccttgggagatgagagacaaggggtgaacgcccattggcatgccttctggggtgctatccattgtgctgtggatatgtgagaagcaggcagcagaggatctacaagcccaaaaggaagggtccattcaaagtacaagtgggcacggagggcagtaacttgccaagaggagccacagaactcaggccggccttgcaaggagaaagtacagtggataaaagtgtactgaagtgccatgaggatcccggacatggacctgacaggcctcttcctgggcagtgggtagatcagttgagtcagggatagtgaccaatgagctggtagggtcaggtctggtagtgtcaaaattctggctcaggccgtttggagtcaagaggactcttctcttagtcgagatcttgaacatcagaaatacaaggaaaatgaaaaccttgtccagggttgaaggtctgactcgggaatatgtaggctgctgggatcctgctgaagtatgagggtgtcacagcaaagttcctccaaactgtgctcctagccttttacatcgtgttttagagacaattcagaatgggttcctagtcctcagaccaagtcgaaagttaaaagaggccaataatagtggcctgagagacggaagctgtaaggaatggcatcttgataaggaagagact

SEQ ID NO: 13 - DNA, 2969 bp, Synthetic construct
  source 1..2969: mol_type = other DNA
agttgtttatcacgacctcaactcagtaaggcctgagattctttcgaaaaggagctttgcttcccatgacgcagagggaagtgtcaactgggatatttctggtaaaactgaaagcaagaaaagcagggtgctagcccctgtgggactgagggtggaggctgggggagtttgggtgccatcctccagtgacagatggatggacctttcatctaagagaaaggaggagacacgttggcaaatcagcctcaagcctaagattgcttgtgaagcaatcataaggaggaacaaaaacagacacaaaaacagagggaaagagtgaaaagacaagaagggcgcaaactgtgacagactcaccgcttcactaactactcacttaaactggaagcaaaatgtccctaaaattgccaaggaactgggatttcaacctgaaagtggaggctgcgaaaatagctcggtcaaggagtgtgatgactggcgagcagatggctgccttccatccatcgtccacccccaacccgctggagaggcccatcaagatgggctggctgaagaagcagaggtccatcgtgaagaactggcagcagaggtactttgtgctgagggcgcagcagctctactactacaaggatgaagaggacacgaagccccagggctgcatgtatctaccaggatgtacaatcaaggagatcgccacaaacccagaagaagctgggaagtttgtctttgaaatcattccagcctcatgggaccagaatcgcatgggacaggactcctatgtcctcatggccagctctcaggcggagatggaggagtgggttaaattcctcaggagagttgctggcacaccctgtggagcagtgtttggccagcgcttggatgagactgtggcctatgaacagaaattcggcccccatctggtgcccatcctggtggagaaatgtgcagagttcatcctggagcacggccggaatgaagagggcatcttccgtctgcctgggcaggacaacctggtgaagcagctgagagacgcttttgatgctggggagcggccctcctttgacagagacacagatgtgcacactgtggcttccctgttaaagctctacctccgagacctcccagagcccgtggttccctggagccagtacgaagggttcctgctctgtgggcagctcacgaatgcggatgaggcaaaggctcagcaggagttgatgaagcagctctccatccttcctcgtgacaactatagtctcctgagctacatctgcaggttcctacatgaaatacagctgaactgtgctgttaacaagatgagtgtggacaacctggctactgtgattggtgtgaatctcatcaggtcgaaggtcgaagaccctgccgtgatcatgagagggactcctcagatccaaagagtgatgactatgatgatcagagaccatgaagtcctcttccccaagtccaaggatatacccctgtcaccccctgcccagaaaaatgaccccaagaaagctccagtggcccgaagctctgtaggctgggatgccactgaagacctccgaatttctaggacagacagcttcagtagcatgacaagcgactctgatacaaccagccccaccggacagcagccgagcgatgcgtttccggaggacagcagcaaagtacccagggaaaagccaggagactggaaaatgcaatctcgtaaaaggactcaaacactccctaaccggaaatgtttcttgacatcagcttttcagggtgccaacagcagcaaaatggagatctttaaaaatgaattctggtcgccttcctcagaggctaaggcaggggaagggcacaggagaacgatgtctcaagacttgcgccaactttctgactcccaacggacttccacctacgataacgtcccttccctgccagggtcccctggggaggaagccagtgcactctcttcccaagcctgtgactccaagggagatactcttgccagtccaaactctgaaactgggcctggaaaaaagaactctggagaagaggaaattgattctttgcagaggatggtccaagagctacgaaaggaaatagaaacacagaagcaaatgtatgaggaacagattaaaaaccttgagaaggaaaattatgacgtttgggctaaagtggtgaggctcaatgaagaactggagaaggaaaagaagaagtctgcagccctagagatcagcctccgcaacatggagcgctcccgggaggatgttgagaagaggaacaaggccttggaagaagaagtcaaggaatttgtcaaatccatgaaggaacccaagaccgaggcttaagggtcccaggagtactgcagggacagccccagagaggcccaactctggcccctttctcagtgctatctgatgacggggaaacaaaattattctctgagagggaaaggacatttgagggaaacatcaaatttccccataaataaatgaatggagtttgcaggaaggtgagggtgagcagagatgtgtgtggacatctctgaccatccatcgctgtattcaaatggattgttttattccattctggtctcaggcatgaccacgtccagtgaagacatttgaggcagcacatctcaggacccaggcaatagactggccccaactcaggctggactaaggtgtgattaattctttgttttttgtgtggaacagctcaccttgtcagacagcctcagggcatctctgagacacaggggcagaaaatgacattcatcttttgagtcctcatccatggagtgctgtgtttggggggctgcatctgctgaagcgagaaccccattctgccaccccaccaggatgcccattctccaggacttctccaacttactattagactaaaccagaacaagcaacaaactgtatttatgcaagcaaaattgatgagaaaattatattcaaataaagcaaaaattaaaaaaaaaa

SEQ ID NO: 14 - DNA, 3695 bp, Synthetic construct
  source 1..3695: mol_type = other DNA
agttgtttatcacaacttcacttcagtaaggcctgagattctgcccagcagggctctgcttcccaagaggcagtgtcaactgggacatttctggtaaatctgaacgcaaagcagggtgccagcccctgggggactgtgggtgggacttggggagtttgggtgccatccccgagtgacagatggatggacggttcatgagagagaaaggaggagacacgtgggcagatcggtctggagcgtaagagtgcctgcaaagcaagcaacaggcagagaaggcagaggaaccgaagtgaagatcagagaaacaagaagggcacagcctgtgacacactgtgagccacttgaccagctactcacccacctcggaagcaaaatgtctctcaagctgccaaggaactgggatttcaatttgaaagcggaggcttcaaaaatagctcggtcgaggagtgtgatgaccggggagcagatggcggcctttcacccacccaccactcccaaccccctggaaaggccaattaaggtgggctggctgaagaagcagaggtccatcgtgaaaaactggcagcagcggtactttgtgttgagagctcagcaactctactactacaaggacgaagaggactcaaagccacagggctgcatgtatctgccaggcagtacagtcaaagaaattgccacaaaccccgaagaagctgggaaatttgtctttgaagttattccagcctcaagtgaccagaaccgcattggacaagactcctacgtcctcatggccagctcccaggtagagatggaggagtgggttaagttcctcaggagagttgctggcacgccctctggagcggtgtttggccagcgtctggatgagactgtggcctatgagcagaagtttggccctcacctggtgcccatcttggtggagaagtgcgctgagttcatcctggaacatggtgtgagtgaagagggcatcttccgcctgcccgggcaggacaaccttgtgaagcagctgagagatgctttcgatgcgggggagcggccctcctttgacagggacacagatgtgcacacggtggcatctctactaaagctctacctccgagacctgccagagcctgtggttccttggagtcagtatgaagggttcctgctctgtgggcagctcatgaacgcagatgaggcaaaggctcagcaggagttggtgaagcagctttctacccttccccgagacaactacaacctcctgagctacatctgcagatttctgcatgaaatccagctgaactgtgccgtcaacaagatgagcgtggacaacctggccactgtgatcggggtgaacctcatcaggtcgaaggttgaagacccagctgtgattatgagagggactcctcagatccaaagagtgatgaccatgatgatcagagaccacgaagtcctcttccccaagtctaaggatgcaccgatctcaccccctgcccagaaaaacgatgccaagaaggctccagtgccgcgaagctctgtggggtgggatgccacggaagacccacccctttctaggacagacagcttcagtaacacagcaagtagtcctgatgccaccagtcccactggaccactgcccagtgaccagcatcaggaagacagcggaaaagcccccagggaaaacccaggagactggaagatgcaatcccgtaaaaggactcaaacgcttcccaaccggaagtgtttcctgacgtccgcattccaaggcaccaccagcagtaaactggaaatctttaaaaatgagttctggtctccatcttcagaggctaaggcaggagaagggcacaggcgaactatgtcccaagacttgcgccacctttccaatgaccagcggacttctacctacgataatgtccccacctcaccacagtcccaagggaacccagcaggtgcactctctccccctgccagtgactccaagagagatgctcttgttagcacagactctgaaatggaggctggaagcaagaactctggcgaggatgaccttgattctctgcagaggacagtccagagcctacagaaggaaatagaaacccagaagcaggtctatgaggaacagattaaaaacctggagaaggaaaattacgatgtctgggctaaggtggtgaggctcaatgaagaactcgagagggagaggaagaaattcgcggccctggaaatcagccttcgaaatgtggagcgctcccgggaggacgttgagaagaggaacagagtcttggaagaagaagtcaaggagtttgtgaagtcgatggagaagcccaagacaaagacggatccttgagggtctgagtggtgtggcagggacagtcaactccactccatttcttcatgctcctagtgagctggaagcagggttacctcctaggatggacagggtatctgagggatgtgccacaatcctcagcaaatacacagaagttgagagtgtacaggagcacaccaggacacttgccaccctgtgctgtgctccaaggattgcgttattccactgtgggaagagtgaactggaatcttccagtgtatgaccaggtccaaaggagacatttaaggtgtgtgacatctcaggaccccaggccagagctgggccaaggcctcagttctttgttgtctctgtggctgtcttgcctagtcagatggttctggtcgctctaaaacatggccagaaaatatttcttcactctccagtgctggccaactgagggctgcttggagaggctgaactgttggaatccaggatcaaacaatatttatgaaagcaaaatgagtgagaaaattatattcaaataaagaaaacagaaaacaaaaattgagcaaggatattggcaatctaatgaaacacttttattagaattagtaaaagggggtttggtaaaattagtaaaagctggctctgtggctgagagcactggctgctctttcagaggactcagcatccacacgatgatggctaacaaacctctgtagctccagttccaggagatctgacaccctctgaggacaccatatatacatatgctgcccatacatatatgcaaacaaacaaacacccgtacacatcaaatacacacacacacacacacatacatacatacatacatacattccaacaacaaagaaaaaaataattagtaaaaggcacctcttgcctgaagcaggtgaatctttcttgtggtacatggctgtgtgaccagggctttgggttccatcctgagtcaccctccagtggacaaaacttgtctgcaaagtctcagagcaaaggtttccaactatgaaatgactttaggggtgtggatttgtatagtgaatagtcaagattattaattattattgtcattgttgttattgctattctacttcaacaaaatgaaaatgggtttatttgctctttagagggggatcaggcacaatcatctgttttctcaactatagctaatgccacctgccttttgctgagttaattataaaaacaaccaaccaacaatatcgctgattactctctggccacaatggtctccctgctcccgtggcttgccccaggtggcagtaccgccccaattgctgctgttggcttgccactgaacctctgtattctcagtcatctattccttgcggctagctgtcacaaatcctctgtaacccagtaaaatcaaaactct

SEQ ID NO: 15 - DNA, 2153 bp, Synthetic construct
  source 1..2153: mol_type = other DNA
gtcgccgccgcggagcggagtcgagccgagctgatttgatcgaggagcgcggttaccggacgggctgggtctatggtcgctccgcgggccgctccgccggctggtgcttttttatcagggcaagctgtgttccatggcagggaacttttggcagagctcccactatttgcaatggattttggataaacaagatctgttgaaggagcgccaaaaggatttaaagtttctctcagaggaagaatattggaagttacaaatattttttacaaatgttatccaagcattaggtgaacatcttaaattaagacaacaagttattgccactgctacggtatatttcaagagattctatgccaggtattctctgaaaagtatagatcctgtattaatggctcctacatgtgtgtttttggcatccaaagtagaggaatttggagtagtttcaaatacaagattgattgctgctgctacttctgtattaaaaactagattttcatatgcctttccaaaggaatttccttataggatgaatcatatattagaatgtgaattctatctgttagaactaatggattgttgcttgatagtgtatcatccttatagacctttgctccagtatgtgcaggacatgggccaagaagacatgttgcttccccttgcatggaggatagtgaatgatacctacagaacggatctttgcctactgtatcctcctttcatgatagctttagcttgcctacatgtagcctgtgttgtacagcagaaagatgccaggcaatggtttgctgagctttctgtggatatggaaaagattttggaaataatcagggttattttaaaactatatgagcagtggaagaatttcgatgagagaaaagagatggcaaccattcttagtaagatgccaaaaccaaaaccacctccaaacagtgaaggagagcagggtccaaatggaagtcagaactctagctacagccaatcttaaaacattccgaagaattccatagtggaccacttggaaataaaccattggacagatttcagtaatgtcttcagtggaacacaaatgaaaatgaatagcttgtttctgtcaagcatattggaaagtgattttatttttgcaaaataagtttttctttaatatgattctagtacataattgattaaaatctcttgattataaatgtttggaaaggttctaaggggacctacagacagacatacatagacatttcaaaattaatagcttttgattagtataatatttcttaatttggataataaaaattgtagctttttattaagccaggaaacatgaagcataatttgtttaaaattctctttggtcattgagggaccaaaaaaggacgtaaaatttacagtcaatctatgagggtttttttccctccataagtttaactttaaaactgtatttaaggaatcaaatcttacaaaatcctggaagattttggtaatgatgttgataatttcagggaaattaatcaagtacctatattgatttaaaagtgtattttattcagtagtttgaggatcttgccatggaagatactagcccagcctagcagaaaagtgcaatatgtatagcatactttgacattttaaacatgatagtccataaccattttgaaatgctgggcaaactacatgaagttatttataattaattcacagctaatcaggcattttgaaagcttaattggattcaaaaaccataatgttggaatttggtaaaattttaatgttgatttttactgtgaaaaggtttttataagatatacacaccctagtttaatgttgtgtcttggtgtggatttacagatttactacaggtattctgaaccaggaacacaatcaggtttcaggccagtttgatactggctgtccttaattctaatatgagagtaggacatcatactaaatgttatgtcagtgggactgtactgtctgtggaacttagcaaattaatcattttcttcagacttgaaggagagtgataaataaaatttggagtcataggatattgatgcacaatttaaggattaaacatttttaatcaattgtggatgatggcttattaaatgttgacttcctagtataaaactgcaagaataaaagtaacttctccagctgta

SEQ ID NO: 16 - DNA, 3391 bp, Synthetic construct
  source 1..3391: mol_type = other DNA
gctcagtgttgtcgaccggcgtcaaggcgacgagggtgctgttctcttcctccggctgcggtggctgtggagttcagcccagccagtctggtaggagcacagtcgtccggactgtgtctatggtcgctccgcgggccgctttgctggctggtgcatttgtatcagggcaagcagtattattccatggcagggaacttctggcagagctcccactatttgcagtggattttggataaacaagatctgttgaaagagcgccaaaaggacctaaagtttctctcagaagaggagtattggaagttacaaatattttttacaaatgttatccaagcattaggtgaacatcttaaattaagacaacaagttattgctactgctacagtctatttcaagagattctatgctaggtattctctgaaaagtatagatcctgtattaatggcgcctacatgtgtgtttctggcatccaaagtagaggaatttggtgttgtctcaaatacaagattgattgctgctactacttctgtattaaaaactagattttcatatgcttttccaaaggaatttccttacaggatgaatcatatactagaatgtgaattttacctcttagaattaatggactgttgcttgatagtgtatcatccttatagacctttgctccagtatgtgcaggacatgggccaggaagacgtgctgcttccccttgcatggaggatagtgaatgatacctacaggacggatctctgtctgctgtaccctccgttcatgatcgctttagcttgcctacatgtagcctgtgtcgtacaacagaaagatgctagacagtggtttgcagaactttctgtggatatggagaagattttggaaataatcagggttattttaaaactgtatgagcagtggaagaattttgatgagagaaaagagatggcaactattcttagtaagatgccgaaaccaaaaccacctccaaacagtgaaggtgagcagggtccaaatggaagtcagaactctagctacagccaatcttaaaacaccctgaagaatcccgtagtggaccacttggaagtaaagcattggacagtttgagtaatgtcttcattggaagcagatgcaaatgaatagcttgtttctgtcaagcatattgggaattgacttaattttcacaaaataaggttttttcctaacttgatttagtgtgtaatttattaatgcttaattacaaaatttagtaaggctctaaaggaacctatagccatacacagacatttcagggttaacctctcttacatacacatatgtatatagacacatactaatactggtttttgattagataattgaagggttttttttaattaataataataactttttattaagtcaggaaacatgaaatgtcatttgttgaaatttttctttcagcattgagggaccaaaagaggacataaaaagtcaataaaactacccccagcccagttaaattttaaaactggatataagaaattaaatcttagaaaatccttagaaggttttagaaatgataattacagtttgagggagattaatcaagaacttacttattttaaaattgattgtatttaatactttttgtatcattacagaagatactggcccagcctatcaaaaagtgcaatatatatatagcatactttgacattttaaaggttggattgacttactagctgttttgaaaacctaaaagaaaaggttatttgcaactaatttatagttaaattagtttaacttgattaaacacttaaggtaaaatttggtgaaattttaatgtttaatctgtcctgaaaattttttatgagacatatacacccggtttaattttatgttttattgtgaactaacatatgtacagcaagtattctaaatcaggaaggaacctagtcaggttttagaccagtgagattcttaattctcatgggtatagacttcagaataaatgttacatttgtgggacttcataaattaattaattttcttcagactcaaagggagaagaataaataaagcttggagtaatgagatattattgtgtagttttgcaccttttaaaaaataaatcatgaaaaaaattactaagtgttgacttcctggttaaaattggaagaataaaagttaagtttttcaggtatattaactaatgtcttgaattcctgttgctaagtttgacttaggttctctgagaatctttgtgcgtgttaaattagagccctaaactctgtaaagaggtgatttttgtatagtttatgaatagattgtgaggaaggttctgcttcctatggggaatcagatgcaggtgtgctgaaaaagagacttacattggtacaaacatggactctcctaaggccgtataaggctaagtaacaaacaaacagaggaacaaacaggctctccccgaactccacttggatgcttggtaattgatttgcttatcccagccgttaactatagttttatcttcaagcttttttaaatccagtttcttttttcagattttttttttttttttgctttattattttctttgcaaatgctcttgtggatgattaagactttgcaaaagcagacaaagccgtgaaatagatctccatgtgtgtatgctctagcctcagccattactgattcatagccagtttcaccttaatgtaaacttccacaccagtctccagctcatacattatcttgaagaaaattctagatatatcattttacccatacataatttcatactgctaataggtgaaataattatttactattatattaaaattctcaattccctgaacattgcagagcattctgtattcatttataaatccttttaggtttagattcatggattattcctccatgctataaaaaacattccctgttttcatttcttcatgtattaagtttcccatagcctaagttttactgtgagcatctttagcatgccatttagcgagggtttttctcctttgtatttctgtaaattgataattgaatatgaaggctgatcacaccgagattctttttgttttggctagactactgtagggtggccatctcttgtattaatcagcacatagtggctgttgctgtcctcatttatgacaagtactgtttcagaatcagtcagtcttaggcacactgtccagtctcatagccaccagccacttagggctgaacagttactatgtggcttgtctgaatgcagatatgctataaatgtgcaaacatgttgggtttcaggtacttattaatataaaacaaaataatgttaaaaatctcaatttgtttataaccattatgtgttttaaatgataatattttggatacatagaattaaataaaacattattaaaatttt

SEQ ID NO: 17 - DNA, 3864 bp, Synthetic construct
  source 1..3864: mol_type = other DNA
gtcacttgccctccccgctcggagctgccgcctggcttgtcgcggctctgccacaggggcaggtgttgaggggctcccggtccggctgccgccgctcccccgctccggacccggggctccccctagcgccgctgaggagccgcctctgcggctccaggagggcgcaggagcgggactgagagcgcctggaggctcgagcggagggtaattcatttgcacacctgttagcaagaaacagaagttgaaggactggaacaagtgaactaggaaagagggaacgccaatccaaggatagaaggacaaggacagaatcaccagcactggctgaaggtaccttaacatggggaatcttcttaaagttttgacatgcacagaccttgagcaggggccaaattttttccttgattttgaaaatgcccagcctacagagtctgagaaggaaatttataatcaggtgaatgtagtattaaaagatgcagaaggcatcttggaggacttgcagtcatacagaggagctggccacgaaatacgagaggcaatccagcatccagcagatgagaagttgcaagagaaggcatggggtgcagttgttccactagtaggcaaattaaagaaattttacgaattttctcagaggttagaagcagcattaagaggtcttctgggagccttaacaagtaccccatattctcccacccagcatctagagcgagagcaggctcttgctaaacagtttgcagaaattcttcatttcacactccggtttgatgaactcaagatgacaaatcctgccatacagaatgatttcagctattatagaagaacattgagtcgtatgaggattaacaatgtaccggcagaaggagaaaatgaagtaaataatgaattggcaaatcgaatgtctttgttttatgctgaggcaactccaatgctgaaaaccttgagtgatgccacaacaaaatttgtatcagagaataaaaatttaccaatagaaaataccacagattgtttaagcacaatggctagtgtatgcagagtcatgctggaaacaccggaatacagaagcagatttacaaatgaagagacagtgtcattctgcttgagggtaatggtgggtgtcataatactctatgaccacgtacatccagtgggagcatttgctaaaacttccaaaattgatatgaaaggttgtatcaaagttcttaaggaccaacctcctaatagtgtggaaggtcttctaaatgctctcaggtacacaacaaaacatttgaatgatgagactacctccaagcaaattaaatccatgctgcaataacaattctggaataagcacctgctgtagacagaagacagtattctgcaatgactgagaatgcagttttttagtgattgcaattactatctcatttattcttgcttttatttctttcctctgttcctcttccctcttttttaatcatgttcttaagacttcttttctgtgccaaaatcagtaaagttacactctgaagggatatcatcctttcaaacgggccatctaaggcagctaattatgcattgcattggggtctctactgagaaaaattctgtgacttgaactaaatatttttaaatgtggattttttttgaaactaatatttaatattgcttctcctgcatggcaaaactgcctattctgctatttaaaaaccctcaatgactttattttctactgccgcctttttcatgtgcaaccaaaatgaaaatgtttaaattaactgtgttgtacaaatggtacccaacacaaactttttttaaattagtaatacttttgtttaaagttttaagtttgcattttgactttttttgtaaggatgtatgttgtgtgtttaacctttattaactaacgttaaaagctgtgatgtgtgcgtagaatattacgtatgcatgttcatgtctaaagaatggctgttgatgataaaataaaaatcagctttcatttttctaagtgtggcttggtttactgatgttttgtttttaggcttctgaagttttccccatgtatgtttttatggaatgcatatatacattacatgattttttcttcttttaggggagagccgggtcttttcattagaattttgatcattgtctatgtatatacttttaattgcctttaaaattttgctaagtatattaagaccagtgatcttttcattaactggacttttattcttgatttactcttgtatggatgtagtaatggaaggaaaaccgcatccttgcttgattctccatttccaggggagcgggtagcaacatgaaggcagagtagccctttaatcatgggaagagagtgattataagagggacttcccaggagaaaagcagaggccatgatctcactagtggtagaaaactaacaggtttcagtttaagatcataaaacctaaatcatgaaatctaaatctcatggttggaacaaactacttaaaggttgtctagttcaaccatgccagtgttttaaatatttaggaatatccgaggtataatacaattaaaatgtttaaccatggaagagaatcacagaggcttccatcagacccttctggattaatagggccagatcatggcacacacagcttctcaagatcacatccctgctgatctctctggccctcctgatcactgctcccccacttcacaccctgtcctgcagccaggccctactgcttgctgttcacaaattgttccatgtgatgtcacttccttagctctggtctggctggtccctgaacctgacattcccttcctccccttgtgaccctctaatgaacgccaactttcatctttgaagggttagcttctattacctccttcatgaagccttctggataacattggccatatccttctaggcatcttctaagaatagtgtaataggctgaatagtggcccccaggagatacattcactgggaatctccacgtgtgacattatttggaaaaagtttttgcagatgaaatcaaggacctggaaatgagatcaacctgtagtagggtgggccctaaattcaatgactagctcttagaaaagaaggggaaatagacacacagagctgaaggccatgggaagatggaggcagagattagagcaatgcatctatgagccaaagaaccctaagaattggaggaagtcactagaagctaggaaaacaggcatgtcacagattctccctcagagtctcgagaaggaaccaaccctaccaatatcttgatttcagacatctggcctccaggattgtgagggaataaatttctgttgttttaagccatgcagtttgtggtattttggtacagcagccttaagaatctgatacatgtatctcgtaaaacacctctgatacttacctgcgcttgtggctgtgttttctgatactatgagagccgtagtatctggataccggagagtctgcgaacttgagagaagggccgtgtctgattttccttcagtgtcccaaggaaggatcttgcacagtacccagatttggagaggtgccaagtgaatgcttcggtggactgaatgaatacaaatcaaaaataatgatgcagcgggaatggatgcagtaagattgctgtggcctatgcagttcacacagttcacactctgcatgttgaggagtaagtggacagatctcttaagcccaggggctagcaaaccggtctgtgggccaatttactcaccgtctgtttttgtacagcttgtgagctaaaaatggtttttaaaaaggttgaatacaatcaaaagaataatattttgtggcatgagaaaagcatatgaaattcaaattttagtgttcataaataaaattttattggaacctaa

SEQ ID NO: 18 - DNA, 3715 bp, Synthetic construct
  source 1..3715: mol_type = other DNA
gccgctgtcgcctggctcttcgcggctccgccagaggggcaggtgcggaggggctcccggtccggccgccgccgccgccgccccgctccgggcccggggctccccctagcgccgtgacgagccgccggcggccgagagaagcgcggccgagtgcccgagctcgcggcggatagaaggacaaggacaaaatcaccagcactggctgaaggtaccttaacatggggaatcttcttaaagttttgacatgcacagaccttgagcaggggccaaattttttccttgattttgaaaatgcccagcctacagagtctgagaaggaaatatataatcaggtgaatgtagtgttaaaagatgcagaaggcatcttggaggatttgcagtcatacagaggagctggccacgaaatacgagaggcaatccagcatccagccgatgagaagttgcaagagaaggcgtggggtgcagtcgtgccactagtaggcaaattaaagaagttttacgaattttctcagagactagaagcagcactaagaggccttctgggagccttgactagtacgccatactcgcccacccaacatctagagcgagagcaggctctcgctaaacagtttgcagagattcttcacttcacactccggtttgatgagctcaagatgacaaatcctgccatacaaaatgacttcagctactacagaagaacattgagtcgtatgagaattaataatgtcccggcagaaggagaaaatgaagtaaataatgaattggcaaatcgaatgtcattgttttacgctgaggccaccccaatgctgaaaaccttaagtgatgcaacaacaaaatttgtatcggagaataaaaatttgccaatagaaaataccacagattgtttaagcaccatggcaagtgtatgcagagtcatgctggagacaccggaatacagaagcagatttaccaatgaagagacagtatcattctgcttgagggtaatggtgggtgtcataatactctatgaccacgtccatccagtgggagcatttgccaaaacttctaagattgatatgaaaggttgtatcaaagttcttaaggaccaacctcctaatagtgtagaaggtcttctcaatgctctcaggtacacaacaaaacatttgaatgatgagactacctccaagcaaattaggtccatgctgcagtaagagctgtggagcaagcccctactgcagatggagcacacagtgttcttcagtgaccgagaatgcacagtcttagtgaatgccatcaaatctccttattcttgctttggtttctttcctctgttcctcttcccactttttaaatcatgttcttgttcttaagacttcttttctgtgccaaaatcagtaaagttatactttgaaagggattttgtcctttcaaaacaggccatctaaggcagctaattacgcattgcattgaggtctctactgagaaaagttctgtgacttgaactaaatatttttaaatgtggattttttttgaaaactaatatttaatattgcttctcctgcatggcgaaactgcctattctgctatttaaaaccctcataaatttattttctactgccgctttttcatgtgcagccaaaatggaaatgtttaaattaactgtgttgtacaaatggtacccaacacaaaaccttttttaaattagtaagactttgtttaaagttttaagtttgcaggttgactttttttttaaggttgtatgttgtgtgtttaacatttattaactaacgttaaaaactgtgatgtgtgcgtagaatattacgtatgcatgttcatgtttaagaatggctgttgataaaataaaaatcagctttcctttttctaagtgtggcttggtttactgaggtttctggaagttttccctgtatatatttctatggaatgcatatacacattacataatttttctttctcttaggcaatagctatgtctttttcagttgttttgctttactttttgtctatgtacataagttagactgcttttaaaactgttgtgtataagatgaccagtgattgtgtcgacattaacttgacttctgttctgatttaatctttgtgagcgtagtcatagaaggaaacccgtactttctcagctctcggtttcaggaatgaaagggcggtgacaggaagcacatggtgtctcgaaggcacgaggagcccttgacattggaggagagtgagcaataagagtacttccagaggaaataaatgaaagtccgagatctcactggtcgtaggaagtgacatgagttacaatttaaaatcataaagcccaaggcaaagaaatgtgtctcagttgagagaagctgcttgaaagttgtcgaattcagccttgtcagggacttaatgacaaggtgacctgaggtgggacacagttaaagtgcttaaccatgggagatgatcacaggctgccctctggaccctctccatcagagcgccagattgtggtgcacacggcacttgatggtaaagttcctgttgacctctctggcctcctgcttactcgtgttccacttcctaccctgctctccagccaagcttaactacttgctgtttacagattgttccgtgtggtgttgcttcgtaactgatctggctacaacctgaacctggtcttccttcttctctccttcatgtttgtctgaccaacaccagctgtcatctttcaaaaatcagcttcttttacttcctccaaaaaccatttagatgacattggccatgtcttccaggaggcttctacagtgttataatgggtttaatagtttactccataaagggtaatgtctggaacctgtacatatggcctcattttgaagaagggcattttcaggtgaatgtaaggacctatgtatgtatggcatcgtcctggatgagagtgggtcctagccctactgacaagtccttttaagagaaaagaaggggaaacaagcagtggaggtcctgggaggatagaggcaaaggtaacatcatatcttgaaatttcctggaccacagaagccagaagaaggtcatggaacactttccatcagagtttctagaaggaagcgaccctgttgctgtttttatttcagacttctattatgatgaaaaaaataagtttctattctaagccacctagtttgtggtaatttgttacagtcaacttaagcattgaatacaactgtctcacaaaacagcctgatacatcactacacttttatatcttctctgatactcatgagaagttacaatgtctagatgctgcagagactgagaacctgagaagagccatgtcttattccttcagtgtctcaagcaaggatcttgcatagtacctaaatatggagagctgccaagtgagtaattgaatggagtgagtgaggactgataaatgaagtagatgtagtaatcctgctgtggcctttcaggtaacatggttcacactctgcatgttgaagattaagttggcaaaaaaatctttaactctagcctaggggtcagtagcccagtcggtgggccaattcacctccctctggacctacacaacctgtgaattaagaatgatttttaaaatgtcgggagaataaaggataacaaatgtttttctaaaagttgaaaaatattttgtagcatgagaaaagtatatgaaattcaaactccagtgttcataaataaaattttattgaacata

SEQ ID NO: 19 - DNA, 7077 bp, Synthetic construct
  source 1..7077: mol_type = other DNA
ggaggggttcgctgccagcaattgccaagctgctgcggtcgtggcgggatggtatagccgggcctgagggcgggcggcggggcgcgggcgggggcgaggcggccgggaagggccgacgccgcctgactggcttgggagcagcggcagcaagctgagggggcgtcgccgccgccggcccccggcgccgccgcgggaggccgcgcccgccatggcggcccgggcggtgctggacgagttcactgcgccagctgagaaggcggagctgctggagcagagccgcggccgtatcgagggcctgtttggcgtgagcctagccgtgctcggcgcgctaggggctgaggagccgctgcccgcgcgcatctggctgcagctctgcggggcgcaggaggcggtgcacagcgccaaggaatatattaaaggaatctgtgaacctgaactagaagaaagagaatgttaccccaaggacatgcactgcatttttgttggggcagagagcctgtttctgaaaagcttgattcaggatacttgtgctgacctctgcattctggacattggccttcttggcatcagaggaagtgctgaggctgtggtcatggctaggagtcacattcaacaatttgtaaagctctttgaaaataaagagaacctacccagtagtcagaaagaatcagaggtgaaaagggaattcaaacaatttgttgaagcccatgcagacaattacacaatggatttgttgattttgcccacttccttgaaaaaagaacttttgacactcacacaaggtgaggagaatctctttgaaacaggagatgatgaggttattgaaatgagagattctcaacaaacagagtttacacagaatgctgccacagggctgaatatttctagagatgaaactgttttgcaggaagaggcaagaaataaagctgggactcctgtttctgagcttacaaaacaaatggacacagtcctttctagctcaccagatgtgctttttgatccaataaatggtctaaccccagatgaagaggcactttccaatgagagaatttgtcagaaaaggagattttctgattctgaagaaaggcatacgaagaagcagttttctttggaaaatgttcaggagggggagattttacacgatgctaagacattggctggaaatgtaatagctgacctatctgattcttctgctgattctgaaaatttaagtccagatataaaagaaactactgaggaaatggaatacaacatcctcgtaaacttttttaaaaccatgggctactcccaagaaattgttgaaaaggtcattaaggtgtatggaccatctactgaaccattattgctcttagaggaaattgaaaaagaaaataaaagattccaagaagacagagaattttcagctggtacagtgtatccagagaccaacaaaaccaaaaataaaggtgtttatagcagcacaaatgagcttacaactgattccactccaaagaaaacacaggctcacacacagcaaaatatggtagaaaaattttctcagttaccattcaaagtggaagctaaaccatgtacctcaaattgtagaattaatactttcagaacagtgccaatagaacagaaacatgaagtctggggttcaaaccagaactacatttgtaacacagaccctgaaactgatggcctttcaccttctgttgcctctccaagtcccaaagaagtcaattttgtttcaaggggagcttcaagtcaccagcccagagttccactttttcctgaaaatggtttacaccagcagccagaacccttgcttccaaataatatgaaatctgcctgtgaaaaacgtttaggatgttgtagttctcctcattctaagccaaattgctcaaccctttctccaccaatgccactgccccagctgttaccttcggttactgatgcaaggtcggcaggaccttctgatcatattgattcctcagttactggggttcaaaggtttcgagatactctaaaaataccctacaagctggaattaaaaaatgaaccagggagaacggatttgaaacacattgttatagatgggagcaatgttgcaattacccatggtctgaaaaagttcttttcttgtcgtggaattgcaattgcagttgaatatttttggaagcttggcaacagaaacatcactgtatttgtccctcagtggagaacaaggcgtgatcctaatgtcacagaacagcacttcttaacccagctccaggagctcggaatattatctttaactcctgcccggatggtctttggagaaagaattgcttctcatgatgacaggtttctactacacttagcggacaaaactggtggcataattgtgacaaatgataatttcagagaatttgtgaatgagtcagtctcttggagagaaattattacaaaaaggctgctgcagtacacgttcgtgggggacatatttatggttcccgatgatcctctgggaagaagtggacctcgattagaggagtttcttcagaaggaagtctgtcttagagatatgcagcccctactcagtgccctgccaaatgtgggcatgtttgaccccagcttcagagtccctggcacccaggcagccagcaccagccaccagcctccgacccggattcagggagccccttcaagccactggctccctcagcagccccacttcccactgctgccagccctccccagtctccagcagaacctgcccatgccagctcagagatcttctgcagaaaccaacgagctgagggaagcccttctgaagatcttccctgactcagagcaaagactgaaaatagaccagatcttggtggcccacccatacatgaaagatctaaatgcgctttctgccatggtgttggattgaagattgtgctgagcgctgagcttgcgctcagggctggctgatcaaactcaagctcacacctgtacctgatgggaatgaacataatgtgaagaaacttatttcccagtgtaaacgcagttgtacagaaaaaaaatgttttgggtataaatctgggttttcaaaaccagcttttttctatatataaattatgctgctattacagatttaacattttctgttaaaggaaagtctacattttctgcctgttcagaactgtttaatagcagttactcttgagtgtatttacctttttatgttttttaaactattttccttaaagccgaaaatattgacaaatggatatggttagcctttataaataaataaaaaaaaaagtgttgctttcttaaggaaagcaagacgtcttaaagtatattttcttgagatgagtatgtcccatcactaccatgaagtgtgctcacccttccagcctcctctgccctcacccccggagttaaagtgggtaagagttggtttgttctgcagtctttggttggagtgttgtgaaagtggacccgcggtgccactagatggcacttggtgcctagccatggtgaatgaccagggcgaagttagtctcacagaataccagggctagaagacatcgtggagcccatggagtcccaccctccttcccccttccaccctgcgcaaaaggggacggcccagtgtgcacgccgtccggccatgcccacagccacagccacagctgaggagtggccgggcaggcagcaggctccccagccagggcttgacacgcccacagtcaggggcctccacatgcctggcctggctctggaagtcacgtcttagcttctgaacaccgtagcgggttatgaggaagtttaatgaagagcccggtcactgtggccatcttgtgtccccaactgcaggagacgccctgatgtggagtttgtacagctttgccaaaaaatggttttctattctcaaaagtgacccaagccaataaatagcattagtagcttctgtggggggatcccagagcccctgtatttattttccctgtgtttgtctctagtgtcctcctaaacagcctttcctgtgagtctttctcagaattgatatcttaatattgtctgttctaggtgttgcccaaattcagtgtcagtgaagttcttttcctgggcaatcttaacatctttactcttattgtctggaccttcaaagggctttgtattttacacctgcgcccccaggctgctcacagtcggcttgctgcctgtcctgcctgtccggatcttgtctcctagtcctgaggagcggaggctgagaactcagtgctggtttgtaaatggtgaggggacttgggggcatcccagagtgctccctccaggcctgcttcttggttttgtttgatcactgcgttcttcaagggatgaatccagagccctccatgaggccaagcttgtccttcaatcatgtttcctctcagatgcgtccgtgatgcctcctaatgtggaactggttgtccattgtttgggcctatggccaagtcacccagctgtggaagcagaggtagaagacgaggccagccaggagggcgacttcagtcacagctcccatgcctcagctttgtacctgttttcaaaagcacaactgaggtgtgcgggctggagctgtcttgcagtgattctggccttctggctcatggttcagtccagcagcctggctgacccactatttctcctctgcttcagaggaaacccaggaaatgcccttactgccaggctgagtctccacccatgctggttggtgctggctaggctgagggggccaccacttttcctggctagaagctacttgacctttgatgtttgagttctgtaagtcttcgtgttctgacttactgcttcagagggattggcctgtcccctttccctttctcggctatgggaaggaaggattgctcattggttgccttcatcagttacagcatgagacggaattcatcattccttccgaaacccctgatatttaatatttaatatttaaaaacccaaattatcaaaccattaagaactcattactggttctcagcctcctccagtactagcctcagtgtggctgctgcataagtatctgtagcctgtctacctcctgcagtggggccgctcgcctcttccctgtctactgctcaggctctcccacttcgtggcatccatgtaaagtaggtggcagggcagagatgtcactctcattcaacagggaggatgtctgttgctcagagaggttgtcctgaggggctgggtgattcctgggcctacattcttcccgaggctccaggccgctgtctctggaagtaaaagagccttgtctgaccttaatgcaagcagtctgtttgaacccctgtaggctgcattcaggagacagaaggtgtctgggccatcctgggcggccggtcagcgttgctaggcaggctcggctgtctggccgggacttgggcctgggtggcttttgagaccagtgaagaagggagagccggcctcatgccgatgcggcttgtggcacggctgggatgtgagggaggactcagatctacacacagaaacccctcttctccccgccctcccccagctcctacctgcctcccacgcctcaggtgtggctgcctgtgggaccatcccccaacccctttcctcgcacctccttgtcctcacccagttcctgcagtgtctctgacccacgcctcccgcctcctggccgacttgcccaggaggtgtctctggctcacctccgtctgttcatcaccttcctccccagtgtttccacttatcttggatgttttagattgaaacagcctgattcccggaagaatcctcttcattcattgctagtgcttcccctcacctcccactctccacttcccagtttgcaaatgtggctttcgcccaccaagtgaaagcggactgagagcagcccttggggacggcccggtgcctggctgcaaggccgcgctggggctctgtcttggtgcacatggcttgaccggactttccctgcttcccaccacttccctcactcccagacctccctcattctttttgtctcttctttttgcctaaagccagtccttaacaccctattcttcctctgcaggtggcttgcagacttttccccacctttggggctcgtggtggtggagagggcagctggtgttaagaatgtaggttaccgggcatgacccggcagatgcttgcccagtagttctggaggaaggcccgggaatctgcaaatgagcgcattccccaggcagttcccatgcaggtgatccacggaccacatgttgagaaactgcagtcacccttagggccacaccgtccctctcctcgctgtcccctctctgtagtgactggccctgaccttcaggagtgcactttccactctaccaggaagccctatgacatcctcaggctccccagacctgcagcttgcatggggcccctcccttcttccacacccaccctccgtatggtcccctgctctgccctcgtgctttgctggcccctgcccgctactcccactctcagacacccaggggtggtgggccctaactggctggcccctcccagcgctgccctctgccgtccagatgctgcagtgtggccagatttaccttccagtaacatacttctagtcacccctcctcctgcgaagtgatctgcagtggctgtttgaccagaccacaaagttcacatctcctgagcttagtgtccgtggctgtccacctcccagccatacttgactgtccccaaactctccctgcagccacatgtttcccatgacctgtgggctctgcagatggacctctctccgctagagatgcccttctcccaaatggcttccctcctggaaggcccagcctgagtcctcgtctcctttccagtgcttctgccagaagcatccccatgatgttgtgaccgcacagcactttgtgtctcgctttgagcacttgccactctggctggtgctgctgccactgattgtgtactgtcttgctgccctttctagactgtgagctcctcgtgggcagggaccgcctgtgttctctgtatttcccacggcgcctagcacagtgccttgcacttgataggtgcttaataaatgtctgctcaactgaa

SEQ ID NO: 20 - DNA, 6469 bp, Synthetic construct
  source 1..6469: mol_type = other DNA
gtggcggcggcggcggcggccgtggcggggcagcggggcagcggggcagcggggtggcggggagtctctgcccgcagttgctaagctgcggcggccgtgtcgtgatggtgtagcggggcctgcgggcgggcgaggaggccggcacggcccacgcctcctgagcggctcggtgcagcggcggcacgctgagggggcgtcgccgccgctggcccccggcgccccgggaggccgcgcccgccatggcggcccgggtggtgctggatgagttcacggcgcccgcggagaaggcggcgctgctggagcggagccgcgggcgcatcgaggctctgttcggggtgggcttggccgtgctcggcgccctcggggcggaggagccgctgcccgcgcgtatctggctgcaactccgcggcgcgcaggaggcggtgcacagcgccaaggagtatatcaaggggatctgcgagccggagctggaggagaaagagtgctaccccaaggccatgcactgcatttttgtcggggcacaaagcctctttttgaagagcttaattcaggatacgtgtgctgacctctgtgttttagatactgggcttcttggcatccgaggaagcgctgaagctgtggtcatggccaggagtcacattcagcagtttgtgaagctctttgagagcaacgagaacctccccagtaatcagagagagtcggaaatcaaacgggaattcagacagtttgttgaagcccacgcagacagttatacaatggacctgttgattctgcctacttccttgaagaaggaactactgagtctcacacaaggggaagagagtctgtttgaaacagatgatgatgtcattactgttggagatgtacggccaccagagtacacacagagtgctgccacaggaccgagtagtgctagagatgaagttgttgtacaggaagattctagaaataaagctaggacccctgtttctgagcttacaaaacacatggacacagtcttctctagttcaccagatgtgctttttgttccggtgaatggcctaagtccagacgaagatgctctttccaaagacagagtttgtcacaagaggagatcttctgatacggaagaaaggcacaccaagaagcagttttccttggagaatgttccagaaggggagcttttgccagatggtaaggggtcagctggaaatgaagttattgacctgtctgatcctgcttcaaactctacaaatctaagccccgatggaaaagacactacagaagaaatggaatacaatatccttgttaacttctttaaaaccatgggctattcccaagagattgttgaaaaggtcattagggagtatgggccttctacagaaccgttactgctcttagaggaaattgaaaaagaaaataaaaggctccaagaagaccgagactttccaccttgtactgtgtatccagatgccagtcagagcagaaatgcaggggttgggagcaccacaaatgagctcacagcagactccacgccaaagaaagcacagagtcacacagagcaaagtatggtggagagattctcgcagttaccgttcaaagactctaaacactgtacctcaaattgcaaagttaattctttcagaacagtgccagtaggacaaaaacaggaaatctggggttcaaagcagaactcaagttgtactgtcgacctggaaactgatggccactcggcctctgccgcctctgccagccctaaagacatcagttttgtttcaaggggagcttcagggcaccaacagagaaacccagcttttcctgaaaatggttttcaacagcaaacagaacctttgcttccaaataatacgaagcctgcctgtgaaaagcgttctggctcctgtagctctcctcagcctaagccaaattatcccccactttctccaccactgccgctgccccagctgctaccttcagttactgaggctcgattgggagggtcctctgaccatatcgattcctcagttacaggggttcaaagatttcgagataccttaaaaataccatacaagctggaattaaaaaacgaaccaggacgagcagatttgaagcatattgttatagatggaagtaatgttgcaattacccacggtctgaaaaagttctttagttgccgtggaatagcaattgcagttgagtatttttggaagctcggcaacaggaacatcactgtgtttgtcccacagtggagaacaagacgggatcctaatatcacagaacagcacttcttaacccagctccaggagcttggaatattatctttaactcctgcccggatggtctttggagaaagaattgcttctcatgatgacaggtttctactgcacttagcggacaagactgggggtataattgtaacaaacgataacttcagagagtttgtgaccgagtcggtgtcctggagagaaattattaccaaaagactgcttcagtacacgttcgtgggggacatatttatggttcctgatgaccctctgggaagaaacggacctcggctagaagaatttcttcgaaaggaggcctttcttagacacatgcagccgctactcaatgccctgccaagtgtgggcacatttgaccctggcttccggagtcccagcacgcaggtagccaacaacagccaccagcctccaccccggatccagacctcttcaagcccttggcttccccagcagtcccacttcacagctctggccacccttcccagtatgcagcagaacccacccctgccagcacagagatcttctgcagagaccagcgagctgagggaggcgctgctaaagatcttccctgactctgagcaaaagctgaagatcgaccagatcctggcagctcatccgtacatgaaagacctgaatgccctgtcggccctggtgttggactgacggctgggctggagcccactgatgctggggaactgccaaccgtgggatggaccataacgtgaagaagccttcccggaaagcattgctgtgttgtatagaaaaaggagggaaatgttttatgtatacaaaagtctggggtttcaaagccagcttttttctatatataaattatgctgctattacagacttaacgttttctgtaaaaggaaagtactctttctgcctgttcagaactgtttaatagcagttactcttgagtgtatttacagttttatgttttttaaactattttccttaaagctgaagaaaaaatgacaaataaatatggttagcctttctaaataaaattttaaaaaaaaattaagaaaaaaaaaggagttgcttccttaaggaaagcaagacttcttaaagtctattttcttgaggtaatttgtcctgttcactaaacatggcgtgtgctcccctgccttcatccctagagtgggcctgctctgctcttgtggggccttctcagcgagccattatgagagtgaagaagtggagccactagatggcatctgacacccgggctgtcaccagtggccagacaaagtcagactgaggagcaggtgggaagtcttagaggacgtacagccccagcatgctccctgactggcgtagctccgtagtgcactcaccagccatggccgccatgctcccaccctggctccacagctaccttagcctcagaagacaaggtaggcactgaagcctctggtgaggagacccccccccccccatgtggcgtttgtacagctttgccaaaaagttttctattctcaaggatgacccaagccaataaatagcattagtcctttcagaagggcctcaaagcccctgcatttcccttcttttgtctacttagaggtttcttagcagcaattcctgagtctttcagaaccagttatctcagtattgtctcttctcggtgtagcttgaagtctgtgtctgtgaaccccctcctggtcattcttagtgtcttggctctcacctggaccttcaaagggctttgtgttctccatccatgcagtctccaggcccttgtggttaaattcactgccctgtggattgttgttctcccggtcccgcaggtaaccgtggacttggtgctggtttgttatccgtagtgcagggactgtgtagatcacagaatgctgccaccatgccgtgtcttggttcatttggtccctgcacccatcaggagatgaacagagccttctaggtggccatacttacgtgatctctcctgaatttgttgatgatccagcctgctgggaagctgtttgtcatctgtccctagggggtttcagacctgcaggctgtgtgagtacagaacagagacttctggccagccccaggagttgacccaccacagtcccccacccagtgcctcagcttgggagcacagcaaaggtgccaaggtggagctgtctccagggcttcctggtgtggtcaatcccagcccagcactgctgggaaccaggagagccgggagtactccatagctgcctcagcctgatgctggcctgaggagcacaggctctcaaaggcccttcggctagcctgatctttgagttgtgtgcctctgtgttcttagtattgtttctgagagatacctgcccatttccccttgccccatataggagggaggaaaaagctggaggaaaccaaaaggcattaattactttgtagattataatgtgaaatataattctagattctttgtgaaatctgtcacatttaggtataacatttacagcccagttatccagccactgaagaaagctcacaacttgcttctttgcctcatccaaagctctgttgctgctaggtgcttgtagatgcttgacctccatccctccagtgcctcctcagtgaacttgggagcgtcttggaattcctccctgtgtggagtgcctctgcctcctgtgtatcctggcatttggttgaggcggccagtgcagctaaggctgatacctttggtcattggacaaggtgtcgtgtgtgtgtgtgtgtgtgtgtgtgtgtgtgtgtgtgtgtgtgtgtgtgtcctggtccagtgtggttgacaggctcagctgtatttctaggacatgttaggcatgaaagagagctccacctctccttgccagtcccaagtacctagccctccccctgggaggggcatctactgcaggccctctgcaccaggctcacccaggcccttgcctgctccccatacctctccagggctgtccctcctcagcattctgagccacctcagctgggtccatcagttgcactgttcgcccgaatcgttacttggttcctctgatccctttagttaaaacacccttcgttctgttgctaatgcttctctctggcttcccactttcccctcccagatttggaaacttggcttctgcccaccacatgggagtagcctgggaaatcactggttgccatttcatcattaatggagtagctacagacagcaaagccattggcctccctttctctgttttccttcttgaagcgctccattcctttcttcttgaaggcctagccttaattcagtttctcagcaggtggctctcaaggtgaaccaccccttgggcttacagagaaaagggcaacccttgctacccttgctaaaaggcaggctgtcaatctgtcaccgccctgagggatggactcagtgctttagaggaaagctggtggcttgtcactgccacacgccttgagtgcagggccctggtaggtggtctggaactggacttccttaggagtcgccactctcctgactaccctccactctgtgacaccagtcctctcactaaccctgtgggatcttggctcccagaacctcagcctgcatcctgccatctctcacctaactctgaggtcccttaaatccttcttggcccttggcttgctactgcctaccacctgttcccacaatcctccgtgacaccgttccctccctcattggctaccttacagctcttccctctccctaattccaggggactccaagttcatctagagggccattttgcatcatcctcttcctctgtgatcttcgaccagagcatcacgttcctaccctctgagccatgtgtccgcggctgctgccccatggccgcacttgactgtctgctttctcttgccatgatccttactccagccacccagatctaggttccccataggccctgttcatgtttcttcctctcatagacatgcccttcttccctacagcctgcctcctcaaaggcccagccctgagttctctttgtgttcagagcagaagcagctctatgctattctgtggccccatggcaatttggtgtcttgcttctggctcttaccactctggccggtgtagctgcagccaactgtgtactgtgttgcctccctctagactgtgagctcctcgtgggcagggaccatatggtcactctgtatctcttgtggcacctaaaagtgccttgcacttggtaggtgctcaataaatgtctgttcaagtg

SEQ ID NO: 21 - DNA, 1340 bp, Synthetic construct
  source 1..1340: mol_type = other DNA
ggagctgggccgggcccgagcggatcgcgggctcgggctgcggggctccggctgcgggcgctgggccgcgaggcgcggagcttgggagcggagcccaggccgtgccgcgcggcgccatgaagggcaaggaggagaaggagggcggcgcacggctgggcgctggcggcggaagccccgagaagagcccgagcgcgcaggagctcaaggagcagggcaatcgtctgttcgtgggccgaaagtacccggaggcggcggcctgctacggccgcgcgatcacccggaacccgctggtggccgtgtattacaccaaccgggccttgtgctacctgaagatgcagcagcacgagcaggccctggccgactgccggcgcgccctggagctggacgggcagtctgtgaaggcgcacttcttcctggggcagtgccagctggagatggagagctatgatgaggccatcgccaatctgcagcgagcttacagcctggccaaggagcagcggctgaacttcggggacgacatccccagcgctcttcgaatcgcgaagaagaagcgctggaacagcattgaggagcggcgcatccaccaggagagcgagctgcactcctacctctccaggctcattgccgcggagcgtgagagggagctggaagagtgccagcgaaaccacgagggtgatgaggacgacagccacgtccgggcccagcaggcctgcattgaggccaagcacgacaagtacatggcggacatggacgagcttttttctcaggtggatgagaagaggaagaagcgagacatccccgactacctgtgtggcaagatcagctttgagctgatgcgggagccgtgcatcacgcccagtggcatcacctacgaccgcaaggacatcgaggagcacctgcagcgtgtgggtcattttgaccccgtgacccggagccccctgacccaggaacagctcatccccaacttggctatgaaggaggttattgacgcattcatctctgagaatggctgggtggaggactactgaggttccctgccctacctggcgtcctggtccaggggagccctgggcagaagcccccggcccctatacatagtttatgttcctggccaccccgaccgcttcccccaagttctgctgttggactctggactgtttcccctctcagcatcgcttttgctgggccgtgatcgtccccctttgtgggctggaaaagcaggtgagggtgggctgggctgaggccattgccgccactatctgtgtaataaaatccgtgagcacgaggtgggacgtgctggtgtgtgaccggcagtcctgccagctgttttggctagccga

SEQ ID NO: 22 - DNA, 1232 bp, Synthetic construct
  source 1..1232: mol_type = other DNA
gatcgctgcgcgggctgcgagatctaggtggccgggcgcggagcccaagccgtgccgcgcggcgccatgaagggcaaggaggaaaaggagggcggcgcgcggctgggcactggtggcggcggcagccctgataagagcccgagtgcgcaagagctcaaggagcagggaaaccggctcttcgtgggccgcaagtacccggaggcggcggcctgctacggccgcgccatcactcggaacccacttgtggcagtgtactacactaaccgggccctgtgctatctgaagatgcagcagcctgaacaggcacttgctgactgccggcgagccctggagctggacgggcagtctgtgaaggcgcacttcttcctggggcagtgccagctggagatggagagttatgatgaggccattgccaatctgcagcgagcctatagtttggccaaggagcagcgactcaactttggggatgatattcctagtgcccttcgcattgctaagaagaagcgctggaacagtatcgaggaacggcgcatccaccaggagagtgagctgcattcatatctcaccaggctcattgctgctgagcgagagagggaactggaggagtgtcagcggaaccacgagggtcatgaagatgatggccacatccgggcccagcaggcctgcattgaggccaagcacgataaatacatggcagatatggatgagctcttctctcaggtggacgagaaaagaaagaagcgagatatccctgactacttgtgtggcaagattagctttgagctgatgcgggaaccctgcattacacccagtggtatcacctatgaccgcaaggacattgaggagcacctgcagcgtgtgggccactttgaccctgtgacccggagccctctgacccaggaacagctcatccccaacttggccatgaaggaagtcattgacgctttcatctctgagaacggctgggtagaggactattgaggccccatgtcctgcctggcaccctggcccaggaggatctggagacggaagctccagtccctgtatagtttgtgtccctgggcctgcccccatcggccctgctgatgggttctgaactgctccccttctcagcataccccttgctggaccatgagcctcccttgtcccccttctgggctggagagtgggtgagggtgggctgaggttgctgctgctgccactgtcctgtaataaagtctgtgagcactaca

SEQ ID NO: 23 - RNA, 20 nt, Synthetic construct
  source 1..20: mol_type = other RNA
cggggacacaggatccctgg

SEQ ID NO: 24 - RNA, 20 nt, Synthetic construct
  source 1..20: mol_type = other RNA
cccctggaagatgccatgac

SEQ ID NO: 25 - RNA, 20 nt, Synthetic construct
  source 1..20: mol_type = other RNA
attcccagggccggttaatg

SEQ ID NO: 26 - RNA, 20 nt, Synthetic construct
  source 1..20: mol_type = other RNA
agacgaccctggggacatag

SEQ ID NO: 27 - RNA, 20 nt, Synthetic construct
  source 1..20: mol_type = other RNA
ctggtactcctcgggatcaa

SEQ ID NO: 28 - RNA, 20 nt, Synthetic construct
  source 1..20: mol_type = other RNA
ggggtctgacagcaatgaag

SEQ ID NO: 29 - RNA, 20 nt, Synthetic construct
  source 1..20: mol_type = other RNA
taaaagcatatattgttgaa

SEQ ID NO: 30 - RNA, 20 nt, Synthetic construct
  source 1..20: mol_type = other RNA
tcaactagagattactttaa

SEQ ID NO: 31 - RNA, 20 nt, Synthetic construct
  source 1..20: mol_type = other RNA
ggaagacagtattgttcgaa

SEQ ID NO: 32 - RNA, 20 nt, Synthetic construct
  source 1..20: mol_type = other RNA
cgaggacgttggcagctggc

SEQ ID NO: 33 - RNA, 20 nt, Synthetic construct
  source 1..20: mol_type = other RNA
ttgacgaggatatctccacc

SEQ ID NO: 34 - RNA, 20 nt, Synthetic construct
  source 1..20: mol_type = other RNA
tcgaccctgggcagcaaaaa

SEQ ID NO: 35 - RNA, 20 nt, Synthetic construct
  source 1..20: mol_type = other RNA
cggagaggaaacctgcgccc

SEQ ID NO: 36 - RNA, 20 nt, Synthetic construct
  source 1..20: mol_type = other RNA
ctccctcaagaagtggaacg

SEQ ID NO: 37 - RNA, 20 nt, Synthetic construct
  source 1..20: mol_type = other RNA
cgatacgtgcgccatctgca

SEQ ID NO: 38 - RNA, 20 nt, Synthetic construct
  source 1..20: mol_type = other RNA
agtaaactgaagcgtgacga

SEQ ID NO: 39 - RNA, 20 nt, Synthetic construct
  source 1..20: mol_type = other RNA
aagtttgactggagagtctt

SEQ ID NO: 40 - RNA, 20 nt, Synthetic construct
  source 1..20: mol_type = other RNA
agagacaaattgcttgttaa

SEQ ID NO: 41 - RNA, 20 nt, Synthetic construct
  source 1..20: mol_type = other RNA
ctgctgccagttcttcacga

SEQ ID NO: 42 - RNA, 20 nt, Synthetic construct
  source 1..20: mol_type = other RNA
gatgggcctctccagcgggt

SEQ ID NO: 43 - RNA, 20 nt, Synthetic construct
  source 1..20: mol_type = other RNA
tgtgatgactggcgagcaga

SEQ ID NO: 44 - RNA, 20 nt, Synthetic construct
  source 1..20: mol_type = other RNA
attggttcaaattgtatagt

SEQ ID NO: 45 - RNA, 20 nt, Synthetic construct
  source 1..20: mol_type = other RNA
agagaaactttaaatccttt

SEQ ID NO: 46 - RNA, 20 nt, Synthetic construct
  source 1..20: mol_type = other RNA
ttctagtttgcaatggattt

SEQ ID NO: 47 - RNA, 20 nt, Synthetic construct
  source 1..20: mol_type = other RNA
gctttacctctcgtatttcg

SEQ ID NO: 48 - RNA, 20 nt, Synthetic construct
  source 1..20: mol_type = other RNA
agatgcagaaggcatcttgg

SEQ ID NO: 49 - RNA, 20 nt, Synthetic construct
  source 1..20: mol_type = other RNA
cttctcagactctgtaggct

SEQ ID NO: 50 - RNA, 20 nt, Synthetic construct
  source 1..20: mol_type = other RNA
ctctgattctttctgactac

SEQ ID NO: 51 - RNA, 20 nt, Synthetic construct
  source 1..20: mol_type = other RNA
cacttcctctgatgccaaga

SEQ ID NO: 52 - RNA, 20 nt, Synthetic construct
  source 1..20: mol_type = other RNA
gtttctgaaaagcttgattc

SEQ ID NO: 53 - RNA, 20 nt, Synthetic construct
  source 1..20: mol_type = other RNA
gcgcggcgccatgaagggca

SEQ ID NO: 54 - RNA, 20 nt, Synthetic construct
  source 1..20: mol_type = other RNA
cgagaagagcccgagcgcgc

SEQ ID NO: 55 - RNA, 20 nt, Synthetic construct
  source 1..20: mol_type = other RNA
cgtagcaggccgccgcctcc

SEQ ID NO: 56 - RNA, 20 nt, Synthetic construct
  source 1..20: mol_type = other RNA
ttctagacctcgtcctttgc

SEQ ID NO: 57 - RNA, 20 nt, Synthetic construct
  source 1..20: mol_type = other RNA
caagggctgcatgactggct

SEQ ID NO: 58 - RNA, 20 nt, Synthetic construct
  source 1..20: mol_type = other RNA
tcctctgggtccagcacctt

SEQ ID NO: 59 - protein, 493 aa, Synthetic construct
MALPVTALLLPLALLLHAARPDIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 60 - protein, 513 aa, Synthetic construct
MGILPSPGMPALLSLVSLLSVLLMGCVAESLLAWITQVGCGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGSHSMRYFFTSVSRPGRGEPRFIAVGYVDDTQFVRFDSDAASQRMEPRAPWIEQEGPEYWDGETRKVKAHSQTHRVDLGTLRGCYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPAGDGTFQKWAAVVVPSGQEQRYTCHVQHEGLPKPLTLRWEPSSQPTIPIVGIIAGLVLFGAVITGAVVAAVMWRRKSSDRKGGSYSQAASSDSAQGSDVSLTACKV

SEQ ID NO: 61 - protein, 612 aa, Synthetic construct
MSIGLLCCAALSLLWAGPVNAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSYVGNTGELFFGEGSRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRGRAKRSGSGATNFSLLKQAGDVEENPGPMETLLGLLILWLQLQWVSSKQEVTQIPAALSVPEGENLVLNCSFTDSAIYNLQWFRQDPGKGLTSLLLIQSSQREQTSGRLNASLDKSSGRSTLYIAASQPGDSATYLCAVRPLYGGSYIPTFGRGTSLIVHPYIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 62 - protein, 784 aa, Synthetic construct
MEHSTFLSGLVLATLLSQVSPFKIPIEELEDRVFVNCNTSITWVEGTVGTLLSDITRLDLGKRILDPRGIYRCNGTDIYKDKESTVQVHYRMCQSCVELDPATVAGIIVTDVIATLLLALGVFCFAGHETGRLSGAADTQALLRNDQVYQPLRDRDDAQYSHLGGNWARNKVKQTLNFDLLKLAGDVESNPGPMEQGKGLAVLILAIILLQGTLAQSIKGNHLVKVYDYQEDGSVLLTCDAEAKNITWFKDGKMIGFLTEDKKKWNLGSNAKDPRGMYQCKGSQNKSKPLQVYYRMCQNCIELNAATISGFLFAEIVSIFVLAVGVYFIAGQDGVRQSRASDKQTLLPNDQLYQPLKDREDDQYSHLQGNQLRRNEGRGSLLTCGDVEENPGPMQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRIQCTNYALLKLAGDVESNPGPMKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

(19) United States
(12) Patent Application Publication
(10) Pub. No.: US 2025/0032546 A1
(43) Pub. Date: Jan. 30, 2025

(54) GENETICALLY MODIFIED CELLS AND METHODS OF MAKING AND USING SAME

(71) Applicant: oNKo-Innate Pty Ltd, Moonee Ponds (AU)

(72) Inventors: Iva NIKOLIC, Melbourne (AU); Joseph CURSONS, Melbourne (AU); Nicholas David HUNTINGTON, Melbourne (AU)

(21) Appl. No.: 18/785,507

(22) Filed: Jul. 26, 2024

Related U.S. Application Data
(60) Provisional application No. 63/515,894, filed on Jul. 27, 2023.
(60) Provisional application No. 63/593,086, filed on Oct. 25, 2023.

(51) Int. Cl. A61K 35/17; A61K 39/00; C12N 5/0783; C12N 9/22; C12N 15/11

(52) U.S. Cl. CPC A61K 35/17; A61K 39/4613; A61K 39/4631; A61K 39/4632; C12N 5/0646; C12N 9/22; C12N 15/111; C12N 2310/20; C12N 2510/00

(57) ABSTRACT

Provided herein are genetically modified hematopoietic cells with reduced expression or activity of: (i) one or more CRL5 complex genes, and/or (ii) one or more genes orthogonal to the CRL5 complex, that exhibit improved therapeutic potential compared to wild-type hematopoietic cells, as well as methods of generating and using genetically modified hematopoietic cells for the treatment of a disease, such as cancer.

### CROSS-REFERENCE

This application claims priority to U.S. Provisional Application No. 63/515,894, filed on Jul. 27, 2023, and U.S. Provisional Application No. 63/593,086, filed on Oct. 25, 2023, which are incorporated herein by reference in their entirety.

### SEQUENCE LISTING

The instant application contains a Sequence Listing which is being submitted electronically in XML format and is hereby incorporated by reference in its entirety. The XML copy, created on Jul. 23, 2024, is named 51400_4002_SL_US.xml and is 139,516 bytes in size.

### BACKGROUND OF THE INVENTION

T-cells and natural killer (NK) cells are cytotoxic lymphocytes that play an important role in anti-tumor immunity, inter alia. The reactivity of T-cells towards a tumor is predominantly governed by the presentation of tumor-associated antigens (TAAs) on the surface of the cancer cells that constitute the tumor. Alternatively, NK cells detect and eliminate transformed cells, such as, for example, a cancer cell, through the use of a limited array of germline-encoded receptors. However, despite their strong anti-tumor potential, the potency of these immune cells is often limited by cellular dysfunction and exhaustion induced by a hostile tumor microenvironment (TME) in the settings of an oncological paradigm.

One treatment approach that has been employed to restore and/or induce anti-tumor response is adoptive cell therapy (ACT) using T-cells engineered to express chimeric antigen receptors (CARs) against certain TAAs. Further research and innovation in the field has resulted in the investigation of additional immune cell types (such as, for example, NK cells, tumor-infiltrating lymphocytes (TILs), natural killer T-(NKT-) cells, gamma-delta-(γδ-) T-cells, and the like) from various sources (such as, for example, autologous, allogeneic, cell-line derived, and the like), as well as the development of various other cellular engineering concepts (such as, for example, the expression of engineered T-cell receptor (TCR) or the expression of biological ‘logic’ gates) to improve the safety and response rates to ACT. Despite such advances, the persistence and function of these therapeutic cells is often greatly reduced upon infusion into a human subject (i.e., a patient). Hence, there is a need for improved ACT with improved proliferative capacity and resistance to immunosuppression.

### BRIEF SUMMARY OF THE INVENTION

In one aspect, the present disclosure provides a genetically modified hematopoietic cell, comprising: (A) one or more genetic modifications that inhibit expression or activity of polypeptide products encoded by one or more CRL5 complex-independent genes, wherein: (i) the one or more CRL5 complex-independent genes are selected from a group comprising: (a) ARHGAP25, (b) CCNC, (c) FAM49B, (d) N4BP1, (e) STUB1, or (f) any combination thereof, and (ii) expression or activity of the one or more polypeptide products is inhibited by at least about 50% compared to a control hematopoietic cell; and (B) one or more genetic modifications that inhibit expression or activity of polypeptide products encoded by one or more CRL5 complex genes, wherein: (i) the one or more CRL5 complex genes are selected from a group comprising: (a) ARIH2, (b) CUL5, (c) DCUN1D3, (d) RNF7, (e) UBE2F, (f) CISH, or (g) any combination thereof; and (ii) expression or activity of the one or more polypeptide products is inhibited by at least about 50% compared to a control hematopoietic cell.

In a further embodiment and in accordance with the above, the genetic modifications inactivate (i) the one or more CRL5 complex-independent genes, and/or (ii) the one or more CRL5 complex-independent genes.

In a further embodiment and in accordance with any of the above, (1) the one or more CRL5 complex-independent genes are inhibited or inactivated using a first genetic modification system selected from a group comprising: (i) a clustered, regularly interspaced, short palindromic repeats (CRISPR) system, (ii) a transcription activator-like effector nuclease (TALEN) system, (iii) a zinc finger nuclease (ZFN) system, (iv) a meganuclease system, (v) a short hairpin RNA (shRNA) system, (vi) a small interfering RNA (siRNA) system, (vii) a microRNA (miRNA) system, or (viii) an antisense RNA system; and (2) the one or more CRL5 complex genes are inhibited or inactivated using a second genetic modification system selected from a group comprising: (i) a clustered, regularly interspaced, short palindromic repeats (CRISPR) system, (ii) a transcription activator-like effector nuclease (TALEN) system, (iii) a zinc finger nuclease (ZFN) system, (iv) a meganuclease system, (v) a short hairpin RNA (shRNA) system, (vi) a small interfering RNA (siRNA) system, (vii) a microRNA (miRNA) system, or (viii) an antisense RNA system.

In a further embodiment and in accordance with any of the above, the genetically modified hematopoietic cell comprises a natural killer (NK) cell. In some further embodiments, the NK cell comprises a CAR-NK cell.

In a further embodiment and in accordance with any of the above, the genetic modifications to (i) the one or more CRL5 complex-independent genes, and (ii) the one or more CRL5 complex genes improve proliferation of the genetically modified hematopoietic cell by about 125% to about 150%, about 250% to about 300%, about 400% to about 500%, about 850% to about 900%, about 1600% to about 1650%, or about 2200% to about 2250% compared to proliferation of a control hematopoietic cell, as determined by a first assay. In some other embodiments, the genetic modifications to (i) the one or more CRL5 complex-independent genes, and (ii) the one or more CRL5 complex genes improve proliferation of the genetically modified hematopoietic cell by about 1.25-fold to about 1.50-fold, about 2.5-fold to about 3.0-fold, about 4.0-fold to about 5.0-fold, about 8.5-fold to about 9.0-fold, about 16.0-fold to about 16.5-fold, or about 22.0-fold to about 22.5-fold compared to proliferation of a control hematopoietic cell, as determined by a first assay.

In a further embodiment and in accordance with either of the above, the first assay comprises: (i) a quantification of cells and/or cell clusters using a cell imaging system (e.g., Incucyte), (ii) a 5-ethynyl-2′-deoxyuridine (EdU) incorporation assay, (iii) a bromodeoxyuridine (BrdU) incorporation assay, (iv) a tetrazolium salt reduction assay, (v) an ATP assay, or (vi) a Ki-67 staining assay.

In a further embodiment and in accordance with any of the above, the genetic modifications to (i) the one or more CRL5 complex-independent genes, and (ii) the one or more CRL5 complex genes improve resistance to immune suppression of the genetically modified hematopoietic cell by about 60.00% to about 90.00%, about 90.00% to about 15.0%, about 15.0% to about 21.0%, about 21.0% to about 27.0%, about 45.0% to about 48.0%, about 5⁴0.0% to about 69.0%, about 60.0% to about 70.0%, or about 81.0% to about 84.0% compared to resistance to immune suppression of a control hematopoietic cell, as determined by a second assay. In some other embodiments, the genetic modifications to (i) the one or more CRL5 complex-independent genes, and (ii) the one or more CRL5 complex genes improve resistance to immune suppression of the genetically modified hematopoietic cell by about 0.06-fold to about 0.09-fold, about 0.09-fold to about 0.15-fold, about 0.15-fold to about 0.21-fold, about 0.21-fold to about 0.27-fold, about 0.45-fold to about 0.48-fold, about 0.54-fold to about 0.69-fold, about 0.60-fold to about 0.70-fold, or about 0.81-fold to about 0.84-fold compared to immune suppression of a control hematopoietic cell, as determined by a second assay.

In a further embodiment and in accordance with either of the above, the second assay comprises (i) a serial challenge assay, or (ii) a cytokine release assay.

In a further embodiment and in accordance with any of the above, the genetic modifications to (i) the one or more CRL5 complex-independent genes, and (ii) the one or more CRL5 complex genes improve the cytotoxic capacity of the genetically modified hematopoietic cell by about 67.0% to about 72.0%, about 78.0% to about 87.0%, or about 90.0% to about 100.0% compared to cytotoxic capacity of a control hematopoietic cell, as determined by a third assay. In some other embodiments, the genetic modifications to (i) the one or more CRL5 complex-independent genes, and (ii) the one or more CRL5 complex genes improve the cytotoxic capacity of the genetically modified hematopoietic cell by about 0.67-fold to about 0.72-fold, about 0.78-fold to about 0.87-fold, or about 0.9-fold to about 1.0-fold compared to cytotoxic capacity of a control hematopoietic cell, as determined by a third assay.

In a further embodiment and in accordance with either of the above, the third assay comprises: (i) a quantification of cells and/or cell clusters using a cell imaging system (e.g., Incucyte), (ii) a serial challenge assay, (iii) an enzymatic cytotoxicity assay, (iv) an apoptosis assay, (v) an autophagy assay, or (vi) a cell viability assay.

In a further embodiment and in accordance with any of the above, the genetic modifications to (i) the one or more CRL5 complex-independent genes, and (ii) the one or more CRL5 complex genes improve the killing efficiency of cancer cells by the genetically modified hematopoietic cell by about 67.0% to about 72.0%, about 78.0% to about 87.0%, or about 90.0% to about 100.0% compared to the killing efficiency of cancer cells by a control hematopoietic cell, as determined by a fourth assay. In some other embodiments, the genetic modifications to (i) the one or more CRL5 complex-independent genes, and (ii) the one or more CRL5 complex genes improve the killing efficiency of cancer cells by the genetically modified hematopoietic cell by about 0.67-fold to about 0.72-fold, about 0.78-fold to about 0.87-fold, or about 0.9-fold to about 1.0-fold compared to the killing efficiency of cancer cells by a control hematopoietic cell, as determined by a fourth assay.

In a further embodiment and in accordance with either of the above, the fourth assay comprises: (i) a quantification of cells and/or cell clusters using a cell imaging system (e.g., Incucyte), (ii) a serial challenge assay, (iii) an enzymatic cytotoxicity assay, (iv) an apoptosis assay, (v) an autophagy assay, or (vi) a cell viability assay.

In a further embodiment and in accordance with any of the above, the genetic modifications to (i) the one or more CRL5 complex-independent genes, and (ii) the one or more CRL5 complex genes improve capability to stimulate production of one or more cytokines by the genetically modified hematopoietic cell by about 5.0% to about 10.0%, about 15.0% to about 20.0%, about 25.0% to about 30.0%, about 55.0% to about 60.0%, about 70.0% to about 75.0%, about 80.0% to about 85.0%, about 90.0% to about 100.0%, about 120.0% to about 125.0%, about 160.0% to about 165.0%, about 205.0% to about 210.0%, about 280.0% to about 300.0%, about 340.0% to about 350.0%, or about 505.0% to about 510.0% compared to capability to stimulate production of one or more cytokines of a control hematopoietic cell, as determined by a fifth assay. In some other embodiments, the genetic modifications to (i) the one or more CRL5 complex-independent genes, and (ii) the one or more CRL5 complex genes improve capability to stimulate production of one or more cytokines by the genetically modified hematopoietic cell by about 0.05-fold to about 0.10-fold, about 0.15-fold to about 0.20-fold, about 0.25-fold to about 0.30-fold, about 0.55-fold to about 0.60-fold, about 0.70-fold to about 0.75-fold, about 0.80-fold to about 0.85-fold, about 0.90-fold to about 1.00-fold, about 1.20-fold to about 1.25-fold, about 1.60-fold to about 1.65-fold, about 2.05-fold to about 2.10-fold, about 2.80-fold to about 3.00-fold, about 3.40-fold to about 3.5-fold, or about 5.05-fold to about 5.10-fold compared to capability to stimulate production of one or more cytokines of a control hematopoietic cell, as determined by a fifth assay.

In a further embodiment and in accordance with either of the above, the one or more cytokines are selected from the group including: (i) granulocyte colony-stimulating factor (G-CSF), (ii) granulocyte-macrophage colony-stimulating factor (GM-CSF), (iii) interferon alpha (IFN-α), (iv) interferon gamma (IFN-γ), (v) interleukin-1 beta (IL-1β), (vi) IL-2, (vii) IL-4, (viii) IL-5, (ix) IL-6, (x) IL-8, (xi) IL-10, (xii) interleukin-12 p70 (IL-12 p70), (xiii) IL-13, (xiv) IL-17A, (xv) IL-18, (xvi) interferon gamma-induced protein 10 (IP-10), (xvii) monocyte chemoattractant protein-1 (MCP-1), (xviii) macrophage inflammatory protein-1 alpha (MIP-la), (xix) MIP-1 beta (MIP-1β), (xx) tumor necrosis factor alpha (TNF-α), (xxi) TNF-β, and (xxii) any combination thereof.

In a further embodiment and in accordance with the above, the fifth assay comprises: (i) a ProcartaPlex multiplex cytokine assay, (ii) a Human LEGENDplex TH1 panel, (iii) an AlphaLISA assay, (iv) an intracellular cytokine stain assay, (v) a Luminex bead-based cytokine release assay, (vi) a MACS cytokine secretion assay, (vii) an ELISA, or (viii) an ELISpot assay.

In a further embodiment and in accordance with any of the above, the control hematopoietic cell comprises a wild-type NK cell.

In another aspect, the present disclosure provides a method of generating a genetically modified cell population for treatment of a subject that has cancer, the method comprising: (a) obtaining a plurality of hematopoietic cells; (b) inhibiting expression of one or more CRL5 complex-independent genes, wherein the one or more CRL5 complex-independent genes is selected from a group comprising: (i) ARHGAP25, (ii) CCNC, (iii) FAM49B, (iv) N4BP1, (v) STUB1, and (vi) any combination thereof, (c) inhibiting expression of one or more CRL5 complex genes, wherein the one or more one or more CRL5 complex genes is selected from a group comprising: (i) ARIH2, (ii) CUL5, (iii) DCUN1D3, (iv) RNF7, (v) UBE2F, (vi) CISH, or (vii) any combination thereof; (d) selecting hematopoietic cells in which the one or more CRL5 complex-independent genes and the one or more CRL5 complex genes are inhibited; and (e) expanding the selected hematopoietic cells ex vivo, thereby generating a genetically modified cell population for treatment of a subject that has cancer.

In a further embodiment and in accordance with the above, the plurality of hematopoietic cells is derived from (i) the subject that has cancer, (ii) a donor that does not have cancer, or (iii) a cell line.

In a further embodiment and in accordance with the above, the cell line is selected from the group including: (i) an induced pluripotent stem cell (iPSC) line, and (ii) a hematopoietic stem cell (HSC) line.

In another aspect, the present disclosure provides a method of treating cancer, comprising: administering a population of cells comprising a genetically modified hematopoietic cell, as described above, to a subject that has cancer.

In a further embodiment and in accordance with the above, the population of cells is derived from (i) the subject that has cancer, (ii) a donor that does not have cancer, or (iii) a cell line.

In a further embodiment and in accordance with the above, the cell line is selected from the group including: (i) an induced pluripotent stem cell (iPSC) line, and (ii) a hematopoietic stem cell (HSC) line.

In a further embodiment and in accordance with any of the above, the population of cells comprises about 100,000 (one hundred thousand) cells to about 100,000,000,000 (one hundred billion) cells.

In a further embodiment and in accordance with any of the above, prior to the administering, the subject has received a myeloablative conditioning regimen, a nonmyeloablative conditioning regimen, or a reduced intensity conditioning regimen.

In another aspect, the present disclosure provides a genetically modified hematopoietic cell, comprising: a genetic modification to one or more CRL5 complex genes that inhibits expression or activity of the polypeptide encoded by the one or more CRL5 complex genes, wherein expression or activity of the polypeptide product is inhibited by at least about 50% compared to a control hematopoietic cell.

In a further embodiment and in accordance with the above, the one or more CRL5 complex genes are selected from a group including: (i) ARIH2, (ii) CUL5, (iii) DCUN1D3, (iv) RNF7, (v) UBE2F, and (vi) any combination thereof.

In a further embodiment and in accordance with any of the above, the genetic modification of the one or more CRL5 complex genes inactivates the one or more CRL5 complex genes.

In a further embodiment and in accordance with any of the above, the one or more CRL5 complex genes are inhibited using: (i) a clustered, regularly interspaced, short palindromic repeats (CRISPR) system, (ii) a transcription activator-like effector nuclease (TALEN) system, (iii) a zinc finger nuclease (ZFN) system, (iv) a meganuclease system, (v) a short hairpin RNA (shRNA) system, (vi) a small interfering RNA (siRNA) system, (vii) a microRNA (miRNA) system, or (viii) an antisense RNA system.

In a further embodiment and in accordance with any of the above, the genetically modified hematopoietic cell is a cell selected from a group including: (i) a T-cell, (ii) a natural killer (NK) cell, and (iii) a tumor-infiltrating lymphocyte (TIL).

In a further embodiment and in accordance with the above, the T-cell comprises a chimeric antigen receptor-(CAR-) T-cell or a T-cell receptor-(TCR-) T-cell. Alternatively, in a separate further embodiment and in accordance with the above, the NK cell comprises a CAR-NK cell or a TCR-NK cell.

In a further embodiment and in accordance with any of the above, the genetic modification to one or more CRL5 complex genes improves proliferation of the modified hematopoietic cell by about 10% to about 100% compared to proliferation of a control hematopoietic cell, as determined by a first assay.

In a further embodiment and in accordance with any of the above, the genetic modification to one or more CRL5 complex genes improves proliferation of the modified hematopoietic cell by about 1.0-fold to about 15.0-fold compared to proliferation of a control hematopoietic cell, as determined by a first assay.

In a further embodiment and in accordance with the above, the first assay comprises: (i) a 5-ethynyl-2′-deoxyuridine (EdU) incorporation assay, (ii) a bromodeoxyuridine (BrdU) incorporation assay, (iii) a tetrazolium salt reduction assay, (iv) an ATP assay, or (v) a Ki-67 staining assay.

In a further embodiment and in accordance with any of the above, the genetic modification to one or more CRL5 complex genes improves resistance to immune suppression by about 10% to about 100% compared to resistance to immune suppression of a control hematopoietic cell, as determined by a second assay.

In a further embodiment and in accordance with any of the above, the genetic modification to one or more CRL5 complex genes improves resistance to immune suppression by about 1.0-fold to about 10.0-fold compared to immune suppression of a control hematopoietic cell, as determined by a second assay.

In a further embodiment and in accordance with the above, the second assay comprises (i) a serial challenge assay, or (ii) a cytokine release assay.

In a further embodiment and in accordance with any of the above, the genetic modification to one or more CRL5 complex genes improves the cytotoxic capacity by about 10% to about 100% compared to cytotoxic capacity of a control hematopoietic cell, as determined by a third assay.

In a further embodiment and in accordance with any of the above, the genetic modification to one or more CRL5 complex genes improves the cytotoxic capacity by about 0.5-fold to about 10.0-fold compared to cytotoxic capacity of a control hematopoietic cell, as determined by a third assay.

In a further embodiment and in accordance with the above, the third assay comprises: (i) a serial challenge assay, (ii) an enzymatic cytotoxicity assay, (iii) an apoptosis assay, (iv) an autophagy assay, or (v) a cell viability assay.

In a further embodiment and in accordance with any of the above, the genetic modification to one or more CRL5 complex genes improves the killing efficiency of cancer cells by the genetically modified hematopoietic cells by about 10% to about 100% compared to the killing efficiency of cancer cells by a control hematopoietic cell, as determined by a fourth assay.

In a further embodiment and in accordance with any of the above, the genetic modification to one or more CRL5 complex genes improves the killing efficiency of cancer cells by the genetically modified hematopoietic cells by about 0.5-fold to about 10.0-fold compared to the killing efficiency of cancer cells by a control hematopoietic cell, as determined by a fourth assay.

In a further embodiment and in accordance with the above, the fourth assay comprises: (i) a serial challenge assay, (ii) an enzymatic cytotoxicity assay, (iii) an apoptosis assay, (iv) an autophagy assay, or (v) a cell viability assay.

In a further embodiment and in accordance with any of the above, the genetic modification to one or more CRL5 complex genes improves capability to stimulate production of one or more cytokines by about 10% to about 100% compared to capability to stimulate production of one or more cytokines of a control hematopoietic cell, as determined by a fifth assay.

In a further embodiment and in accordance with any of the above, the genetic modification to one or more CRL5 complex genes improves capability to stimulate production of one or more cytokines by about 0.5-fold to about 20.0-fold compared to capability to stimulate production of one or more cytokines of a control hematopoietic cell, as determined by a fifth assay.

In a further embodiment and in accordance with the above, the one or more cytokines are selected from a group including: (i) granulocyte colony-stimulating factor (G-CSF), (ii) granulocyte-macrophage colony-stimulating factor (GM-CSF), (iii) interferon alpha (IFN-α), (iv) interferon gamma (IFN-γ), (v) interleukin-1 beta (IL-1β), (vi) IL-2, (vii) IL-4, (viii) IL-5, (ix) IL-6, (x) IL-8, (xi) IL-10, (xii) interleukin-12 p70 (IL-12 p70), (xiii) IL-13, (xiv) IL-17A, (xv) IL-18, (xvi) interferon gamma-induced protein 10 (IP-10), (xvii) monocyte chemoattractant protein-1 (MCP-1), (xviii) macrophage inflammatory protein-1 alpha (MIP-la), (xix) MIP-1 beta (MIP-10), (xx) tumor necrosis factor alpha (TNF-α), (xxi) TNF-β, and (xxii) any combination thereof.

In a further embodiment and in accordance with the above, the fifth assay comprises: (i) a ProcartaPlex multiplex cytokine assay, (ii) a Human LEGENDplex TH1 panel, (iii) an AlphaLISA assay, (iv) an intracellular cytokine stain assay, (v) a Luminex bead-based cytokine release assay, (vi) a MACS cytokine secretion assay, (vii) an ELISA, or (viii) an ELISpot assay.

In a further embodiment and in accordance with any of the above, the control hematopoietic cell is selected from a group including: (i) a wild-type T-cell, (ii) a wild-type NK cell, and (iii) a wild-type TIL.

In another aspect, the present disclosure provides a method of generating a genetically modified cell population for treatment of a subject that has cancer, the method comprising: (a) obtaining a plurality of hematopoietic cells; (b) inhibiting expression of one or more CRL5 complex genes, wherein the one or more CRL5 complex genes is selected a group including: (i) ARIH2, (ii) CUL5, (iii) DCUN1D3, (iv) RNF7, (v) UBE2F, and (vi) any combination thereof; (c) selecting hematopoietic cells in which the one or more CRL5 complex genes are inhibited; and (d) expanding the selected hematopoietic cells ex vivo, thereby generating a genetically modified cell population for treatment of a subject that has cancer.

In a further embodiment and in accordance with the above, the plurality of hematopoietic cells is derived from (i) the subject that has cancer, (ii) a donor that does not have cancer, or (iii) a cell line.

In a further embodiment and in accordance with the above, the cell line is selected from a group including: (i) an induced pluripotent stem cell (iPSC) line, and (ii) a hematopoietic stem cell (HSC) line.

In another aspect, the present disclosure provides a method of treating cancer, comprising: administering a population of cells comprising a genetically modified hematopoietic cell according to any of the aspects and embodiments described herein to a subject that has cancer.

In a further embodiment and in accordance with the above, the population of cells is derived from (i) the subject that has cancer, (ii) a donor that does not have cancer, or (iii) a cell line.

In a further embodiment and in accordance with the above, the cell line is selected from a group including: (i) an induced pluripotent stem cell (iPSC) line, and (ii) a hematopoietic stem cell (HSC) line.

In a further embodiment and in accordance with any of the above, the population of cells comprises about 100,000 (one hundred thousand) cells to about 100,000,000,000 (one hundred billion) cells.

In a further embodiment and in accordance with any of the above, prior to the administering, the subject has received a myeloablative conditioning regimen, a nonmyeloablative conditioning regimen, or a reduced intensity conditioning regimen.

In another aspect, the present disclosure provides a genetically modified hematopoietic cell, comprising: one or more genetic modifications that inhibit expression or activity of polypeptide products encoded by one or more CRL5 complex-independent genes, wherein: (i) the one or more CRL5 complex-independent genes are selected from a group comprising: (a) ARHGAP25, (b) CCNC, (c) FAM49B, (d) N4BP1, (e) STUB1, or (f) any combination thereof, and (ii) expression or activity of the one or more polypeptide products is inhibited by at least about 50% compared to a control hematopoietic cell.

In a further embodiment and in accordance with the above, the one or more genetic modifications of the one or more CRL5 complex-independent genes inactivates the one or more CRL5 complex-independent genes.

In a further embodiment and in accordance with any of the above, the one or more CRL5 complex-independent genes are inhibited using: (i) a clustered, regularly interspaced, short palindromic repeats (CRISPR) system, (ii) a transcription activator-like effector nuclease (TALEN) system, (iii) a zinc finger nuclease (ZFN) system, (iv) a meganuclease system, (v) a short hairpin RNA (shRNA) system, (vi) a small interfering RNA (siRNA) system, (vii) a microRNA (miRNA) system, or (viii) an antisense RNA system.

In a further embodiment and in accordance with any of the above, the genetically modified hematopoietic cell comprises a natural killer (NK) cell. In some further embodiments, the NK cell comprises a CAR-NK cell.

In a further embodiment and in accordance with any of the above, the one or more genetic modifications to one or more CRL5 complex-independent genes improves proliferation of the genetically modified hematopoietic cell by about 50% to about 75%, about 75% to about 100%, about 250% to about 300%, or about 625% to about 650% compared to proliferation of a control hematopoietic cell, as determined by a first assay. In some other embodiments, the one or more genetic modifications to one or more CRL5 complex-independent genes improves proliferation of the genetically modified hematopoietic cell by about 0.5-fold to about 0.75-fold, about 0.75-fold to about 1.0-fold, about 2.5-fold to about 3.0-fold, or about 6.25-fold to about 6.50-fold compared to proliferation of a control hematopoietic cell, as determined by a first assay.

In a further embodiment and in accordance with either of the above, the first assay comprises: (i) a quantification of cells and/or cell clusters using a cell imaging system (e.g., Incucyte), (ii) a 5-ethynyl-2′-deoxyuridine (EdU) incorporation assay, (iii) a bromodeoxyuridine (BrdU) incorporation assay, (iv) a tetrazolium salt reduction assay, (v) an ATP assay, or (vi) a Ki-67 staining assay.

In a further embodiment and in accordance with any of the above, the one or more genetic modifications to one or more CRL5 complex-independent genes improves resistance to immune suppression of the genetically modified hematopoietic cell by about 1.5% to about 6.0%, about 21.0% to about 24.0%, about 27.0% to about 30.0%, about 42.0% to about 45.0%, or about 53.0% to about 55.0% compared to resistance to immune suppression of a control hematopoietic cell, as determined by a second assay. In some other embodiments, the one or more genetic modifications to one or more CRL5 complex-independent genes improves resistance to immune suppression of the genetically modified hematopoietic cell by about 0.015-fold to about 0.060-fold, about 0.21-fold to about 0.24-fold, about 0.27-fold to about 0.30-fold, about 0.42-fold to about 0.45-fold, or about 0.53-fold to about 0.55-fold compared to immune suppression of a control hematopoietic cell, as determined by a second assay.

In a further embodiment and in accordance with either of the above, the second assay comprises (i) a serial challenge assay, or (ii) a cytokine release assay.

In a further embodiment and in accordance with any of the above, the one or more genetic modifications to one or more CRL5 complex-independent genes improves the cytotoxic capacity of the genetically modified hematopoietic cell by about 18.0% to about 24.0%, about 60.0% to about 63.0%, about 72.0% to about 75.0%, or about 87% to about 90% compared to cytotoxic capacity of a control hematopoietic cell, as determined by a third assay. In some other embodiments, the one or more genetic modifications to one or more CRL5 complex-independent genes improves the cytotoxic capacity of the genetically modified hematopoietic cell by about 0.18-fold to about 0.24-fold, about 0.60-fold to about 0.63-fold, about 0.72-fold to about 0.75-fold, or about 0.87-fold to about 0.90-fold compared to cytotoxic capacity of a control hematopoietic cell, as determined by a third assay.

In a further embodiment and in accordance with either of the above, the third assay comprises: (i) a quantification of cells and/or cell clusters using a cell imaging system (e.g., Incucyte), (ii) a serial challenge assay, (iii) an enzymatic cytotoxicity assay, (iv) an apoptosis assay, (v) an autophagy assay, or (vi) a cell viability assay.

In a further embodiment and in accordance with any of the above, the one or more genetic modifications to one or more CRL5 complex-independent genes improves the killing efficiency of cancer cells by the genetically modified hematopoietic cell by about 18.0% to about 24.0%, about 60.0% to about 63.0%, about 72.0% to about 75.0%, or about 87% to about 90% compared to the killing efficiency of cancer cells by a control hematopoietic cell, as determined by a fourth assay. In some other embodiments, the one or more genetic modifications to one or more CRL5 complex-independent genes improves the killing efficiency of cancer cells by the genetically modified hematopoietic cell by about 0.18-fold to about 0.24-fold, about 0.60-fold to about 0.63-fold, about 0.72-fold to about 0.75-fold, or about 0.87-fold to about 0.90-fold compared to the killing efficiency of cancer cells by a control hematopoietic cell, as determined by a fourth assay.

In a further embodiment and in accordance with either of the above, the fourth assay comprises: (i) a quantification of cells and/or cell clusters using a cell imaging system (e.g., Incucyte), (ii) a serial challenge assay, (iii) an enzymatic cytotoxicity assay, (iv) an apoptosis assay, (v) an autophagy assay, or (vi) a cell viability assay.

In a further embodiment and in accordance with any of the above, the one or more genetic modifications to one or more CRL5 complex-independent genes improves capability to stimulate production of one or more cytokines by the genetically modified hematopoietic cell by about 5.0% to about 10.0%, about 10.0% to about 15.0%, about 15.0% to about 20.0%, about 20.0% to about 25.0%, or about 25.0% to about 30.0% compared to capability to stimulate production of one or more cytokines of a control hematopoietic cell, as determined by a fifth assay. In some other embodiments, the one or more genetic modifications to one or more CRL5 complex-independent genes improves capability to stimulate production of one or more cytokines by the genetically modified hematopoietic cell by about 0.05-fold to about 0.10-fold, about 0.10-fold to about 0.15-fold, about 0.15-fold to about 0.20-fold, about 0.20-fold to about 0.25-fold, or about 0.25-fold to about 0.30-fold compared to capability to stimulate production of one or more cytokines of a control hematopoietic cell, as determined by a fifth assay.

In a further embodiment and in accordance with either of the above, the one or more cytokines are selected from the group including: (i) granulocyte colony-stimulating factor (G-CSF), (ii) granulocyte-macrophage colony-stimulating factor (GM-CSF), (iii) interferon alpha (IFN-α), (iv) interferon gamma (IFN-γ), (v) interleukin-1 beta (IL-1β), (vi) IL-2, (vii) IL-4, (viii) IL-5, (ix) IL-6, (x) IL-8, (xi) IL-10, (xii) interleukin-12 p70 (IL-12 p70), (xiii) IL-13, (xiv) IL-17A, (xv) IL-18, (xvi) interferon gamma-induced protein 10 (IP-10), (xvii) monocyte chemoattractant protein-1 (MCP-1), (xviii) macrophage inflammatory protein-1 alpha (MIP-la), (xix) MIP-1 beta (MIP-1β), (xx) tumor necrosis factor alpha (TNF-α), (xxi) TNF-β, and (xxii) any combination thereof.

In a further embodiment and in accordance with the above, the fifth assay comprises: (i) a ProcartaPlex multiplex cytokine assay, (ii) a Human LEGENDplex TH1 panel, (iii) an AlphaLISA assay, (iv) an intracellular cytokine stain assay, (v) a Luminex bead-based cytokine release assay, (vi) a MACS cytokine secretion assay, (vii) an ELISA, or (viii) an ELISpot assay.

In a further embodiment and in accordance with any of the above, the control hematopoietic cell comprises a wild-type NK cell.

In another aspect, the present disclosure provides a method of generating a genetically modified cell population for treatment of a subject that has cancer, the method comprising: (a) obtaining a plurality of hematopoietic cells; (b) inhibiting expression of one or more CRL5 complex-independent genes, wherein the one or more CRL5 complex genes is selected from a group comprising: (i) ARHGAP25, (ii) CCNC, (iii) FAM49B, (iv) N4BP1, (v) STUB1, or (vi) any combination thereof; (c) selecting hematopoietic cells in which the one or more CRL5 complex-independent genes are inhibited; and (d) expanding the selected hematopoietic cells ex vivo, thereby generating a genetically modified cell population for treatment of a subject that has cancer.

In a further embodiment and in accordance with the above, the plurality of hematopoietic cells is derived from (i) the subject that has cancer, (ii) a donor that does not have cancer, or (iii) a cell line.

In a further embodiment and in accordance with the above, the cell line is selected from the group including: (i) an induced pluripotent stem cell (iPSC) line, and (ii) a hematopoietic stem cell (HSC) line.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Fig.”, “FIG.”, “Figure”, “Figures”, “Figs.”, and “FIGs.” herein) of which:

FIG. 1 shows data for primary human natural killer (NK) cells edited using a CRISPR system and the subsequent effect on K562 target cells. Genes that were edited in these figures include: select components of the CRL5 complex (ARIH2, UBE2F, CUL5, DCUN1D3, or RNF7), CISH or RASA2 (positive control genes), or the negative control gene AAVS1. FIG. 1A shows the results of a proliferation assay measured by EdU uptake in primary human NK cells CRISPR edited with the indicated sgRNA. The assay was performed in the presence of either a high concentration (5 ng/mL) or a low concentration (1 ng/mL) of interleukin-15 (IL-15). FIG. 1B shows the expression of CD122 (IL2Rβ) in primary human NK cells edited with the indicated sgRNA. FIG. 1C shows Western blot results showing the efficiency of the gene target knockout for the indicated sgRNA in primary human NK cells. FIG. 1D shows the results of a serial challenge assay using primary human NK cells edited with the indicated sgRNA and K562 target cells. The assay was conducted using low IL-15 conditions (1 ng/mL) in the absence (top panel) or presence (bottom panel) of the immunosuppressive factor adenosine (NECA). “R1” represents the first assessment point in the serial challenge assay, “R2” represents the second assessment point. “R3” represents the third assessment point, and “R4” represents the fourth assessment point. FIG. 1E shows a summary of the results of FIG. 1D represented as a percentage of increased killing compared to the control NK cells (wherein AAVS1 was targeted using a CRISPR system) in the fourth round of the serial challenge assay. FIG. 1F shows measurements of the NK cell degranulation marker CD107 during the first round of the serial challenge assay.

FIG. 1G shows a quantification of various cytokines released after the second round of the serial challenge assay. Bars represent mean±standard deviation (SD), and symbols represent the individual data points. “ns” indicates not significant, * indicates p<0.05, ** indicates p<0.01, *** indicates p<0.001, and **** indicates p<0.0001.

FIG. 2 shows data for ABECMA CAR-NK cells edited using a CRISPR and the subsequent effect on target cells (Nalm6) engineered to express BCMA. Genes that were edited in these figures include: select components of the CRL5 complex (ARIH2, UBE2F, CUL5, DCUN1D3, or RNF7), CISH or RASA2 (positive control genes), or the negative control gene AAVS1. FIG. 2A shows a schematic describing the manufacturing of NK cells modified using an ABECMA CAR construct followed by editing of the CRL5 complex, CISH, RASA2, or the control gene AAVS1 using a CRISPR system prior to the performing of a serial challenge assay using target cells (Nalm6) engineered to express high levels of BCMA (Nalm6-BCMA). FIG. 2B show quality control fluorescence-activated cell sorting (FACS) plots for the described CAR-NK cells. The plots display the percentage of CAR transduction as measured by mScarlet fluorescence in the primary NK cells before (top panel) and after (bottom panel) CRISPR ribonucleoprotein (RNP) editing. High CAR expression was maintained over the course of the experiment and was not affected by the RNP editing. FIG. 2C shows the expression of CD122 (IL2Rβ) in CAR-NK cells edited with the indicated sgRNA. FIG. 2D shows the results of a serial challenge assay using NK cells (top panel) or CAR-NK cells (bottom panel) edited with the indicated sgRNA and Nalm6-BCMA target cells as determined by the number of live targets normalized to the initial number of cells (using Incucyte). Cells were plated at an effector:target (E:T) ratio of 1:4 or 1:6 (data for E:T ratio of 1:4 not shown). FIG. 2E shows a summary of the results of FIG. 2D represented as a percentage of increased killing compared to the control CAR-NK cells (wherein AAVS1 was targeted using a CRISPR system). FIG. 2F shows a quantification of various cytokines released after the second round of the serial challenge assay with edited CAR-NK cells (E:T ratio of 1:4). Bars represent mean±standard deviation (SD), and symbols represent the individual data points. “ns” indicates not significant, * indicates p<0.05, ** indicates p<0.01, *** indicates p<0.001, and **** indicates p<0.0001.

FIG. 3 shows data for ABECMA CAR-NK cells edited using a CRISPR system and the subsequent effect on target cells (RPMI-8226) that exhibit endogenous expression of BCMA. Genes that were edited in these figures include: select components of the CRL5 complex (ARIH2, UBE2F, CUL5, DCUN1D3, or RNF7), CISH or RASA2 (positive control genes), or the negative control gene AAVS1. FIG. 3A shows a schematic describing the manufacturing of NK cells modified using an ABECMA CAR construct followed by editing of the CRL5 complex, CISH, RASA2, or the control gene AAVS1 using a CRISPR system prior to the performing of a serial challenge assay using target cells (RPMI-8226) that endogenously express BCMA. FIG. 3B shows the expression of CD122 (IL2Rβ) in CAR-NK cells edited with the indicated sgRNA. FIG. 3C shows the results of a serial challenge assay using NK cells (top panel) or CAR-NK cells (bottom panel) edited with the indicated sgRNA, and RPMI-8226 target cells as determined by the number of live targets normalized to the initial number of cells (using Incucyte). Cells were plated at an E:T ratio of 1:2. FIG. 3D shows a summary of the results of FIG. 3C represented as a percentage of increased killing compared to the control NK cells (wherein AAVS1 was targeted using a CRISPR system; top panel shows the summary of results for mock transduced NK cells; bottom panel shows the summary of results for CAR-NK cells). FIG. 3E shows a quantification of IFN-γ released after the third round of the serial challenge assay with the CAR-NK cells. Bars represent mean±standard deviation (SD), and symbols represent the individual data points. “ns” indicates not significant, * indicates p<0.05, ** indicates p<0.01, *** indicates p<0.001, and **** indicates p<0.0001.

FIG. 4 shows data for 1G4-TCR-transduced T-cells edited using a CRISPR system and the subsequent effect on HCT116-NY-Eso1-GFP target cells. Genes that were edited in these figures include: AAVS1 (control), ARIH2, UBE2F, CUL5, DCUN1D3, RNF7, and CISH. FIG. 4A shows a schematic describing the manufacturing of 1G4-TCR T-cells followed by editing of the CRL5 complex, or the control gene AAVS1 using a CRISPR system prior to performing subsequent assays. FIG. 4B shows the percentage of the 1G4-TCR transduction (after CRISPR editing, but before performing validation assays). FIG. 4C shows a quantification of EdU uptake by edited CD4 T-cells (first and second panels) and CD8 T-cells (third and fourth panels) in the presence (second and fourth panels) or absence (first and third panels) of the immunosuppressive factor adenosine (“+NECA”). FIG. 4D shows the results of a short-term killing assay (24 h; Incucyte) using the edited 1G4 T-cells and HCT116-NY-Eso1-GFP target cells in the presence (bottom panel) or absence (top panel) of adenosine. FIG. 4E shows a summary of the results of FIG. 4D expressed as a percentage of increased killing compared to AAVS1 control cells. FIG. 4F shows a quantification of IFNγ release during the short-term killing assay (see, e.g., FIG. 4D) in the presence (right panel) or absence (left panel) of adenosine. Bars represent mean±standard error of the mean (SEM), and symbols represent the individual data points. “ns” indicates not significant, * indicates p<0.05, ** indicates p<0.01, *** indicates p<0.001, and indicates p<0.0001.

FIG. 5 shows data for Abecma CAR-transduced T-cells edited using a CRISPR system and the subsequent effect on Nalm6 target cells designed to express high levels of BCMA antigen (Nalm6-BCMA-GFP). Genes that were edited in these figures include: AAVS1 (control), ARIH2, UBE2F, CUL5, DCUN1D3, RNF7, and CISH. FIG. 5A shows a schematic describing the manufacturing of Abecma CAR-T-cells followed by editing of the CRL5 complex, or the control gene AAVS1 using a CRISPR system prior to performing subsequent assays. FIG. 5B shows the percentage of Abecma CAR transduction (after CRISPR editing, but before performing validation assays). FIG. 5C shows a quantification of EdU uptake by edited CD4 T-cells (first and second panels) and CD8 T-cells (third and fourth panels) in the presence (second and fourth panels) or absence (first and third panels) of the immunosuppressive factor adenosine (“+NECA”). FIG. 5D shows the results of a serial challenge assay (R1, R2, R3, and R4) performed by FACS with the edited CAR-T-cells against the Nalm6-BCMA-GFP target cells in the presence (bottom panel) or absence (top panel) of adenosine. FIG. 5E shows a summary of the results of FIG. 5E (R4) expressed as a percentage of increased killing compared to AAVS1 control cells. FIG. 5F shows a quantification of IFNγ release during the serial challenge assay (R4; see, e.g., FIG. 5D) in the presence (right panel) or absence (left panel) of adenosine. Bars represent mean±standard error of the mean (SEM), and symbols represent the individual data points. “ns” indicates not significant, * indicates p<0.05, ** indicates p<0.01, *** indicates p <0.001, and **** indicates p<0.0001.

FIG. 6 depicts CRISPR screen results from human NK cells highlighting ranks and overall significance (represented as false discovery rate (FDR) values) for positive controls in each of the performed screens. FIG. 6A depicts the results of the first high-throughput CRISPR screen (IL-15 cytokine sensitivity). FIG. 6B depicts the results of the second high-throughput CRISPR screen (survival under oxidative stress). FIG. 6C depicts the results of the third high-throughput CRISPR screen (NKG2D expression under TGF-β pressure). FIG. 6D depicts the results of the fourth high-throughput CRISPR screen (IFN-γ production under TGF-β pressure). FIG. 6E depicts the cumulative enrichment score for indicated gene candidates across all screens, as well as an intersection matrix graph depicting significant low rank with respect to the four screens.

FIG. 7 shows the results of initial hit validation experiments in NK cells obtained from human donors. FIG. 7A shows a quantification of live target cells/well and live NK cells/well in response to a serial challenge assay (-TGF-β). FIG. 7B shows a quantification of live target cells/well and live NK cells/well in response to a serial challenge assay (+ TGF-β). FIG. 7C shows a quantification of live target cells/well and live NK cells/well in response to a serial challenge assay (-Kynurenine). FIG. 7D shows a quantification of live target cells/well and live NK cells/well in response to a serial challenge assay (+Kynurenine). FIG. 7E shows a quantification of IFN-γ secretion in response to stimulation from the cytokines IL-12 and IL-18 (±TGF-β). FIG. 7F shows a quantification of IFN-γ secretion in response to co-culturing with K562 target cells (±TGF-β). N=2 per group. Bars represent mean±standard deviation (SD). *P <0.05, **P<0.01, ***P<0.001, ****P<0.0001.

FIG. 8 shows data for primary human natural killer (NK) cells (derived from Donor 1) edited using a CRISPR system and subsequent effects on Nalm6-BCMA target cells. Genes that were edited in the CAR-NK cells include: CUL5, genes orthogonal to the CRL5 complex alone (ARHGAP25, CCNC, FAM49B, N4BP1, STUB1) or in combination with CUL5 (ARHGAP25/CUL5, CCNC/CUL5, FAM49B/CUL5, N4BP1/CUL5, STUB1/CUL5), or the negative control gene AAVS1. FIG. 8A shows a schematic describing the production of NK cells modified using an ABECMA CAR construct followed by editing of the aforementioned genes prior to performing a serial killing assay. FIG. 8B shows representative FACS plots displaying the percentage of CAR transduction, as measured by mScarlet fluorescence, in the CAR-NK cells after CRISPR RNP editing. A quantification of CAR-NK cell expansion is shown in FIG. 8C, expressed as fold change (FC) difference in comparison to the negative control (AAVS1). FIG. 8D shows graphs depicting the amount of live BCMA targets per well (top panel) and the amount of live CAR-NK cells per well (bottom panel) for the four rounds (R1, R2, R3, and R4) of the serial challenge assay. FIG. 8E shows a summary of the results of FIG. 8D (R4) presented as (1) a percentage of increased killing compared to the negative control, and (2) CAR-NK cell FC compared to the negative control. FIG. 8F shows a quantification of live-cell imaging of the CAR-NK cell clustering (represented as CAR+NK cell clusters per well) while killing target cells during the serial challenge assay using an Incucyte. FIG. 8G shows a summary of the results of FIG. 8F (R4) presented as FC in NK cell clusters per well at the end point of the serial challenge assay on the Incucyte. FIG. 8H shows the results of the AlphaLISA human IFN-γ assay and the LEGENDplex TH1 panel (IL-10) performed on cell culture supernatants collected during the first round (R1) of the serial challenge assay. N=2 per group. Bars represent mean standard deviation (SD). *P<0.05, **P<0.01, ***P<0.001, ****P<0.0001.

FIG. 9 shows data for primary human natural killer (NK) cells (derived from Donor 2) edited using a CRISPR system and subsequent effects on Nalm6-BCMA target cells. Genes that were edited in the CAR-NK cells include: CUL5, genes orthogonal to the CRL5 complex alone (ARHGAP25, CCNC, FAM49B, N4BP1, STUB1) or in combination with CUL5 (ARHGAP25/CUL5, CCNC/CUL5, FAM49B/CUL5, N4BP1/CUL5, STUB1/CUL5), or the negative control gene AAVS1. FIG. 9A shows a schematic describing the production of NK cells modified using an ABECMA CAR construct followed by editing of the aforementioned genes prior to performing a serial killing assay. FIG. 9B shows representative FACS plots displaying the percentage of CAR transduction, as measured by mScarlet fluorescence, in the CAR-NK cells after CRISPR RNP editing. A quantification of CAR-NK cell expansion is shown in FIG. 9C, expressed as FC difference in comparison to the negative control. FIG. 9D shows graphs depicting the amount of live BCMA targets per well (top panel) and the amount of live CAR-NK cells per well (bottom panel) for the four rounds (R1, R2, R3, and R4) of the serial challenge assay. FIG. 9E shows a summary of the results of FIG. 9D (R4) presented as (1) a percentage of increased killing compared to the negative control, and (2) CAR-NK cell FC compared to the negative control. FIG. 9F shows a quantification of live-cell imaging of the CAR-NK cell clustering (represented as CAR⁺ NK cell clusters per well) while killing target cells during the serial challenge assay using an Incucyte. FIG. 9G shows a summary of the results of FIG. 9F (R4) presented as FC in NK cell clusters per well at the end point of the serial challenge assay on the Incucyte. FIG. 9H shows the results of the AlphaLISA human IFN-γ assay and the LEGENDplex TH1 panel (IL-10) performed on cell culture supernatants collected during the first round (R1) of the serial challenge assay. N=2 per group. Bars represent mean±standard deviation (SD). *P<0.05, **P<0.01, ***P<0.001, ****P<0.0001.

FIG. 10 shows the results of CAR-NK cell validation assays under immunosuppressive conditions. N=2 per group. Bars represent mean±standard deviation (SD). *P<0.05, **P<0.01, ***P<0.001, ****P<0.0001.

FIG. 11 shows data for 1G4-TCR-transduced NK cells edited using a CRISPR system and the subsequent effect on Nalm6 target cells designed to express high levels of BCMA antigen (Nalm6-BCMA-GFP). Genes that were edited in these figures include: AAVS1, CUL5 (alone), STUB1 (alone), and CUL5+STUB1. FIG. 11A shows a schematic describing the manufacturing of 1G4-TCR NK cells followed by editing of the CRL5 complex and/or a gene orthogonal to the CRL5 complex, or the control gene AAVS1, using a CRISPR system prior to performing subsequent assays. FIG. 11B shows the percentage of 1G4-TCR transduction (After CRISPR editing, but before performing validation assays at Day 27). FIG. 11C shows the results of a short-term killing assay (24 h; Incucyte) using the edited 1G4 NK cells derived from one of two donors (Donor 1-left panel; Donor 2-right panel) and Nalm6-NY-Eso1-GFP target cells. FIG. 11D shows the results of a serial challenge assay (R1 and R2; Incucyte) using the edited 1G4 NK cells against the Nalm6-BCMA-GFP target cells (Donor 1-top panel; Donor 2-bottom panel).

FIG. 11E shows a summary of the results of FIG. 11D (R2) expressed as a percentage of increased killing compared to AAVS1 control cells. FIG. 11F shows a quantification of IFNγ release during the short-term killing assay (Donor 1-left panel; Donor 2-right panel). Bars represent mean standard deviation (SD), and symbols represent the individual data points. “ns” indicates not significant, * indicates p<0.05, ** indicates p<0.01, *** indicates p<0.001, and indicates p<0.0001.

FIG. 12 shows data for Abecma CAR-transduced γδ-T-cells edited using a CRISPR system and the subsequent effect on SKOV3 target cells engineered to express high levels of BCMA antigen (SKOV3-BCMA-GFP). Genes that were edited in these figures include: AAVS1, CUL5 (alone), STUB1 (alone), and CUL5+STUB1. FIG. 12A shows a schematic describing the manufacturing of Abecma CAR-γδ-T-cells followed by editing of the CRL5 complex and/or a gene orthogonal to the CRL5 complex, or the control gene AAVS1, using a CRISPR system prior to performing subsequent assays. FIG. 12B shows a representative FACS plot showing the purity of Vδ2+ T-cells after isolation (left panel), and the percentage of Abecma CAR transduction (after CRISPR editing, but before performing validation assays at Day 14). FIG. 12C shows the results of a short-term killing assay (24 h; Incucyte) using the edited Abecma CAR-γδ-T-cells and SKOV3-BCMA-GFP target cells in the presence (second and third panels) or absence (first panel) of the immunosuppressive factors TGF-β (“+ TGFβ”; second panel) or adenosine (“+NECA”; third panel). FIG. 12D shows the results of a serial challenge assay (R1, R2, and R3; Incucyte) using the edited γδ-T-cells against the SKOV3-BCMA-GFP target cells.

FIG. 12E shows a summary of the results of FIG. 12D (R3) expressed as a percentage of increased killing compared to AAVS1 control cells. FIG. 12F shows a quantification of IFNγ release during the serial challenge assay (R3). FIG. 12G shows the results of a modified serial challenge assay (R1, R2, and R3; left panel) similar to that shown and described for FIG. 12A except that the isolated T-cells were incubated in the presence of the cytokine IL-15 instead of the cytokine IL-2; and a quantification of IFNγ release during the serial challenge assay (R3; right panel). Bars represent mean±standard deviation (SD), and symbols represent the individual data points. “ns” indicates not significant, * indicates p<0.05, ** indicates p<0.01, *** indicates p<0.001, and **** indicates p<0.0001.

FIG. 13 shows a list of exemplary nucleotide sequences for the ARIH2 gene, including human ARIH2 (FIG. 13A and FIG. 13B) and mouse Arih2 (FIG. 13C) sequences. Additional human ARIH2 gene sequences include, but are not limited to, those described in NCBI reference numbers NM 001317333.2, NM_006321.4, NM_001317334.2, NM_001349209.2, NM 001349210.2, NM 001349211.2, NM 001349212.2, NM_001349213.2, NM 001349215.2, NM 001349223.2, NM 001349219.2, NM_001349217.2, NM 001349227.2, NM 001349222.2, NM 001349228.2, NM_001349214.2, NM_001349221.2, NM_001349230.2, NM_001349225.2, NM_001349226.2, NM 001349218.2, NM 001349229.2, NM 001349216.2, NM_001349224.2, NM 001349220.2, NR_146083.2, NR 146082.2, NR_146084.2, and NR_146085.2, each of which is herein incorporated by reference in their entirety. Additional mouse Arih2 gene sequences include, but are not limited to, those described in NCBI reference numbers NM_011790.5, NM_001357285.1, NM_001357286.1, NM_001357283.1, and NR_151664.1, each of which is herein incorporated by reference in their entirety.

FIG. 14 shows a list of exemplary nucleotide sequences for the CUL5 gene, including human CUL5 (FIG. 14A and FIG. 14B) and mouse Cu15 (FIG. 14C and FIG. 14D) sequences. Additional human CUL5 gene sequences include, but are not limited to, those described in NCBI reference numbers NM_003478.6, XM_017018363.3, XM_005271682.3, XM_047427640.1, XM_047427641.1, and XM_011543013.3, each of which is herein incorporated by reference in their entirety. Additional mouse Cu15 gene sequences include, but are not limited to, those described in NCBI reference numbers NM_027807.4, NM_001161618.2, and XM_017313680.3, each of which is herein incorporated by reference in their entirety.

FIG. 15 shows a list of exemplary nucleotide sequences for the DCUN1D3 gene, including human DCUN1D3 (FIG. 15A and FIG. 15B; NCBI reference sequence NM_173475.4) and mouse Dcund13 (FIG. 15C and FIG. 15D) sequences. Additional mouse Dcund13 gene sequences include, but are not limited to, those described in NCBI reference numbers NM_173408.3, NM_001163703.1, XM_006507681.4, XM_011241751.2, XM_036153006.1, XM 036153005.1, XM_011241748.4, and XM_011241749.2, each of which is herein incorporated by reference in their entirety.

FIG. 16 shows a list of exemplary nucleotide sequences for the RNF7 gene, including human RNF7 (FIG. 16A) and mouse Rnf7 (FIG. 16B) sequences. Additional human RNF7 gene sequences include, but are not limited to, those described in NCBI reference numbers NM_014245.5, NR_037703.2, NR 037702.2, NM_183237.3, and NM_001201370.2, each of which is herein incorporated by reference in their entirety. Additional mouse Rnf7 gene sequences include, but are not limited to, those described in NCBI reference numbers NM_011279.3 and NM_001311135.1, each of which is herein incorporated by reference in their entirety.

FIG. 17 shows a list of exemplary nucleotide sequences for the UBE2F gene, including human UBE2F (FIG. 17A) and mouse Ube2f (FIG. 17B) sequences. Additional human UBE2F gene sequences include, but are not limited to, those described in NCBI reference numbers NM_080678.3, NM_001278308.2, NM_001278306.2, NM_001278307.2, NM_001278305.2, NR_103498.2, NR_103499.2, and NR_103500.2, each of which is herein incorporated by reference in their entirety. Additional mouse Ube2f gene sequences include, but are not limited to, those described in NCBI reference numbers NM_026454.4, NM_001355764.1, NM 001355763.1, NM 001355761.1, NM_001355762.1, and NR_149825, each of which is herein incorporated by reference in their entirety.

FIG. 18 shows a list of exemplary nucleotide sequences for the CISH (control) gene, including human CISH (FIG. 18A) and mouse Cish (FIG. 18B) sequences. Additional human CISH gene sequences include, but are not limited to, those described in NCBI reference numbers NM_013324.7 and NM_145071.4, each of which is herein incorporated by reference in their entirety. Additional mouse Cish gene sequences include, but are not limited to, those described in NCBI reference numbers NM_009895.4 and NM_001317354.1, each of which is herein incorporated by reference in their entirety.

FIG. 19 shows a list of exemplary nucleotide sequences for the ARHGAP25 gene, including human ARHGAP25 (FIG. 19A) and mouse Arhgap25 (FIG. 19B) sequences. Additional human ARHGAP25 gene sequences include, but are not limited to, those described in NCBI reference numbers NM_014882.3, NM_001166276.2, NM_001166277.2, NM_001364819.1, NM 001364820.1, NM_001364821.1, XM 011533207.4, XM_011533209.3, XM 011533210.4, XM 017005426.2, XM 054344775.1, XM_054344776.1, and XM_054344777.1, each of which is herein incorporated by reference in their entirety. Additional mouse Arhgap25 gene sequences include, but are not limited to, those described in NCBI reference numbers NM_175476.4, NM_001286610.1, NR_104483.1, XM_017321531.2, XM_036166049.1, and XM_006505980.4, each of which is herein incorporated by reference in their entirety.

FIG. 20 shows a list of exemplary nucleotide sequences for the CCNC gene, including human CCNC (FIG. 20A) and mouse Ccnc (FIG. 20B) sequences. Additional human CCNC gene sequences include, but are not limited to, those described in NCBI reference numbers NM 001013399.2, NM 001363537.2, XM 011536232.4, XM_017011436.3, XM 047419484.1, XM 047419485.1, XM 047419487. 1, XM_047419489.1, XM 047419486.1, XM_054356691.1, XM 054356692.1, XM_054356693.1, XM 054356694.1, XM 054356695.1, XM_054356696.1, and XM_054356697.1, each of which is herein incorporated by reference in their entirety. Additional mouse Ccnc gene sequences include, but are not limited to, those described in NCBI reference numbers NM_001122982.2, NM 001290420.1, NM 001290422.1, XM_036164233.1, and XM_036164234.1, each of which is herein incorporated by reference in their entirety.

FIG. 21 shows a list of exemplary nucleotide sequences for the FAM49B gene, including human FAM49B (FIG. 21A) and mouse Fam49b (FIG. 21B) sequences. Additional human FAM49B gene sequences include, but are not limited to, those described in NCBI reference numbers NM 001353258.2, NM_016623.5, NM_001330612.2, NM_001353242.2, NM 001353243.2, NM 001353244.2, NM 001353245.1, NM_001353246.1, NM 001353247.1, NM 001353248.2, NM 001353249.1, NM_001353250.1, NM_001353251.1, NM 001353252.1, NM_001353253.1, NM_001353254.2, NM 001353255.1, NM_001353256.2, and NM_001353257.2, each of which is herein incorporated by reference in their entirety. Additional mouse Fam49b gene sequences include, but are not limited to, those described in NCBI reference numbers NM_001360036.1, NM_144846.5, NM_001360037.1, NM_001360038.1, XM_006520762.5, XM_006520763.4, XM 006520764.5, XM 006520765.3, XM 006520766.3, XM_011245564.3, XM 030248468.1, XM 030248469.1, XM_030248470.2, XM_030248471.2, XM 030248472.1, XM 030248473.2, XM 030248474.2, XM_030248475.1, XM 030248476.2, XM 030248477.2, XM 030248478. 1, XM_036159326.1, and XM_036159327.1, each of which is herein incorporated by reference in their entirety.

FIG. 22 shows a list of exemplary nucleotide sequences for the N4BP1 gene, including human N4BP1 (FIG. 22A and FIG. 22B) and mouse N4bp1 (FIG. 22C and FIG. 22D) sequences. Additional human N4BP1 gene sequences include, but are not limited to, those described in NCBI reference numbers XM_011523482.2, XM_054314484.1, XR_007064930.1, and XR_008484752.1, each of which is herein incorporated by reference in their entirety. Additional mouse N4bp1 gene sequences include, but are not limited to, those described in NCBI reference numbers XM 006531516.4, XM 006531517.4, XM_036154391.1, XM_036154392.1, XM_036154393.1, and XR_004934903.1, each of which is herein incorporated by reference in their entirety.

FIG. 23 shows a list of exemplary nucleotide sequences for the STUB1 gene, including human STUB1 (FIG. 23A) and mouse Stub1 (FIG. 23B) sequences. Additional human STUB1 gene sequences include, but are not limited to, NCBI reference number NM_001293197.2, which is herein incorporated by reference in its entirety. Additional mouse Stub1 gene sequences include, but are not limited to, those described in NCBI reference numbers NR_157361.1 and NR_157362.1, each of which is herein incorporated by reference in their entirety.

FIG. 24 (FIG. 24A, FIG. 24B, and FIG. 24C) shows a list of exemplary CRISPR gRNA target sequences for: (i) the CRL5 genes (CISH, ARIH2, CUL5, DCUN1D3, RNF7, and UBE2F), (ii) the CRL5 complex-independent genes, and (iii) exemplary CRISPR gRNA target sequences for the control gene AAVS1.

FIG. 25 shows the amino acid sequence for: (i) the ABECMA CAR construct, (ii) a single chain trimer of HLA-A2, 02-microglobulin, and NY-ESO-1 A4 variant peptide, (ii) 1G4 TCR beta chain-P2A-1G4 TCR alpha chain, and (iv) CD3 (delta-gamma-epsilon-zeta).

FIG. 26 shows persistence data for ABECMA CAR-NK cells edited using a CRISPR system transplanted into HCT-116-BCMA-bearing NSG mice. Genes that were edited in these figures include: AAVS1 (control), CUL5 (alone), STUB1 (alone), and CUL5+STUB1. FIG. 26A shows a schematic describing the isolation, expansion, and manufacturing of NK cells modified using an ABECMA CAR construct followed by editing of the aforementioned genes. Subsequently, the edited NK cells were transplanted into HCT-116-BCMA-bearing NSG mice. Tumor(s) and organs were collected at Day 29 post-isolation. FIG. 26B shows a quantification of hCD45⁺ cells per L blood (collected at Day 22 post-isolation (Day 7 after adoptive transfer)), the percentage of cells expressing mScarlet, and CD122 expression compared to the AAVS1 control group. FIG. 26C shows a quantification of hCD45⁺ cells (collected at Day 29 post-isolation (Day 14 after adoptive transfer)), the percentage of cells expressing mScarlet, and CD122 expression compared to the AAVS1 control group in samples obtained from blood (top row), spleen (middle row), or tumor (bottom row). FIG. 26D shows a quantification of cytokine release by CAR-NK cells in the tumor microenvironment (TME; IFNγ—first panel; TNFα—second panel; IL-10-third panel; IL-6-fourth panel).

### DETAILED DESCRIPTION OF THE INVENTION

The description is presented to enable one of ordinary skill in the art to make and use the invention and is provided in the context of a patent application and its requirements. The section headings used herein are for organization purposes only and are not to be construed as limiting the subject matter described. While various embodiments of the invention(s) of the present disclosure have been shown and described herein, it will be obvious to those skilled in the art that such embodiments are provided by way of example only. Numerous variations, changes, and substitutions may occur to those skilled in the art without departing from the invention(s). It should be understood that various alternatives to the embodiments of the invention(s) described herein may be employed in practicing any one of the inventions(s) set forth herein.

All patents, published patent applications, other publications, and sequences from GenBank, and other databases referred to herein are incorporated by reference in their entirety with respect to the related technology.

### L. Definitions

Unless defined otherwise, technical, and scientific terms used herein have the same meaning as commonly understood by one of ordinary skill in the art to which the present disclosure belongs. For purposes of the present disclosure, the following terms are defined below. The definitions provided are intended to apply to a given term, as well as other derivative linguistic re-phrasings and grammatical equivalents of the term.

As used herein, the term “CRL5 complex genes” refers to a group of genes involved in the CRL5 signaling pathway, specifically: (i) ARIH2 (i.e., the gene encoding Protein Ariadne-2 Homolog), (11) CUL5 (i.e., the gene encoding Cullin-5), (iii) DCUN1D3 (i.e., the gene encoding Defective in Cullin Neddylation 1 Domain Containing 3), (iv) RNF7 (i.e., the gene encoding RING-box protein 2), (v) UBE2F (i.e., the gene encoding Ubiquitin Conjugating Enzymes E2 F), or, optionally, (vi) CISH (i.e., the gene encoding cytokine inducible SH2 containing protein), as well as any homologs, orthologs, paralogs, or variants thereof. In some instances, the term “CRL5 complex genes” may refer to only a subset of the aforementioned genes. Similarly, the terms “CRL5 complex proteins” and “polypeptides encoded by the one or more CRL5 complex genes,” which can be used interchangeably, refer to a group of proteins encoded by the genes: (i) ARIH2, (ii) CUL5, (iii) DCUN1D3, (iv) RNF7, (v) UBE2F, or, optionally, (vi) CISH, as well as any homologs, orthologs, paralogs, variants, or functional derivatives thereof.

While CISH is generally not considered to be a core part of the CRL5 complex, it is known to act as a substrate receptor for the CRL5 complex. As such, CISH is involved in the CRL5 signaling pathway and is expressly contemplated to be included within the definition of “CRL5 complex genes” in certain embodiments described herein.

As used herein, the terms “CRL5 complex-independent genes,” “genes independent of the CRL5 complex,” and “genes orthogonal to the CRL5 complex”, which can be used interchangeably, refer to a group of genes “orthogonal” to the CRL5 signaling pathway as enumerated below. While the term “CRL5 complex-independent genes” could generally refer to any gene not considered to be directly involved in signaling via the CRL5 pathway, it is expressly contemplated that the term, as used herein, specifically refers to the genes: (i) ARHGAP25 (i.e., the gene encoding rho GTPase activating protein 25), (ii) CCNC (i.e., the gene encoding cyclin C), (iii) FAM49B (i.e., the gene encoding family with sequence similarity 49, member B, which is also known as CYFIP related Rac1 interactor B (CYRIB)), (iv) N4BP1 (i.e., the gene encoding NEDD4 binding protein 1), or (v) STUB1 (i.e., the gene encoding STIP1 homology and U-Box containing protein 1)) (as well as any homologs, orthologs, paralogs, or variants thereof), as these genes are generally not considered to be directly involved in signaling via the CRL5 complex. In some instances, the term “CRL5 complex-independent genes” may refer to only a subset of the aforementioned genes. Similarly, the terms “CRL5 complex-independent proteins” and “polypeptides encoded by the one or more CRL5 complex-independent genes,” which can also be used interchangeably, refer to a group of proteins encoded by the genes: (i) ARHGAP25, (ii) CCNC, (iii) FAM49B, (iv) N4BP1, or (v) STUB1, as well as any homologs, orthologs, paralogs, variants, or functional derivatives thereof.

In some embodiments, a genetically modified hematopoietic cell is provided wherein one or more of the CRL5 complex genes are subjected to one or more genetic modifications that inhibit expression or activity of the polypeptide products encoded by the one or more CRL5 complex genes, and in some embodiments the genetically modified hematopoietic cell further comprises one or more genetic modifications that inhibit expression or activity of polypeptide peptide products encoded by one or more CRL5 complex-independent genes. Alternatively, in some embodiments, a genetically modified hematopoietic cell is provided wherein one or more of the CRL5 complex-independent genes are subjected to one or more genetic modifications that inhibit expression or activity of the polypeptide products encoded by one or more of the CRL5 complex-independent genes, and in some embodiments the genetically modified hematopoietic cell further comprises one or more genetic modifications that inhibit expression or activity of the polypeptide products encoded by one or more CRL5 complex genes. As yet another alternative, in some embodiments, a genetically modified hematopoietic cell is provided wherein: (i) one or more CRL5 complex genes are subjected to one or more genetic modifications that inhibit expression or activity of the polypeptide products encoded by the one or more CRL5 complex genes, and (ii) one or more CRL5 complex-independent genes are subjected to one or more genetic modifications that inhibit expression or activity of the polypeptide products encoded by the one or more CRL5 complex-independent genes. It is expressly contemplated that the one or more CRL5 complex genes may be subjected to one or more genetic modifications in a temporal manner that is prior to, subsequent to, or concurrent with one or more genetic modifications to one or more CRL5 complex-independent genes.

For the sake of brevity, when discussing elements that can be applicable to either or both of (i) the one or more CRL5 complex genes, and (ii) the one or more CRL5 complex-independent genes, the term “CRL5 complex(-independent) genes,” or other derivative linguistic rephrasing thereof, may be used to indicate embodiments that apply to: (i) one or more CRL5 complex genes (alone), (ii) one or more CRL5 complex-independent genes (alone), or (iii) a combination of one or more CRL5 complex genes and one or more CRL5 complex-independent genes, as if each and every embodiment were set forth individually herein.

As used herein, the term “orthogonal,” when discussed in the context of transduction of a signal or cellular signaling pathways, is meant to refer to one or more genes that are non-overlapping, uncorrelated, or otherwise independent of a given signaling pathway.

As used herein, the terms “polynucleotide” and “nucleic acid” are used interchangeably to refer to a polymeric form of nucleotides of any length, either deoxyribonucleotides or ribonucleotides. The terms include RNA, DNA, and synthetic forms and mixed polymers of the above. In particular embodiments, a nucleotide refers to a ribonucleotide, deoxynucleotide or a modified form or analog of either type of nucleotide, or combinations thereof. In addition, a polynucleotide may include either or both naturally occurring and modified nucleotides linked together by naturally occurring and/or non-naturally occurring nucleotide linkages. The nucleic acid molecules may be modified chemically or biochemically or may contain non-natural or derivatized nucleotide bases. Such modifications include, for example, labels, methylation, substitution of one or more of the naturally occurring nucleotides with an analogue, internucleotide modifications such as uncharged linkages (e.g., methyl phosphonates, phosphotriesters, phosphoramidates, carbamates, etc.), charged linkages (e.g., phosphorothioates, phosphorodithioates, etc.), pendent moieties (e.g., polypeptides), intercalators (e.g., acridine, psoralen, etc.), chelators, alkylators, and modified linkages (e.g., alpha anomeric nucleic acids, etc.).“Polynucleotide” and “nucleic acid” are also intended to include any topological conformation, including single-stranded, double-stranded, partially duplexed, triplex, hair-pinned, circular and padlocked conformations. A reference to a nucleic acid sequence encompasses its complement unless otherwise specified. Thus, a reference to a nucleic acid molecule having a particular sequence should be understood to encompass its complementary strand, with its complementary sequence. Reference to a “polynucleotide” or “nucleic acid” that encodes a polypeptide sequence also includes codon-optimized nucleic acids and nucleic acids that comprise alternative codons that encode the same polypeptide sequence.

As used herein, the terms “complementary” or “complementarity” refers to specific base pairing between nucleotides or nucleic acids. Base pairing may be perfectly complementary (i.e., 100% complementary) or partially complementary (i.e., <100% complementary).

As used herein, the term “gene” can refer to the segment of DNA involved in producing or encoding a polypeptide chain. It may include regions preceding and following the coding region (leader and trailer) as well as intervening sequences (introns) between individual coding segments (exons). Genes are defined by symbol and nomenclature for the human gene as assigned by the HUGO Gene Nomenclature Committee.

As used herein, the term “promoter” refers to one or more nucleic acid control sequences that direct transcription of a nucleic acid. As used herein, a promoter includes necessary nucleic acid sequences near the start site of transcription. A promoter also optionally includes distal enhancer or repressor elements, which can be located as much as several thousand base pairs from the start site of transcription.

As used herein, the term “homolog” refers to a first amino acid sequence or nucleic acid sequence (e.g., gene (DNA or RNA) or protein sequence) that is related to a second amino acid sequence or nucleic acid sequence by descent from a common ancestral sequence. Homologs may arise through speciation events (giving rise to “orthologs”), through gene duplication events (giving rise to “paralogs”), or through horizontal gene transfer events. Homologs may be identified by phylogenetic methods, through identification of common functional domains in aligned nucleic acid or protein sequences, or through sequence comparisons. Most often, homologs will have functional, structural, and/or genomic similarities.

As used herein, the term “ortholog” refers to a gene (or a protein encoded by a gene) that is part of a group of genes (or part of a group of proteins) that are predicted to have evolved from a common ancestral gene by speciation. Typically, orthologs retain the same or similar function despite differences in their primary structure.

As used herein, the term “paralog” refers to homologs in the same species that have evolved by genetic duplication of a common ancestral gene. In many cases, paralogs exhibit related but not always identical functions. To the extent that a particular species has evolved multiple related genes (or proteins encoded by the genes) from an ancestral DNA sequence shared with another species, the term ortholog can encompass the term paralog.

As used herein, the term “functional derivative” includes, but is not limited to, fragments of a native sequence and derivatives of a native sequence polypeptide and its fragments, provided that they have a biological activity in common with a corresponding native sequence polypeptide. A biological activity contemplated herein is the ability of the functional derivative to hydrolyze a DNA substrate into fragments. Generally, biological activity can refer to the ability of a particular gene or protein, such as, for example, one or more CRL5 complex genes or the proteins encoded by the one or more CRL5 complex genes (or, for example, one or more CRL5 complex-independent genes or the proteins encoded by the one or more CRL5 complex-independent genes). In some instances, the biological activity of a native sequence, such as, for example, the protein product of one or more CRL5 complex genes, can be inhibited through the use of a genetic modification system as compared to the biological activity of a reference. The term “derivative” encompasses both amino acid sequence variants of a polypeptide, covalent modifications, and fusions thereof, such as, for example, a derivative Cas protein. Suitable derivatives of a Cas polypeptide or a fragment thereof include, but are not limited to, mutants, fusions, and/or covalent modifications of a Cas protein (or a fragment thereof).

As used herein, the term “Cas9 ribonucleoprotein complex” (“Cas9 RNP complex”) refers to a complex between a Cas9 and a guide RNA (gRNA), the Cas9 protein and a CRISPR RNA (a “crRNA”), the Cas9 protein and a trans-activating crRNA (a “tracrRNA”), or any combination thereof. It is understood that in any of the embodiments described herein, a Cas9 nuclease can be substituted with another RNA-mediated nuclease (e.g., an alternative Cas protein or a Cpf1 nuclease). In some embodiments, the Cas protein is introduced into a hematopoietic cell used (i) to develop the compositions, and/or (ii) in the methods disclosed herein in a polypeptide form. Thus, for example, in certain embodiments, the Cas proteins can be conjugated to or fused to a cell-penetrating polypeptide or cell-penetrating peptide that is known in the relevant art.

As used herein, a “guide RNA sequence” (gRNA sequence) refers to a sequence that interacts with a site-specific or targeted nuclease and specifically binds to or hybridizes to a target nucleic acid (e.g., one or more CRL5 complex genes, one or more CRL5 complex-independent genes, etc.) within the genome of a cell (e.g., a hematopoietic cell used (i) to develop the compositions, and/or (ii) in the methods disclosed herein), such that the gRNA and the targeted nuclease co-localize to the target nucleic acid in the genome of the cell. Each gRNA includes a DNA targeting sequence or protospacer sequence of about 10 to about 50 nucleotides in length that specifically binds to or hybridizes to a target DNA sequence (e.g., one or more CRL5 complex genes, one or more CRL5 complex-independent genes, etc.) in the genome of a cell (e.g., a hematopoietic cell used (i) to develop the compositions, and/or (ii) in the methods disclosed herein). In some embodiments, the target sequence can be about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length. In some embodiments, the gRNA comprises a crRNA sequence and a tracrRNA sequence, and the gRNA may be referred to as a single guide RNA (sgRNA). In some embodiments, the gRNA does not comprise a tracrRNA sequence. The gRNA and/or sgRNA can be selected depending on the particular CRISPR/Cas system employed and/or the sequence of the target polynucleotide (e.g., one or more CRL5 complex genes, one or more CRL5 complex-independent genes, etc.).

As used herein, the term “zinc finger” refers to a small protein structural motif stabilized by one or more zinc ions. A zinc finger can comprise, for example, Cys2His2, and can recognize a nucleotide sequence of approximately 3 base pairs in length. Various zinc fingers of known specificity can be combined to produce multi-finger polypeptides which can recognize nucleotides of various length (e.g., about 6, about 9, about 12, about 15, about 18, or more).

As used herein, the terms “inhibits expression,” “reduces expression,” “decreases expression,” and the like, refer to inhibiting or reducing the expression of a gene or a protein. To inhibit or reduce the expression of a gene (e.g., one or more CRL5 complex genes, one or more CRL5 complex-independent genes, etc.), the sequence and/or structure of the gene may be modified such that the gene would not be transcribed (for DNA) or translated (for RNA) or would not be transcribed or translated to produce a functional protein (e.g., a transcription factor). Various methods for inhibiting or reducing expression of a gene are described in detail further herein. Some methods may introduce nucleic acid substitutions, insertions, and/or deletions into the wild-type gene. Some methods may also introduce single or double strand breaks into the gene. To inhibit or reduce the expression of a protein (e.g., one or more proteins encoded by one or more CRL5 complex genes, one or more proteins encoded by one or more CRL5 complex-independent genes, etc.), one may inhibit or reduce the expression of the gene or polynucleotide encoding the protein, as described above. In other embodiments, one may target the protein directly to inhibit or reduce the protein's expression using, e.g., an antibody or a protease. “Inhibited” expression refers to a decrease by at least 10% as compared to a reference control level, for example a decrease by at least about 20%, or at least about 30%, or at least about 40%, or at least about 50%, or at least about 60%, or at least about 70%, or at least about 80%, or at least about 90% or up to and including a 100% decrease (i.e., absent level as compared to a reference sample).

As used herein, the term “inactivate,” when used in reference to the activity or expression of a gene or protein, means that a gene (or a protein encoded by the gene) has been modified such that the gene (or the protein encoded by the gene) is either not expressed (i.e., expression is inhibited 100%), or if expression remains, the protein encoded by the gene is not functional compared to a wild-type reference. Methods for inhibiting the expression or activity of a gene (or protein encoded by the gene) are described throughout and are, generally, equally applicable to the inactivation of a gene or protein, unless the context clearly dictates otherwise.

As used herein, the terms “modifying” or “modification,” when used in the context of modifying a genome of a cell (e.g., one or more genetic modifications to one or more genes independent of the CRL5 complex, one or more genetic modifications to one or more CRL5 complex genes, etc.), refers to inducing a structural change in the sequence of the genome at a target genomic region through the insertion, deletion, or substitution of one or more nucleotides. The terms may further refer to combinations of one or more insertions, one or more deletions, and/or one or more substitutions that effectuate a desired outcome or effect (such as, for example, an inhibition in the expression or activity of polypeptide products encoded by one or more genes). For example, the modifying can take the form of inserting a nucleotide sequence into the genome of the cell. For example, a nucleotide sequence encoding a polypeptide can be inserted into the genomic sequence encoding one or more CRL5 complex(-independent) genes. The nucleotide sequence can encode a functional domain or a functional fragment thereof. Such modifying can be performed, for example, by inducing a double stranded break within a target genomic region, or a pair of single stranded nicks on opposite strands and flanking the target genomic region. Methods for inducing single or double stranded breaks at or within a target genomic region include the use of a nuclease domain, e.g., Cas9, or a derivative thereof, and a guide, e.g., guide RNA, directed to the target genomic region.

As used herein, the term “gene deletion” (or “deletion” in the context of a genetic modification) refers to the removal of at least a portion of a DNA sequence from, or in proximity to, a gene (e.g., one or more genes independent of the CRL5 complex, one or more CRL5 complex genes, etc.). In some further embodiments, the sequence subjected to gene deletion can comprise an exonic sequence of a gene. Alternatively, or in addition to, the sequence subjected to gene deletion can comprise a promoter sequence of the gene. In some further embodiments, the sequence subjected to gene deletion can comprise a flanking sequence of a gene. In some embodiments, a portion of a gene sequence is removed from a gene. Alternatively, or in addition to, the complete gene sequence can be removed from a chromosome. In some embodiments, a host cell, such as, for example, a hematopoietic cell used (i) to develop the compositions, and/or (ii) in the methods disclosed herein, is modified to possess a gene deletion as described in any of the embodiments herein. In some embodiments, a deletion can result in the inhibition of a gene (or a protein encoded by the gene). In other embodiments, a deletion can result in the inactivation of a gene (or a protein encoded by the gene).

As used herein, the term “insertion” in the context of a genetic modification, refers to the addition of a nucleotide (or multiple nucleotides) at a particular position in a parent polynucleotide sequence. In some instances, the insertion may result in a frameshift mutation, a nonsense mutation, a premature stop codon, or other genetic perturbations that inhibits (or inactivates) the expression or activity of a gene, such as, for example, one or more genes independent of the CRL5 complex, one or more CRL5 complex genes, etc. In some embodiments, an insertion can result in the inhibition of a gene (or a protein encoded by the gene). In other embodiments, an insertion can result in the inactivation of a gene (or a protein encoded by the gene).

As used herein, the term “substitution” in the context of a genetic modification, refers to the replacement of a nucleotide (or multiple nucleotides) at a particular position (or, in the case of multiple nucleotide substitutions, particular positions) in a parent polynucleotide sequence. In some instances, the substitution may result in a nonsense mutation, a premature stop codon, an alternatively spliced transcript, or other genetic perturbations that inhibits (or inactivates) the expression or activity of a gene, such as, for example, one or more genes orthogonal to the CRL5 complex, one or more CRL5 complex genes, etc. In some embodiments, a substitution can result in the inhibition of a gene (or a protein encoded by the gene). In other embodiments, a substitution can result in the inactivation of a gene (or a protein encoded by the gene).

The terms “patient,” “subject,” “individual,” and the like may be used interchangeably herein. As used herein, these terms can generally refer to any animal classified as a mammal (including, but not limited to, humans, primates, and/or non-human primates), domestic animals, farm animals, zoo animals, research animals, sports animals, and/or pet animals, such as dogs, horses, cats, cows, etc. Unless specified otherwise, the terms “patient,” “subject,” “individual,” and the like refer to human subjects. In some instances, the human subject is a “donor subject.” Generally, the term “donor subject” refers to an individual from whom one or more tissues and/or one or more biological fluids are collected, extracted, or otherwise obtained from, wherein the one or more tissues and/or one or more biological fluids are to be used in, or as a part of, a treatment in a “recipient subject.” In some instances, the donor subject is alive, whereas in other instances the donor subject may be deceased. Generally, the term “recipient subject” refers to an individual that will receive, has received, or is in the process of receiving one or more tissues and/or one or more biological fluids from a donor subject. In some instances, the donor subject is also the recipient subject (i.e., the recipient is to receive, has received, or is in the process of receiving an autologously-sourced biological tissue and/or biological fluid). In other instances, the donor subject is not the recipient subject (i.e., the recipient is to receive, has received, or is in the process of receiving biological tissue and/or biological fluid from an allogeneic source).

As used herein, the term “allogeneic” refers to any material derived from a first individual (e.g., a biological tissue, a biological fluid), which is then introduced to another individual of the same species (e.g., an allogeneic transplantation of a hematopoietic cell).

As used herein, the term “autologous” refers to any material derived from a first individual (e.g., a biological tissue, a biological fluid), which is then re-introduced into the same individual.

As used herein, the phrase “compatible HLA-typing” is used to refer to a configuration of major histocompatibility genes from a cellular source (i.e., a donor) that does not trigger the immune system of the subject in need of therapy (or that is suffering from a disease (e.g., a cancer); i.e., a recipient) resulting in rejection (e.g., transplant rejection, hyperacute rejection, acute rejection, chronic rejection, graft failure, etc.).

As used herein, the terms “treatment,” “treating,” and the like are used herein to generally mean obtained a desired pharmacologic and/or physiologic effect in response to an adoptive cell therapy (ACT), and/or in response to a method employing an adoptive cell therapy. The effect may be prophylactic, in terms of completely or partially preventing a disease, condition, or symptoms thereof, and/or may be therapeutic in terms of a partial or complete cure for a disease or condition and/or an adverse effect, such as a symptom, attributable to the disease or condition. “Treatment” as used herein covers any treatment of a disease or condition of a subject and includes: (a) preventing the disease or condition from occurring in a subject which may be predisposed to the disease or condition but has not yet been diagnosed as having it; (b) inhibiting the disease or condition (e.g., arresting its development); or (c) relieving the disease or condition (e.g., causing regression of the disease or condition, providing improvement in one or more symptoms).

As used herein, the term “cancer” refers to a broad group of various diseases characterized by the uncontrolled growth of abnormal cells in the body. Unregulated cell division and growth results in the formation of malignant tumors that invade neighboring cells or tissues and may metastasize to distant parts of the body through the lymphatic system or bloodstream. As used herein the terms “cancer” or “cancer tissue” include both solid and liquid tumors. Examples of cancers that can be treated by the methods of the present invention include, but are not limited to, sarcomas, carcinomas, cancers of the immune system including lymphoma, leukemia, myeloma, and other leukocyte malignancies, and the like. In some embodiments, the methods of the present invention can be used to reduce the tumor size of a tumor derived from, for example, breast cancer, colon cancer, colorectal cancer, bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, multiple myeloma, Hodgkin's Disease, non-Hodgkin's lymphoma (NHL), primary mediastinal large B cell lymphoma (PMBC), diffuse large B cell lymphoma (DLBCL), follicular lymphoma (FL), transformed follicular lymphoma, splenic marginal zone lymphoma (SMZL), cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, chronic or acute leukemia, acute myeloid leukemia, chronic myeloid leukemia, acute lymphoblastic leukemia (ALL) (including non T cell ALL), chronic lymphocytic leukemia (CLL), solid tumors of childhood, lymphocytic lymphoma, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally induced cancers including those induced by asbestos, other B cell malignancies, and the like, as well as any combination(s) thereof.

As used herein, the term “conditioning regimen” refers to the administration of one or more chemotherapies, one or more instances of a radiation therapy, or a combination thereof to a human subject (e.g., a cancer patient) prior to the administration of an ACT, such as, for example, administering a population of cells comprising a genetically modified hematopoietic cell, wherein the genetically modified hematopoietic cell comprises a genetic modification to one or more CRL5 complex(-independent) genes. The term “conditioning regimen” may refer to one or more of: (i) a “myeloablative conditioning regimen,” (ii) a “nonmyeloablative conditioning regimen,” and (iii) a “reduced intensity conditioning regimen.” As used herein, the term “myeloablative conditioning regimen” refers to the administration of one or more chemotherapies, one or more instances of a radiation therapy, or a combination thereof, at doses and/or frequencies that cause irreversible cytopenia and require one or more infusions of stem cells from a donor for the survival of the subject. Nonlimiting examples of a myeloablative conditioning regimen include: (i) a single administration of total body irradiation (TBI) >5 Grays (Gy), (ii) a fractionated administration of TBI >8 Gy (i.e., two or more administrations of TBI that cumulatively are >8 Gy over a fixed period of time), (iii) oral administration of >8 mg/kg of Busulfan (or an equivalent IV dosage), and others known in the relevant art. As used herein, the term “nonmyeloablative conditioning regimen” refers to the administration of one or more chemotherapies, one or more instances of a radiation therapy, or a combination thereof, at doses and/or frequencies that cause minimal cytopenia and do not require one or more infusions of stem cells from a donor for the survival of the subject. Nonlimiting examples of a nonmyeloablative conditioning regimen include: (i) a single administration of TBI <2 Gy (with or without concomitant administration of a purine analog), (ii) a combination of fludarabine and cyclophosphamide (with or without concomitant administration of anti-thymocyte globulin (ATG)), (iii) a combination of fludarabine, cytarabine, and idarubicin, (iv) a combination of cladribine and cytarabine, (v) total lymphoid irradiation with concomitant administration of ATG, and others known in the relevant art. As used herein, the term “reduced intensity conditioning regimen” refers to a conditioning regimen that does not fit the criteria for a myeloablative conditioning regimen or a nonmyeloablative conditioning regimen. Generally, reduced intensity conditioning regimens cause cytopenia of a finite duration, but still require one or more infusions of stem cells from a donor. Nonlimiting examples of reduced intensity conditioning regimens include: (i) a combination of busulfan, fludarabine, and ATG prior to the infusion of stem cells from a donor, (ii) a combination of fludarabine, melphalan, and ATG prior to the infusion of stem cells from a donor, and others known in the relevant art.

As used herein, the terms “exogenous” and “heterologous” refer to a molecule or substance (e.g., a compound, a nucleic acid, a protein, a cytokine, a chemokine, etc.) in a host organism (e.g., a genetically modified hematopoietic cell, NK cell, T-cell, TIL, stem cell, etc.) that does not naturally have that molecule or substance. For example, an “exogenous nucleic acid” as referred to herein is a nucleic acid that is not naturally occurring in a host organism. An exogenous nucleic acid may be produced by transforming, transducing, transfecting, infecting, or otherwise incorporating into a cell (such as, for example, a genetically modified hematopoietic cell) a plasmid, a vector, a virus, or another genetic construct including the subject nucleic acid encoding the exogenous molecule. Conversely, the term “endogenous nucleic acid” refers to a molecule or substance that is naturally occurring within a given host organism. However, it is also contemplated that the terms “exogenous” and “heterologous” can refer to a molecule or substance that is produced at a supraphysiological level in response to the incorporation of a plasmid/vector/virus/genetic construct into a host cell that endogenously exhibits a relatively lower expression of the molecule or substance, as compared to a host cell that does not have a plasmid/vector/virus/genetic construct incorporated into the host cell encoding the molecule or substance. For example, NK cells are known to produce and secrete a given amount of IFN-γ in response to various cellular signaling pathways. While IFN-γ is naturally occurring within NK cells, IFN-γ production can be increased through the incorporation of, for example, a CAR construct containing a coding sequence for IFN-γ under the influence of a promoter (e.g., an inducible promoter, a constitutive promoter). In such a scenario, IFN-γ would be considered an “exogenous cytokine” due to the supraphysiological production of IFN-γ in the NK cell in response to the incorporation and expression of the CAR construct.

As used herein, the singular forms “a,” “an”, and “the” include plural referents unless the context clearly dictates otherwise. Thus, for example, reference to “an antigen” includes mixtures of antigens; reference to “a pharmaceutically acceptable carrier” includes mixtures of two or more such carriers, and the like. As such, the terms “a” (or “an”), “one or more,” and “at least one” can be used interchangeably herein.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A (alone)”, and “B (alone)”.

As used herein, the term “about” a value (or parameter) refers to ±10% of a stated value. When referring to a range of values (or parameters), the term “about” refers to +10% of the upper limit and −10% of the lower limit of a stated range of values. When a range of values is provided, it is to be understood that each intervening value between the upper and lower limit of that range, and any other stated or intervening value in that stated range, is encompassed within the scope of the present disclosure. Where the stated range includes upper and/or lower limits, ranges excluding either of those included limits are also included in the present disclosure.

As used herein, the use of ordinal terms, such as, for example, “first,” “second,” “third,” “fourth”, “fifth,” etc., to modify an element of the disclosure does not by itself connote any priority, precedence, or order of one element over another or the temporal order in which acts of a method are performed, but are used merely as labels to distinguish one element having a certain name (e.g., an assay) from another element having a same name but for the use of the ordinal term to distinguish the elements (e.g., “a first assay,” “a second assay,” “a third assay,” “a fourth assay,” “a fifth assay,” etc.). For example, a “first assay” may be performed to detect changes in the proliferative capacity of a genetically modified hematopoietic and a “second assay” may be performed to detect changes in resistance to immune suppression. In such an example, the terms “first assay” and “second assay” are not used to describe any priority, precedence, or order in which the assays are performed, but rather to denote that the elements are distinct despite having the same name except for the use of the ordinal terms “first” and “second.” As a separate example, a “third assay” may be performed to detect a particular effect of the genetic modification to the one or more CRL5 complex(-independent) genes (e.g., cytotoxic capacity) without a “first assay” or a “second assay” having been performed. Alternatively, it is also contemplated that a “first assay” could be performed before or after the “third assay,” and that the “second assay” of the example may not be performed, may be performed before or after the “first assay,” or before or after the “third assay.” While the examples set forth above utilize specific ordinal terms, it is contemplated that the concepts described above apply mutatis mutandis to other examples using any number of ordinal terms in various configurations.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination. All combinations of the embodiments pertaining to the disclosure are specifically embraced by the present disclosure and are disclosed herein just as if each and every combination was individually and explicitly disclosed. In addition, all sub-combinations of the various embodiments and elements thereof are also specifically embraced by the present disclosure and are disclosed herein just as if each and every such sub-combination was individually and explicitly disclosed herein.

### II. Overview

The present application generally relates to compositions and methods for treating cancer. In particular, the disclosure provides novel, genetically modified hematopoietic cells, wherein the genetically modified hematopoietic cells comprise one or more genetic modifications to: (i) one or more Cullin-(CUL-) Really Interesting New Gene (RING) ubiquitin Ligase 5 (CRL5) complex genes ((a) ARIH2; (b) CUL5; (c) DCUN1D3; (d) RNF7; (e) UBE2F; (f) CISH; or a combination thereof) and/or (ii) one or more genes orthogonal to the CRL5 complex ((a) ARHGAP25; (b) CCNC; (c) FAM49B (which is also known as CYRIB); (d) N4BP1; (e) STUB1; or a combination thereof), that inhibits expression or activity of the polypeptide products encoded by the one or more CRL5 complex genes and/or the one or more CRL5 complex-independent genes by at least about 50% compared to a control hematopoietic cell, as well as methods of making and using the genetically modified hematopoietic cells.

As described above, current ACTs can have limited proliferative capacity and be susceptible to varying degrees of immune suppression. As a result of such deficiencies, the cytotoxic capacity and the ability of ACTs to stimulate the production of cytokines can be limited.

The present disclosure provides novel compositions and methods for improving ACT by developing genetically engineered cells by targeting: (i) the CRL5 complex, and/or (ii) one or more genes orthogonal to the CRL5 complex. In some embodiments, the compositions and methods described herein result in the generation of an improved ACT product by developing genetically modified cells, such as genetically modified hematopoietic cells. Non-limiting examples of improvements to ACT provided by the methods and compositions described herein include: (i) an increase in proliferative capacity of the ACT product, (ii) an increase in resistance to immunosuppression of the ACT product, (iii) an increase in cytotoxic capacity of the ACT product, (iv) an increase in the killing efficiency of cancer cells by the ACT product, and (v) an increase in the ability of the ACT product to stimulate the production and/or release of cytokines, when compared to hematopoietic cells that have not been genetically modified.

In some embodiments, the genetically modified hematopoietic cell comprises a T-cell or a NK cell. In some preferred embodiments, the genetically modified hematopoietic cell comprises a γδ-T-cell, whereas in other preferred embodiments the genetically modified hematopoietic cell comprises a NK cell. γδ-T-cells are considered innate immune cells due to: (1) their MHC unrestricted antigen recognition, (2) expression of natural killer receptors (NKRs), (3) expression of Toll-like receptors (TLRs), and (4) capacity for rapid cytokine production. More specifically, Vγ9Vδ2 cells, which are the dominant subtype of γδ-T-cells in peripheral blood, recognize phosphoantigens (PAgs) expressed on tumor cells in an MHC-independent manner mediated by butyrophilin and butyrophilin-like molecules (BTN). In addition, they are known to express NK receptors NKG2D and DNAM1, which recognize stress-induced MHC class I chain-related molecules (such as, for example, MICA/B, ULBPs, etc.) on tumor cells, similar to NK cells. Cytokine production, including IFNγ, is a major effector function in γδ-T-cells, similar to NK cells.

γδ-T-cell survival and effector function is dependent on proinflammatory cytokines, such as, for example, IL-2 and IL-15, as they produce less endogenous IL-2 compared to αβ-T-cells. The γδ TCR is critical for initial stages if differentiation and proliferation, but both processes require cytokine signals to promote cell survival and full effector function. Consequently, it was hypothesized that negative the negative regulators of IL-2/IL-15 signaling axis, discovered in NK cells, might enhance γδ-T-cell effector function. Indeed, genetic ablation of CUL5 (alone), STUB1 (alone), and CUL5+STUB1 all enhanced γδ-T-cell cytotoxic capacity and IFNγ production, with the combination of CUL5+STUB1 eliciting the strongest response (similar to what was observed with NK cells, as is explained in more detail below). Conversely, in αβ-T-cells, genetic ablation of STUB1 did not exhibit the phenotypic changes shown in γδ-T-cells.

### II. Genetically Modified Hematopoietic Cells

As will be appreciated by one skilled in the art, any of the aspects and embodiments of the compositions described herein can be used in any of the aspects and/or embodiments of the methods of making described herein and can further be used in any of the aspects and/or embodiments of the methods of use also described herein.

In one aspect, the present disclosure provides a genetically modified hematopoietic cell, comprising: one or more genetic modifications to one or more CRL5 complex genes that inhibits expression or activity of the polypeptide products encoded by the one or more CRL5 complex genes, wherein expression or activity of the polypeptide products is inhibited by at least about 50% compared to a control hematopoietic cell. In another aspect, the present disclosure provides a genetically modified hematopoietic cell, comprising: one or more genetic modifications to one or more CRL5 complex-independent genes that inhibits expression or activity of the polypeptide products encoded by the one or more CRL5 complex-independent genes, wherein expression or activity of the polypeptide products is inhibited by at least about 50% compared to a control hematopoietic cell. In yet another aspect, the present disclosure provides a genetically modified hematopoietic cell, comprising: (1) one or more genetic modifications to one or more CRL5 complex genes that inhibits expression or activity of the polypeptide products encoded by the one or more CRL5 complex genes, and (2) one or more genetic modifications to one or more CRL5 complex-independent genes that inhibits expression or activity of the polypeptide products encoded by the one or more CRL5 complex-independent genes, wherein expression or activity of the polypeptide products is inhibited by at least about 50% compared to a control hematopoietic cell.

For the sake of brevity, these cells may be referred to as “genetically modified hematopoietic cells” when describing features, elements, characteristics, or the like, which are equally applicable to the various aspects described immediately above.

### A. Inhibition of Target Genes

### 1. CRL5 Complex Genes:

In accordance with some of the aspects and embodiments described herein, the present disclosure provides a genetically modified hematopoietic cell, comprising: one or more genetic modifications to one or more CRL5 complex genes that inhibits expression or activity of the polypeptide products encoded by the one or more CRL5 complex genes, wherein expression or activity of the polypeptide product is inhibited by at least about 50% compared to a control hematopoietic cell.

In some embodiments, the one or more CRL5 complex genes are selected from a group including: (i) Protein Ariadne-2 Homolog (ARIH2), (ii) Cullin-5 (CUL5), (iii) Defective in Cullin Neddylation 1 Domain Containing 3 (DCUN1D3), (iv) RING-box protein 2 (RNF7), (v) Ubiquitin Conjugating Enzyme E2 F (UBE2F), or any combination thereof. In some further embodiments, expression or activity of the polypeptide products of the one or more CRL5 complex genes are inhibited by at least about 50% to about 60%, about 60% to about 70%, about 70% to about 80%, about 80% to about 90%, about 90% to about 100%, about 50% to about 70%, about 60% to about 80%, about 70% to about 90%, about 80% to about 100%, about 50% to about 75%, about 75% to about 100%, about 50% to about 80%, about 60% to about 90%, about 70% to about 100%, about 50% to about 90%, or about 60% to about 100%. Alternatively, in some other further embodiments, the one or more genetic modifications of the one or more CRL5 complex genes inactivates the one or more CRL5 complex genes. As described above, the term “inactivates” refers to (1) a complete inhibition (i.e., 100% inhibition) in the expression of a gene, or (2) if expression remains, the protein encoded by the gene is not functional compared to a wild-type reference.

In some embodiments, the one or more genetic modifications comprise one or more gene deletions, resulting in the inactivation of the one or more CRL5 complex genes. As used herein, the term “gene deletion” refers to the removal of at least a portion of a DNA sequence from, or in proximity to, a gene. In some further embodiments, the sequences subjected to gene deletion can comprise one or more exonic sequences of the genes. Alternatively, or in addition to, the sequences subjected to gene deletion can comprise one or more promoter sequences of the genes.

In some further embodiments, the sequences subjected to gene deletion can comprise one or more flanking sequences of the genes. In some embodiments, a portion of one or more gene sequences is removed from the genes. Alternatively, or in addition to, the complete gene sequence can be removed from a chromosome. In some embodiments, a host cell, such as, for example, a hematopoietic cell, is modified to possess one or more gene deletions as described in any of the embodiments herein. In some embodiments, the gene is inactivated by a gene deletion, wherein deletion of at least one nucleotide to a gene sequence results in a gene product that no longer has the original gene product function or activity, or is a dysfunctional gene product.

In some embodiments, the one or more genetic modifications comprise one or more insertions and/or one or more substitutions, wherein incorporation or substitution of at least one nucleotide or nucleotide base pair into the gene sequence results in a non-functional gene product. In some embodiments, the one or more genetic modifications comprise one or more insertions and/or one or more substitutions, wherein incorporation or substitution of at least one nucleotide to the gene sequence results in a dysfunctional gene product. In some embodiments, a host cell, such as, for example, a hematopoietic cell, is modified to possess at least one additional nucleotide or nucleotide base pair as described in any of the embodiments herein. In some embodiments, a host cell, such as, for example, a hematopoietic cell, is modified such that one or more nucleotides or nucleotide base pairs is substituted for one or more different nucleotides or nucleotide base pairs as described in any of the embodiments herein.

In some embodiments, the one or more genetic modifications comprise one or more deletions, one or more insertions, and/or one or more substitutions, resulting in the inhibition of the one or more CRL5 complex genes (or polypeptide products encoded thereby). Alternatively, in some other embodiments, the one or more genetic modifications comprise one or more deletions, one or more insertions, and/or one or more substitutions, resulting in the inactivation of the one or more CRL5 complex genes. In embodiments where the genetically modified hematopoietic cell comprises one or more genetic modifications to at least two CRL5 complex genes, the genetic modifications to the at least two CRL5 complex genes may separately result in an inhibition or inactivation of the first CRL5 complex gene, and an inhibition or inactivation of the second CRL5 complex gene.

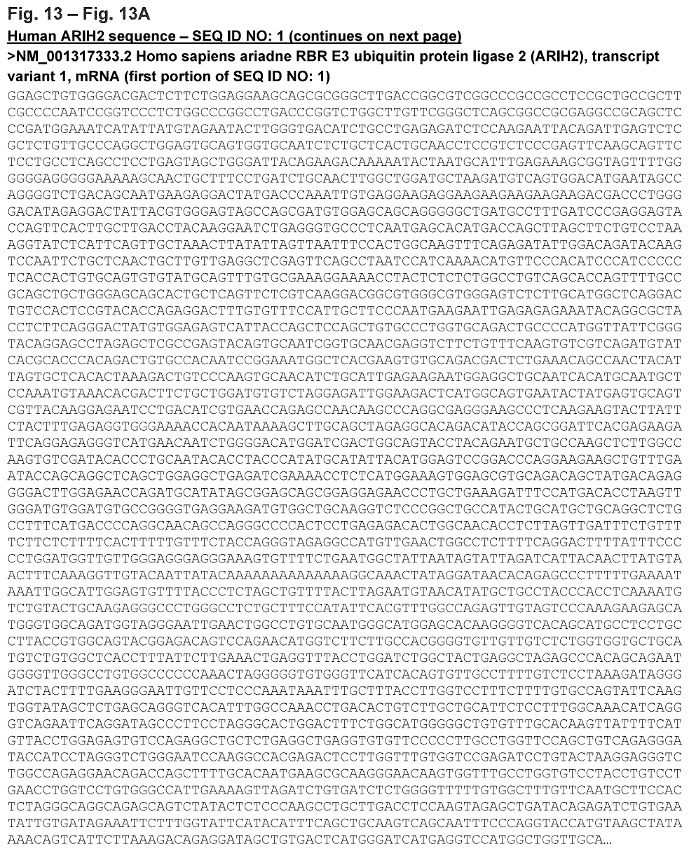

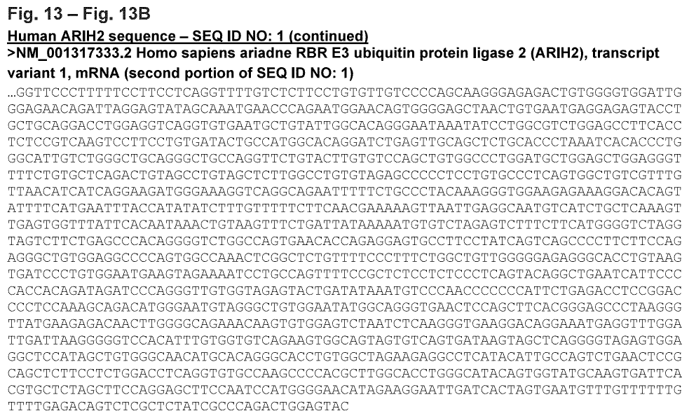

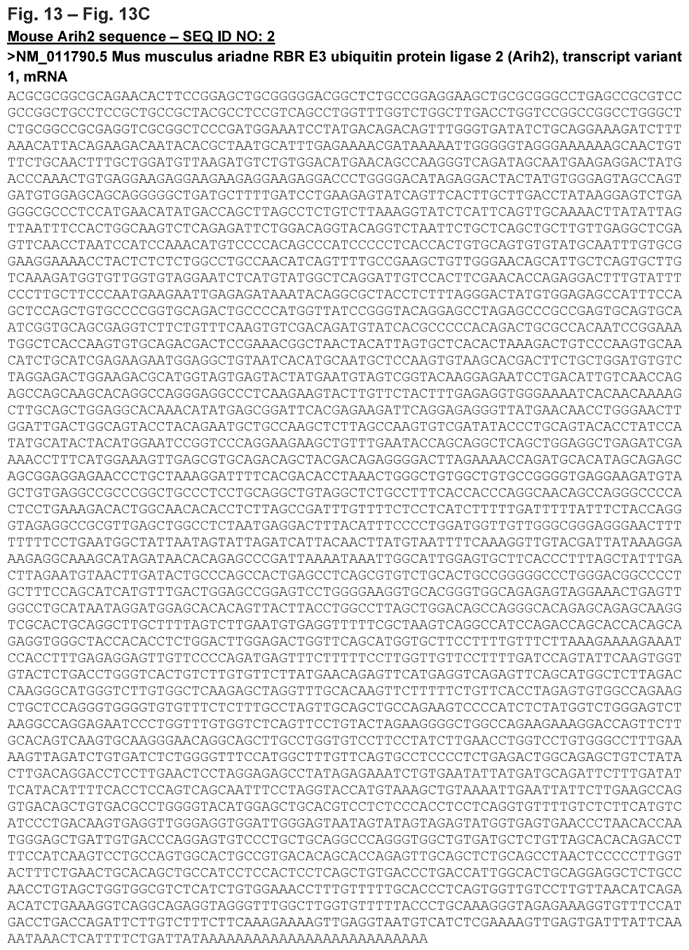

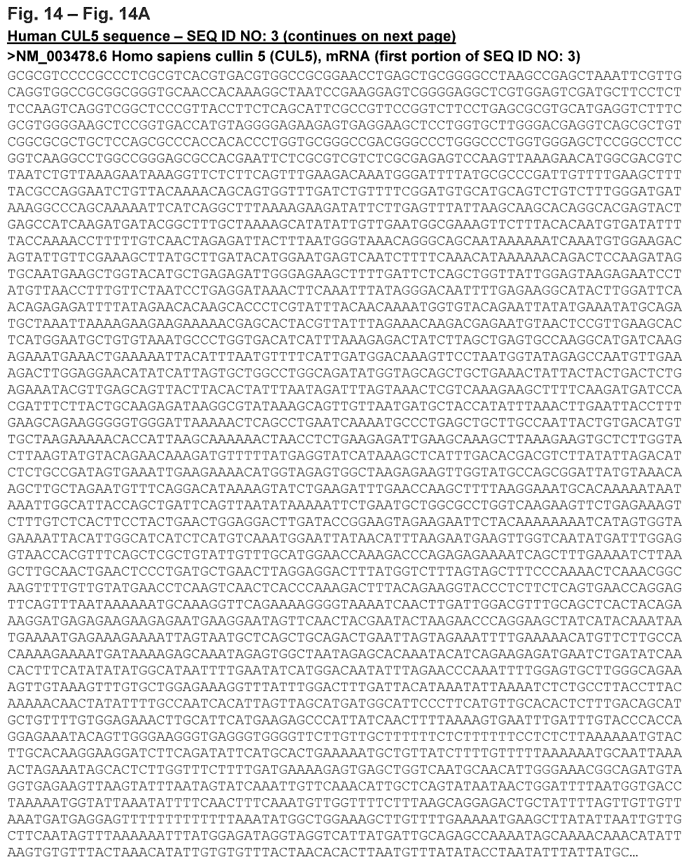

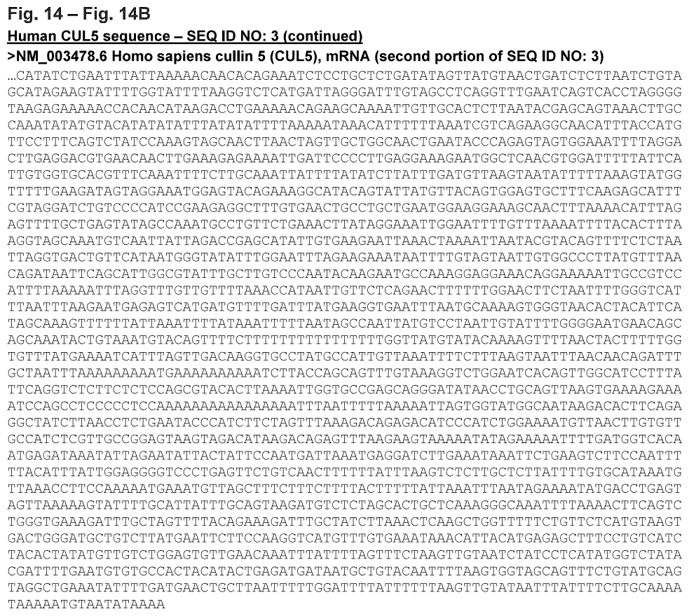

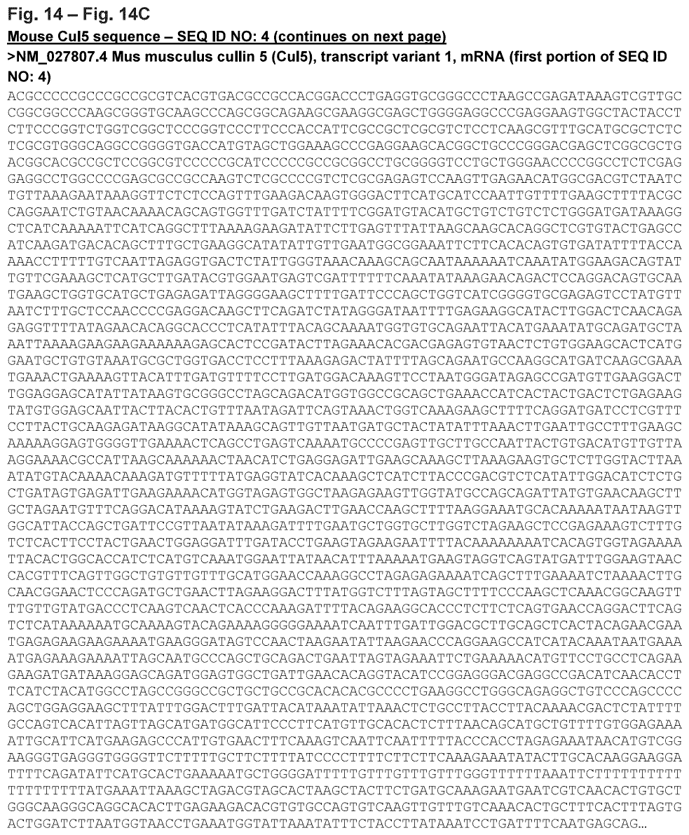

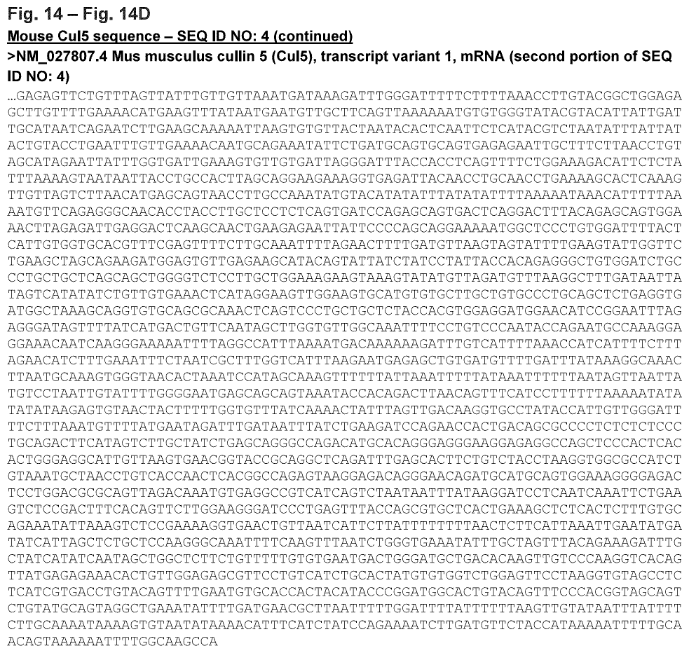

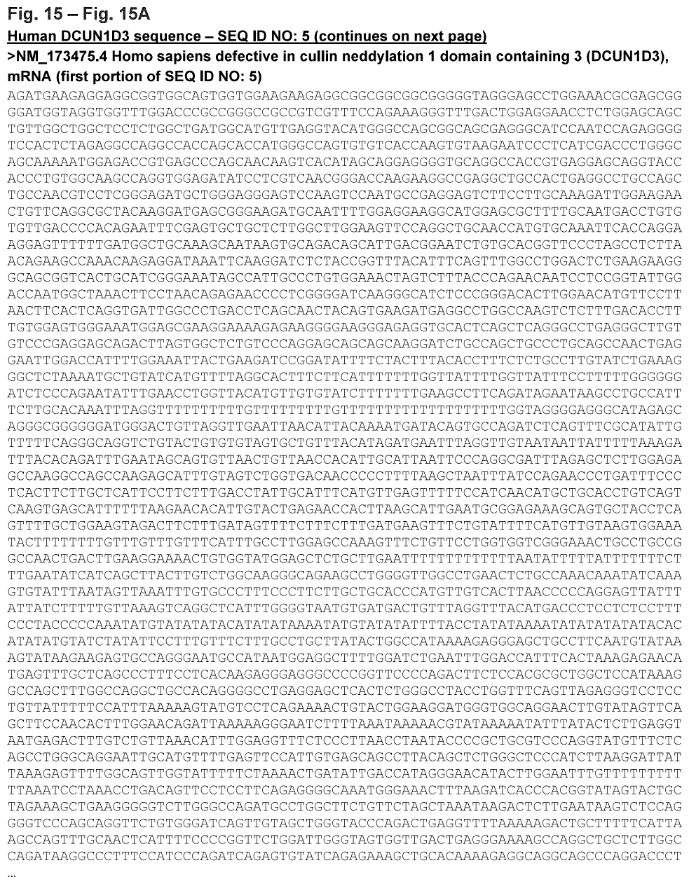

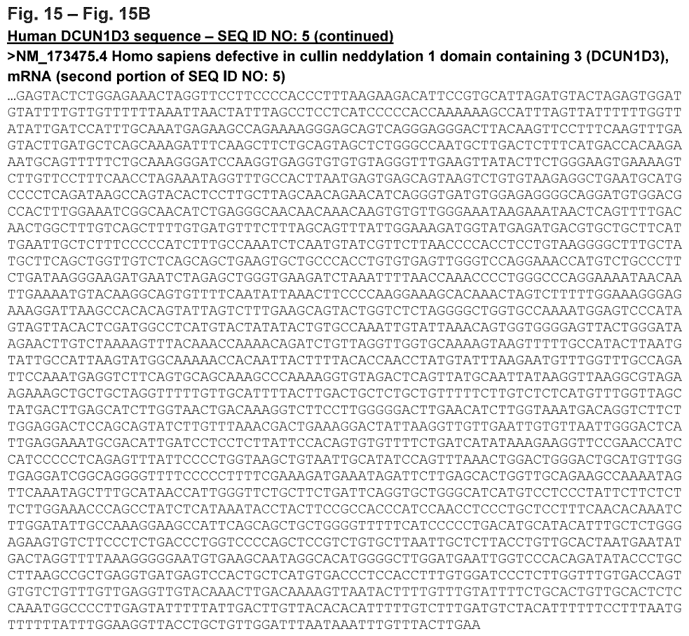

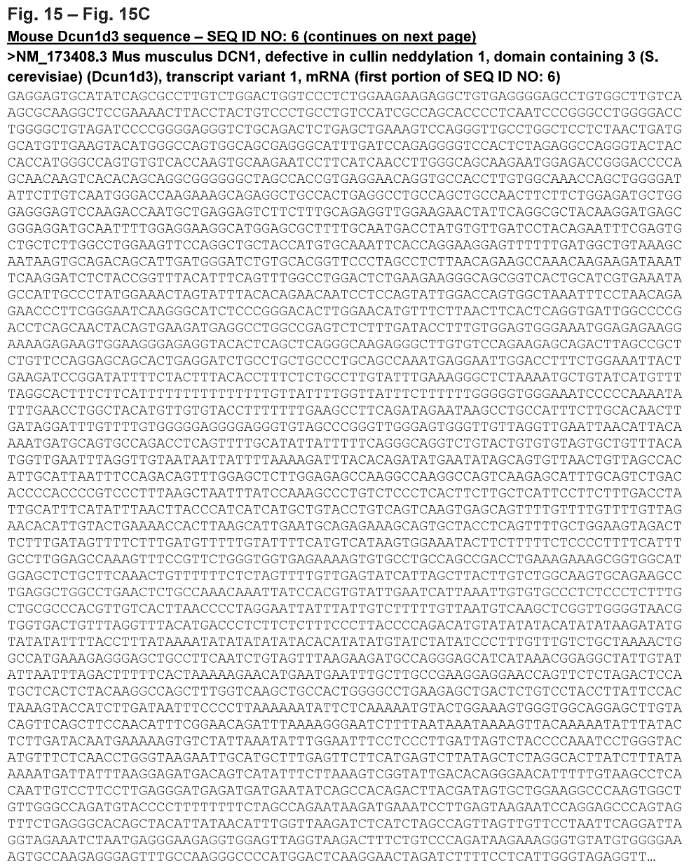

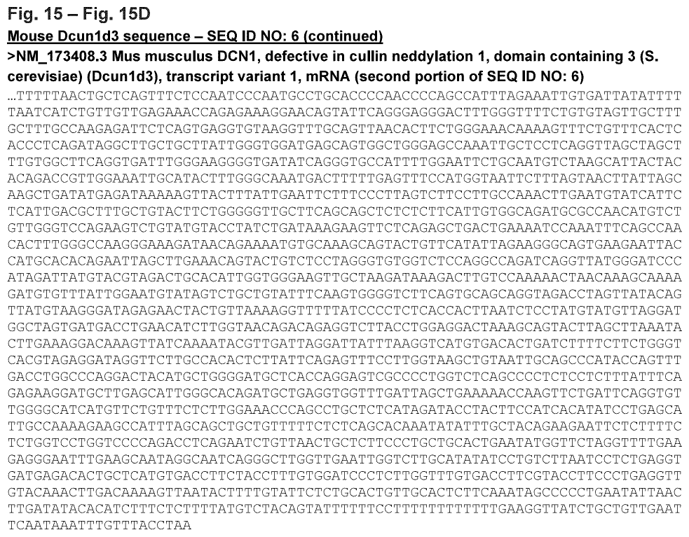

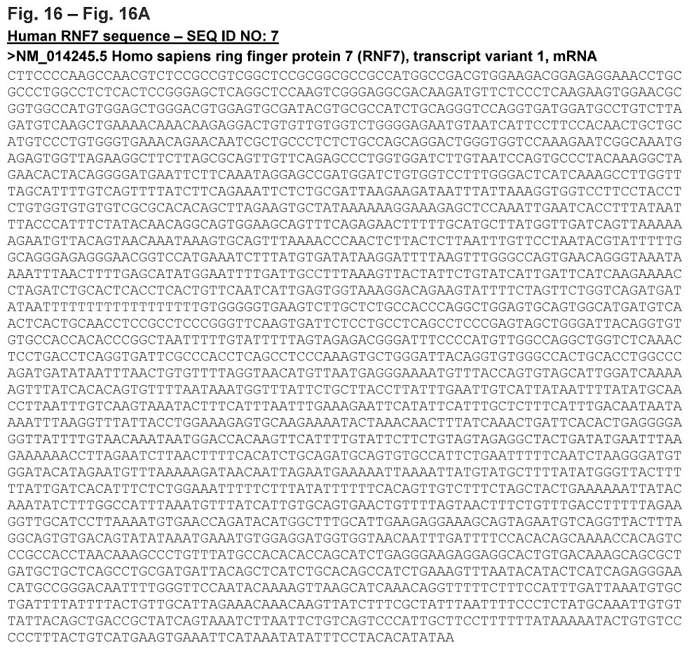

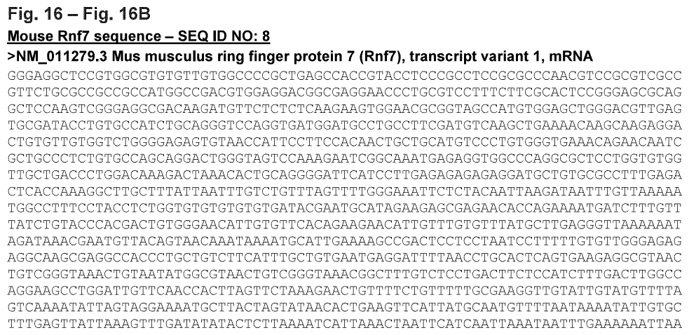

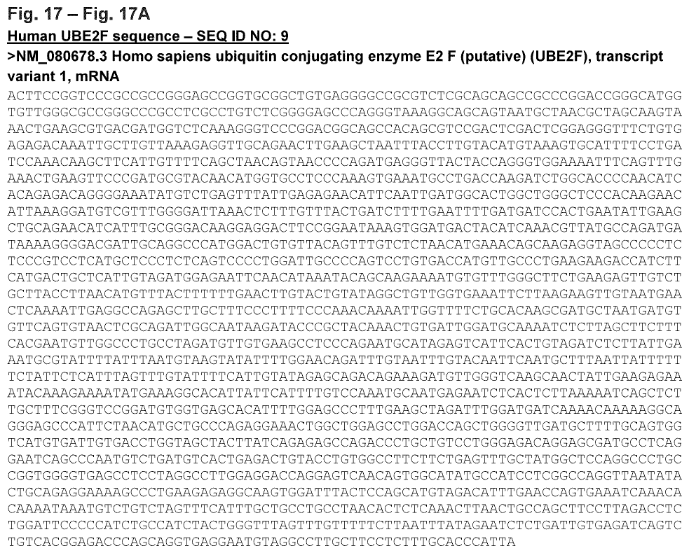

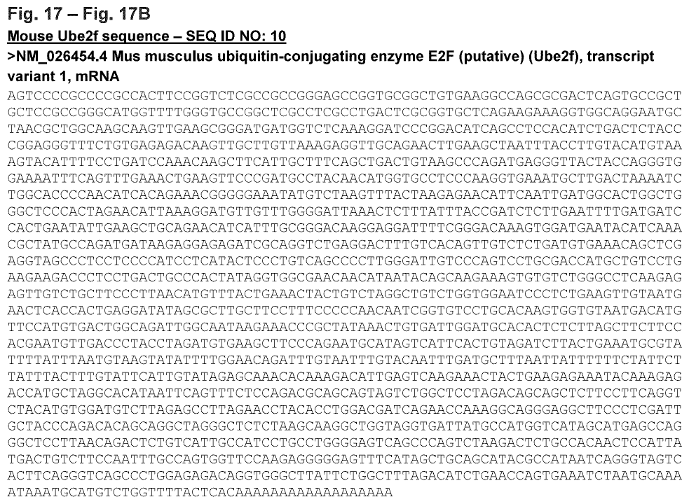

In some embodiments, the one or more CRL5 complex genes are inhibited using one or more genetic modification systems, as discussed in further detail below. In some embodiments, a target (or, targets) of a genetic modification system (or, genetic modification systems) can comprise a genetic region flanking the one or more CRL5 complex genes. In some embodiments, the target (or, targets) of the genetic modification system comprises a promoter region of the one or more CRL5 complex genes. In some embodiments, the target (or, targets) of the genetic modification system comprises a coding region of the one or more CRL5 complex genes. In some embodiments, the target (or, targets) of the genetic modification system comprises a non-coding region of the one or more CRL5 complex genes. In some embodiments, the target (or, targets) of the genetic modification system comprises one or more specific nucleic acid residues encoding the one or more CRL5 complex genes. In some embodiments, the target (or, targets) of the genetic modification system comprises one or more of SEQ ID NOs: 1-10, as shown in FIGS. 13-17. In some embodiments, the target (or, targets) of the genetic modification system comprises the entire length of one or more of SEQ ID NOs: 1-10. In some embodiments, the target (or, targets) of the genetic modification system may comprise one or more nucleic acid residues of one or more of SEQ ID NOs: 1-10.

### 2. CRL5 Complex-Independent Genes:

In accordance with some of the aspects and embodiments described herein, the present disclosure provides a genetically modified hematopoietic cell, comprising: one or more genetic modifications to one or more CRL5 complex-independent genes that inhibit expression or activity of the one or more polypeptide products encoded by the one or more CRL5 complex-independent genes, wherein expression or activity of the polypeptide product is inhibited by at least about 50% compared to a control hematopoietic cell.

In some embodiments, the one or more CRL5 complex-independent genes are selected from a group including: (a) ARHGAP25, (b) CCNC, (c) FAM49B, (d) N4BP1, (e) STUB1, or any combination thereof. In some further embodiments, expression or activity of the polypeptide product (of the one or more CRL5 complex-independent genes) is inhibited by at least about 50% to about 60%, about 60% to about 70%, about 70% to about 80%, about 80% to about 90%, about 90% to about 100%, about 50% to about 70%, about 60% to about 80%, about 70% to about 90%, about 80% to about 100%, about 50% to about 75%, about 75% to about 100%, about 50% to about 80%, about 60% to about 90%, about 70% to about 100%, about 50% to about 90%, or about 60% to about 100%. Alternatively, in some other further embodiments, the one or more genetic modifications of the one or more CRL5 complex-independent genes inactivates the one or more CRL5 complex-independent genes. As described above, the term “inactivates” refers to (1) a complete inhibition (i.e., 100% inhibition) in the expression of a gene, or (2) if expression remains, the protein encoded by the gene is not functional compared to a wild-type reference.

In some embodiments, the one or more genetic modifications comprise one or more gene deletions, resulting in the inactivation of the one or more CRL5 complex-independent genes. As used herein, the term “gene deletion” refers to the removal of at least a portion of a DNA sequence from, or in proximity to, a gene. In some further embodiments, the sequences subjected to gene deletion can comprise one or more exonic sequences of the genes. Alternatively, or in addition to, the sequences subjected to gene deletion can comprise one or more promoter sequences of the genes. In some further embodiments, the sequences subjected to gene deletion can comprise one or more flanking sequences of the genes. In some embodiments, a portion of one or more gene sequences is removed from the genes. Alternatively, or in addition to, the complete gene sequence can be removed from a chromosome. In some embodiments, a host cell, such as, for example, a hematopoietic cell, is modified to possess one or more gene deletions as described in any of the embodiments herein. In some embodiments, the gene is inactivated by a gene deletion, wherein deletion of at least one nucleotide to a gene sequence results in a gene product that no longer has the original gene product function or activity, or is a dysfunctional gene product.

In some embodiments, the one or more genetic modifications comprise one or more insertions and/or one or more substitutions, wherein incorporation or substitution of at least one nucleotide or nucleotide base pair into the gene sequence results in a non-functional gene product. In some embodiments, the one or more genetic modifications comprise one or more insertions and/or one or more substitutions, wherein incorporation or substitution of at least one nucleotide to the gene sequence results in a dysfunctional gene product. In some embodiments, a host cell, such as, for example, a hematopoietic cell, is modified to possess at least one additional nucleotide or nucleotide base pair as described in any of the embodiments herein. In some embodiments, a host cell, such as, for example, a hematopoietic cell, is modified such that one or more nucleotides or nucleotide base pairs is substituted for one or more different nucleotides or nucleotide base pairs as described in any of the embodiments herein.

In some embodiments, the one or more genetic modifications comprise one or more deletions, one or more insertions, and/or one or more substitutions, resulting in the inhibition of the one or more CRL5 complex-independent genes (or polypeptide products encoded thereby). Alternatively, in some other embodiments, the one or more genetic modifications comprise one or more deletions, one or more insertions, and/or one or more substitutions, resulting in the inactivation of the one or more CRL5 complex-independent genes. In embodiments where the genetically modified hematopoietic cell comprises one or more genetic modifications to at least two CRL5 complex-independent genes, the genetic modifications to the at least two CRL5 complex-independent genes may separately result in an inhibition or inactivation of the first CRL5 complex-independent gene, and an inhibition or inactivation of the second CRL5 complex-independent gene.

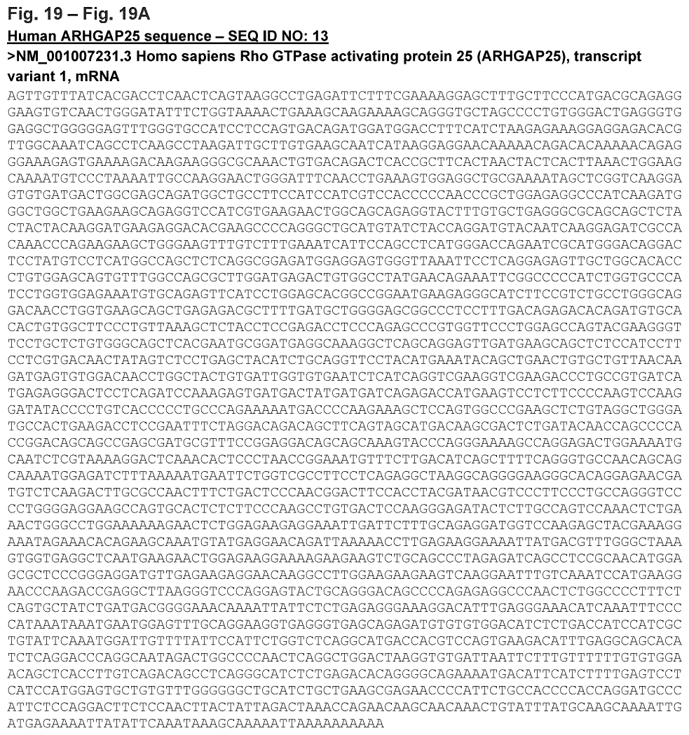

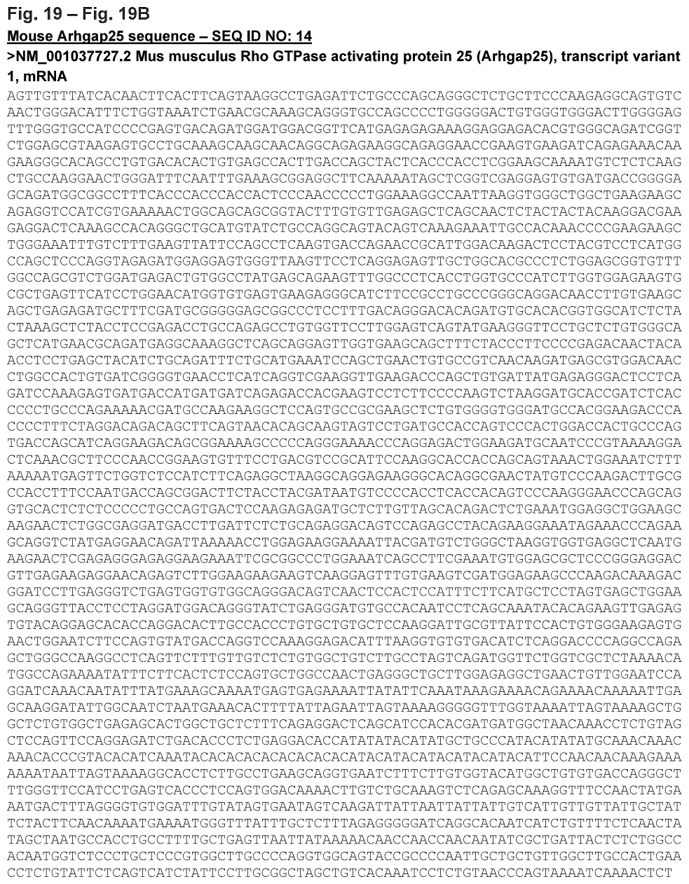

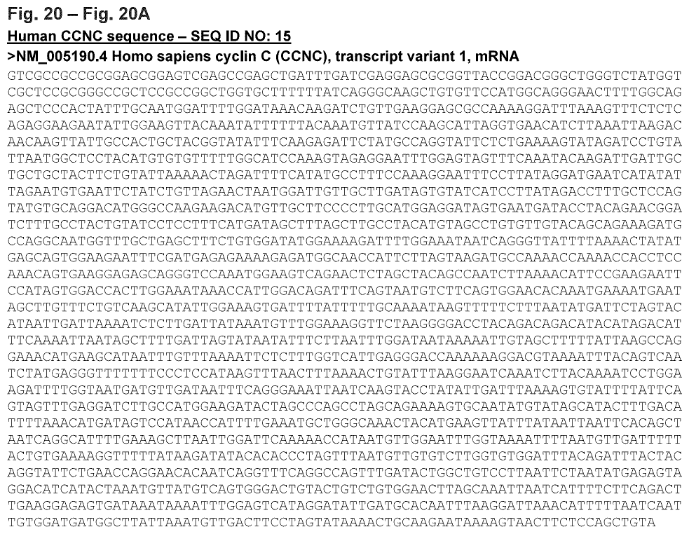

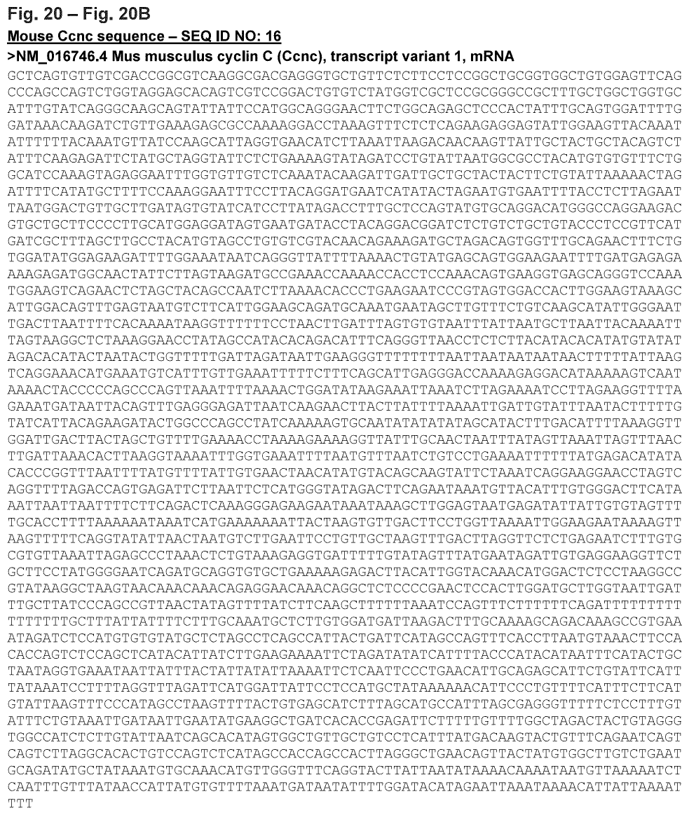

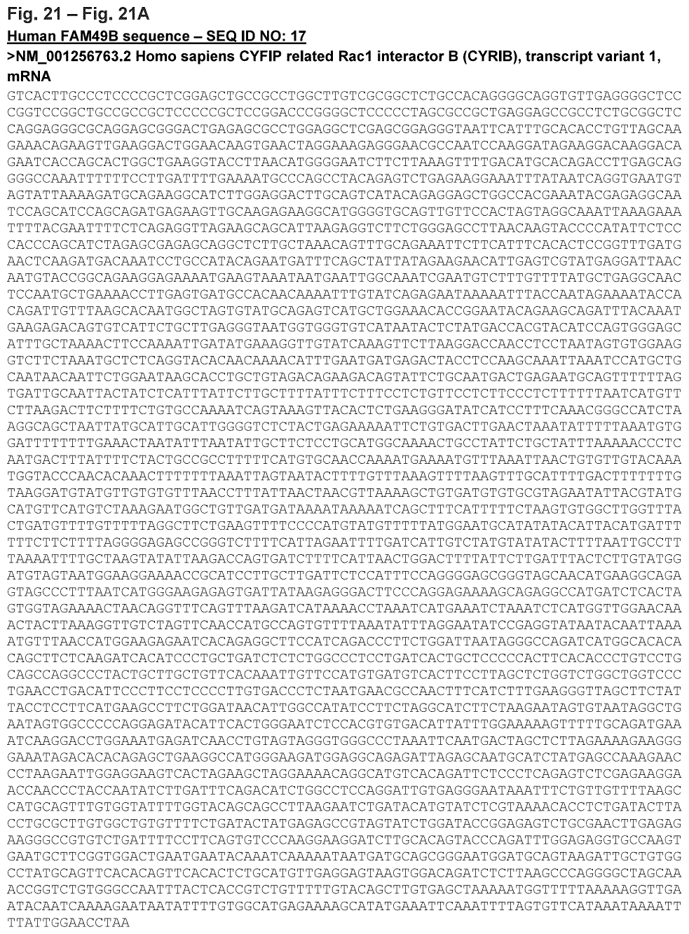

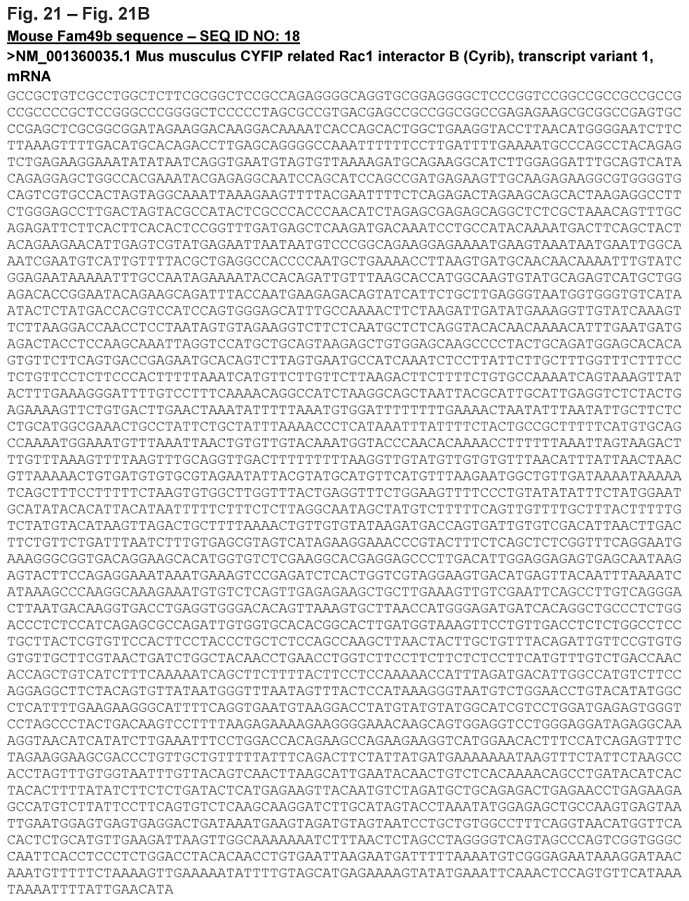

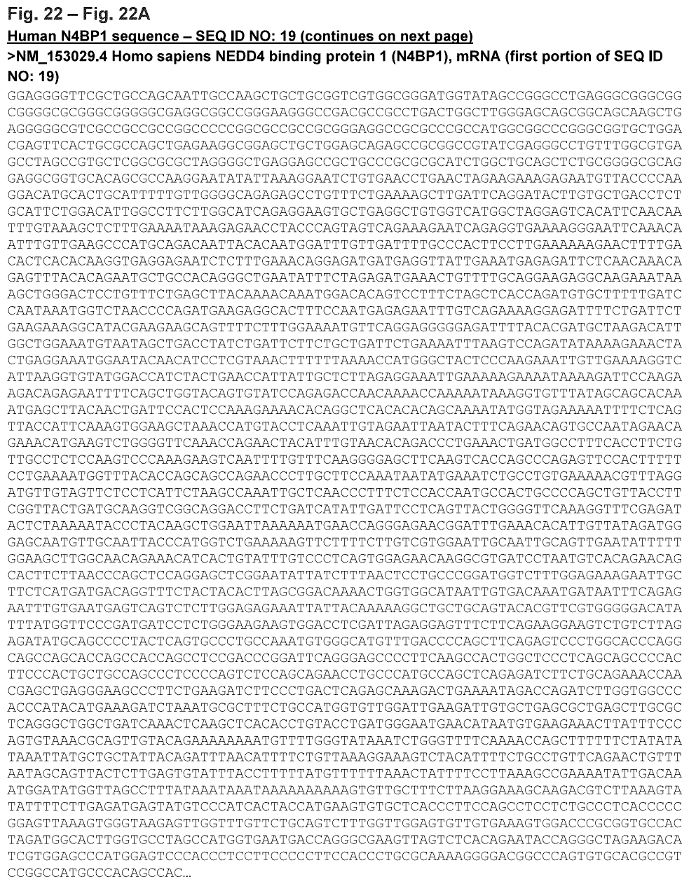

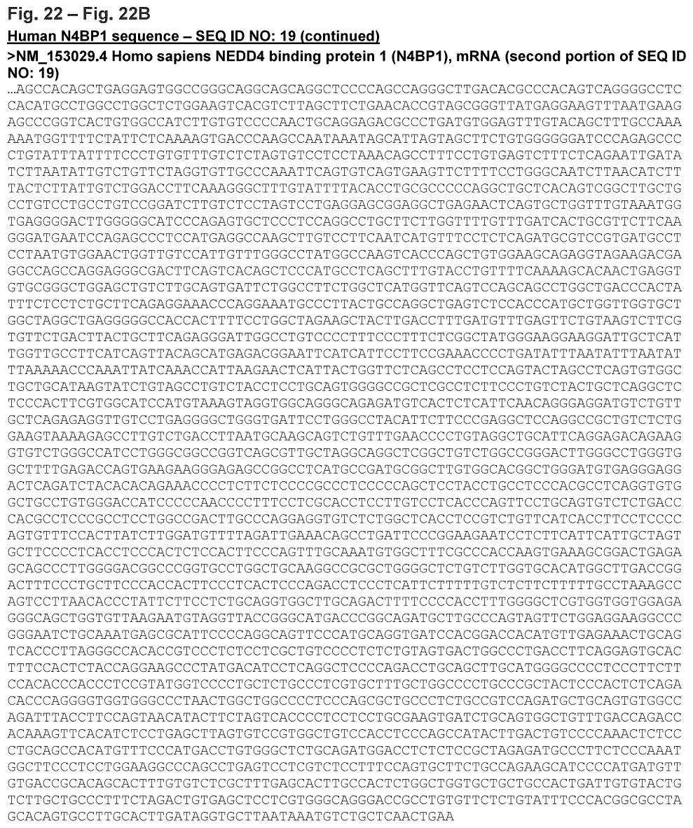

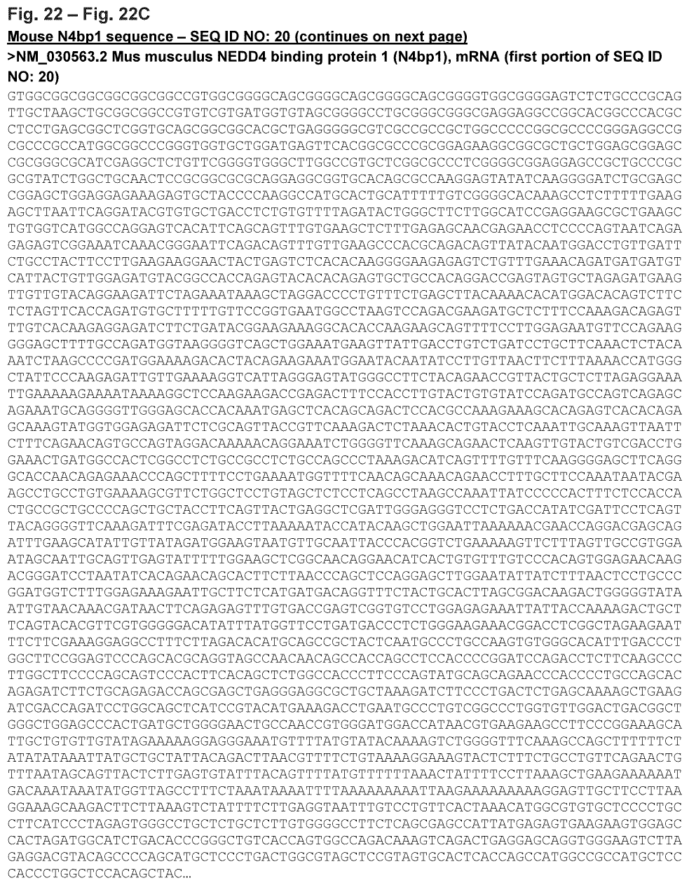

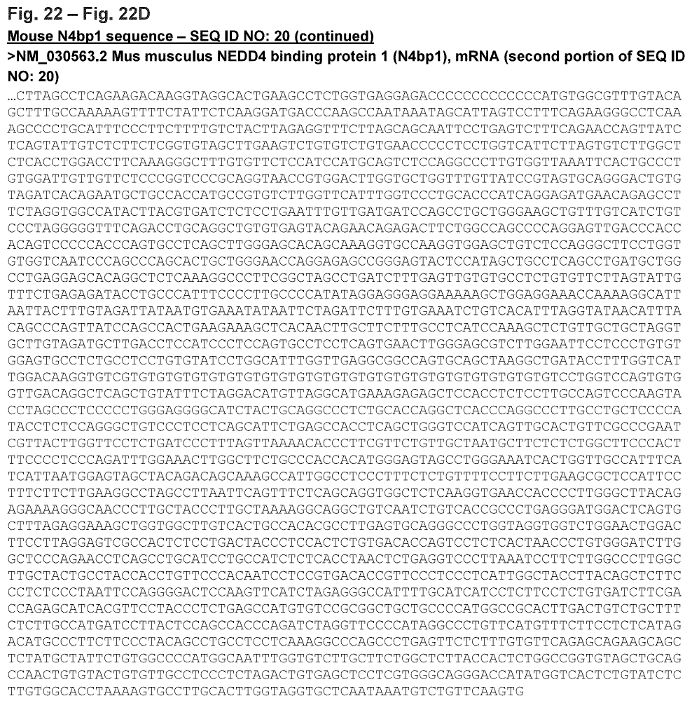

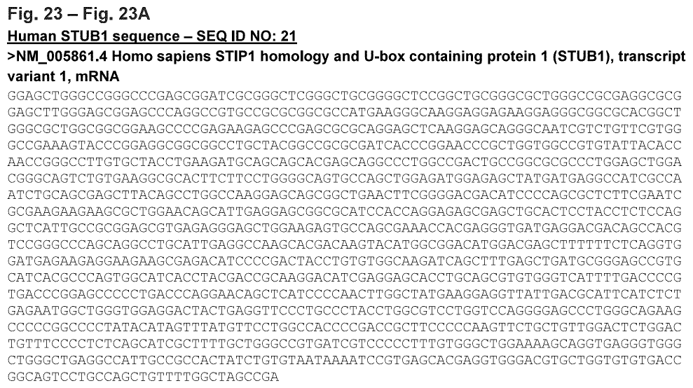

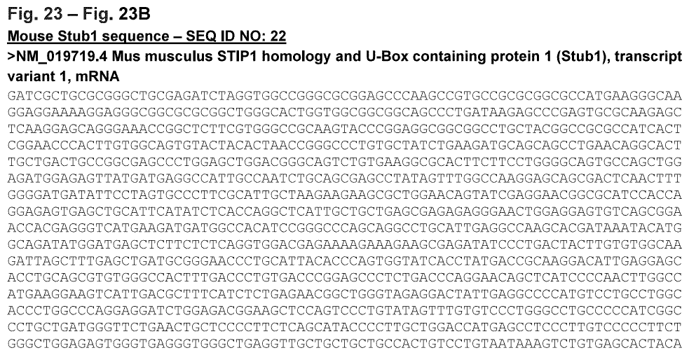

In some embodiments, the one or more CRL5 complex-independent genes are inhibited using one or more genetic modification systems, as discussed in further detail below. In some embodiments, a target (or, targets) of a genetic modification system (or, genetic modification systems) can comprise a genetic region flanking the one or more CRL5 complex-independent genes. In some embodiments, the target (or, targets) of the genetic modification system comprises a promoter region of the one or more CRL5 complex-independent genes. In some embodiments, the target (or, targets) of the genetic modification system comprises a coding region of the one or more CRL5 complex-independent genes. In some embodiments, the target (or, targets) of the genetic modification system comprises a non-coding region of the one or more CRL5 complex-independent genes. In some embodiments, the target (or, targets) of the genetic modification system comprises one or more specific nucleic acid residues encoding the one or more CRL5 complex-independent genes. In some embodiments, the target (or, targets) of the genetic modification system comprises one or more of SEQ ID NOs: 13-22, as shown in FIGS. 19-23. In some embodiments, the target (or, targets) of the genetic modification system comprises the entire length of one or more of SEQ ID NOs: 13-22. In some embodiments, the target (or, targets) of the genetic modification system may comprise one or more nucleic acid residues of one or more of SEQ ID NOs: 13-22.

### 3. Combination of CRL5 Complex Genes and CRL5 Complex-Independent Genes:

In accordance with some of the aspects and embodiments described herein, the present disclosure provides a genetically modified hematopoietic cell, comprising: (1) one or more genetic modifications to one or more CRL5 complex genes that inhibits expression or activity of the polypeptide products encoded by the one or more CRL5 complex genes, and (2) one or more genetic modifications to one or more CRL5 complex-independent genes that inhibits expression or activity of the polypeptide products encoded by the one or more CRL5 complex-independent genes, wherein expression or activity of each polypeptide product is inhibited by at least about 50% compared to a control hematopoietic cell.

In some embodiments, the one or more CRL5 complex genes are selected from a group including: (i) Protein Ariadne-2 Homolog (ARIH2), (ii) Cullin-5 (CUL5), (iii) Defective in Cullin Neddylation 1 Domain Containing 3 (DCUN1D3), (iv) RING-box protein 2 (RNF7), (v) Ubiquitin Conjugating Enzyme E2 F (UBE2F), (vi) CISH, or any combination thereof. In some further embodiments, expression or activity of the polypeptide products of the one or more CRL5 complex genes are inhibited by at least about 50% to about 60%, about 60% to about 70%, about 70% to about 80%, about 80% to about 90%, about 90% to about 100%, about 50% to about 70%, about 60% to about 80%, about 70% to about 90%, about 80% to about 100%, about 50% to about 75%, about 75% to about 100%, about 50% to about 80%, about 60% to about 90%, about 70% to about 100%, about 50% to about 90%, or about 60% to about 100%. Alternatively, in some other further embodiments, the one or more genetic modifications of the one or more CRL5 complex genes inactivates the one or more CRL5 complex genes. As described above, the term “inactivates” refers to (1) a complete inhibition (i.e., 100% inhibition) in the expression of a gene, or (2) if expression remains, the protein encoded by the gene is not functional compared to a wild-type reference.

In some embodiments, the one or more CRL5 complex-independent genes are selected from a group including: (a) ARHGAP25, (b) CCNC, (c) FAM49B, (d) N4BP1, (e) STUB1, or any combination thereof. In some further embodiments, expression or activity of the polypeptide product of the one or more CRL5 complex-independent genes is inhibited by at least about 50% to about 60%, about 60% to about 70%, about 70% to about 80%, about 80% to about 90%, about 90% to about 100%, about 50% to about 70%, about 60% to about 80%, about 70% to about 90%, about 80% to about 100%, about 50% to about 75%, about 75% to about 100%, about 50% to about 80%, about 60% to about 90%, about 70% to about 100%, about 50% to about 90%, or about 60% to about 100%. Alternatively, in some other further embodiments, the one or more genetic modifications of the one or more CRL5 complex-independent genes inactivates the one or more CRL5 complex-independent genes. As described above, the term “inactivates” refers to (1) a complete inhibition (i.e., 100% inhibition) in the expression of a gene, or (2) if expression remains, the protein encoded by the gene is not functional compared to a wild-type reference.

In some embodiments, the one or more CRL5 complex genes are inhibited (or, inactivated) using a first genetic modification system, and the one or more CRL5 complex-independent genes are inhibited (or, inactivated) using a second genetic modification system. In some further embodiments, the first genetic modification system and the second genetic modification system are the same “type” of genetic modification system (such as, for example, a first CRISPR system and a second CRISPR system). In some other further embodiments, the first genetic modification system and the second genetic modification system are not the same “type” of genetic modification system. For example, in some embodiments, the first genetic modification system may comprise a CRISPR system, and the second genetic modification system is not a CRISPR system.

In some embodiments, the one or more genetic modifications of the CRL5 complex(-independent) genes comprise one or more gene deletions, resulting in the inactivation of the one or more CRL5 complex(-independent) genes. As used herein, the term “gene deletion” refers to the removal of at least a portion of a DNA sequence from, or in proximity to, a gene. In some further embodiments, the sequences subjected to gene deletion can comprise one or more exonic sequences of the genes. Alternatively, or in addition to, the sequences subjected to gene deletion can comprise one or more promoter sequences of the genes. In some further embodiments, the sequences subjected to gene deletion can comprise one or more flanking sequences of the genes.

In some embodiments, a portion of one or more gene sequences is removed from the genes.

Alternatively, or in addition to, the complete gene sequence can be removed from a chromosome. In some embodiments, a host cell, such as, for example, a hematopoietic cell, is modified to possess one or more gene deletions as described in any of the embodiments herein. In some embodiments, the gene is inactivated by a gene deletion, wherein deletion of at least one nucleotide to a gene sequence results in a gene product that no longer has the original gene product function or activity, or is a dysfunctional gene product.

In some embodiments, the one or more genetic modifications of the CRL5 complex(-independent) genes comprise one or more insertions and/or one or more substitutions, wherein incorporation or substitution of at least one nucleotide or nucleotide base pair into the gene sequence results in a non-functional gene product. In some embodiments, the one or more genetic modifications comprise one or more insertions and/or one or more substitutions, wherein incorporation or substitution of at least one nucleotide to the gene sequence results in a dysfunctional gene product. In some embodiments, a host cell, such as, for example, a hematopoietic cell, is modified to possess at least one additional nucleotide or nucleotide base pair as described in any of the embodiments herein. In some embodiments, a host cell, such as, for example, a hematopoietic cell, is modified such that one or more nucleotides or nucleotide base pairs is substituted for one or more different nucleotides or nucleotide base pairs as described in any of the embodiments herein.

In some embodiments, the one or more genetic modifications comprise one or more deletions, one or more insertions, and/or one or more substitutions, resulting in the inhibition of the one or more CRL5 complex(-independent genes) (or polypeptide products encoded thereby). Alternatively, in some other embodiments, the one or more genetic modifications comprise one or more deletions, one or more insertions, and/or one or more substitutions, resulting in the inactivation of the one or more CRL5 complex(-independent) genes. In embodiments where the genetically modified hematopoietic cell comprises one or more genetic modifications to at least two CRL5 complex(-independent) genes, the genetic modifications to the at least two CRL5 complex(-independent) genes may separately result in an inhibition or inactivation of the first CRL5 complex-independent gene, and an inhibition or inactivation of the second CRL5 complex gene.

In some embodiments, the one or more genetic modifications of the one or more CRL5 complex genes may comprise one or more deletions, one or more insertions, and/or one or more substitutions, resulting in the inhibition of the one or more CRL5 complex genes (or polypeptide products encoded thereby); and, the one or more genetic modifications of the one or more CRL5 complex-independent genes may comprise one or more deletions, one or more insertions, and/or one or more substitutions, resulting in the inhibition of the one or more CRL5 complex-independent genes (or polypeptide products encoded thereby). Alternatively, the one or more genetic modifications of the one or more CRL5 complex genes may comprise one or more deletions, one or more insertions, and/or one or more substitutions, resulting in the inactivation of the one or more CRL5 complex genes (or polypeptide products encoded thereby); and, the one or more genetic modifications of the one or more CRL5 complex-independent genes may comprise one or more deletions, one or more insertions, and/or one or more substitutions, resulting in the inactivation of the one or more CRL5 complex-independent genes (or polypeptide products encoded thereby). As yet another alternative, the one or more genetic modifications of the one or more CRL5 complex genes may comprise one or more deletions, one or more insertions, and/or one or more substitutions, resulting in the inhibition or inactivation of the one or more CRL5 complex genes (or polypeptide products encoded thereby); and, the one or more genetic modifications of the one or more CRL5 complex-independent genes may comprise one or more deletions, one or more insertions, and/or one or more substitutions, resulting in the inhibition or inactivation of the one or more CRL5 complex-independent genes (or polypeptide products encoded thereby).

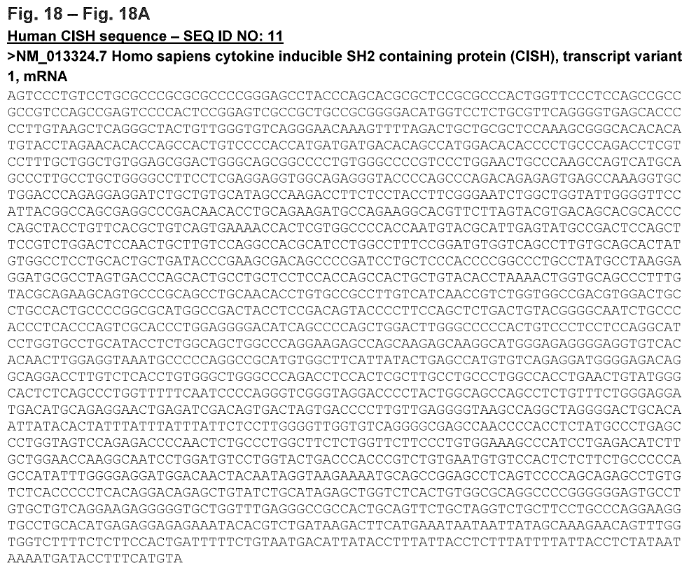

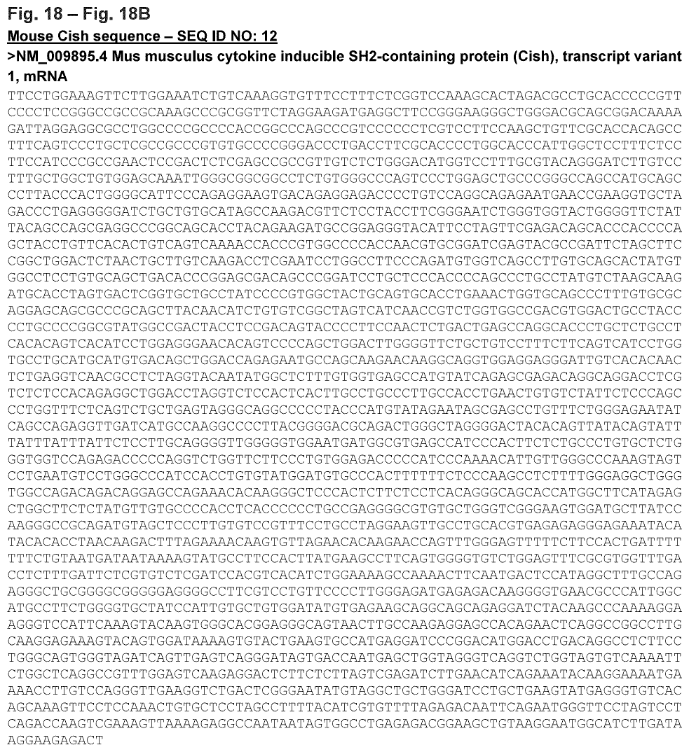

In some embodiments, a target (or, targets) of a genetic modification system (e.g., the first genetic modification system, the second genetic modification system, etc.) can comprise a genetic region flanking the one or more CRL5 complex(-independent) genes. In some embodiments, the target (or, targets) of a genetic modification system comprises a promoter region of the one or more CRL5 complex(-independent) genes. In some embodiments, the target (or, targets) of a genetic modification system comprises a coding region of the one or more CRL5 complex(-independent) genes. In some embodiments, the target (or, targets) of a genetic modification system comprises a non-coding region of the one or more CRL5 complex(-independent) genes. In some embodiments, the target (or, targets) of a genetic modification system comprises one or more specific nucleic acid residues encoding the one or more CRL5 complex(-independent) genes. In some embodiments, the target (or, targets) of the genetic modification system comprises one or more of SEQ ID NOs: 1-22, as shown in FIGS. 13-23. In some embodiments, the target (or, targets) of the genetic modification system comprises the entire length of one or more of SEQ ID NOs: 1-22. In some embodiments, the target (or, targets) of the genetic modification system may comprise one or more nucleic acid residues of one or more of SEQ ID NOs: 1-22.

### B. Genetic Modification Systems

In some embodiments, the one or more CRL5 complex genes are inhibited using: (i) a clustered, regularly interspaced, short palindromic repeats (CRISPR) system, (ii) a transcription activator-like effector nuclease (TALEN) system, (iii) a zinc finger nuclease (ZFN) system, (iv) a meganuclease system, (v) a short hairpin RNA (shRNA; also referred to as a “small hairpin RNA”) system, (vi) a small interfering RNA (siRNA) system, (vii) a microRNA (miRNA) system, or (viii) an antisense RNA system.

Efficiency of the inhibition (or inactivation) of expression of any of the one or more CRL5 complex genes described herein can be assessed by measuring the amount of mRNA or protein using methods known in the relevant art, such as, for example, quantitative PCR (qPCR), Western blotting, flow cytometry, and the like. In some embodiments, protein levels of the one or more CRL5 complex genes are evaluated to assess efficiency of inhibiting gene expression of the one or more CRL5 complex genes. In certain embodiments, the efficiency of the reduction in target gene expression (i.e., the one or more CRL5 complex genes) is at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99%, or greater as compared to a control hematopoietic cell. In certain embodiments, the efficiency of the reduction in target gene expression (i.e., the one or more CRL5 complex genes) is between about 5% to about 20%, between about 20% to about 40%, between about 40% to about 60%, between about 60% to about 80%, or between about 80% to 99% or greater.

In some embodiments, the one or more CRL5 complex-independent genes are inhibited using: (i) a clustered, regularly interspaced, short palindromic repeats (CRISPR) system, (ii) a transcription activator-like effector nuclease (TALEN) system, (iii) a zinc finger nuclease (ZFN) system, (iv) a meganuclease system, (v) a short hairpin RNA (shRNA; also referred to as a “small hairpin RNA”) system, (vi) a small interfering RNA (siRNA) system, (vii) a microRNA (miRNA) system, or (viii) an antisense RNA system.

Efficiency of the inhibition (or inactivation) of expression of any of the one or more CRL5 complex-independent genes described herein can be assessed by measuring the amount of mRNA or protein using methods known in the relevant art, such as, for example, quantitative PCR (qPCR), Western blotting, flow cytometry, and the like. In some embodiments, protein levels of the one or more CRL5 complex-independent genes are evaluated to assess efficiency of inhibiting gene expression of the one or more CRL5 complex-independent genes. In certain embodiments, the efficiency of the reduction in target gene expression (i.e., the one or more CRL5 complex-independent genes) is at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99%, or greater as compared to a control hematopoietic cell. In certain embodiments, the efficiency of the reduction in target gene expression (i.e., the one or more CRL5 complex-independent genes) is between about 5% to about 20%, between about 20% to about 40%, between about 40% to about 60%, between about 60% to about 80%, or between about 80% to 99% or greater.

In some embodiments, the one or more CRL5 complex genes are inhibited using a first genetic modification system, and the one or more CRL5 complex-independent genes are inhibited using a second genetic modification system. The first genetic modification system and the second genetic modification system may be optionally and independently selected from: (i) a clustered, regularly interspaced, short palindromic repeats (CRISPR) system, (ii) a transcription activator-like effector nuclease (TALEN) system, (iii) a zinc finger nuclease (ZFN) system, (iv) a meganuclease system, (v) a short hairpin RNA (shRNA; also referred to as a “small hairpin RNA”) system, (vi) a small interfering RNA (siRNA) system, (vii) a microRNA (miRNA) system, or (viii) an antisense RNA system. In some further embodiments, the first genetic modification system and the second genetic modification system are substantially identical (i.e., the first genetic modification system and the second genetic modification system are the same “type” except for the fact that the first and second genetic modification systems target different genes utilizing different “guide sequences”). In some other further embodiments, the first genetic modification system and the second genetic modification system are different (e.g., the first genetic system may comprise a CRISPR system, and the second genetic modification system does not comprise a CRISPR system). It is expressly contemplated that any of the genetic modification systems desired herein can be utilized as the first genetic modification system, the second genetic modification system, or a genetic modification system of a higher integer value (such as, for example, in instances where three or more genes are targeted).

### 1. Genetic Modifications Using a CRISPR System:

In some embodiments, the one or more CRL5 complex genes and/or the one or more CRL5 complex-independent genes are inhibited using a CRISPR system. Illustrative methods of using the CRISPR system (also referred to herein as a “CRISPR/Cas” system) to reduce gene expression are described in various publications (see, e.g., U.S. Patent Application Publication No. 2014/0170753; incorporated herein by reference in its entirety, with particularity for relevant disclosure pertaining to CRISPR systems). Generally, a CRISPR/Cas system includes a Cas protein and at least one to two ribonucleic acids that hybridize to a target motif (such as, for example, a CRL5 complex gene, a CRL5 complex-independent gene, etc.) and direct the Cas protein to the target motif. Any CRISPR/Cas system that is capable of altering a target polynucleotide sequence in a cell (such as, for example, a hematopoietic cell used (i) to develop the compositions, and/or (ii) in the methods disclosed herein) can be used. In some embodiments, the CRISPR/Cas system comprises a CRISPR class 1 system. In some further embodiments, the CRISPR class 1 system is selected from a group including: CRISPR class 1 type I, CRISPR class 1 type III, and CRISPR class 1 type IV. In yet further embodiments, the CRISPR class 1 type I is selected from a group including: CRISPR class 1 type I subtype I-A, subtype I-B, subtype I-C, subtype I-D, subtype I-E, subtype I-F, and subtype I-G. In other embodiments, the CRISPR class 1 type III is selected from a group including: CRISPR class 1 type III subtype III-A, subtype III-B, subtype III-C, subtype III-D, subtype III-E, and subtype III-F. In still other embodiments, the CRISPR class 1 type IV is selected from a group including: CRISPR class 1 type IV subtype IV-A, subtype IV-B, and subtype IV-C. Alternatively, or in addition to, the CRISPR/Cas system may comprise a CRISPR class 2 system. In some further embodiments, the CRISPR class 2 system is selected from a group including: CRISPR class 2 type II, CRISPR class 2 type V, and CRISPR class 2 type VI. In yet further embodiments, the CRISPR class 2 type II is selected from a group including: CRISPR class 2 type II subtype II-A, subtype II-B, and subtype II-C. In other embodiments, the CRISPR class 2 type V is selected from a group including: CRISPR class 2 type V subtype V-A, subtype V-B, subtype V-C, subtype V-D, subtype V-E, subtype V-F, subtype V-G, subtype V-H, subtype V-I, subtype V-K, and subtype V-U. In still other embodiments, the CRISPR class 2 type VI is selected from a group including: CRISPR class 2 type VI subtype VI-A, subtype VI-B, subtype VI-C, and subtype VI-D.

The Cas protein used in the invention can be a naturally occurring Cas protein or a functional derivative thereof. As used herein, the term “functional derivative” includes, but is not limited to, fragments of a native sequence and derivatives of a native sequence polypeptide and its fragments, provided that they have a biological activity in common with a corresponding native sequence polypeptide. A biological activity contemplated herein is the ability of the functional derivative to hydrolyze a DNA substrate into fragments. The term “derivative” encompasses both amino acid sequence variants of a polypeptide, covalent modifications, and fusions thereof, such as, for example, a derivative Cas protein. Suitable derivatives of a Cas polypeptide or a fragment thereof include, but are not limited to, mutants, fusions, and/or covalent modifications of a Cas protein (or a fragment thereof).

Generally, there are three main types of Cas nucleases (type I, type II, and type III), and a plurality of subtypes of the various types of Cas nucleases. However, any suitable Cas nuclease (i.e., a type I Cas protein, a type II Cas protein, a type III Cas protein, a type IV Cas protein, a type V Cas protein, or a type VI Cas protein, as well as homologs thereof, variants thereof, mutants thereof, and derivatives thereof) may be used in accordance with the aspects and embodiments described herein. Type I Cas proteins include, but are not limited to, Cas3, Cas8a, Cas5, Cas8b, Cas8c, Cas10d, Cse1, Cse2, Csy1, Csy2, Csy3, GSU0054, and the like. Type III Cas proteins include, but are not limited to, Cas10, Csm2, Cmr5, Csx11, Csx10, and the like. Type IV Cas proteins include, but are not limited to, Csf1, Csf2, Csf3, and the like. Type V Cas proteins include, but are not limited to, Cas12, Cas12a (Cpf1), Cas12b (C2c1), Cas12c (C2c3), Cas12d (CasY), Cas12e (CasX), Cas12f (Cas14; C2c10), Cas12g, Cas12h, Cas12i, Cas12k (C2c5), C2c4, C2c8, C2c9, and the like. Type VI Cas proteins include, but are not limited to, Cas13, Cas13a (C2c2), Cas13b, Cas13c, Cas13d, and the like. These Cas nucleases are known to those skilled in the art. For example, the amino acid sequence of the Streptococcus pyogenes wild-type Cas9 polypeptide is set forth, for example, in NCBI Ref. Seq. No. WP_032462936.1, and the amino acid sequence of the Streptococcus thermophilus wild-type Cas9 polypeptide is set forth, for example, in NCBI Ref. Seq. No. WP_011681470.1.

Generally, Cas9 homologs are found in a wide variety of eubacteria, including, but not limited to, bacteria of the following taxonomic groups: Actinobacteria, Aquificae, Bacteroidetes-Chlorobi, Chlamydiae, Verrucomicrobia, Chloroflexi, Cyanobacteria, Firmicutes, Proteobacteria, Spirochaetes, and Thermotogae. An exemplary Cas9 protein is the Streptococcus pyogenes Cas9 protein. Additional Cas9 proteins and homologs thereof are described in, for example, Chylinksi, et al., RNA Biol. 2013 May 1; 10(5): 726-737; Nat. Rev. Microbiol. 2011 June; 9(6): 467-477; Hou, et al., Proc Natl Acad Sci USA. 2013 Sep. 24; 110(39): 15644-9; Sampson et al., Nature. 2013 May 9; 497(7448):254-7; and Jinek, et al., Science. 2012 Aug. 17; 337(6096):816-21, all of which are herein incorporated by reference in their entirety, with particularity for their relevant disclosures pertaining to Cas9 proteins and homologs thereof. Variants of any of the Cas9 nucleases provided herein can be optimized for efficient activity or enhanced stability in a host cell, such as, for example, a hematopoietic cell used (i) to develop the compositions, and/or (ii) in the methods disclosed herein. Thus, engineered Cas9 nucleases are also contemplated. Cas9 from Streptococcus pyogenes contains 2 endonuclease domains, including a RuvC-like domain that cleaves target DNA that is non-complementary to crRNA, and an HNH nuclease domain that cleaves target DNA that is complementary to crRNA. The double-stranded endonuclease activity of Cas9 also involves a short, conserved sequence (between about 2 to about 6 nucleotides in length), known as a protospacer-associated motif (PAM; or a “PAM sequence”), and is generally located about 2 to about 6 nucleotides downstream of the target motif.

Additionally, Cas nucleases (e.g., Cas9 polypeptides) can be derived from a variety of bacterial species, including, but not limited to, Veillonella atypical, Fusobacterium nucleatum, Filifactor alocis, Solobacterium moorei, Coprococcus catus, Treponema denticola, Peptoniphilus duerdenii, Catenibacterium mitsuokai, Streptococcus mutans, Listeria innocua, Staphylococcus pseudintermedius, Acidaminococcus intestine, Olsenella uli, Oenococcus kitaharae, Bifidobacterium bifidum, Lactobacillus rhamnosus, Lactobacillus gasseri, Finegoldia magna, Mycoplasma mobile, Mycoplasma gallisepticum, Mycoplasma ovipneumoniae, Mycoplasma canis, Mycoplasma synoviae, Eubacterium rectale, Streptococcus thermophilus, Eubacterium dolichum, Lactobacillus coryniformis subsp. Torquens, Ilyobacter polytropus, Ruminococcus albus, Akkermansia muciniphila, Acidothermus cellulolyticus, Bifidobacterium longum, Bifidobacterium dentium, Corynebacterium diphtheria, Elusimicrobium minutum, Nitratifractor salsuginis, Sphaerochaeta globus, Fibrobacter succinogenes subsp. Succinogenes, Bacteroides fragilis, Capnocytophaga ochracea, Rhodopseudomonas palustris, Prevotella micans, Prevotella ruminicola, Flavobacterium columnare, Aminomonas paucivorans, Rhodospirillum rubrum, Candidatus Puniceispirillum marinum, Verminephrobacter eiseniae, Ralstonia syzygii, Dinoroseobacter shibae, Azospirillum, Nitrobacter hamburgensis, Bradyrhizobium, Wolinella succinogenes, Campylobacter jejuni subsp. Jejuni, Helicobacter mustelae, Bacillus cereus, Acidovorax ebreus, Clostridium perfringens, Parvibaculum lavamentivorans, Roseburia intestinalis, Neisseria meningitidis, Pasteurella multocida subsp. Multocida, Sutterella wadsworthensis, proteobacterium, Legionella pneumophila, Parasutterella excrementihominis, and Francisella novicida.

As used herein, the term “Cas9 ribonucleoprotein complex” (and the like) refers to a complex between a Cas9 and a guide RNA (gRNA), the Cas9 protein and a CRISPR RNA (a “crRNA”), the Cas9 protein and a trans-activating crRNA (a “tracrRNA”), or any combination thereof. It is understood that in any of the embodiments described herein, a Cas9 nuclease can be substituted with another RNA-mediated nuclease (e.g., an alternative Cas protein or a Cpf1 nuclease). In some embodiments, the Cas protein is introduced into a hematopoietic cell in a polypeptide form, or into a genetically modified hematopoietic cell in a polypeptide form. Thus, for example, in certain embodiments, the Cas proteins can be conjugated to or fused to a cell-penetrating polypeptide or cell-penetrating peptide that is known in the relevant art. Non-limiting examples of cell-penetrating peptides are discussed in, for example, Milletti F, Drug Discov. Today 17: 850-860, 2012; the relevant disclosure of which is hereby incorporated by reference in its entirety.

In some embodiments, a Cas nuclease (such as, for example, a Cas9 nuclease) and a gRNA are introduced into a cell (e.g., a hematopoietic cell used (i) to develop the compositions, and/or (ii) in the methods disclosed herein) as a ribonucleoprotein (RNP) complex. In some embodiments, the RNP complex may be introduced into about 1×105 to about 2×10⁶ hematopoietic cells. In some embodiments, the cells are cultured under conditions effective for expanding the population of genetically modified cells. Also disclosed herein is a genetically modified cell population, in which the genome of at least about 20% about 30%, about 40%, about 50%, about 60%, about 70%, about 80%, about 90%, about 95%, about 99% or more of the cells comprises a genetic modification or heterologous polynucleotide that inhibits expression of one or more CRL5 complex genes and/or one or more CRL5 complex-independent genes as described herein. In some embodiments, the population comprises one or more subpopulations of cells, each of which subpopulations have a different genetic modification to inhibit expression of the one or more CRL5 complex genes and/or one or more CRL5 complex-independent genes described herein. Any suitable method for introducing the RNP complex into a cell (such as, for example, a hematopoietic cell used (i) to develop the compositions, and/or (ii) in the methods disclosed herein) can be used. For example, in some embodiments, the RNP complex is introduced using electroporation, as described in PCT Publication Nos. WO 2016/123578, WO 2006/001614, and Kim, J. A. et al. Biosens. Bioelectron. 23, 1353-1360 (2008), each of which is incorporated by reference in their entirety, with particularity for their relevant disclosures pertaining to CRISPR systems. Additional or alternative methods, compositions, and devices for electroporating cells to introduce a RNP complex can be found in, for example, U.S. Patent Application Publication Nos. 2006/0094095; 2005/0064596; or 2006/0087522; Li, L. H. et al. Cancer Res. Treat. 1, 341-350 (2002); U.S. Pat. Nos. 6,773,669; 7,186,559; 7,771,984; 7,991,559; 6,485,961; 7,029,916; and U.S. Patent Appl. Pub. Nos: 2014/0017213; and 2012/0088842; Geng, T. et al., J. Control Release 144, 91-100 (2010); and Wang, J., et al. Lab. Chip 10, 2057-2061 (2010); each of which is incorporated by reference in their entirety.

In some embodiments, the Cas9 protein is a nickase, such that when bound to a target nucleic acid (e.g., one or more CRL5 complex genes, one or more CRL5 complex-independent genes), as part of a complex with a guide RNA, a single strand break or “nick” is introduced into the target nucleic acid (e.g., a CRL5 complex gene, a CRL5 complex-independent gene, etc.). A pair of Cas9 nickases, each bound to a structurally different gRNA, can be targeted to two proximal sites of a target genomic region, such as, for example, one or more CRL5 complex(-independent) genes, and thus can introduce a pair of proximal single stranded breaks into the target genomic region (e.g., a CRL5 complex gene, a CRL5 complex-independent gene, etc.). Nickase pairs can provide enhanced specificity because off-target effects are likely to result in single nicks, which are generally repaired without lesion by endogenous base-excision repairs mechanisms. Non-limiting examples of Cas9 nickases include Cas9 nucleases having a D10A or H840A substitution mutation (see, e.g., Jinek et al, Science 337:816-821, 2012; Qi et al., Cell, 152(5): 1173-1183, 2012; Ran et al, Cell 154: 1380-1389, 2013; herein incorporated by reference in its entirety, with particularity for disclosure pertaining to Cas9 nucleases and modifications conferring one or more properties of a “nickase”). In some embodiments, the Cas9 polypeptide is from Streptococcus pyogenes and comprises a mutation at one or more of the following amino acid positions: D10, G12, G17, E762, H840, N854, N863, H982, H983, A984, D986, A987, as well as any combination thereof. Descriptions of such dCas9 polypeptides and variants thereof are provided in, for example, International Patent Publication No. WO 2013/176772. The Cas9 protein may contain a mutation at D10, E762, H983, or D986, as well as a mutation at H840 or N863. In some instances, the Cas9 protein may contain a D10A or D01N substitution mutation. Alternatively, or in addition to, the Cas9 protein may comprise a H840A, H840Y, or H840N substitution mutation. The substitutions can be conservative or non-conservative substitutions to render the Cas9 polypeptide catalytically inactive and able to bind to target DNA, such as, for example, one or more CRL5 complex genes, one or more CRL5 complex-independent genes, etc.

In some embodiments, the Cas nuclease comprises a high-fidelity Cas9 polypeptide or an enhanced specificity Cas9 polypeptide, wherein the high-fidelity or enhanced specificity Cas9 polypeptide has reduced off-target effects and robust on-target cleavage. Non-limiting examples of Cas9 polypeptide variants with improved on-target specificity include, but are not limited to, the SpCas9 (K855A), SpCas9 (K810A/K1003A/R1060A; also referred to as eSpCas9(1.0)), and SpCas9 (K848A/K1003A/R1060A; also referred to as eSpCas9(1.1)) described in Slaymaker et al, Science, 351(6268):84-8 (2016), and the SpCas9 variants containing one, two, three, or four of the following mutations: N497A, R661A, Q695A, and Q926A (e.g., SpCas9-HFl contains all four mutations) described in Kleinstiver et al, Nature, 529(7587): 490-5 (2016); each of which is herein incorporated by reference in their entirety, with particularity for relevant disclosure pertaining to Cas9 proteins and variants thereof.

In some embodiments, the target motifs (i.e., one or more regions of the one or more CRL5 complex genes and/or the one or more CRL5 complex-independent genes) can be selected to minimize off-target effects of the CRISPR systems of the present invention. For example, in some embodiments, the target motif is selected such that it contains at least two mismatches when compared with all other genomic nucleotide sequences in the cell. In some embodiments, the target motif is selected such that it contains at least one mismatch when compared with all other genomic nucleotide sequences in the cell. Those skilled in the art will appreciate that a variety of techniques can be used to select suitable target motifs for minimizing off-target effects (such as, for example, one or more bioinformatic analyses known in the relevant art).

As used herein, a “guide RNA sequence” (gRNA sequence) refers to a sequence that interacts with a site-specific or targeted nuclease and specifically binds to or hybridizes to a target nucleic acid (e.g., a CRL5 complex gene, a CRL5 complex-independent gene, etc.) within the genome of a cell (e.g., a hematopoietic cell), such that the gRNA and the targeted nuclease co-localize to the target nucleic acid in the genome of the cell. Each gRNA includes a DNA targeting sequence or protospacer sequence of about 10 to about 50 nucleotides in length that specifically binds to or hybridizes to a target DNA sequence (e.g., a CRL5 complex gene, a CRL5 complex-independent gene, etc.) in the genome of a cell (e.g., a hematopoietic cell used (i) to develop the compositions, and/or (ii) in the methods disclosed herein). In some embodiments, the target sequence can be about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length. In some embodiments, the gRNA comprises a crRNA sequence and a tracrRNA sequence, and the gRNA may be referred to as a single guide RNA (sgRNA). In some embodiments, the gRNA does not comprise a tracrRNA sequence. The gRNA and/or sgRNA can be selected depending on the particular CRISPR/Cas system employed and/or the sequence of the target polynucleotide (e.g., a CRL5 complex gene, a CRL5 complex-independent gene, etc.).

As indicated above, in some embodiments, the one to two ribonucleic acids (i.e., the crRNA sequence and/or the tracrRNA sequence) can be selected to minimize hybridization with nucleic acid sequences other than the target polynucleotide sequence (i.e., to minimize hybridization with nucleic acid sequences that do not target a subject CRL5 complex gene and/or a subject CRL5 complex-independent gene). In some embodiments, the one to two ribonucleic acids (i.e., the crRNA sequence and/or the tracrRNA sequence) hybridize to a target motif (e.g., a CRL5 complex gene, a CRL5 complex-independent gene, etc.) that contains at least two mismatches when compared with all other genomic nucleotide sequences in the cell (e.g., a hematopoietic cell used (i) to develop the compositions, and/or (ii) in the methods disclosed herein). In some embodiments, the one to two ribonucleic acids (i.e., the crRNA sequence and/or the tracrRNA sequence) hybridize to a target motif (e.g., a CRL5 complex gene, a CRL5 complex-independent gene, etc.) that contains at least one mismatch when compared with all other genomic nucleotide sequences in the cell. In some embodiments, the one to two ribonucleic acids (i.e., the crRNA sequence and/or the tracrRNA sequence) are designed to hybridize to a target motif (e.g., a CRL5 complex gene, a CRL5 complex-independent gene, etc.) immediately adjacent to a deoxyribonucleic acid motif recognized by the Cas protein. In some embodiments, each of the one to two ribonucleic acids (i.e., the crRNA sequence and/or the tracrRNA sequence) are designed to hybridize to target motifs immediately adjacent to deoxyribonucleic acid motifs recognized by the Cas protein that flank a mutant allele located between the target motifs. Guide RNAs also be designed using software that is readily available, such as, for example, at the website crispr.mit.edu, and the like. The one or more sgRNAs can be transfected into a cell in which a Cas protein is present by transfection, according to methods known in the relevant art.

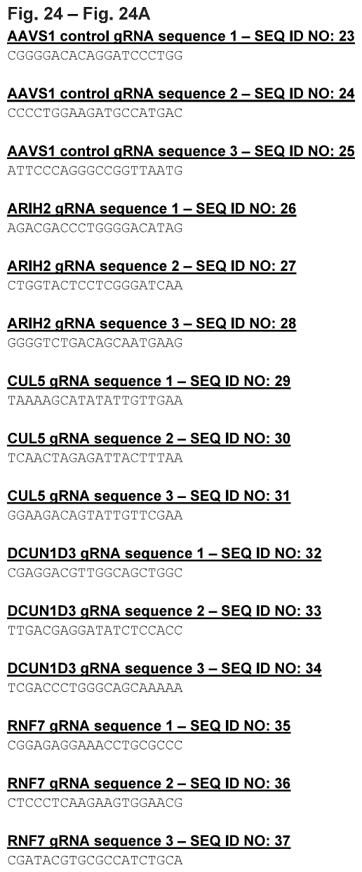

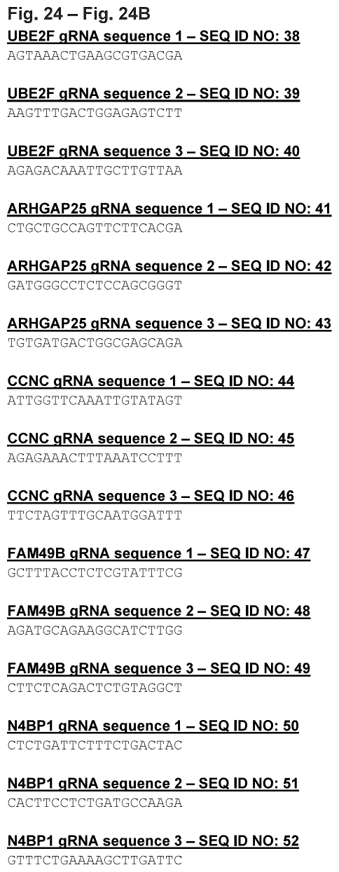

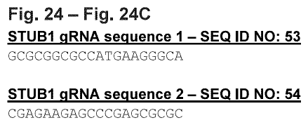

In some embodiments, the gRNA sequence targets one or more CRL5 complex genes (or, in some embodiments, one or more CRL5 complex-independent genes), a region flanking one or more CRL5 complex(-independent) genes, a promoter sequence of the one or more CRL5 complex(-independent) genes, a coding region of the one or more CRL5 complex(-independent) genes, or a non-coding region of the one or more CRL5 complex(-independent) genes. In some embodiments, a plurality of gRNA sequences is used. In some embodiments, the gRNA sequence comprises any one of SEQ ID NOs: 26-58, as shown in FIG. 24. In some embodiments, a pool of 3 or more gRNA sequences (e.g., 3, 4, 5, 6, 7, 8, 9, 10, 11, 12, 13, 14, 15, or more) is used. In some embodiments, the pool of 3 or more gRNA sequences comprises three or more of SEQ ID NOs: 26-58. In some instances, the pool of 3 or more gRNA sequences targets one of the CRL5 complex genes (see, e.g., SEQ ID NOs: 26-40 and 56-58). In some instances, the pool of 3 or more gRNA sequences targets a plurality of the CRL5 complex genes (see, e.g., SEQ ID NOs: 26-40 and 56-58). In some other instances, the pool of 3 or more gRNA sequences targets a plurality of the CRL5 complex-independent genes (see, e.g., SEQ ID NOs: 41-55). In still some other instances, at least one of the 3 or more gRNA sequences targets at least one of the CRL5 complex genes (see, e.g., SEQ ID NOs: 26-40 and 56-58), and the remainder of the gRNA sequences targets at least one of the CRL5-complex independent genes (see, e.g., SEQ ID NOs: 41-55). In some embodiments, a pool of 3 or more gRNA sequences is employed per target. For example, in an embodiment wherein one CRL5 complex gene and one CRL5 complex-independent gene are each targets for inhibition/inactivation, a first pool of 3 or more gRNA sequences would be utilized to effectuate inhibition or inactivation of the CRL5 complex gene (see, e.g., SEQ ID NOs: 26-40 and 56-58), and a second pool of 3 or more gRNA sequences would be utilized to effectuate inhibition or inactivation of the CRL5 complex-independent gene (see, e.g., SEQ ID NOs: 41-55).

In some embodiments, a DNA targeting sequence can incorporate “wobble” or degenerate bases to bind one or more genetic elements (such as, for example, 2, 3, 4, or more genetic elements). In some embodiments, the 19 nucleotides located at the 3′ or 5′ end of a binding region are perfectly complementary to the target genetic element or elements (such as, for example, one or more portions of the genetic sequences of the one or more CRL5 complex(-independent) genes). In some embodiments, a binding region can be altered to increase stability.

For example, non-naturally occurring nucleotides can be incorporated to increase the resistance of RNA to degradation. In some embodiments, a binding region can be altered or designed to avoid or reduce secondary structure formation in the binding region. In some embodiments, a binding region can be designed to optimize the GC content. In some further embodiments, GC content of a binding region is between about 20% to about 80%, between about 30% to about 70% or about 40% to about 60%.

In some embodiments, the sequence of the gRNA, or a portion thereof, is designed to complement (e.g., to be perfectly complementary) or substantially complement (e.g., to be at least 50%, 60%, 70%, 80%, 90%, 91%, 92%, 93%, 94% 95%, 96%, 97%, 98%, or 99% complementary) to a target region in the one or more CRL5 complex(-independent) genes. In some embodiments, the portion of the gRNA that complements and binds the targeting region in the one or more CRL5 complex(-independent) genes is, or is about, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or more nucleotides in length. In some embodiments, the portion of the gRNA that complements and binds the targeting region in the one or more CRL5 complex(-independent) genes is between about 17 to about 23 nucleotides, about 18 to about 22 nucleotides, or about 19 to about 21 nucleotides in length. In some embodiments, the gRNA may incorporate “wobble” or degenerate bases to bind one or more target regions (i.e., one or more target regions of the one or more CRL5 complex(-independent) genes). In some embodiments, the gRNA can be altered to increase stability. For example, non-naturally occurring nucleotides can be incorporated to increase the resistance of RNA to degradation. In some embodiments, the gRNA can be altered or designed to avoid or reduce secondary structure formation. In some embodiments, the gRNA can be designed to optimize GC content. In some further embodiments, GC content of the gRNA is between about 20% to about 80%, between about 30% to about 70% or about 40% to about 60%.

In some embodiments, the gRNA can be optimized for expression by substituting, deleting, and/or adding one or more nucleotides. In some embodiments, a nucleotide sequence that provides inefficient transcription from an encoding template nucleic acid can be deleted or substituted. For example, in some embodiments, the gRNA is transcribed from a nucleic acid operably linked to an RNA polymerase III promoter. In such instances, gRNA sequences that result in inefficient transcription by RNA polymerase III, such as those described in, for example, Nielsen et al., Science. 2013 Jun. 28; 340(6140): 1577-80, can be deleted or substituted. By way of further example, one or more consecutive uracils can be deleted or substituted from the gRNA sequence.

In some embodiments, the gRNA can be optimized for stability. Stability can be enhanced by optimizing the stability of the gRNA nuclease interaction, optimizing assembly of the gRNA nuclease complex, removing, or altering RNA destabilizing sequence elements, adding RNA stabilizing sequence elements, and other methods for enhancing stability known in the relevant art.

Generally, gRNAs can be modified by any suitable method known in the relevant art. In some embodiments, the modifications to the gRNA can include, but are not limited to, one or more of the following: a 5′ cap (e.g., a 7-methylguanylate cap), a 3′ polyadenylated tail, a riboswitch sequence, a stability control sequence, a hairpin, a subcellular localization sequence, a detection sequence or a detectable label, a binding site for one or more proteins, and others known in the relevant art. Alternatively, or in addition to, modifications to the gRNA can include the introduction of non-naturally occurring nucleotides, such as, for example, fluorescent nucleotides and/or methylated nucleotides.

Compositions and methods of the disclosure may further comprise one or more expression cassettes and/or vectors for producing gRNAs in a host cell. The one or more expression cassettes can contain a promoter (e.g., a heterologous promoter) operably linked to a polynucleotide encoding a gRNA. The promoter can be an inducible promoter or a constitutive promoter. Alternatively, or in addition to, the promoter can be tissue specific. In some embodiments, the promoter is a U6, HI, or spleen focus-forming virus (SFFV) long terminal repeat promoter. In some embodiments, the promoter is a “weak” mammalian promoter (as compared to the human elongation factor 1 (EF1A) promoter. In some embodiments, the weak mammalian promoter is a ubiquitin C promoter or a phosphoglycerate kinase 1 (PGK) promoter. In some embodiments, the weak mammalian promoter is a “Tet-On” promoter, optionally in the presence or absence of an inducer, such as, for example, tetracycline or a tetracycline derivative (e.g., doxycycline). In some further embodiments, when a Tet-On promoter is utilized, a host cell is also contacted with a tetracycline transactivator. In some embodiments, the strength of the selected gRNA promoter is selected to express an amount of gRNA that is proportional to the amount of Cas9 or dCas9, or other suitable Cas protein as described herein. The one or more expression cassettes can be in a vector, such as, for example, a plasmid, a viral vector, a lentiviral vector, and the like. In some embodiments, the expression cassette is in a host cell. The gRNA expression cassette can be episomal or integrated in the host cell.

### 2. Genetic Modifications Using Alternative Targeted Nuclease Systems:

In some embodiments, the one or more CRL5 complex genes and/or the one or more CRL5 complex-independent genes are inhibited using a TALEN system. Generally, TALENS bind as a pair around a genomic site and direct a non-specific nuclease (e.g., FokI) to cleave the genome at a specific site. Methods of using TALENS to reduce gene expression are disclosed in, for example, U.S. Pat. No. 9,005,973; Christian et al. “Genetics 186(2): 757-761, 2010; Zhang et al. 2011 Nature Biotech. 29: 149-53, 2011; Geibler et al. 2011 PLoS ONE 6: e19509, 2011; Boch et al. 2009 Science 326: 1509-12; Moscou et al. 2009 Science 326: 3501; each of which is herein incorporated by reference in their entirety, with particularity for their disclosures pertaining to TALENs.

Generally, to produce a TALEN, a TALE protein is typically fused to a FokI endonuclease, which can be a wild-type or mutated FokI endonuclease. Several mutations to FokI have been made for use in TALENs and are known in the relevant art. Nonlimiting examples of FokI endonucleases that can be used in the generation of a TALEN system are described in, for example, Cermak et al, Nucl. Acids Res. 39(12):e82, 2011; Miller et al, Nature Biotech. 29: 143-8, 2011; Hockemeyer et al, Nature Biotech. 29:731-734, 2011; Wood et al. Science 333:307, 2011; Doyon et al, Nature Methods 8:74-79, 2010; Szczepek et al, Nature Biotech. 25:786-793, 2007; and Guo et al, J. Mol Biol. 200:96, 2010; each of which is herein incorporated by reference in their entirety, with particularity for their disclosures pertaining to FokI and TALENs.

In some embodiments, the one or more CRL5 complex genes and/or the one or more CRL5 complex-independent genes are inhibited using a ZFN system. Methods of using ZFNs to inhibit gene expression are described in, for example, U.S. Pat. No. 9,045,763, and in Durai et al, Nucleic Acid Research 33:5978-5990, 2005; Carroll et al. Genetics Society of America 188: 773-782, 2011; and Kim et al. Proc. Natl. Acad. Sci. USA 93: 1156-1160; each of which is incorporated by reference in their entirety, with particularity for their relevant disclosures pertaining to ZFN systems.

Generally, a ZFN comprises a FokI (or “Fok1”) nuclease domain (or derivative thereof) fused to a DNA-binding domain. With respect to ZFNs, the DNA-binding domain comprises one or more “zinc fingers.” As used herein, the term “zinc finger” refers to a small protein structural motif stabilized by one or more zinc ions. A zinc finger can comprise, for example, Cys2His2, and can recognize a nucleotide sequence of approximately 3 base pairs in length. Various zinc fingers of known specificity can be combined to produce multi-finger polypeptides which can recognize about nucleotides of various length (e.g., about 6, about 9, about 12, about 15, about 18, or more). Various selection and modular assembly techniques are available to generate zinc fingers (and combinations thereof) recognizing specific sequences (such as, for example, at least a portion of the one or more CRL5 complex(-independent) genes).

Generally, a ZFN dimerizes to cleave DNA. Thus, a pair of ZFNs can be used to target non-palindromic DNA sites. The individual ZFNs bind opposite strands of the DNA with their nucleases properly spaced apart (see, e.g., Bitinaite et al, Proc. Natl. Acad. Sci. USA 95: 10570-5, 1998; herein incorporated by reference in its entirety, with particularity for relevant disclosure pertaining to ZFNs). A ZFN can create a double-stranded break in DNA, which can create a frame-shift mutation if improperly repaired, leading to a decrease in the expression and level of expression of a target gene (such as, for example, one or more CRL5 complex(-independent) genes) in a cell (such as, for example, a hematopoietic cell used (i) to develop the compositions, and/or (ii) in the methods disclosed herein).

In some further embodiments, the one or more CRL5 complex genes and/or the one or more CRL5 complex-independent genes are inhibited using a meganuclease system. Meganucleases are a relatively rare group of cutting endonucleases or homing endonucleases that can be highly specific, recognizing DNA target sites ranging from about 10 to about 60 base pairs in length, about 12 to about 60 base pairs in length, or about 12 to about 40 base pairs in length. Meganucleases can be modular DNA-binding nucleases, such as, for example, any fusion protein comprising at least on catalytic domain of an endonuclease and at least one DNA binding domain or protein-specifying nucleic acid target sequence. The DNA-binding domain can contain at least one motif that recognizes single- or double-stranded DNA. The meganuclease can be monomeric or dimeric.

In some embodiments, the one or more CRL5 complex genes and/or the one or more CRL5 complex-independent genes are inhibited using a meganuclease system, and in some further embodiments, the meganuclease system comprises a naturally occurring or “wild-type” meganuclease. Alternatively, or in addition to, the meganuclease can comprise a non-natural meganuclease, an artificial meganuclease, an engineered meganuclease, a synthetic meganuclease, or a rationally designed meganuclease. Non-limiting examples of meganucleases that can be using in any of the embodiments described herein include: an I-CreI meganuclease, an I-CeuI meganuclease, an I-MsoI meganuclease, an I-SceI meganuclease, variants thereof, mutants thereof, and derivatives thereof. Detailed descriptions of meganucleases and their application in gene editing can be found, for example, in Silva et al, Curr Gene Ther, 2011, 11(1): 11-27; Zaslavoskiy et at, BMC Bioinformatics, 2014, 15:191; Takeuchi et al, Proc Natl Acad Sci USA, 2014, 111(11): 4061-4066, and U.S. Pat. Nos. 7,842,489; 7,897,372; 8,021,867; each of which is herein incorporated by reference in their entirety, with particularity for relevant disclosure pertaining to meganucleases.

### 3. Genetic Modifications Using an Inhibitory RNA Systern:

In some embodiments, the one or more CRL5 complex genes and/or the one or more CRL5 complex-independent genes are inhibited using a shRNA system. In some embodiments, the shRNA system comprises a nucleotide sequence that is about 10 to about 200 nucleotides in length, about 10 to about 100 nucleotides in length, about 10 to about 50 nucleotides in length, or about 10 to about 25 nucleotides in length. In some embodiments, the shRNA system comprises a nucleotide sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or 100% complementary to a region of a target mRNA molecule (such as, for example, the one or more CRL5 complex(-independent) genes). In some embodiments, the shRNA system comprises a nucleotide sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or 100% complementary to a region of a target pro-mRNA molecule (such as, for example, the one or more CRL5 complex(-independent) genes). In some embodiments, the shRNA system comprises a double-stranded RNA molecule. Alternatively, or in addition to, the shRNA system can comprise a single-stranded RNA molecule. In some embodiments, a host cell, such as, for example, a hematopoietic cell used (i) to develop the compositions, and/or (ii) in the methods disclosed herein, comprises a shRNA system as described in any of the embodiments described herein. Alternatively, or in addition to, a host cell (e.g., a hematopoietic cell used (i) to develop the compositions, and/or (ii) in the methods disclosed herein) can comprise a pro-shRNA nucleotide sequence that is processed into an active shRNA molecule as described in any of the embodiments described herein. In some embodiments, the host cell (e.g., a hematopoietic cell used (i) to develop the compositions, and/or (ii) in the methods disclosed herein) comprises a shRNA nucleotide sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or 100% complementary to a region of the target mRNA molecule (i.e., the one or more CRL5 complex(-independent) genes). In some embodiments, the host cell comprises an expression vector encoding a shRNA molecule as described in any of the embodiments herein. Alternatively, or in addition to, the host cell can comprise an expression vector encoding a pro-shRNA molecule as described in any of the embodiments herein. In some embodiments, the shRNA system comprises a delivery vector. In some embodiments, the host cell comprises a delivery vector. In some embodiments, the delivery vector comprises the pro-shRNA and/or the shRNA molecule. In some embodiments, the delivery vector is a viral vector. In some embodiments, the delivery vector is a lentivirus. In some embodiments, the delivery vector is an adenovirus. In some embodiments, the vector comprises a promoter.

In some embodiments, the one or more CRL5 complex genes and/or the one or more CRL5 complex-independent genes are inhibited using a siRNA system. In some embodiments, the siRNA system comprises a siRNA nucleotide sequence that is about 10 to about 200 nucleotides in length, about 10 to about 100 nucleotides in length, about 10 to about 50 nucleotides in length, or about 10 to about 25 nucleotides in length. In some embodiments, the siRNA system comprises a nucleotide sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or 100% complementary to a region of a target mRNA molecule (such as, for example, the one or more CRL5 complex(-independent) genes). In some embodiments, the siRNA system comprises a nucleotide sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or 100% complementary to a region of a target pro-mRNA molecule (such as, for example, the one or more CRL5 complex(-independent) genes). In some embodiments, the siRNA system comprises a double-stranded RNA molecule. Alternatively, or in addition to, the siRNA system can comprise a single-stranded RNA molecule. In some embodiments, a host cell, such as, for example, a hematopoietic cell used (i) to develop the compositions, and/or (ii) in the methods disclosed herein, comprises a siRNA system as described in any of the embodiments described herein. Alternatively, or in addition to, a host cell (e.g., a hematopoietic cell used (i) to develop the compositions, and/or (ii) in the methods disclosed herein) can comprise a pro-siRNA nucleotide sequence that is processed into an active siRNA molecule as described in any of the embodiments described herein. In some embodiments, the host cell comprises a siRNA nucleotide sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or 100% complementary to a region of the target mRNA molecule (i.e., the one or more CRL5 complex(-independent) genes). In some embodiments, the host cell comprises an expression vector encoding a siRNA molecule as described in any of the embodiments herein. Alternatively, or in addition to, the host cell can comprise an expression vector encoding a pro-siRNA molecule as described in any of the embodiments herein. In some embodiments, the siRNA system comprises a delivery vector. In some embodiments, the host cell (e.g., a genetically modified hematopoietic cell) comprises a delivery vector. In some embodiments, the delivery vector comprises the pro-siRNA and/or the siRNA molecule.

Various considerations can be taken into account when designing the siRNA sequence to be used to inactivate the one or more CRL5 complex(-independent) genes, such as, for example: (1) the length of the siRNA (generally, it is preferably for a siRNA to have a nucleotide length of about 25, 24, 23, 22, 21, 20, 19, 18, 17, 16, 15, 14, 13, 12, 11, 10 or fewer nucleotides), (2) proximity of a potential target region to the start codon and/or termination codon (generally, it is preferable to avoid regions within about 50 to about 100 nucleotides of the start codon and/or termination codon), (3) avoidance of intronic regions, (4) avoidance of polynucleotide repeats (i.e., a continuous portion of the potential target region comprising 4 or more of the same nucleotide (such as, for example, 5′-AAAA-3′, 5′-TTTT-3′, 5′-CCCC-3′, 5′-GGGG-3′, as well as polynucleotide repeats comprising 5, 6, 7, 8, 9, 10 or more repeats of the same nucleotide, and the like)), (5) avoidance of genomic regions with GC content that is less than 30% or greater than 60%, (6) avoidance of palindromic sequences (such as, for example, perfect palindromes, quasipalindromes (with or without a central spacer region), and the like), (7) avoidance of low sequence complexity regions, (8) avoidance of single nucleotide polymorphic sites, (9) avoidance of sequences that are complementary to sequences in other off-target genes, and others known in the art (see, e.g., Rules of siRNA design for RNA interference, Protocol Online, May 29, 2004; and Reynolds et al., Nat Biotechnol, 22:3236-330 2004; herein incorporated by reference in its entirety, with particularity for relevant disclosure pertaining to considerations for designing siRNA sequences).

In some further embodiments, the one or more CRL5 complex genes and/or the one or more CRL5 complex-independent genes are inhibited using a miRNA system.

In some further embodiments, the one or more CRL5 complex genes and/or the one or more CRL5 complex-independent genes are inhibited using an antisense RNA.

### C. Cells and Sources Thereof

In some embodiments, the hematopoietic cell used (i) to develop the compositions, and/or (ii) in the methods disclosed herein is isolated from a subject, such as, for example, a human subject. The hematopoietic cell can be obtained from a subject of interest, such as, for example, a subject having or who is suspected of having a particular disease or condition, a subject who has or is suspected of having a predisposition to a particular disease or condition, a subject who is undergoing therapy for a particular disease or condition, a subject who has been diagnosed as having a particular disease or condition, or a subject who has received a clinical impression indicating that they may have or are likely to be diagnosed with a particular disease or condition. Alternatively, or in addition to, the hematopoietic cell can be obtained from a donor subject. Alternatively, or in addition to, the hematopoietic cell can be obtained from or generated from a cell line (see, e.g., below).

The hematopoietic cell to be used (i) to develop the compositions, and/or (ii) in the methods disclosed herein can be collected from any location in which they reside in a subject, including, but not limited to: peripheral blood (before, during, or after cells have been stimulated for release using a mobilizing agent, or in the absence of such a mobilization agent), umbilical cord blood (“cord blood”), the spleen, the thymus, one or more lymph nodes, bone marrow, or a combination thereof. As used herein, the terms “mobilization agent” or “mobilizing agent” refer to any number of drugs, medicaments, pharmacological preparations, recombinant proteins, and/or cytokines that stimulate the release of one or more stem cells (such as, for example, hematopoietic stem cells) from their cellular niche (e.g., from the bone marrow) into circulation within peripheral blood. Non-limiting examples of mobilizing agents include: G-CSF, GM-CSF, mozobil, and combinations thereof. Generally, when the hematopoietic cell is obtained from bone marrow, the bone in which the bone marrow resides is not particularly limited. In certain embodiments, the source of the bone marrow accessed to obtain the hematopoietic cell is located within one or both iliac bones. In some further embodiments, the bone marrow is within one or both of the iliac crests (with particular utility in obtaining the hematopoietic cell from the posterior superior iliac crest of one or both iliac bones).

Following isolation, the hematopoietic cell may be used directly, or can be stored for a period of time, such as, for example, by freezing according to a cell storage protocol known in the relevant art. Alternatively, or in addition to, the hematopoietic cell may be enriched and/or purified from any tissue where it resides, including, but not limited to: peripheral blood (i.e., peripheral blood obtained from a subject and/or a “blood bank”), cord blood (i.e., cord blood obtained from an umbilical cord and/or a placenta after birth, and/or a “cord blood bank”), the spleen, bone marrow, tissues removed and/or exposed during a surgical procedure, tissues obtained via a biopsy, or any combination thereof. Tissues and/or organs from which the hematopoietic cell is enriched, isolated, and/or purified from may be isolated from living subjects and/or non-living subjects, wherein the non-living subject is an appropriately human leukocyte antigen (HLA)-typed organ donor. In some embodiments, the hematopoietic cell is obtained from pooled blood and/or pooled cord blood. The pooled blood (and/or pooled cord blood) may be from 2 or more sources, such as, for example, 3, 4, 5, 6, 7, 8, 9, 10 or more sources (e.g., donor subjects).

The hematopoietic cell can be obtained from a subject in need of therapy or a subject suffering from a disease (e.g., a cancer). Thus, the hematopoietic cell will be autologous to the subject in need of therapy (or the subject suffering from a disease). Alternatively, or in addition to, the hematopoietic cell can be obtained from a living subject and/or a non-living subject that is not the subject in need of therapy (or that is suffering from a disease (e.g., a cancer)), with several embodiments finding utility in a “donor” that has a “compatible HLA-typing.” As used herein, the phrase “compatible HLA-typing” is used to refer to a configuration of major histocompatibility genes from a cellular source (i.e., a donor) that does not trigger the immune system of the subject in need of therapy (or that is suffering from a disease (e.g., a cancer); i.e., a recipient) resulting in rejection (e.g., transplant rejection, hyperacute rejection, acute rejection, chronic rejection, graft failure, etc.). When the hematopoietic cell is obtained from a donor that is distinct from the subject in need of therapy (or is suffering from a disease (e.g., a cancer)), the donor is preferably allogeneic, provided the cells obtained are subject-compatible in that they can be introduced into the subject. Donor cells that are allogeneic may or may not be HLA-compatible. In instances where the donor cells are not HLA-compatible, they may be treated to reduce immunogenicity and render them subject-compatible.

In some embodiments, the hematopoietic cell used (i) to develop the compositions, and/or (ii) in the methods disclosed herein is a cell selected from a group including: (i) a NK cell, (ii) a T-cell, and (iii) a TIL. In some exemplary embodiments, the hematopoietic cell comprises a NK cell. In some other exemplary embodiments, the hematopoietic cell comprises a T-cell, wherein the T-cell comprises a gamma-delta-(γδ-) T-cell. In some embodiments, the hematopoietic cell used (i) to develop the compositions, and/or (ii) in the methods disclosed herein is a stem cell or is derived from a stem cell.

### 1. NK Cells:

In some embodiments, the hematopoietic cell used (i) to develop the compositions, and/or (ii) in the methods disclosed herein is a NK cell.

Generally, NK cells are a subpopulation of lymphocytes that have spontaneous cytotoxicity against a variety of tumor cells, virally infected cells, certain normal cells located in the bone marrow and thymus, and the like. In some embodiments, the NK cells are derived from blood, cord blood, bone marrow, or a cell line. In some embodiments, the NK cells are human NK cells. Alternatively, the NK cells can be mouse-derived NK cells. Generally, the NK cells are primary cells, such as, for example, those isolated directly from a subject (and optionally may be frozen for future use according to any number of cell storage protocols known in the relevant art).

NK cells can be detected by the expression of specific surface markers, such as, for example, CD16, CD56, and CD8 in humans, or NK1.1 or NK1.2 in mice (such as, for example, C57BL/6J mice). Generally, NK cells do not express T-cell antigen receptors, the pan T-cell marker CD3, or surface immunoglobulin B-cell receptors. Therefore, in some embodiments, one or more NK cell populations can be enriched for or depleted of cells that are positive for a specific marker (e.g., CD16, CD56, CD8, and the like) and/or that are negative for a specific marker (e.g., CD3). In some embodiments, the NK cells comprise one or more subsets of NK cells, such as, for example, a whole NK cell population (i.e., a population of NK cells that is not specifically isolated for a particular subtype), CD56^{high} (also referred to as CD56^{bright})/CD16⁺ NK cells, CD56^{low} (also referred to as CD56^{dim})/CD16^{high} NK cells, and the like. NK cells differentiate and mature in the bone marrow, lymph nodes, the spleen, one or more of the tonsils, and the thymus. Non-limiting examples of NK cell maturation markers include: CD56+, CD16+, CD57+, NK1.1+, NK1.2+, CD94+, IL-2+, CD217+, NKG2A+, NKG2D+, and the like.

With reference to the subject in need of treatment, the NK cells may be allogeneic or autologous. Alternatively, or in addition to, the NK cells may be obtained from a cell line (such as, for example, a HSC, an iPSC, or a commercially available cell line (e.g., NK-92 cells (ATCC CRL-2407), NK92MI cells (ATCC CRL-2408), and derivatives thereof). In some embodiments, the NK cells are isolated from a donor subject and/or a recipient subject, and may further be subjected to one or more of preparing, processing, culturing, cellular engineering (e.g., the introduction of a CAR or TCR construct into the cell), and/or re-introduction into a subject in need of treatment (before or after cryopreservation of the NK cells).

In some embodiments, one or more NK cell populations are enriched for or depleted of cells that are positive for a specific marker, such as, for example, a surface marker, or that are negative for a specific marker. In some embodiments, the specific marker can be those that are absent or expressed at relatively low levels on certain populations of NK cells (e.g., CD56¹“w NK cells) but are present or expressed at relatively higher levels on certain other populations of NK cells (e.g., CD56^{high} NK cells).

In some embodiments, the NK cells are separated from a PBMC sample by negative selection of markers expressed on non-NK cells (e.g., CD3), such as, for example, T-cells, B-cells, monocytes, or other white blood cells. In some embodiments, the NK cells are separated from a PBMC sample by positive selection of markers expressed on NK cells (e.g., CD16, CD56, CD8, and the like). In some embodiments, the NK cells are separated from a PBMC sample by a combination of negative selection of markers expressed on non-NK cells and positive selection of markers expressed on NK cells. In some embodiments, the NK cells are further enriched for or depleted of one or more subtypes of NK cells.

Once the NK cells have been obtained from a source (e.g., an allogeneic donor, from the subject in need of therapy (i.e., an autologous source), a cell line, and the like), the NK cells can be processed (e.g., to remove non-NK cells, to remove cellular debris collected along with the NK cells, to create a single-cell suspension, to prepare cells for cryopreservation, to prepare cells for culturing and/or expansion, and the like), and subsequently be cultured in a suitable cell culture medium. The cultured NK cells can be pooled and expanded. Expansion provides an increase in the number of NK cells of at least about 50-fold, about 60-fold, about 70-fold, about 80-fold, about 90-fold, about 100-fold or greater over a period of about 10 to about 14 days, to about up to 12 weeks, depending on the method of expansion utilized. More preferably, expansion can provide an increase of at least about 150-fold, about 200-fold, about 250-fold, about 300-fold, about 350-fold, about 400-fold, about 450-fold, about 500-fold, about 600-fold, about 700-fold, about 800-fold, about 900-fold, about 1000-fold or greater over a period of about 10 to about 14 days, to about up to 12 weeks, depending on the method of expansion utilized.

Expansion of NK cells can be accomplished using any number of methods as are known in the relevant art (see, e.g., Lapteva et al. Crit. Rev. Oncog. 19:121-132, 2014; Miller et al. Blood 105(8):3051 7, 2005; Lapteva et al. Cytotherapy 14(9): 1131-43, 2012; Spanholtz et al. PLoS One 6(6):e20740, 2011; Knorr et al. Stem Cells TransL Med. 2(4):274-83, 2013; Pfeiffer et al. Leukemia 26(11):2435 9, 2012; Shi et al. Br. J. Haematol. 143 (5): 641-53, 2008; Passweg et al. Leukemia 18(11): 1835-8, 2004; Koehl et al. Klin. Padiatr. 217(6):345-50, 2005; and Klingemann et al. Transfusion 53 (2):412 8, 2013.; each of which is incorporated herein by reference in its entirety, with particularity for relevant disclosure pertaining to methods for expanding NK cells). In some embodiments, NK cell can be expanded or enriched from large volumes of peripheral blood, such as, for example, apheresis products (e.g., mobilized peripheral blood stem cells (PBSCs) or unmobilized PBSCs). Alternatively, or in addition to, NK cells can be expanded or enriched from a smaller amount of blood or stem cells. Further, alternatively, or in addition to, NK cells can be expanded using feeder cell-based technology.

Approaches that generate NK cells for allogeneic use, generally, aim to minimize CD3+T-lymphocyte populations that may cause graft-versus-host disease (GVHD). This often involves the depletion of CD3+ T-cells, which increases the total number of starting cells required, particularly if depletion is performed at the end of a manufacturing procedure. Generally, most protocols use between about 1.0×10⁹ to about 20.0×10⁹ mononuclear cells as the starting material. However, expansion from other sources (e.g., the buffy coat fraction of a blood sample, cord blood, embryonic stem cells (ESCs), and the like) is also possible. NK cells in blood samples (e.g., peripheral blood, cord blood) and apheresis products can be detected by flow cytometry as CD45⁺ CD56⁺ CD3⁻ cells. In some instances, NK cells can be enriched from apheresis products by one or two rounds of depletion of CD3+ T-cells using magnetic beads (e.g., CLINIMACS magnetic beads) coated with anti-CD3 antibody (e.g., CLINIMACS CD3 reagent), with or without overnight activation using IL-2 and/or IL-15. This method can produce up to 2×10⁹ NK cells with approximately 20% purity, while contaminating CD19⁺ B-cells and CD14+monocytes can comprise greater than 50% of the product. Additional depletion of CD19⁺ B-cells with anti-CD19 antibody-coated magnetic beads (e.g., CLINIMACS CD19 reagent) can further improve the purity of the NK cells, resulting in an average of 40% CD56⁺ CD3⁻ cells in the final product. Alternatively, NK cells can be enriched by isolating CD56⁺ cells using anti-CD56 monoclonal antibody (e.g., CLINIMACS CD56 reagent) with or without CD3⁺ T-cell depletion. Without CD3⁺ T cell depletion, this method can yield more than 95% NK cell purity while retaining CD56⁺ CD3⁺ natural killer-like T-cells, which also may contribute to anti-tumor immune responses, whereas the inclusion of CD3⁺ T-cell depletion can yield up to 99% purity.

In some embodiments, NK cells can be expanded using a feeder cell-based technology (see, e.g., Berg et al. Cytotherapy 11 (3): 341-55, 2009; Lapteva et al. 2012, supra, and Lapteva et al. Crit. Rev. Oncog. 19: 121-132, 2014; each of which is incorporated herein by reference in its entirety, with particularity for relevant disclosure pertaining to feeder cell-based technology methods). Because the therapeutic use of NK cells can demand high NK cell doses and/or several infusions, one cellular source (e.g., an apheresis product) may not contain sufficient numbers of NK cells. As such, technically complicated NK cell expansion protocols have been developed. Expansion of NK cells with cytokines (e.g., IL-2 and/or IL-15) to produce about 1,000-fold expansion may require a culture period of up to about 12 weeks. By contrast, feeder cell-based NK expansion approaches are rapid and robust, as large numbers of NK cells become available for infusion within about 10 to about 14 days (see, e.g., Lapteva et al., 2012, supra). Feeder-cell methods generally require cytokines as well as irradiated feeder cells, such as, for example, EBV-LCLs or genetically modified K562 cells, to produce large numbers of CD3-CD56* NK cells from PBMCs with purity that is at least about 65%, about 70%, about 75%, about 80%, about 85%, about 90%, about 95%, or higher.

In some embodiments, NK cells can be expanded using a genetically modified feeder cell expansion system, such as, for example, those described in Yang et al. (Mol. Therapy 18:428-445, 2020). In such methods, human primary NK cells can be expanded directly from PBMCs and/or cord blood (from, e.g., an allogeneic or an autologous source), as well as from tumor tissue, using an irradiated, genetically engineered 721.221 cell line (i.e., a B-cell line derived through mutagenesis that does not express dominant MHC class I molecules or expresses a relatively low amount of MHC class I molecules) that expresses a membrane-bound cytokine known to facilitate expansion of NK cells (such as, for example, IL-21). When combined with additional cytokines (e.g., IL-15 and/or IL-2), primary NK cells can be expanded up to about 100,000-fold (e.g., up to about 100-, about 200-, about 300-, about 400-, about 500-, about 600-, about 700-, about 800-, about 900-, about 1000-, about 5000-, about 10,000-, about 20,000-, about 30,000-, about 40,000-, about 50,000-, about 60,000-, about 70,000-, about 80,000-, about 90,000-, or about 100,000-fold) after about 14 days to about 21 days of expansion.

Alternatively, or in addition to the above, in some embodiments, NK cells are obtained by differentiating one or more stem cells according to any number of methods known in the relevant art. In some instances, NK cells are differentiated from one or more of: (i) iPSCs, (ii) ESCs, (iii) mesenchymal stem cells (MSCs), and (iv) hematopoietic stem cells (HSCs). Protocols for the differentiation of NK cells from various stem cell sources are described in, for example, Bock et al. J. Vis. Exp. (74):e50337, 2013; Knorr et al. Stem Cells Transl. Med. 2(4):274-83, 2013; Ni et al. Methods Mol. Biol. 1029:33-41, 2013; Zhu and Kaufman (Methods Mol. Biol. 2048: 107-19, 2019); each of which is herein incorporated by reference in their entirety, with particularity for relevant disclosure pertaining to methods of differentiating stem cells.

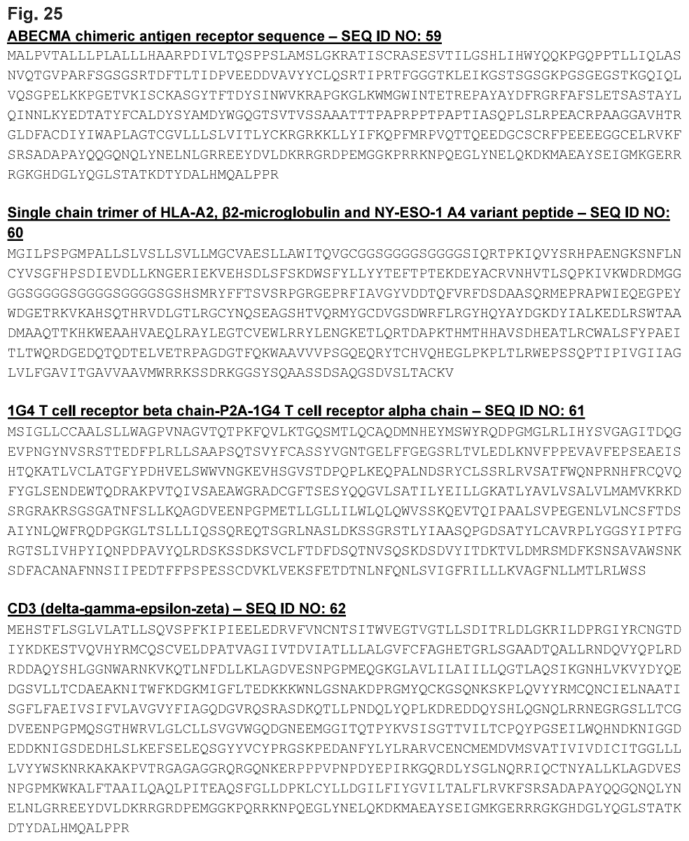

Generally, NK cells will bind to an APC upon recognition of an antigen by one or more endogenous receptors. However, NK cells may be engineered to express one or more additional receptors to facilitate treatment of a subject in need thereof. In some embodiments, the NK cell is engineered to express one or more additional receptors. In some further embodiments, the NK cell is engineered to express one or more additional receptors using a CAR construct. Generally, a CAR construct comprises (i) an extracellular domain, wherein the extracellular domain comprises an antigen recognition domain that specifically binds to an antigen on the surface of a cell (e.g., a cancer cell), (ii) a transmembrane domain, and (iii) an intracellular domain, wherein the intracellular domain comprises one or more costimulatory domains. Several CAR construct backbones are known in the relevant, such as, for example, the ABECMA CAR construct (see, e.g., SEQ ID NO: 59, as shown in FIG. 25). In the ABECMA CAR construct, the extracellular domain is configured to specifically bind to the cell-surface antigen BCMA. Further, the intracellular domain of the ABECMA CAR construct comprises a 4-1BB costimulatory domain (which is associated with enhanced activation, expansion, and persistence of the cell comprising the ABECMA CAR construct) and a CD3((CD3zeta) signaling domain (which results in the activation of the cell comprising the ABECMA CAR construct upon binding of BCMA-expressing target cells). Any number of suitable CAR construct backbones known in the relevant art may be utilized; and may further be cloned into any number of suitable expression vectors (e.g., a lentiviral expression vector). Alternatively, or in addition to, the NK cell can be engineered to express one or more additional receptors using a TCR construct. Generally, a TCR construct comprises (i) extracellular alpha and beta peptide chains to recognize polypeptide fragments presented by MHC molecules, (ii) four extracellular CD3 chains (one gamma chain, one delta chain, and two epsilon chains), (iii) a transmembrane domain, and (iv) an intracellular domain, wherein the intracellular domain comprises one or more immunoreceptor tyrosine-based activation motifs (ITAMs) that facilitate the activation of downstream targets in response to antigen-mediated activation of the extracellular alpha and beta peptide chains. Any number of suitable TCR construct backbones known in the relevant art may be utilized.

Further, the NK cells can be modified to express a NK cell growth factor that promotes the growth and/or activation of NK cells. Non-limiting examples of suitable NK cell growth factors include: IL-2, IL-7, IL-12, IL-15, IL-27, and IL-35. Suitable modification systems are known in the relevant art (see, e.g., Sambrook et al., Molecular Cloning: A Laboratory Manual, 3rd ed., Cold Spring Harbor Press, Cold Spring Harbor, N.Y. 2001; and Ausubel et al., Current Protocols in Molecular Biology, Greene Publishing Associates and John Wiley & Sons, N Y, 1994; each of which is incorporated herein by reference in its entirety).

### 2. T-Cells:

In some embodiments, the hematopoietic cell used (i) to develop the compositions, and/or (ii) in the methods disclosed herein is a T-cell. In some further embodiments, the T-cell comprises a γδ-T-cell.

In some embodiments, the T-cells are derived from blood, cord blood, bone marrow, one or more lymph nodes, or a lymphoid organ (e.g., the spleen or thymus). In some embodiments, the T-cells are human T-cells. Alternatively, the T-cells can be mouse-derived T-cells. Generally, the T-cells are primary cells, such as, for example, those isolated directly from a subject (and optionally may be frozen for future use according to any number of cell storage protocols known in the relevant art).

In some embodiments, the T-cells comprise one or more subsets of T-cells, such as, for example, a whole T-cell population (i.e., a population of T-cells that is not specifically isolated for a particular subtype), CD4⁺ T-cells, CD8⁺ T-cells, NKT-cells, as well as subpopulations thereof (i.e., one or more subpopulations of T-cells that are defined by: function, activation state, maturity, potential for differentiation, expansion, recirculation, localization, and/or persistence capacities, antigen-specificity, type of antigen receptor, presence in a particular organ or compartment, marker or cytokine secretion profile, and/or degree of differentiation). Further exemplary subsets of T-cells include, but are not limited to: naive T-cells (TN cells), effector T-cells (TEFF), memory T-cells and sub-types thereof (e.g., T memory stem cells (TSC_{M}), central memory T-cells (TC_{M}), effector memory T-cells (T_{EM}), terminally differentiated effector memory T-cells, and the like), immature T-cells, mature T-cells, helper T-cells (e.g., TH1 cells, TH2 cells, TH3 cells, TH17 cells, TH9 cells TH22 cells, follicular helper T cells, and the like), cytotoxic T-cells, mucosa-associated invariant T (MAIT)-cells naturally occurring and adaptive regulatory T-cells (Treg), alpha beta (αβ) T-cells, gamma delta (γδ) T-cells, and the like. In some preferred embodiments, the T-cell comprises a γδ-T-cell.

With reference to the subject in need of treatment, the T-cells (such as, for example, γδ-T-cells) may be allogeneic or autologous. Alternatively, or in addition to, the T-cells may be obtained from a cell line (such as, for example, a hematopoietic stem cell (HSC) or an induced pluripotent stem cell (iPSC) line). In some embodiments, the T-cells are isolated from a donor subject and/or a recipient subject, and may further be subjected to one or more of preparing, processing, culturing, cellular engineering (e.g., the introduction of a CAR or TCR construct into the cell), and/or re-introduction into a subject in need of treatment (before or after cryopreservation of the T-cells).

In some embodiments, one or more T-cell populations are enriched for or depleted of cells that are positive for a specific marker, such as, for example, a surface marker, or that are negative for a specific marker. In some embodiments, the specific marker can be those that are absent or expressed at relatively low levels on certain populations of T-cells (e.g., non-memory T-cells) but are present or expressed at relatively higher levels on certain other populations of T-cells (e.g., memory T-cells).

In some embodiments, the T-cells are separated from a peripheral blood mononuclear cell (PBMC) sample by negative selection of markers expressed on non-T-cells (e.g., CD14), such as, for example, B-cells, monocytes, or other white blood cells. In some embodiments, the T-cells are separated from a PBMC sample by positive selection of markers expressed on T-cells (e.g., CD4 or CD8). In some embodiments, the T-cells are separated from a PBMC sample by a combination of negative selection of markers expressed on non-T-cells and positive selection of markers expressed on T-cells. In some embodiments, the T-cells are further enriched for or depleted of one or more subtypes of T-cells (such as, for example, γδ-T-cells).

In some embodiments, the T-cells are autologous. In such embodiments, the T-cells can be obtained from one or more tumor samples obtained from a subject, and a single cell suspension can be generated. The single cell suspension can be generated in any suitable manner, such as, for example, mechanical means for disaggregating the one or more tumor samples and/or enzymatic means for disaggregating the one or more tumor samples. The single-cell suspension produced from the one or more tumors can subsequently be cultured in a suitable cell culture medium, optionally with the addition of interleukin-2 (IL-2). The cultured T-cells can be pooled and rapidly expanded. Rapid expansion provides an increase in the number of T-cells of at least about 50-fold, about 60-fold, about 70-fold, about 80-fold, about 90-fold, about 100-fold or greater over a period of about 10 to about 14 days. More preferably, rapid expansion can provide an increase of at least about 150-fold, about 200-fold, about 250-fold, about 300-fold, about 350-fold, about 400-fold, about 450-fold, about 500-fold, about 600-fold, about 700-fold, about 800-fold, about 900-fold, about 1000-fold or greater over a period of about 10 to about 14 days.

Expansion of T-cells (such as, for example, γδ-T-cells) can be accomplished using any number of methods as are known in the relevant art. For example, T-cells can be rapidly expanded using non-specific T-cell receptor stimulation (e.g., the OKT3 monoclonal antibody) in the presence of feeder lymphocytes and/or in the presence of IL-2 or IL-15, with IL-2 finding additional utility in certain embodiments. Alternatively, T-cells can be rapidly expanded by stimulation of PBMCs in vitro with one or more antigens (including antigenic portions thereof, such as epitopes or one or more cells expressing an antigen) of a cancer, which optionally can be expressed from a vector, in the presence of a T-cell growth factor (e.g., IL-2 or IL-15). The in vitro-induced T-cells are rapidly expanded by re-stimulation with the same antigens of the cancer. Alternatively, or in addition to, the T-cells can be re-stimulated with irradiated, autologous lymphocytes or with irradiated allogeneic lymphocytes and a T-cell growth factor.

Generally, T-cells will bind to an antigen-presenting cell (APC) upon recognition of an antigen by one or more endogenous T-cell receptors. However, T-cells may be engineered to express one or more additional receptors to facilitate treatment of a subject in need thereof. In some embodiments, the T-cell is engineered to express one or more additional receptors (in some instances, the T-cell is a γδ-T-cell engineered to express one or more additional receptors). In some further embodiments, the T-cell is engineered to express one or more additional receptors using a CAR construct. Generally, a CAR construct comprises (i) an extracellular domain, wherein the extracellular domain comprises an antigen recognition domain that specifically binds to an antigen on the surface of a cell (e.g., a cancer cell), (ii) a transmembrane domain, and (iii) an intracellular domain, wherein the intracellular domain comprises one or more costimulatory domains. Several CAR construct backbones are known in the relevant, such as, for example, the ABECMA CAR construct (see, e.g., SEQ ID NO: 59, as shown in FIG. 25). In the ABECMA CAR construct, the extracellular domain is configured to specifically bind to the cell-surface antigen BCMA. Further, the intracellular domain of the ABECMA CAR construct comprises a 4-1BB costimulatory domain (which is associated with enhanced activation, expansion, and persistence of the cell comprising the ABECMA CAR construct) and a CD3((CD3zeta) signaling domain (which results in the activation of the cell comprising the ABECMA CAR construct upon binding of BCMA-expressing target cells). Any number of suitable CAR construct backbones known in the relevant art may be utilized; and may further be cloned into any number of suitable expression vectors (e.g., a lentiviral expression vector). Alternatively, or in addition to, the T-cell can be engineered to express one or more additional receptors using a TCR construct. Generally, a TCR construct comprises (i) extracellular alpha and beta peptide chains to recognize polypeptide fragments presented by MHC molecules, (ii) four extracellular CD3 chains (one gamma chain, one delta chain, and two epsilon chains), (iii) a transmembrane domain, and (iv) an intracellular domain, wherein the intracellular domain comprises one or more immunoreceptor tyrosine-based activation motifs (ITAMs) that facilitate the activation of downstream targets in response to antigen-mediated activation of the extracellular alpha and beta peptide chains. Any number of suitable TCR construct backbones known in the relevant art may be utilized.

Further, the T-cells can be modified to express a T-cell growth factor that promotes the growth and/or activation of T-cells. Non-limiting examples of suitable T-cell growth factors include: IL-2, IL-7, IL-12, and IL-15. Suitable modification systems are known in the relevant art (see, e.g., Sambrook et al., Molecular Cloning: A Laboratory Manual, 3rd ed., Cold Spring Harbor Press, Cold Spring Harbor, N.Y. 2001; and Ausubel et al., Current Protocols in Molecular Biology, Greene Publishing Associates and John Wiley & Sons, N Y, 1994; each of which is incorporated herein by reference in its entirety).

### 3. TILs:

In some embodiments, the hematopoietic cell used (i) to develop the compositions, and/or (ii) in the methods disclosed herein is a tumor-infiltrating lymphocyte (TIL).

In some embodiments, the TILs are derived from a tumor sample. In some embodiments, the tumor sample comprises a human tumor sample. In some further embodiments, the human tumor sample is obtained from a subject with cancer (i.e., the tumor sample is autologously sourced). In other embodiments, the tumor sample comprises a mouse tumor sample. In some further embodiments, the mouse tumor sample is obtained from a mouse with cancer, wherein the cancer occurs as a result of (i) the genetic background of the mouse (e.g., Apc^{Min/+} mice), (ii) an injection with one or more murine cancer cells (e.g., an OT-1 mouse injected with one or more murine cancer cells), or (iii) an injection with one or more human cancer cells (e.g., a NSG mouse injected with one or more human cancer cells).

In some embodiments, the TILs are derived from a tumor sample, wherein the tumor sample is surgically removed, biopsied, or otherwise extracted from an appropriate source (e.g., a subject with cancer, a mouse with cancer, etc.). Following extraction, the tumor sample can be prepared to produce a single cell suspension. The single cell suspension can be generated in any suitable manner, such as, for example, mechanical means for disaggregating the tumor sample and/or enzymatic means for disaggregating the tumor sample.

Once a single cell suspension has been produced, the TILs may be isolated from the single cell suspension according to any number of methods known in the relevant art (see, e.g., Tan, Y., & Lei, Y. (2019). Isolation of Tumor-Infiltrating Lymphocytes by Ficoll-Paque Density Gradient Centrifugation. Mouse Models Of Innate Immunity, 93-99.; U.S. Patent Appl. Publ. Nos. 2022/0282215; PCT Publication No. 2022/212888; all of which are incorporated by reference in their entirety, with particularity for their relevant disclosures pertaining to methods for isolating, extracting, or otherwise purifying TILs).

Following isolation, the TILs may subsequently be cultured in a suitable cell culture medium and expanded over a period of time sufficient to produce a population of TILs for autologous transplantation. In some embodiments, the cell culture medium comprises one or more of: (i) a plurality of irradiated feeder cells, (ii) one or more anti-CD3 antibodies, and (iii) one or more cytokines (e.g., IL-2). As is known to those skilled in the relevant art, the period of time sufficient to produce a population of TILs for autologous transplantation will depend upon, inter alia, the methodology employed to isolate the TILs, the number of TILs present following isolation, the weight of the subject with cancer, the overall tumor burden, and other factors known in the relevant art. Generally, the population of TILs may be generated in vitro after an expansion period of about 2 weeks to about 3 weeks.

### 4. Stern Cells:

In some embodiments, the hematopoietic cell used (i) to develop the compositions, and/or (ii) in the methods disclosed herein is derived from a stem cell. In some embodiments, the stem cell is a multipotent stem cell, such as, for example, an MSC. In some embodiments, the stem cell is a pluripotent stem cell, such as, for example, an HSC. In some embodiments, the pluripotent stem cell is an induced pluripotent stem cell (iPSC). The induction of pluripotency was originally achieved by the reprogramming of somatic cells via the introduction of one or more transcription factors that are linked to pluripotency (e.g., the Yamanaka factors (Oct3/4, Sox2, Klf4, and c-Myc), Nanog, Lin28). The use of iPSCs circumvents most of the ethical and practical problems associated with large-scale clinical use of embryonic stem cells (ESCs), and patients with iPSC-derived autologous transplants may not require lifelong immunosuppressive treatments to prevent rejection (e.g., transplant rejection, hyperacute rejection, acute rejection, chronic rejection, graft failure, etc.).

Generally, with the exception of germ cells, any cell can be used as a starting point for iPSCs. Non-limiting examples of cells that may be used to generate iPSCs include: fibroblasts, keratinocytes, any number of hematopoietic cells, mesenchymal cells, cells from the liver, cells from the stomach, and the like. There is no limitation on the degree of cell differentiation or the age of an animal (including, but not limited to, mice, rats, pigs, cows, humans, primates, and non-human primates) from which cells are collected. For example, undifferentiated progenitor cells (including, but not limited to, somatic stem cells) and/or differentiated mature cells can be used as sources of somatic cells in the methods disclosed herein. However, as the potential for the accrual of mutations and cellular decay increases with time and each round of cellular division, it may be preferable to obtain cells from sources that are relatively young in age (such as, for example, less than about 30 or less than about 40 years of age in instances where the cells are derived from humans).

Somatic cells can be reprogrammed to produce iPSCs using methods known to one of skill in the art (see, e.g., U.S. Patent Appl. Publ. Nos. 2009/0246875, 2010/0210014, 2011/0104125, and 2012/0276636; U.S. Pat. Nos. 8,058,065, 8,129,187, 8,268,620, 8,546,140, 9,175,268, 8,741,648, and 8,691,574; and PCT Publication No. WO 2007/069666; all of which are incorporated herein by reference, with particularity for relevant disclosure pertaining to methods for reprogramming cells). Generally, nuclear reprograming factors are used to produce pluripotent stem cells from a somatic cell (e.g., the Yamanaka factors). In some embodiments, at least three of (i.e., at least three, at least four, at least five, or all six): (i) Klf4, (ii) c-Myc, (iii) Oct3/4, (iv) Sox2, (v) Nanog, and (vi) Lin28, are utilized. Mouse and human cDNA sequences of these nuclear reprogramming substances are available with reference to the NCBI accession numbers mentioned in PCT Publication No. WO 2007/069666 and U.S. Pat. No. 8,183,038, which are incorporated herein by reference in their entirety, with particularity for cDNA sequences and NCBI accession numbers described therein. Methods for introducing one or more reprogramming substances, or nucleic acids encoding these reprogramming substances, are generally known in the relevant art, and described in, for example, U.S. Pat. Nos. 8,268,620, 8,691,574, 8,741,648, 8,546, 140, 8,900,871 and 8,071,369, all of which are incorporated herein by reference in their entirety, with particularity for methods of introducing reprogramming substances and nucleic acids encoding such reprogramming substances.

Once derived, iPSCs can be cultured in a medium sufficient to maintain pluripotency.

The iPSCs may be used with various media and techniques developed to culture pluripotent stem cells (e.g., embryonic stem cells), such as, for example, the methods described in U.S. Pat. No. 7,442,548 and U.S. Patent Pub. No. 2003/0211603. Such cell culture media and techniques, generally, include one or more differentiation suppression factors (such as, for example, leukemia inhibitory factor (LIF) in the case of mouse-derived iPSCs, or basic fibroblast growth factor (bFGF) in the case of human-derived iPSCs).

In some embodiments, a desired stem cell for use in the generation of a genetically modified hematopoietic cell is selected (e.g., an iPSC, an HSC, or the like). Subsequently, the desired stem cell may be generated as described above, or isolated from an appropriate source, as is generally known in the relevant art. The selected stem cell may subsequently be stimulated for differentiation into a desired hematopoietic cell (e.g., a T-cell, a NK cell, or the like) by selectively culturing the selected stem cell in the presence and/or absence of any number of factors (e.g., growth factors, cellular differentiation factors, etc.) as is generally known in the art (see, e.g., Nishimura, T., & Nakauchi, H. (2019). Generation of Antigen-Specific T Cells from Human Induced Pluripotent Stem Cells. Methods In Molecular Biology, 25-40.).

### D. Exogenous Cytokines

In some embodiments, the hematopoietic cell used (i) to develop the compositions, and/or (ii) in the methods disclosed herein is engineered to further express an exogenous cytokine. In some further embodiments, the hematopoietic cell comprises a NK cell. In other embodiments, the hematopoietic cell can comprise a T-cell, a TIL, or a stem cell.

In some embodiments, engineering the hematopoietic cell to express an exogenous cytokine comprises transforming, transducing, transfecting, infecting, or otherwise incorporating a plasmid, a vector, a virus, or any other genetic construct comprising a nucleic acid sequence encoding the exogenous cytokine, wherein the exogenous cytokine is under the control of a suitable promoter, as are generally known in the relevant art. In some further embodiments, the exogenous cytokine is encoded by a CAR expression construct. In still further embodiments, the CAR expression construct is incorporated into the hematopoietic cell by transduction (e.g., using vectofusin). In some embodiments, the CAR expression construct comprises an ABECMA CAR construct.

In some embodiments, expression of the exogenous cytokine is under the control of an inducible promoter. Alternatively, in other embodiments, expression of the exogenous cytokine is under the control of a constitutive promoter. Phrased differently, in some instances the exogenous cytokine is constitutively expressed, whereas in other instances the exogenous cytokine is expressed following the inducible activation of a promoter.

In some embodiments, the exogenous cytokine comprises interleukin-15 (IL-15). In some further embodiments, IL-15 comprises human IL-15. Alternatively, in some other embodiments, IL-15 comprises mouse IL-15.

### E. Effects of Genetic Modification (CRL5 Complex Genes—Alone)

In some embodiments, the genetically modified hematopoietic cell is subjected to one or more assays to determine the effects of the genetic modifications to the one or more CRL5 complex genes with respect to one or more properties of a cell (such as, for example, a genetically modified hematopoietic cell), including, but not limited to: proliferation, resistance to immune suppression, cytotoxic capacity, killing efficiency, the production of cytokines, and the like.

### 1. Proliferation:

In some embodiments, the one or more genetic modifications to the one or more CRL5 complex genes improves proliferation of the resultant genetically modified hematopoietic cell by about 10% to about 100% compared to proliferation of a control hematopoietic cell, as determined by an assay. In some embodiments, the one or more genetic modifications to the one or more CRL5 complex genes improves proliferation of the resultant genetically modified hematopoietic cell by about 10% to about 30%, about 20% to about 40%, about 30% to about 50%, about 40% to about 60%, about 50% to about 70%, about 60% to about 80%, about 70% to about 90%, or about 80% to about 100% compared to proliferation of a control hematopoietic cell, as determined by an assay.

Alternatively, or in addition to, the one or more genetic modifications to the one or more CRL5 complex genes improves proliferation of the resultant genetically modified hematopoietic cell by about 1.0-fold to about 15.0-fold compared to proliferation of a control hematopoietic cell, as determined by an assay. In some embodiments, the one or more genetic modifications to the one or more CRL5 complex genes improves proliferation of the resultant genetically modified hematopoietic cell by about 1.0-fold to about 3.0-fold, about 2.0-fold to about 4.0-fold, about 3.0-fold to about 5.0-fold, about 4.0 to about 6.0-fold, about 5.0-fold to about 7.0-fold, about 6.0-fold to about 8.0-fold, about 7.0-fold to about 9.0-fold, about 8.0-fold to about 10.0-fold, about 9.0-fold to about 11.0-fold, about 10.0-fold to about 12.0-fold, about 11.0-fold to about 13.0-fold, about 12.0-fold to about 14.0-fold, or about 13.0-fold to about 15.0-fold compared to proliferation of a control hematopoietic cell, as determined by an assay.

In further embodiments, the assay comprises: (i) a 5-ethynyl-2′-deoxyuridine (EdU) incorporation assay, (ii) a bromodeoxyuridine (BrdU) incorporation assay, (iii) a tetrazolium salt reduction assay, (iv) an ATP assay, or (v) a Ki-67 assay. Non-limiting examples of tetrazolium salts that may be used in the assay include: 3-(4,5-dimethylthiazal-2-yl)-2,5-diphenyltetrazolium bromide (MTT), 2,3-bis-(2-methoxy-4-nitro-5-sulfophenyl) (sodium salt hydrate) (XTT), 3-(4,5-dimethylthiazol-2-yl)-5-(3-carboxymethoxyphenyl)-2-(4-sulfophenyl)-2H-tetrazolium (MTS), as well as the water-soluble tetrazolium salts (WST) 5-(2,4-disulfophenyl)-2-(4-iodophenyl)-3-(4-nitrophenyl)-2H-tetrazolium (monosodium salt) (WST-1) and 5-(2,4-disulfophenyl)-3-(2-methoxy-4-nitrophenyl)-2-(4-nitrophenyl)-2H-tetrazolium (monosodium salt) (WST-8). Generally, tetrazolium salt reduction assays rely on oxidoreductases to reduce a given tetrazolium salt to an insoluble formazan compound, resulting in a colorimetric or fluorometric change that can be detected using methods known in the relevant art.

### 2. Resistance to Immune Suppression:

In some embodiments, the one or more genetic modifications to the one or more CRL5 complex genes improves resistance to immune suppression of the resultant genetically modified hematopoietic cell by about 10% to about 100% compared to resistance to immune suppression of a control hematopoietic cell, as determined by an assay. In some embodiments, the one or more genetic modifications to the one or more CRL5 complex genes improves resistance to immune suppression of the resultant genetically modified hematopoietic cell by about 10% to about 30%, about 20% to about 40%, about 30% to about 50%, about 40% to about 60%, about 50% to about 70%, about 60% to about 80%, about 70% to about 90%, or about 80% to about 100% compared to resistance to immune suppression of a control hematopoietic cell, as determined by an assay.

Alternatively, or in addition to, the one or more genetic modifications to the one or more CRL5 complex genes improves resistance to immune suppression of the resultant genetically modified hematopoietic cell by about 1.0-fold to about 10.0-fold compared to immune suppression of a control hematopoietic cell, as determined by an assay. In some embodiments, the one or more genetic modifications to the one or more CRL5 complex genes improves resistance to immune suppression of the resultant genetically modified hematopoietic cell by about 1.0-fold to about 3.0-fold, about 2.0-fold to about 4.0-fold, about 3.0-fold to about 5.0-fold, about 4.0 to about 6.0-fold, about 5.0-fold to about 7.0-fold, about 6.0-fold to about 8.0-fold, about 7.0-fold to about 9.0-fold, or about 8.0-fold to about 10.0-fold compared to resistance to immune suppression of a control hematopoietic cell, as determined by an assay.

In further embodiments, the assay comprises (i) a serial challenge assay (with and/or without the addition of one or more immunosuppressive factors), or (ii) a cytokine release assay. As used herein, the term “a serial challenge assay” generally refers to an assay in which a plurality of test cells (e.g., a plurality of NK cells (with or without genetic modifications to one or more CRL5 complex genes; with or without genetic modifications to one or more control genes, such as, for example, CISH and/or AAVS1)) is exposed to a plurality of target cells (i.e., a plurality of cancer cells, such as, for example, K562 cells) over a period of time (e.g., once every day for 3, 4, 5, 6, 7, 8, or more days). The assay may optionally include the addition of one or more immunosuppressive factors, such as, for example, adenosine (NECA) and/or prostaglandin (PGE2), to the cell culture medium to facilitate the detection and/or measurement of immune suppression (and/or resistance thereto). The capacity of the test cells to kill the target cells can be measured over the period of time (e.g., once or more every day) to detect and/or measure (i) the number of living and/or dead cells, (ii) changes in the rate at which cells proliferate and/or die, (iii) changes in the amount of living and/or dead cells, and/or (iv) changes to any of the preceding parameters in the presence and/or absence of one or more immunosuppressive factors. Alternatively, or in addition to, cell culture medium may be collected over the period of time to detect and/or measure (i) the presence or absence of one or more cytokines, (ii) changes in the presence or absence of one or more cytokines, (iii) the amount of the one or more cytokines, and/or (iv) changes in the amount of the one or more cytokines. Non-limiting examples of cytokines that may be assessed include: (i) granulocyte colony-stimulating factor (G-CSF), (ii) granulocyte-macrophage colony-stimulating factor (GM-CSF), (iii) interferon alpha (IFN-α), (iv) interferon gamma (IFN-γ), (v) interleukin-1 beta (IL-1β), (vi) IL-2, (vii) IL-4, (viii) IL-5, (ix) IL-6, (x) IL-8, (xi) IL-10, (xii) interleukin-12 p70 (IL-12 p70), (xiii) IL-13, (xiv) IL-17A, (xv) IL-18, (xvi) interferon gamma-induced protein 10 (IP-10), (xvii) monocyte chemoattractant protein-1 (MCP-1), (xviii) macrophage inflammatory protein-1 alpha (MIP-la), (xix) MIP-1 beta (MIP-10), (xx) tumor necrosis factor alpha (TNF-α), (xxi) TNF-β, or any combination thereof.

### 3. Cytotoxic Capacity and Killing Efficiency:

In some embodiments, the one or more genetic modifications to the one or more CRL5 complex genes improves the cytotoxic capacity of the resultant genetically modified hematopoietic cell by about 10% to about 100% compared to cytotoxic capacity of a control hematopoietic cell, as determined by an assay. In some embodiments, the one or more genetic modifications to the one or more CRL5 complex genes improves the cytotoxic capacity of the resultant genetically modified hematopoietic cell by about 10% to about 30%, about 20% to about 40%, about 30% to about 50%, about 40% to about 60%, about 50% to about 70%, about 60% to about 80%, about 70% to about 90%, about 20% to about 50%, about 30% to about 60%, about 20% to about 60%, about 30% to about 70%, about 20% to about 70%, about 30% to about 80%, or about 80% to about 100% compared to cytotoxic capacity of a control hematopoietic cell, as determined by an assay.

Alternatively, or in addition to, the one or more genetic modifications to the one or more CRL5 complex genes improves the cytotoxic capacity of the resultant genetically modified hematopoietic cell by about 0.5-fold to about 10.0-fold compared to cytotoxic capacity of a control hematopoietic cell, as determined by an assay. In some embodiments, the one or more genetic modifications to the one or more CRL5 complex genes improves the cytotoxic capacity of the resultant genetically modified hematopoietic cell by about 0.5-fold to about 1.0-fold, about 1.0-fold to about 2.0-fold, about 2.0-fold to about 3.0-fold, about 3.0-fold to about 4.0-fold, about 4.0-fold to about 5.0-fold, about 5.0-fold to about 10.0-fold compared to cytotoxic capacity of a control hematopoietic cell, as determined by an assay.

In further embodiments, the assay comprises: (i) a serial challenge assay (with and/or without assessment of cells by (a) fluorescence-activated cell sorting (FACS), and/or (b) live cell imaging (e.g., using Incucyte), (ii) a serial challenge assay (with and/or without the addition of one or more immunosuppressive factors to the cell culture medium), (iii) an enzymatic cytotoxicity assay, (iv) an apoptosis assay, (v) an autophagy assay, (vi) a cell viability assay, or (vii) a short-term killing assay.

In some embodiments, the one or more genetic modifications to the one or more CRL5 complex genes improves the killing efficiency of cancer cells by the resultant genetically modified hematopoietic cells by about 10% to about 100% compared to the killing efficiency of cancer cells by a control hematopoietic cell, as determined by an assay. In some embodiments, the one or more genetic modifications to the one or more CRL5 complex genes improves the killing efficiency of cancer cells by the resultant genetically modified hematopoietic cell by about 10% to about 30%, about 20% to about 40%, about 30% to about 50%, about 40% to about 60%, about 50% to about 70%, about 60% to about 80%, about 70% to about 90%, about 20% to about 50%, about 30% to about 60%, about 20% to about 60%, about 30% to about 70%, about 20% to about 70%, about 30% to about 80%, or about 80% to about 100% compared to the killing efficiency of cancer cells by a control hematopoietic cell, as determined by an assay.

Alternatively, or in addition to, the one or more genetic modifications to the one or more CRL5 complex genes improves the killing efficiency of cancer cells by the resultant genetically modified hematopoietic cells by about 0.5-fold to about 10.0-fold compared to the killing efficiency of cancer cells by a control hematopoietic cell, as determined by an assay. In some embodiments, the one or more genetic modifications to the one or more CRL5 complex genes improves the killing efficiency of cancer cells by the resultant genetically modified hematopoietic cell by about 0.5-fold to about 1.0-fold, about 1.0-fold to about 2.0-fold, about 2.0-fold to about 3.0-fold, about 3.0-fold to about 4.0-fold, about 4.0-fold to about 5.0-fold, about 5.0-fold to about 10.0-fold compared to the killing efficiency of cancer cells by a control hematopoietic cell, as determined by an assay.

In further embodiments, the assay comprises: (i) a serial challenge assay (with and/or without assessment of cells by (a) fluorescence-activated cell sorting (FACS), and/or (b) live cell imaging (e.g., using Incucyte), (ii) a serial challenge assay (with and/or without the addition of one or more immunosuppressive factors to the cell culture medium), (iii) an enzymatic cytotoxicity assay, (iv) an apoptosis assay, (v) an autophagy assay, (vi) a cell viability assay, or (vii) a short-term killing assay.

### 4. Cytokine Production:

In some embodiments, the one or more genetic modifications to the one or more CRL5 complex genes improves capability of the resultant genetically modified hematopoietic cell to stimulate production of one or more cytokines by about 10% to about 100% compared to capability to stimulate production of one or more cytokines of a control hematopoietic cell, as determined by an assay. In some embodiments, the one or more genetic modifications to the one or more CRL5 complex genes improves capability of the resultant genetically modified hematopoietic cell to stimulate production of one or more cytokines by about 10% to about 30%, about 20% to about 40%, about 30% to about 50%, about 40% to about 60%, about 50% to about 70%, about 60% to about 80%, about 70% to about 90%, or about 80% to about 100% compared to capability to stimulate production of one or more cytokines of a control hematopoietic cell, as determined by an assay. In some embodiments, the one or more genetic modifications to the one or more CRL5 complex genes improves capability of the resultant genetically modified hematopoietic cell to stimulate production of one or more cytokines by about 100% to about 150%, about 150% to about 200%, about 200% to about 300%, about 1150% to about 1250%, about 2400% to about 2500%, or about 9000% to about 10,000% compared to capability to stimulate production of one or more cytokines of a control hematopoietic cell, as determined by an assay.

Alternatively, or in addition to, the one or more genetic modifications to the one or more CRL5 complex genes improves capability of the resultant genetically modified hematopoietic cell to stimulate production of one or more cytokines by about 0.5-fold to about 20.0-fold compared to capability to stimulate production of one or more cytokines of a control hematopoietic cell, as determined by an assay. In some embodiments, the one or more genetic modifications to the one or more CRL5 complex genes improves capability of the resultant genetically modified hematopoietic cell to stimulate production of one or more cytokines by about 0.5-fold to about 1.0-fold, about 1.0-fold to about 2.0-fold, about 2.0-fold to about 3.0-fold, about 3.0-fold to about 4.0-fold, about 4.0-fold to about 5.0-fold, about 5.0-fold to about 10.0-fold, about 10.0-fold to about 15.0-fold, about 1.0-fold to about 4.0-fold, about 4.0-fold to about 7.0-fold, about 7.0-fold to about 10.0-fold, about 10.0-fold to about 13.0-fold, about 13.0-fold to about 16.0-fold, about 16.0-fold to about 20.0-fold or about 15.0-fold to about 20.0-fold, compared to capability to stimulate production of one or more cytokines of a control hematopoietic cell, as determined by an assay. In some embodiments, the one or more genetic modifications to the one or more CRL5 complex genes improves capability of the resultant genetically modified hematopoietic cell to stimulate production of one or more cytokines by about 1.00-fold to about 1.50-fold, about 1.50-fold to about 2.00-fold, about 2.00-fold to about 3.00-fold, about 11.5-fold to about 12.5-fold, about 24.0-fold to about 25.0-fold, or about 90.0-fold to about 100.0-fold compared to capability to stimulate production of one or more cytokines of a control hematopoietic cell, as determined by an assay.

In further embodiments, the one or more cytokines are selected from a group including: (i) granulocyte colony-stimulating factor (G-CSF), (ii) granulocyte-macrophage colony-stimulating factor (GM-CSF), (iii) interferon alpha (IFN-α), (iv) interferon gamma (IFN-γ), (v) interleukin-1 beta (IL-1β), (vi) IL-2, (vii) IL-4, (viii) IL-5, (ix) IL-6, (x) IL-8, (xi) IL-10, (xii) interleukin-12 p70 (IL-12 p70), (xiii) IL-13, (xiv) IL-17A, (xv) IL-18, (xvi) interferon gamma-induced protein 10 (IP-10), (xvii) monocyte chemoattractant protein-1 (MCP-1), (xviii) macrophage inflammatory protein-1 alpha (MIP-1a), (xix) MIP-1 beta (MIP-1β), (xx) tumor necrosis factor alpha (TNF-α), (xxi) TNF-β, or any combination thereof.

In yet further embodiments, the assay comprises: (i) a ProcartaPlex multiplex cytokine assay, (ii) a Human LEGENDplex TH1 panel, (iii) an AlphaLISA assay, (iv) an intracellular cytokine stain assay, (v) a Luminex bead-based cytokine release assay, (vi) a MACS cytokine secretion assay, (vii) an ELISA, (viii) an ELISpot assay, (ix) an assessment of cell culture medium to detect the presence and/or measure the amount of one or more cytokines following any of the assays described herein.

### F. Effects of Genetic Modification (CRL5 Complex-Independent Genes—Alone)

In some embodiments, the genetically modified hematopoietic cell is subjected to one or more assays to determine the effects of the genetic modifications to the one or more CRL5 complex-independent genes with respect to one or more properties of a cell (such as, for example, a genetically modified hematopoietic cell), including, but not limited to: proliferation, resistance to immune suppression, cytotoxic capacity, killing efficiency, the production of cytokines, and the like.

### 1. Proliferation:

In some embodiments, the one or more genetic modifications to the one or more CRL5 complex-independent genes improves proliferation of the resultant genetically modified hematopoietic cell by about 50% to about 650% compared to proliferation of a control hematopoietic cell, as determined by an assay. In some embodiments, the one or more genetic modifications to the one or more CRL5 complex-independent genes improves proliferation of the resultant genetically modified hematopoietic cell by about 50% to about 650%, about 50% to about 75%, about 75% to about 100%, about 625% to about 650%, about 50% to about 100%, about 100% to about 150%, about 150% to about 200%, about 200% to about 250%, about 250% to about 300%, about 300% to about 3 50%, about 3 50% to about 400%, about 400% to about 450%, about 450% to about 500%, about 500% to about 550%, about 550% to about 600%, or about 600% to about 650% compared to proliferation of a control hematopoietic cell, as determined by an assay.

Alternatively, or in addition to, the one or more genetic modifications to the one or more CRL5 complex-independent genes improves proliferation of the resultant genetically modified hematopoietic cell by about 0.5-fold to about 7.0-fold compared to proliferation of a control hematopoietic cell, as determined by an assay. In some embodiments, the one or more genetic modifications to the one or more CRL5 complex-independent genes improves proliferation of the resultant genetically modified hematopoietic cell by about 0.5-fold to about 7.0-fold, about 0.5-fold to about 0.75-fold, about 0.75-fold to about 1.0-fold, about 6.25-fold to about 6.50-fold, about 0.5-fold to about 1.0-fold, about 1.0-fold to about 1.5-fold, about 1.5-fold to about 2.0-fold, about 2.0-fold to about 2.5-fold, about 2.5-fold to about 3.0-fold, about 3.0-fold to about 3.5-fold, about 3.5-fold to about 4.0-fold, about 4.0-fold to about 4.5-fold, about 4.5-fold to about 5.0-fold, about 5.0-fold to about 5.5-fold, about 5.5-fold to about 6.0-fold, about 6.0-fold to about 6.5-fold, or about 6.5-fold to about 7.0-fold compared to proliferation of a control hematopoietic cell, as determined by an assay.

In further embodiments, the assay comprises: (i) a quantification of cells and/or cell clusters using a cell imaging system (e.g., Incucyte), (ii) a 5-ethynyl-2′-deoxyuridine (EdU) incorporation assay, (iii) a bromodeoxyuridine (BrdU) incorporation assay, (iv) a tetrazolium salt reduction assay, (v) an ATP assay, or (vi) a Ki-67 assay. Non-limiting examples of tetrazolium salts that may be used in the assay include: 3-(4,5-dimethylthiazal-2-yl)-2,5-diphenyltetrazolium bromide (MTT), 2,3-bis-(2-methoxy-4-nitro-5-sulfophenyl) (sodium salt hydrate) (XTT), 3-(4,5-dimethylthiazol-2-yl)-5-(3-carboxymethoxyphenyl)-2-(4-sulfophenyl)-2H-tetrazolium (MTS), as well as the water-soluble tetrazolium salts (WST) 5-(2,4-disulfophenyl)-2-(4-iodophenyl)-3-(4-nitrophenyl)-2H-tetrazolium (monosodium salt) (WST-1) and 5-(2,4-disulfophenyl)-3-(2-methoxy-4-nitrophenyl)-2-(4-nitrophenyl)-2H-tetrazolium (monosodium salt) (WST-8).

Generally, tetrazolium salt reduction assays rely on oxidoreductases to reduce a given tetrazolium salt to an insoluble formazan compound, resulting in a colorimetric or fluorometric change that can be detected using methods known in the relevant art.

### 2. Resistance to Immune Suppression:

In some embodiments, the one or more genetic modifications to the one or more CRL5 complex-independent genes improves resistance to immune suppression of the resultant genetically modified hematopoietic cell by about 1.50% to about 55.00% compared to resistance to immune suppression of a control hematopoietic cell, as determined by an assay. In some embodiments, the one or more genetic modifications to the one or more CRL5 complex-independent genes improves resistance to immune suppression of the resultant genetically modified hematopoietic cell by about 1.50% to about 55.00%, about 1.5% to about 6.0%, about 21.0% to about 24.0%, about 27% to about 30%, about 1.50% to about 3 0.00%, about 3 0.00% to about 6.00%, about 6.00% to about 9.00%, about 9.00% to about 12.0%, about 12.0% to about 15.0%, about 15.0% to about 1⁸0.0%, about 1⁸0.0% to about 21.0%, about 21.0% to about 24.0%, about 24.0% to about 27.0%, about 27.0% to about 3 0.0%, about 42.0% to about 4 5.0%, or about 53.0% to about 55.0% compared to resistance to immune suppression of a control hematopoietic cell, as determined by an assay.

Alternatively, or in addition to, the one or more genetic modifications to the one or more CRL5 complex-independent genes improves resistance to immune suppression of the resultant genetically modified hematopoietic cell by about 0.015-fold to about 0.550-fold compared to immune suppression of a control hematopoietic cell, as determined by an assay. In some embodiments, the one or more genetic modifications to the one or more CRL5 complex-independent genes improves resistance to immune suppression of the resultant genetically modified hematopoietic cell by about 0.015-fold to about 0.550-fold, about 0.015-fold to about 0.060-fold, about 0.21-fold to about 0.24-fold, about 0.27-fold to about 0.30-fold, about 0.015-fold to about 0.030-fold, about 0.03-fold to about 0.06-fold, about 0.06-fold to about 0.09-fold, about 0.09-fold to about 0.12-fold, about 0.12-fold to about 0.15-fold, about 0.15-fold to about 0.18-fold, about 0.18-fold to about 0.21-fold, about 0.21-fold to about 0.24-fold, about 0.24-fold to about 0.27-fold, about 0.27-fold to about 0.30-fold, about 0.42-fold to about 0.45-fold, or about 0.53-fold to about 0.55-fold compared to resistance to immune suppression of a control hematopoietic cell, as determined by an assay.

In further embodiments, the assay comprises (i) a serial challenge assay (with and/or without the addition of one or more immunosuppressive factors), or (ii) a cytokine release assay. As used herein, the term “a serial challenge assay” generally refers to an assay in which a plurality of test cells (e.g., a plurality of NK cells) is exposed to a plurality of target cells (e.g., a plurality of cancer cells, such as, for example, K562 cells) over a period of time (e.g., once every day for 3, 4, 5, 6, 7, 8, or more days). The assay may optionally include the addition of one or more immunosuppressive factors, such as, for example, adenosine and/or prostaglandin (PGE2), to the cell culture medium to facilitate the detection and/or measurement of immune suppression (and/or resistance thereto). The capacity of the test cells to kill the target cells can be measured over the period of time (e.g., once or more every day) to detect and/or measure (i) the number of living and/or dead cells, (ii) changes in the rate at which cells proliferate and/or die, (iii) changes in the amount of living and/or dead cells, and/or (iv) changes to any of the preceding parameters in the presence and/or absence of one or more immunosuppressive factors. Alternatively, or in addition to, cell culture medium may be collected over the period of time to detect and/or measure (i) the presence or absence of one or more cytokines, (ii) changes in the presence or absence of one or more cytokines, (iii) the amount of the one or more cytokines, and/or (iv) changes in the amount of the one or more cytokines. Non-limiting examples of cytokines that may be assessed include: (i) granulocyte colony-stimulating factor (G-CSF), (ii) granulocyte-macrophage colony-stimulating factor (GM-CSF), (iii) interferon alpha (IFN-α), (iv) interferon gamma (IFN-γ), (v) interleukin-1 beta (IL-1β), (vi) IL-2, (vii) IL-4, (viii) IL-5, (ix) IL-6, (x) IL-8, (xi) IL-10, (xii) interleukin-12 p70 (IL-12 p70), (xiii) IL-13, (xiv) IL-17A, (xv) IL-18, (xvi) interferon gamma-induced protein 10 (IP-10), (xvii) monocyte chemoattractant protein-1 (MCP-1), (xviii) macrophage inflammatory protein-1 alpha (MIP-la), (xix) MIP-1 beta (MIP-1β), (xx) tumor necrosis factor alpha (TNF-α), (xxi) TNF-β, or any combination thereof.

### 3. Cytotoxic Capacity and Killing Efficiency:

In some embodiments, the one or more genetic modifications to the one or more CRL5 complex-independent genes improves the cytotoxic capacity of the resultant genetically modified hematopoietic cell by about 15.0% to about 100.0% compared to cytotoxic capacity of a control hematopoietic cell, as determined by an assay. In some embodiments, the one or more genetic modifications to the one or more CRL5 complex-independent genes improves the cytotoxic capacity of the resultant genetically modified hematopoietic cell by about 15.0% to about 100.0%, about 15.0% to about 20.0%, about 1⁸0.0% to about 24.0%, about 60.0% to about 63.0%, about 72.0% to about 75.0%, about 87% to about 90%, about 20.0% to about 30.0%, about 30.0% to about 40.0%, about 40.0% to about 50.0%, about 50.0% to about 60.0%, about 60.0% to about 70.0%, about 70.0% to about 80.0%, about 80.0% to about 90.0%, or about 90.0% to about 100.0% compared to cytotoxic capacity of a control hematopoietic cell, as determined by an assay.

Alternatively, or in addition to, the one or more genetic modifications to the one or more CRL5 complex-independent genes improves the cytotoxic capacity of the resultant genetically modified hematopoietic cell by about 0.15-fold to about 1.00-fold compared to cytotoxic capacity of a control hematopoietic cell, as determined by an assay. In some embodiments, the one or more genetic modifications to the one or more CRL5 complex-independent genes improves the cytotoxic capacity of the resultant genetically modified hematopoietic cell by about 0.15-fold to about 1.00-fold, about 0.15-fold to about 0.20-fold, about 0.18-fold to about 0.24-fold, about 0.60-fold to about 0.63-fold, about 0.72-fold to about 0.75-fold, about 0.87-fold to about 0.90-fold, about 0.20-fold to about 0.30-fold, about 0.30-fold to about 0.40-fold, about 0.40-fold to about 0.50-fold, about 0.50-fold to about 0.60-fold, about 0.60-fold to about 0.70-fold, about 0.70-fold to about 0.80-fold, about 0.80-fold to about 0.90-fold, or about 0.90-fold to about 1.00-fold compared to cytotoxic capacity of a control hematopoietic cell, as determined by an assay.

In further embodiments, the assay comprises: (i) a quantification of cells and/or cell clusters using a cell imaging system (e.g., Incucyte), (ii) a serial challenge assay (with and/or without assessment of cells by (a) fluorescence-activated cell sorting (FACS), and/or (b) live cell imaging (e.g., using Incucyte)), (iii) a serial challenge assay (with and/or without the addition of one or more immunosuppressive factors to the cell culture medium), (iv) an enzymatic cytotoxicity assay, (v) an apoptosis assay, (vi) an autophagy assay, (vii) a cell viability assay, or (viii) a short-term killing assay.

In some embodiments, the one or more genetic modifications to one or more CRL5 complex-independent genes improves the killing efficiency of cancer cells by the genetically modified hematopoietic cell by about 15.0% to about 100.0% compared to the killing efficiency of cancer cells by a control hematopoietic cell, as determined by an assay. In some embodiments, the one or more genetic modifications to one or more CRL5 complex-independent genes improves the killing efficiency of cancer cells by about 15.0% to about 100.0%, about 15.0% to about 20.0%, about 18.0% to about 24.0%, about 60.0% to about 63.0%, about 72.0% to about 75.0%, about 87% to about 90%, about 20.0% to about 30.0%, about 30.0% to about 40.0%, about 40.0% to about 50.0%, about 50.0% to about 60.0%, about 60.0% to about 70.0%, about 70.0% to about 8 0.0%, about 8 0.0% to about 90.0%, or about 90.0% to about 100.0% compared to the killing efficiency of cancer cells by a control hematopoietic cell, as determined by an assay.

Alternatively, or in addition to, the one or more genetic modifications to one or more CRL5 complex-independent genes improves the killing efficiency of cancer cells by the genetically modified hematopoietic cell by about 0.15-fold to about 1.00-fold compared to the killing efficiency of cancer cells by a control hematopoietic cell, as determined by an assay. In some embodiments, the one or more genetic modifications to one or more CRL5 complex-independent genes improves the killing efficiency of cancer cells by about 0.15-fold to about 1.00-fold, about 0.15-fold to about 0.20-fold, about 0.18-fold to about 0.24-fold, about 0.60-fold to about 0.63-fold, about 0.72-fold to about 0.75-fold, about 0.87-fold to about 0.90-fold, about 0.20-fold to about 0.30-fold, about 0.30-fold to about 0.40-fold, about 0.40-fold to about 0.50-fold, about 0.50-fold to about 0.60-fold, about 0.60-fold to about 0.70-fold, about 0.70-fold to about 0.80-fold, about 0.80-fold to about 0.90-fold, or about 0.90-fold to about 1.00-fold compared to the killing efficiency of cancer cells by a control hematopoietic cell, as determined by an assay.

In further embodiments, the assay comprises: (i) a quantification of cells and/or cell clusters using a cell imaging system (e.g., Incucyte), (ii) a serial challenge assay (with and/or without assessment of cells by (a) fluorescence-activated cell sorting (FACS), and/or (b) live cell imaging (e.g., using Incucyte)), (iii) a serial challenge assay (with and/or without the addition of one or more immunosuppressive factors to the cell culture medium), (iv) an enzymatic cytotoxicity assay, (v) an apoptosis assay, (vi) an autophagy assay, (vii) a cell viability assay, or (viii) a short-term killing assay.

### 4. Cytokine Production:

In some embodiments, the one or more genetic modifications to the one or more CRL5 complex-independent genes improves capability of the resultant genetically modified hematopoietic cell to stimulate production of one or more cytokines by about 5.0% to about 30.0% compared to capability to stimulate production of one or more cytokines of a control hematopoietic cell, as determined by an assay. In some embodiments, the one or more genetic modifications to the one or more CRL5 complex-independent genes improves capability of the resultant genetically modified hematopoietic cell to stimulate production of one or more cytokines by about 5.0% to about 3 0.0%, about 5.0% to about 10.0%, about 10.0% to about 15.0%, about 15.0% to about 20.0%, about 20.0% to about 25.0%, or about 25.0% to about 30.0% compared to capability to stimulate production of one or more cytokines of a control hematopoietic cell, as determined by an assay. In some embodiments, the one or more genetic modifications to the one or more CRL5 complex-independent genes improves capability of the resultant genetically modified hematopoietic cell to stimulate production of one or more cytokines by about 150% to about 250%, about 1500% to about 1⁶⁰⁰%, or about 8 100% to about 8200% compared to capability to stimulate production of one or more cytokines of a control hematopoietic cell, as determined by an assay.

Alternatively, or in addition to, the one or more genetic modifications to the one or more CRL5 complex-independent genes improves capability of the resultant genetically modified hematopoietic cell to stimulate production of one or more cytokines by about 0.05-fold to about 0.30-fold compared to capability to stimulate production of one or more cytokines of a control hematopoietic cell, as determined by an assay. In some embodiments, the one or more genetic modifications to the one or more CRL5 complex-independent genes improves capability of the resultant genetically modified hematopoietic cell to stimulate production of one or more cytokines by about 0.05-fold to about 0.30-fold, about 0.05-fold to about 0.10-fold, about 0.10-fold to about 0.15-fold, about 0.15-fold to about 0.20-fold, about 0.20-fold to about 0.25-fold, or about 0.25-fold to about 0.30-fold compared to capability to stimulate production of one or more cytokines of a control hematopoietic cell, as determined by an assay. In some embodiments, the one or more genetic modifications to the one or more CRL5 complex-independent genes improves capability of the resultant genetically modified hematopoietic cell to stimulate production of one or more cytokines by about 1.50-fold to about 2.50-fold, about 15.0-fold to about 16.0-fold, or about 81.0-fold to about 82.0-fold compared to capability to stimulate production of one or more cytokines of a control hematopoietic cell, as determined by an assay.

In further embodiments, the one or more cytokines are selected from a group including: (i) granulocyte colony-stimulating factor (G-CSF), (ii) granulocyte-macrophage colony-stimulating factor (GM-CSF), (iii) interferon alpha (IFN-α), (iv) interferon gamma (IFN-γ), (v) interleukin-1 beta (IL-1β), (vi) IL-2, (vii) IL-4, (viii) IL-5, (ix) IL-6, (x) IL-8, (xi) IL-10, (xii) interleukin-12 p70 (IL-12 p70), (xiii) IL-13, (xiv) IL-17A, (xv) IL-18, (xvi) interferon gamma-induced protein 10 (IP-10), (xvii) monocyte chemoattractant protein-1 (MCP-1), (xviii) macrophage inflammatory protein-1 alpha (MIP-la), (xix) MIP-1 beta (MIP-1β), (xx) tumor necrosis factor alpha (TNF-α), (xxi) TNF-β, or any combination thereof.

In yet further embodiments, the assay comprises: (i) a ProcartaPlex multiplex cytokine assay, (ii) a Human LEGENDplex TH1 panel, (iii) an AlphaLISA assay, (iv) an intracellular cytokine stain assay, (v) a Luminex bead-based cytokine release assay, (vi) a MACS cytokine secretion assay, (vii) an ELISA, (viii) an ELISpot assay, or (ix) an assessment of cell culture medium to detect the presence and/or measure the amount of one or more cytokines following any of the assays described herein.

### G. Effects of Genetic Modification (CRL5 Complex Genes+CRL5 Complex-Independent Genes)

In some embodiments, the genetically modified hematopoietic cell is subjected to one or more assays to determine the effects of the genetic modifications to the one or more CRL5 complex genes and the one or more CRL5 complex-independent genes with respect to one or more properties of a cell (such as, for example, a genetically modified hematopoietic cell), including, but not limited to: proliferation, resistance to immune suppression, cytotoxic capacity, killing efficiency, the production of cytokines, and the like.

### 1. Proliferation:

In some embodiments, the one or more genetic modifications to the one or more CRL5 complex-independent genes and the one or more genetic modifications to the one or more CRL5 complex genes improve proliferation of the resultant genetically modified hematopoietic cell by about 100% to about 2500% compared to proliferation of a control hematopoietic cell, as determined by an assay. In some embodiments, the one or more genetic modifications to the one or more CRL5 complex-independent genes and the one or more genetic modifications to the one or more CRL5 complex genes improve proliferation of the resultant genetically modified hematopoietic cell by about 100% to about 2500%, about 100% to about 300%, about 300% to about 500%, about 500% to about 700%, about 700% to about 900%, about 900% to about 1100%, about 1100% to about 1³⁰⁰%, about 1300% to about 1500%, about 1500% to about 1⁷⁰⁰%, about 1⁷⁰⁰% to about 1⁹⁰⁰%, about 1⁹⁰⁰% to about 2100%, about 2100% to about 2300%, or about 2300% to about 2500% compared to proliferation of a control hematopoietic cell, as determined by an assay.

Alternatively, or in addition to, the one or more genetic modifications to the one or more CRL5 complex-independent genes and the one or more genetic modifications to the one or more CRL5 complex genes improve proliferation of the resultant genetically modified hematopoietic cell by about 1.0-fold to about 25.0-fold compared to proliferation of a control hematopoietic cell, as determined by an assay. In some embodiments, the one or more genetic modifications to the one or more CRL5 complex-independent genes and the one or more genetic modifications to the one or more CRL5 complex genes improve proliferation of the resultant genetically modified hematopoietic cell by about 1.0-fold to about 25.0-fold, about 1.25-fold to about 1.50-fold, about 2.5-fold to about 3.0-fold, about 4.0-fold to about 5.0-fold, about 8.5-fold to about 9.0-fold, about 16.0-fold to about 16.5-fold, about 22.0-fold to about 22.5-fold, about 1.0-fold to about 3.0-fold, about 3.0-fold to about 5.0-fold, about 5.0-fold to about 7.0-fold, about 7.0-fold to about 9.0-fold, about 9.0-fold to about 11.0-fold, about 11.0-fold to about 13.0-fold, about 13.0-fold to about 15.0-fold, about 15.0-fold to about 17.0-fold, about 17.0-fold to about 19.0-fold, about 19.0-fold to about 21.0-fold, about 21.0-fold to about 23.0-fold, or about 23.0-fold to about 25.0-fold compared to proliferation of a control hematopoietic cell, as determined by an assay.

In further embodiments, the assay comprises: (i) a quantification of cells and/or cell clusters using a cell imaging system (e.g., Incucyte), (ii) a 5-ethynyl-2′-deoxyuridine (EdU) incorporation assay, (iii) a bromodeoxyuridine (BrdU) incorporation assay, (iv) a tetrazolium salt reduction assay, (v) an ATP assay, or (vi) a Ki-67 assay. Non-limiting examples of tetrazolium salts that may be used in the assay include: 3-(4,5-dimethylthiazal-2-yl)-2,5-diphenyltetrazolium bromide (MTT), 2,3-bis-(2-methoxy-4-nitro-5-sulfophenyl) (sodium salt hydrate) (XTT), 3-(4,5-dimethylthiazol-2-yl)-5-(3-carboxymethoxyphenyl)-2-(4-sulfophenyl)-2H-tetrazolium (MTS), as well as the water-soluble tetrazolium salts (WST) 5-(2,4-disulfophenyl)-2-(4-iodophenyl)-3-(4-nitrophenyl)-2H-tetrazolium (monosodium salt) (WST-1) and 5-(2,4-disulfophenyl)-3-(2-methoxy-4-nitrophenyl)-2-(4-nitrophenyl)-2H-tetrazolium (monosodium salt) (WST-8).

### 2. Resistance to Immune Suppression:

In some embodiments, the one or more genetic modifications to the one or more CRL5 complex-independent genes and the one or more genetic modifications to the one or more CRL5 complex genes improve resistance to immune suppression of the resultant genetically modified hematopoietic cell by about 6.00% to about 84.00% compared to resistance to immune suppression of a control hematopoietic cell, as determined by an assay. In some embodiments, the one or more genetic modifications to the one or more CRL5 complex-independent genes and the one or more genetic modifications to the one or more CRL5 complex genes improve resistance to immune suppression of the resultant genetically modified hematopoietic cell by about 6.00% to about 840.00%, about 6.00% to about 9.00%, about 9.00% to about 15.0%, about 15.0% to about 21.0%, about 21.0% to about 27.0%, about 4 5.0% to about 48.0%, about 60.0% to about 70.0%, about 54.0% to about 69.0%, about 9.00% to about 12.0%, about 12.0% to about 15.0%, about 15.0% to about 1⁸0.0%, about 1⁸0.0% to about 21.0%, about 21.0% to about 24.0%, about 24.0% to about 27.0%, about 27.0% to about 3 0.0%, about 3 0.0% to about 33.0%, about 33.0% to about 36.0%, about 36.0% to about 39.0%, about 39.0% to about 42.0%, about 42.0% to about 4 5.0%, about 4 5.0% to about 48.0%, about 48.0% to about 51.0%, about 51.0% to about 54.0%, about 54.0% to about 57.0%, about 57.0% to about 60.0%, about 60.0% to about 63.0%, about 63.0% to about 66.0%, about 66.0% to about 69.0%, or about 8 1.0% to about 84.0% compared to resistance to immune suppression of a control hematopoietic cell, as determined by an assay.

Alternatively, or in addition to, the one or more genetic modifications to the one or more CRL5 complex-independent genes and the one or more genetic modifications to the one or more CRL5 complex genes improve resistance to immune suppression of the resultant genetically modified hematopoietic cell by about 0.060-fold to about 0.840-fold compared to immune suppression of a control hematopoietic cell, as determined by an assay. In some embodiments, the one or more genetic modifications to the one or more CRL5 complex-independent genes and the one or more genetic modifications to the one or more CRL5 complex genes improve resistance to immune suppression of the resultant genetically modified hematopoietic cell by about 0.060-fold to about 0.840-fold, about 0.06-fold to about 0.09-fold, about 0.09-fold to about 0.15-fold, about 0.15-fold to about 0.21-fold, about 0.21-fold to about 0.27-fold, about 0.45-fold to about 0.48-fold, about 0.60-fold to about 0.70-fold, about 0.54-fold to about 0.69-fold, about 0.09-fold to about 0.12-fold, about 0.12-fold to about 0.15-fold, about 0.15-fold to about 0.18-fold, about 0.18-fold to about 0.21-fold, about 0.21-fold to about 0.24-fold, about 0.24-fold to about 0.27-fold, about 0.27-fold to about 0.30-fold, about 0.30-fold to about 0.33-fold, about 0.33-fold to about 0.36-fold, about 0.36-fold to about 0.39-fold, about 0.39-fold to about 0.42-fold, about 0.42-fold to about 0.45-fold, about 0.45-fold to about 0.48-fold, about 0.48-fold to about 0.51-fold, about 0.51-fold to about 0.54-fold, about 0.54-fold to about 0.57-fold, about 0.57-fold to about 0.60-fold, about 0.60-fold to about 0.63-fold, about 0.63-fold to about 0.66-fold, about 0.66-fold to about 0.69-fold, or about 0.81-fold to about 0.84-fold compared to resistance to immune suppression of a control hematopoietic cell, as determined by an assay.

In further embodiments, the assay comprises (i) a serial challenge assay (with and/or without the addition of one or more immunosuppressive factors), or (ii) a cytokine release assay. The assay may optionally include the addition of one or more immunosuppressive factors, such as, for example, adenosine and/or prostaglandin (PGE2), to the cell culture medium to facilitate the detection and/or measurement of immune suppression (and/or resistance thereto). The capacity of the test cells to kill the target cells can be measured over the period of time (e.g., once or more every day) to detect and/or measure (i) the number of living and/or dead cells, (ii) changes in the rate at which cells proliferate and/or die, (iii) changes in the amount of living and/or dead cells, and/or (iv) changes to any of the preceding parameters in the presence and/or absence of one or more immunosuppressive factors. Alternatively, or in addition to, cell culture medium may be collected over the period of time to detect and/or measure (i) the presence or absence of one or more cytokines, (ii) changes in the presence or absence of one or more cytokines, (iii) the amount of the one or more cytokines, and/or (iv) changes in the amount of the one or more cytokines. Non-limiting examples of cytokines that may be assessed include: (i) granulocyte colony-stimulating factor (G-CSF), (ii) granulocyte-macrophage colony-stimulating factor (GM-CSF), (iii) interferon alpha (IFN-α), (iv) interferon gamma (IFN-γ), (v) interleukin-1 beta (IL-10), (vi) IL-2, (vii) IL-4, (viii) IL-5, (ix) IL-6, (x) IL-8, (xi) IL-10, (xii) interleukin-12 p70 (IL-12 p70), (xiii) IL-13, (xiv) IL-17A, (xv) IL-18, (xvi) interferon gamma-induced protein 10 (IP-10), (xvii) monocyte chemoattractant protein-1 (MCP-1), (xviii) macrophage inflammatory protein-1 alpha (MIP-la), (xix) MIP-1 beta (MIP-1β), (xx) tumor necrosis factor alpha (TNF-α), (xxi) TNF-β, or any combination thereof.

### 3. Cytotoxic Capacity and Killing Efficiency:

In some embodiments, the one or more genetic modifications to the one or more CRL5 complex-independent genes and the one or more genetic modifications to the one or more CRL5 complex genes improve the cytotoxic capacity of the resultant genetically modified hematopoietic cell by about 30.0% to about 100.0% compared to cytotoxic capacity of a control hematopoietic cell, as determined by an assay. In some embodiments, the one or more genetic modifications to the one or more CRL5 complex-independent genes and the one or more genetic modifications to the one or more CRL5 complex genes improve the cytotoxic capacity of the resultant genetically modified hematopoietic cell by about 30.0% to about 60.0%, about 60.0% to about 100.0%, about 60.0% to about 65.0%, about 67.0% to about 72.0%, about 78.0% to about 87.0%, about 90.0% to about 100.0%, about 65.0% to about 7 0.0%, about 7 0.0% to about 75.0%, about 75.0% to about 80.0%, about 80.0% to about 85.0%, about 85.0% to about 90.0%, about 90.0% to about 95.0%, or about 95.0% to about 100.0% compared to cytotoxic capacity of a control hematopoietic cell, as determined by an assay.

Alternatively, or in addition to, the one or more genetic modifications to the one or more CRL5 complex-independent genes and the one or more genetic modifications to the one or more CRL5 complex genes improve the cytotoxic capacity of the resultant genetically modified hematopoietic cell by about 0.30-fold to about 1.00-fold compared to cytotoxic capacity of a control hematopoietic cell, as determined by an assay. In some embodiments, the one or more genetic modifications to the one or more CRL5 complex-independent genes and the one or more genetic modifications to the one or more CRL5 complex genes improve the cytotoxic capacity of the resultant genetically modified hematopoietic cell by about 0.30-fold to about 0.60-fold, about 0.60-fold to about 1.00-fold, about 0.60-fold to about 0.70-fold, about 0.67-fold to about 0.72-fold, about 0.78-fold to about 0.87-fold, about 0.70-fold to about 0.80-fold, about 0.80-fold to about 0.90-fold, or about 0.90-fold to about 1.00-fold compared to cytotoxic capacity of a control hematopoietic cell, as determined by an assay.

In further embodiments, the assay comprises: (i) a quantification of cells and/or cell clusters using a cell imaging system (e.g., Incucyte), (ii) a serial challenge assay (with and/or without assessment of cells by (a) fluorescence-activated cell sorting (FACS), and/or (b) live cell imaging (e.g., using Incucyte)), (iii) a serial challenge assay (with and/or without the addition of one or more immunosuppressive factors to the cell culture medium), (iv) an enzymatic cytotoxicity assay, (v) an apoptosis assay, (vi) an autophagy assay, (vii) a cell viability assay, or (viii) a short-term killing assay.

In some embodiments, the one or more genetic modifications to the one or more CRL5 complex-independent genes and the one or more genetic modifications to the one or more CRL5 complex genes improve the killing efficiency of cancer cells by the resultant genetically modified hematopoietic cell by about 60.0% to about 100.0% compared to the killing efficiency of cancer cells by a control hematopoietic cell, as determined by an assay. In some embodiments, the one or more genetic modifications to the one or more CRL5 complex-independent genes and the one or more genetic modifications to the one or more CRL5 complex genes improve the killing efficiency of cancer cells by the resultant genetically modified hematopoietic cell by about 60.0% to about 100.0%, about 60.0% to about 65.0%, about 67.0% to about 72.0%, about 78.0% to about 87.0%, about 90.0% to about 100.0% about 65.0% to about 7 0.0%, about 7 0.0% to about 75.0%, about 75.0% to about 8 0.0%, about 8 0.0% to about 85.0%, about 85.0% to about 90.0%, about 90.0% to about 95.0%, or about 95.0% to about 100.0% compared to the killing efficiency of cancer cells by a control hematopoietic cell, as determined by an assay.

Alternatively, or in addition to, the one or more genetic modifications to the one or more CRL5 complex-independent genes and the one or more genetic modifications to the one or more CRL5 complex genes improve the killing efficiency of cancer cells by the resultant genetically modified hematopoietic cell by about 0.60-fold to about 1.00-fold compared to the killing efficiency of cancer cells by a control hematopoietic cell, as determined by an assay. In some embodiments, the one or more genetic modifications to the one or more CRL5 complex-independent genes and the one or more genetic modifications to the one or more CRL5 complex genes improve the killing efficiency of cancer cells by the resultant genetically modified hematopoietic cell by about 0.60-fold to about 1.00-fold, about 0.60-fold to about 0.70-fold, about 0.67-fold to about 0.72-fold, about 0.78-fold to about 0.87-fold, about 0.70-fold to about 0.80-fold, about 0.80-fold to about 0.90-fold, or about 0.90-fold to about 1.00-fold compared to the killing efficiency of cancer cells by a control hematopoietic cell, as determined by an assay.

In further embodiments, the assay comprises: (i) a quantification of cells and/or cell clusters using a cell imaging system (e.g., Incucyte), (ii) a serial challenge assay (with and/or without assessment of cells by (a) fluorescence-activated cell sorting (FACS), and/or (b) live cell imaging (e.g., using Incucyte)), (iii) a serial challenge assay (with and/or without the addition of one or more immunosuppressive factors to the cell culture medium), (iv) an enzymatic cytotoxicity assay, (v) an apoptosis assay, (vi) an autophagy assay, (vii) a cell viability assay, or (viii) a short-term killing assay.

### 4. Cytokine Production:

In some embodiments, the one or more genetic modifications to the one or more CRL5 complex-independent genes and the one or more genetic modifications to the one or more CRL5 complex genes improve capability of the resultant genetically modified hematopoietic cell to stimulate production of one or more cytokines by about 5.0% to about 3 0,000.0% compared to capability to stimulate production of one or more cytokines of a control hematopoietic cell, as determined by an assay. In some embodiments, the one or more genetic modifications to the one or more CRL5 complex-independent genes and the one or more genetic modifications to the one or more CRL5 complex genes improve capability of the resultant genetically modified hematopoietic cell to stimulate production of one or more cytokines by about 5.0% to about 510.0%, about 5.0% to about 10.0%, about 10.0% to about 25.0%, about 15.0% to about 20.0%, about 25.0% to about 3 0.0%, about 55.0% to about 60.0%, about 7 0.0% to about 7 5.0%, about 80.0% to about 8 5.0%, about 90.0% to about 100.0%, about 120.0% to about 125.0%, about 160.0% to about 165.0%, about 205.0% to about 210.0%, about 280.0% to about 3000.0%, about 340.0% to about 3 50.0%, about 25.0% to about 50.0%, about 50.0% to about 100.0%, about 100.0% to about 150.0%, about 150.0% to about 2000.0%, about 2000.0% to about 250.0%, about 250.0% to about 3000.0%, about 3000.0% to about 3 50.0%, or about 505.0% to about 510.0% compared to capability to stimulate production of one or more cytokines of a control hematopoietic cell, as determined by an assay. In some embodiments, the one or more genetic modifications to the one or more CRL5 complex-independent genes and the one or more genetic modifications to the one or more CRL5 complex genes improve capability of the resultant genetically modified hematopoietic cell to stimulate production of one or more cytokines by about 1000% to about 2000%, about 2000% to about 3000%, about 3000% to about 4000%, about 4000% to about 5000%, about 5000% to about 10,000%, about 10,000% to about 15,000%, about 15,000% to about 20,000%, about 20,000% to about 25,000%, or about 25,000% to about 30,000% compared to capability to stimulate production of one or more cytokines of a control hematopoietic cell, as determined by an assay.

Alternatively, or in addition to, the one or more genetic modifications to the one or more CRL5 complex-independent genes and the one or more genetic modifications to the one or more CRL5 complex genes improve capability of the resultant genetically modified hematopoietic cell to stimulate production of one or more cytokines by about 0.05-fold to about 300.0-fold compared to capability to stimulate production of one or more cytokines of a control hematopoietic cell, as determined by an assay. In some embodiments, the one or more genetic modifications to the one or more CRL5 complex-independent genes and the one or more genetic modifications to the one or more CRL5 complex genes improve capability of the resultant genetically modified hematopoietic cell to stimulate production of one or more cytokines by about 0.05-fold to about 5.10-fold, about 0.05-fold to about 0.25-fold, about 0.05-fold to about 0.10-fold, about 0.15-fold to about 0.20-fold, about 0.25-fold to about 0.30-fold, about 0.55-fold to about 0.60-fold, about 0.70-fold to about 0.75-fold, about 0.80-fold to about 0.85-fold, about 0.90-fold to about 1.00-fold, about 1.20-fold to about 1.25-fold, about 1.60-fold to about 1.65-fold, about 2.05-fold to about 2.10-fold, about 2.80-fold to about 3.00-fold, about 3.40-fold to about 3.5-fold, about 0.25-fold to about 0.50-fold, about 0.50-fold to about 1.00-fold, about 1.00-fold to about 1.50-fold, about 1.50-fold to about 2.00-fold, about 2.00-fold to about 2.50-fold, about 2.50-fold to about 3.00-fold, about 3.00-fold to about 3.50-fold, or about 5.05-fold to about 5.10-fold compared to capability to stimulate production of one or more cytokines of a control hematopoietic cell, as determined by an assay. In some embodiments, the one or more genetic modifications to the one or more CRL5 complex-independent genes and the one or more genetic modifications to the one or more CRL5 complex genes improve capability of the resultant genetically modified hematopoietic cell to stimulate production of one or more cytokines by about 10.0-fold to about 20.0-fold, about 20.0-fold to about 30.0-fold, about 30.0-fold to about 40.0-fold, about 40.0-fold to about 50.0-fold, about 50.0-fold to about 100.0-fold, about 100.0-fold to about 150.0-fold, about 150.0-fold to about 200.0-fold, about 200.0-fold to about 250.0-fold, or about 250.0-fold to about 300.0-fold compared to capability to stimulate production of one or more cytokines of a control hematopoietic cell, as determined by an assay.

In further embodiments, the one or more cytokines are selected from a group including: (i) granulocyte colony-stimulating factor (G-CSF), (ii) granulocyte-macrophage colony-stimulating factor (GM-CSF), (iii) interferon alpha (IFN-α), (iv) interferon gamma (IFN-γ), (v) interleukin-1 beta (IL-1β), (vi) IL-2, (vii) IL-4, (viii) IL-5, (ix) IL-6, (x) IL-8, (xi) IL-10, (xii) interleukin-12 p70 (IL-12 p70), (xiii) IL-13, (xiv) IL-17A, (xv) IL-18, (xvi) interferon gamma-induced protein 10 (IP-10), (xvii) monocyte chemoattractant protein-1 (MCP-1), (xviii) macrophage inflammatory protein-1 alpha (MIP-la), (xix) MIP-1 beta (MIP-1β), (xx) tumor necrosis factor alpha (TNF-α), (xxi) TNF-β, or any combination thereof.

In yet further embodiments, the assay comprises: (i) a ProcartaPlex multiplex cytokine assay, (ii) a Human LEGENDplex TH1 panel, (iii) an AlphaLISA assay, (iv) an intracellular cytokine stain assay, (v) a Luminex bead-based cytokine release assay, (vi) a MACS cytokine secretion assay, (vii) an ELISA, (viii) an ELISpot assay, or (ix) an assessment of cell culture medium to detect the presence and/or measure the amount of one or more cytokines following any of the assays described herein.

### H Control Hematopoietic Cells

In some embodiments, the control hematopoietic cell is selected from a group including: (i) a wild-type NK cell, (ii) a wild-type T-cell, and (iii) a wild-type TIL. In some embodiments, the control hematopoietic cell is a wild-type NK cell. In some further embodiments, the wild-type NK cell comprises a human wild-type NK cell or a human-derived wild-type NK cell. Alternatively, in some other embodiments, the wild-type NK cell comprises a mouse wild-type NK cell or a mouse-derived wild-type NK cell. In some embodiments, the control hematopoietic cell is a wild-type T-cell. In some further embodiments, the wild-type T-cell comprises a human wild-type T-cell or a human-derived wild-type T-cell. Alternatively, in some other embodiments, the wild-type T-cell comprises a mouse wild-type T-cell or a mouse-derived wild-type T-cell. In some embodiments, the control hematopoietic cell is a wild-type TIL. In some further embodiments, the wild-type TIL comprises a human wild-type TIL or a human-derived wild-type TIL. Alternatively, in some other embodiments, the wild-type TIL comprises a mouse wild-type TIL or a mouse-derived wild-type TIL.

As used herein, the terms “human-derived” or “mouse-derived,” when discussed in the context of a wild-type hematopoietic cell (such as, for example, a wild-type NK cell, a wild-type T-cell, a wild-type TIL, and the like) refer to any cell or cell line which can be obtained by any means of cultivation and cloning from a human or mouse, respectively. The cultivation and cloning are based on the fact that cell clones can be obtained from primary cell cultures (i.e., the generation of a cell culture using one or more cells isolated, harvested, or otherwise obtained from a subject (such as, for example, a human or a mouse)), cultures of cells (e.g., using an established cell line, optionally one that is immortalized), or even cultures of cell clones by multiple rounds of passaging and cloning of cells using techniques known in the art, including, but not limited to limited dilution or flow cytometry-based cell sorting. As such, it is expressly contemplated that, in some embodiments, the control hematopoietic cell can be a hematopoietic cell obtained directly by sampling one or more tissues, organs, or bodily fluids from a human, or that the control hematopoietic cell can be an established cell line that was derived from the sampling of one or more tissues, organs, or bodily fluids from a human. Similarly, it is also expressly contemplated that, in some embodiments, the control hematopoietic cell can be a hematopoietic cell obtained directly by sampling one or more tissues, organs, or bodily fluids from a mouse, or that the control hematopoietic cell can be an established cell line that was derived from the sampling of one or more tissues, organs, or bodily fluids from a mouse.

### IV. Methods of Making Genetically Modified Hematopoietic Cells

As will be appreciated by one skilled in the art, any aspects and embodiments of the methods of making described herein can utilize any of the aspects and/or embodiments of the compositions described herein and can further be used in any of the aspects and/or embodiments of the methods of use also described herein.

### A. Inhibition of CRL5 Complex Genes

In another aspect, the present disclosure provides a method of generating a genetically modified cell population for treatment of a subject that has cancer, the method comprising: (a) obtaining a plurality of hematopoietic cells; (b) inhibiting expression of one or more CRL5 complex genes, wherein the one or more CRL5 complex genes is selected from a group including: (i) ARIH2, (ii) CUL5, (iii) DCUN1D3, (iv) RNF7, (v) UBE2F, and (vi) any combination thereof; (c) selecting hematopoietic cells in which the one or more CRL5 complex genes are inhibited; and (d) expanding the selected hematopoietic cells ex vivo, thereby generating a genetically modified cell population for treatment of a subject that has cancer.

In some embodiments, the plurality of hematopoietic cells is derived from (i) the subject that has cancer (i.e., the plurality of hematopoietic cells is derived from an autologous source), (ii) a donor that does not have cancer (i.e., the plurality of hematopoietic cells is derived from an allogeneic source), or (iii) a cell line (i.e., the population of cells is derived from a cell line, as further described herein).

In some embodiments, the cell line is selected from a group including: (i) an induced pluripotent stem cell (iPSC) line, and (ii) a hematopoietic stem cell (HSC) line.

### B. Inhibition of CRL5 Complex-Independent Genes

In another aspect, the present disclosure provides a method of generating a genetically modified cell population for treatment of a subject that has cancer, the method comprising: (a) obtaining a plurality of hematopoietic cells; (b) inhibiting expression of one or more CRL5 complex-independent genes, wherein the one or more CRL5 complex-independent genes is selected from a group including: (i) ARHGAP25, (ii) CCNC, (iii) FAM49B, (iv) N4BP1, (v) STUB1, or (vi) any combination thereof; (c) selecting hematopoietic cells in which the one or more CRL5 complex-independent genes are inhibited; and (d) expanding the selected hematopoietic cells ex vivo, thereby generating a genetically modified cell population for treatment of a subject that has cancer.

In some embodiments, the plurality of hematopoietic cells is derived from (i) the subject that has cancer (i.e., the plurality of hematopoietic cells is derived from an autologous source), (ii) a donor that does not have cancer (i.e., the plurality of hematopoietic cells is derived from an allogeneic source), or (iii) a cell line (i.e., the population of cells is derived from a cell line, as further described herein).

In some embodiments, the cell line is selected from a group including: (i) an induced pluripotent stem cell (iPSC) line, and (ii) a hematopoietic stem cell (HSC) line.

### C. Inhibition of CRL5 Complex Genes+CRL5 Complex-Independent Genes

In another aspect, the present disclosure provides a method of generating a genetically modified cell population for treatment of a subject that has cancer, the method comprising: (a) obtaining a plurality of hematopoietic cells; (b) inhibiting expression of one or more CRL5 complex-independent genes, wherein the one or more CRL5 complex-independent genes is selected from a group including: (i) ARHGAP25, (ii) CCNC, (iii) FAM49B, (iv) N4BP1, (v) STUB1, or (vi) any combination thereof, (c) inhibiting expression of one or more CRL5 complex genes, wherein the one or more CRL5 complex genes is selected from a group including: (i) ARIH2, (ii) CUL5, (iii) DCUN1D3, (iv) RNF7, (v) UBE2F, (vi) CISH, and (vii) any combination thereof, (d) selecting hematopoietic cells in which the one or more CRL5 complex-independent genes and the one or more CRL5 complex genes are inhibited; and (e) expanding the selected hematopoietic cells ex vivo, thereby generating a genetically modified cell population for treatment of a subject that has cancer.

In some embodiments, the plurality of hematopoietic cells is derived from (i) the subject that has cancer (i.e., the plurality of hematopoietic cells is derived from an autologous source), (ii) a donor that does not have cancer (i.e., the plurality of hematopoietic cells is derived from an allogeneic source), or (iii) a cell line (i.e., the population of cells is derived from a cell line, as further described herein).

In some embodiments, the cell line is selected from a group including: (i) an induced pluripotent stem cell (iPSC) line, and (ii) a hematopoietic stem cell (HSC) line.

### V. Methods of Use

As will be appreciated by one skilled in the art, any aspects and embodiments of the methods of use described herein can utilize any of the aspects and/or embodiments of the compositions described herein and can further utilize any of the aspects and/or embodiments of the methods of making also described herein.

### A. Methods of Treating Cancer

In one aspect, the present disclosure provides a method of treating cancer, comprising: administering a population of cells comprising a genetically modified hematopoietic cell according to any of the aspects and embodiments of the genetically modified hematopoietic cells compositions and methods of making described herein.

As will be appreciated, any cancers amenable to treatment by cell therapy can be treated using the methods and compositions disclosed herein. Non-limiting examples of cancers that may be treated according to the methods described herein include: sarcoma, carcinoma, lymphoma, leukemia, myeloma, breast cancer, colon cancer, colorectal cancer, head and neck cancer, bone cancer, pancreatic cancer, skin cancer, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, multiple myeloma, Hodgkin's Disease, non-Hodgkin's lymphoma (NHL), primary mediastinal large B cell lymphoma (PMBC), diffuse large B cell lymphoma (DLBCL), follicular lymphoma (FL), transformed follicular lymphoma, splenic marginal zone lymphoma (SMZL), cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, chronic or acute leukemia, acute myeloid leukemia, chronic myeloid leukemia, acute lymphoblastic leukemia (ALL) (including non-T-cell ALL), chronic lymphocytic leukemia (CLL), lymphocytic lymphoma, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally induced cancers including those induced by asbestos, other B cell malignancies, and the like, as well as any combination(s) thereof.

In some embodiments, the population of cells is derived from (i) the subject that has cancer (i.e., the population of cells is derived from an autologous source), (ii) a donor that does not have cancer (i.e., the population of cells is derived from an allogeneic source), or (iii) a cell line (i.e., the population of cells is derived from a cell line, as described herein).

In some embodiments, the cell line is selected from a group including: (i) an induced pluripotent stem cell (iPSC) line, and (ii) a hematopoietic stem cell (HSC) line.

In some embodiments, the population of cells comprises about 100,000 (one hundred thousand) cells to about 100,000,000,000 (one hundred billion) cells. In some embodiments, the population of cells comprises about 100,000 to about 1 million cells, about 1 million to about 10 million cells, about 10 million to about 25 million cells, about 25 million to about 50 million cells, about 50 million to about 100 million cells, about 100 million to about 250 million cells, about 250 million to about 500 million cells, about 500 million to about 1 billion cells, about 1 billion to about 5 billion cells, about 5 billion to about 10 billion cells, about 10 billion to about 25 billion cells, about 25 billion to about 50 billion cells, about 50 billion to about 75 billion cells, or about 75 billion to about 100 billion cells.

The cells can be administered by any suitable means known in the relevant art, including, but not limited to bolus infusion, intravenous injection, intramuscular injection, subcutaneous injection, intraperitoneal injection, spinal injection, or other parental routes of administration. In some embodiments, a given “dose” of cells is administered by a single bolus administration of the cells. Alternatively, or in addition to, the population of cells can be administered by multiple bolus administrations of the cells (e.g., over a period of 1, 2, or 3 days), or by continuous infusion of the cells.

In some embodiments, the population of cells is administered as part of a combination treatment, such as, for example, simultaneously with or sequentially with (in any order) another therapeutic intervention (e.g., an antibody, an engineered cell (such as, for example, a cell comprising a CAR or a TCR construct), or another agent (such as, for example, a cytotoxic agent and/or a therapeutic agent). In some embodiments, the population of cells is co-administered with one or more additional therapeutic agents in connection with another therapeutic intervention, either simultaneously or sequentially (in any order). In some embodiments, the population of cells is co-administered with another therapy sufficiently close in time such that the population of cells enhances the effect of one or more additional therapeutic agents, or vice versa. In some embodiments, the population of cells is administered prior to the one or more additional therapeutic agents. In some embodiments, the population of cells is administered after the one or more additional therapeutic agents.

In some embodiments, the combination treatment comprises a first component comprising the population of cells, and a second component comprising a cytokine. Generally, the combination treatment is administered to the subject in a therapeutically effective amount. In some embodiments, the population of cells is engineered to express the cytokine. Alternatively, in other embodiments, the population of cells is not engineered to express the cytokine and the cytokine is administered simultaneously with or sequentially with (in any order) the population of cells. In some further embodiments, the cytokine comprises IL-15. The population of cells can be administered by any suitable means known in the relevant art, including, but not limited to bolus infusion, intravenous injection, intramuscular injection, subcutaneous injection, intraperitoneal injection, spinal injection, or other parental routes of administration. Similarly, the cytokine (e.g., IL-15) may be administered to the subject according to any suitable route known in the relevant art.

In some embodiments, prior to the administration (of the population of cells or the combination treatment), the subject has received a myeloablative conditioning regimen, a nonmyeloablative conditioning regimen, or a reduced intensity conditioning regimen. As used herein, the term “myeloablative conditioning regimen” refers to the administration of one or more chemotherapies, one or more instances of a radiation therapy, or a combination thereof, at doses and/or frequencies that cause irreversible cytopenia and require one or more infusions of stem cells from a donor for the survival of the subject. The subject may have received any suitable myeloablative conditioning regimen known in the relevant art. As used herein, the term “nonmyeloablative conditioning regimen” refers to the administration of one or more chemotherapies, one or more instances of a radiation therapy, or a combination thereof, at doses and/or frequencies that cause minimal cytopenia and do not require one or more infusions of stem cells from a donor for the survival of the subject. The subject may have received any suitable nonmyeloablative conditioning regimen known in the relevant art. As used herein, the term “reduced intensity conditioning regimen” refers to a conditioning regimen that does not fit the criteria for a myeloablative conditioning regimen or a nonmyeloablative conditioning regimen. Generally, reduced intensity conditioning regimens cause cytopenia of a finite duration, but still require one or more infusions of stem cells from a donor. The subject may have received any suitable reduced intensity conditioning regimen known in the relevant art. Suitable myeloablative, nonmyeloablative, and/or reduced intensity conditioning regimens can be determined by the persons skilled in the art.

### Examples

The following examples are presented in order to more fully illustrate some embodiments of the invention. They should, in no way be construed, however, as limiting the broad scope of the invention. One skilled in the art can readily devise many variations and modifications of the principles disclosed herein without departing from the scope of the invention.

### A. Example 1—Generating CRL5 Complex-Ablated Cells and Exemplary Assays for Assessing the Effects of the Genetic Modifications

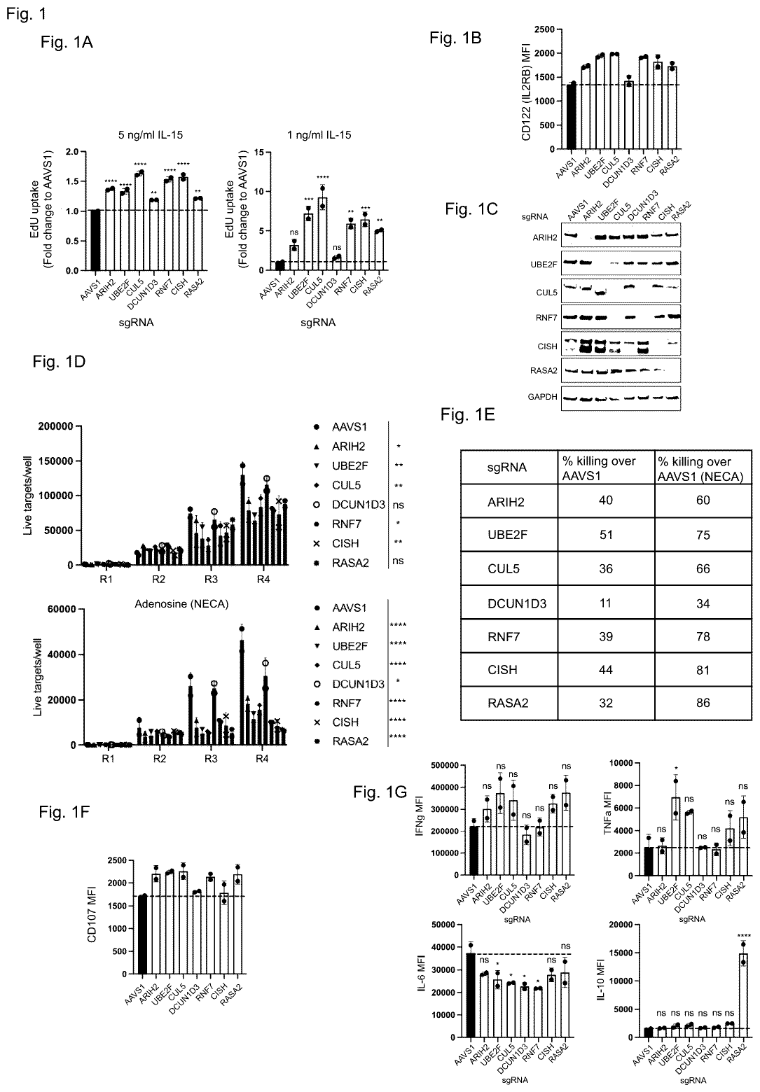

Current ACTs can have limited proliferative capacity and be susceptible to varying degrees of immune suppression. As a result of such deficiencies, the cytotoxic capacity and the ability of ACTs to stimulate the production of cytokines can be limited. It was hypothesized that inhibition of one or more components of the CRL5 complex in hematopoietic cells could result in the production of an ACT product with an increased proliferative capacity that was resistant to immune suppression and capable of stimulating the production of cytokines. In order to assess the effects of CRL5 complex ablation, primary human NK cells were edited using a CRISPR system to inhibit expression or activity of a select component of the CRL5 complex (ARIH2, UBE2F, CUL5, DCUN1D3, or RNF7), one of two positive control genes (CISH or RASA2), or a negative control gene (AAVS1), as shown in FIG. 1.

Methods used to generate the CRL5 complex-ablated hematopoietic cells and downstream assays to assess the effects of inhibiting select components of the CRL5 complex are described below.

1. Primary Human NK Cell Isolation from PBMC Donors:

Human blood samples were obtained and subjected to centrifugation to retrieve the buffy coat fraction of the blood. Subsequently, primary human NK cells were isolated from peripheral blood mononuclear cells (PBMCs) in the buffy coat fraction using a Human NK cell isolation kit (Miltenyi Biotec). The isolated cells were subsequently cultured in NK MACs medium (Miltenyi Biotec) supplemented with 10% horse serum (HS) and 5 ng/mL IL-15 and initially expanded using K562-41BBL-mIL-21 feeder cells. After 7 days of expansion, the NK cells were prepared for various assays, as described herein.

### 2. Primary Human T-Cell Isolation and Expansion:

T-cells were activated and expanded from PBMC donors in RPMI-1640 containing 10% fetal calf serum (FCS), 1× GlutaMAX, 1% pen./strep., 0.1% β-mercaptoethanol, 25 mM HEPES, 1% non-essential amino acids, and 1% sodium pyruvate in the presence of 32 ng/mL soluble OKT3 and 600 U/mL TL-2 for 48 h before RNP editing and TCR/CAR transduction. Vδ2⁺ T-cells were expanded from human PBMC donors in RPMI-1640 containing 10% fetal calf serum (FCS), 1× GlutaMAX, 1% pen./strep., 0.1% β-mercaptoethanol, 25 mM HEPES, 1% non-essential amino acids, and 1% sodium pyruvate in the presence of 5 μM Zoledronate and 100 U/mL IL-2 for 5 days before CAR transduction and RNP editing.

### 3. Viral Production and Lentiviral Transduction:

HEK293T cells were transfected with an ABECMA CAR expression construct or 1G4 TCR expression construct, as well as gag/pol and envelope plasmids, using Lipofectamine 3000. Viral supernatant was filtered (0.45 μM) and concentrated using ultracentrifugation (14,208g for 120 minutes). Concentrated virus was aliquoted and stored at −80° C. Each viral batch was titrated using mScarlet expression in the HEK293T cells to determine the viral titer (transduction units /mL of viral stock).

Following the 7-day expansion period described above, NK cells were seeded in Opti-MEM supplemented with 0.1% heat-inactivated fetal calf serum (FCS), 20 ng/mL IL-15, and 10 g/mL vectofusin (Miltenyi Biotec) in a 48-well plate with or without the packaged ABECMA CAR virus at a multiplicity of infection (MOI) of 5. The plate was centrifuged at 1200g for 60 minutes at 32° C. and allowed to incubate overnight. Following the incubation period, the cells were harvested, washed, and reseeded in standard NK cell culture medium in a G-Rex 48-well plate. To generate 1G4-TCR NK cells, expanded NK cells were double transduced with the 1G4 TCR lentivirus and a lentivirus encoding for CD3 complex as described above.

Activated T-cells were transduced with the packaged Abecma CAR or 1G4 TCR lentivirus using RetroNectin-coated plates and spininfection for 1 h at 1200 g at 32° C. Expanded γδ-T-cells were transduced with the packaged Abecma CAR lentivirus using 10 μg/mL vectofusin and spininfection for 1 h at 1200 g at 32° C.

### 4. CRISPR Ribonucleoprotein Editing:

CRISPR ribonucleoprotein (RNP) complexes were assembled using a purified Cas9 protein (61 μM) and sgRNA (100 μM) against the genes of interest in an electroporation buffer. The complex was added directly into the resuspended NK cells (1×10⁶ cells per reaction), which were subsequently electroporated using the CM137 pulse code (Amaxa, Lonza). Following electroporation, the cells were co-cultured with K562-41BBL-mIL-21 feeder cells for another 7 days to allow the cells to recover and expand. T-cells were electroporated using the same code and protocol for RNP editing.

### 5. Flow Cytometry:

NK cells were stained for 20-30 minutes at room temperature for select surface marker expression. Cells were then analyzed directly on a CytoFLEX S (Beckman Coulter; equipped with a 405 nm laser, a 488 nm laser, a 561 nm laser, and a 638 nm laser). Data were analyzed using FlowJo software (Versions 9.9.6 and 10.5.3; BD Bioscience). The following antibodies were used: CD56 (A700), CD56 (BV421), CD122-APC (Beckman Coulter); CD107a-PE (Miltenyi Biotec), and LIVE/DEAD Fixable Near-IR Dead Cell Stain kit (Invitrogen). For T-cell staining, the following antibodies were used: CD3 BV650, CD122-PE, CD8 BV605, and CD4 FITC. For γδ-T-cell staining, the following antibodies were used: CD3 BV650, and Vδ2 TCR BV421.

### 6. Proliferation Assay:

NK cell proliferation was measured using an EdU uptake assay. RNP-edited NK cells previously cultured either in high (5 ng/mL) or low (1 ng/mL) IL-15 containing medium were exposed to EdU labeling (2 μM final concentration) overnight. Following the incubation period, the cells were collected and the incorporated EdU was detected using the Click-IT reaction and FACS analysis according to the manufacturer's instructions. Statistics were calculated using one-way analysis of variance (ANOVA) with Dunnett's multiple comparison correction.

For quantifying EdU incorporation in T-cells, the same protocol was followed as above but T-cells were cultured in RPMI+10% FCS and 600 U/mL IL-2.

### 7. Serial Challenge Assay:

NK cells (or CAR-NK cells) were co-cultured with targets cells at different effector:target (E:T) ratios in a 96-well format. Culture medium was supplemented with a relatively low IL-15 concentration (1 ng/mL) with or without the inclusion of the immunosuppressive factor adenosine (NECA; 300 μM final concentration). Fresh target cells were added every 24 hours to allow for serial rounds of killing, up to four rounds in total. In the experiments that utilized CAR-NK cells, the initial co-culture E:T ratio was adjusted based on the CAR expression to satisfy the indicated CAR-NK to target cell ratio.

The same protocol was followed for 1G4 TCR NK cells, Abecma CAR-T-cells, and Abecma CAR γδ-T-cells. Adenosine (NECA) was used at 300 μM final concentration.

### 8. Cytokine Release Assay:

Supernatant was collected from the abovementioned co-culture assays following the third or fourth round of target cell killing. Supernatant was aliquoted and frozen at −80° C. IFN-γ, TNF-α, IL-6, and IL-10 cytokines were detected using the Human LEGENDplex Th1 panel with samples assayed in duplicate by flow cytometry (5-plex, BioLegend). In some assays, IFN-γ was measured using AlphaLISA human IFN-γ detection kit (PerkinElmer). Statistics were calculated using one-way ANOVA with Dunnett multiple comparison correction (ns indicates not significant, *P<0.05, **P<0.01, ***P<0.001, ****P<0.0001). For in vivo experiments, supernatants from digested tumors were collected and the cytokine levels were measured using LEGENDplex Th1 panel as described above.

### B. Example 2—CRL5 edits in primary human NK cells

After the CRL5 complex-ablated NK cells were generated, various experiments were performed to assess the effects of the genetic modifications.

To assess the effect of CRL5 complex ablation on the proliferative capacity of NK cells, an EdU uptake assay was performed in primary human NK cells CRISPR edited with one of the indicated sgRNAs (directed to one of the aforementioned components of the CRL5 complex, one of two positive control genes (CISH or RASA2), or a negative control gene (AAVS1)). The assay was performed in the presence of either a high concentration (5 ng/mL) or a low concentration (1 ng/mL) of IL-15, a cytokine known to promote the survival, proliferation, and cytotoxicity of NK cells. As shown in FIG. 1A, the fold change in EdU uptake was significantly increased in the CRL5 complex-ablated NK cells, as well as in NK cells in which the CRISPR system targeted CISH or RASA2 (sometimes referred to as the “positive controls”), compared to NK cells in which the CRISPR system targeted AAVS1 (sometimes referred to as the “negative control”). While CRL5 complex ablation resulted in a significant increase in NK cell proliferation when incubated in the presence of the high and low concentration of IL-15, changes in proliferative capacity were more pronounced for UBE2F-, CUL5-, and RNF7-ablated NK cells under low cytokine conditions (i.e., 1 ng/mL of IL-15).

CD122, also known as IL2Rβ, is an important marker for the later stages of NK cell development. As such, expression of CD122 was subsequently examined in the human NK cells edited with the indicated sgRNA. Except for DCUN1D3, the CRL5 complex-ablated NK cells and the positive controls exhibited an increase in CD122 compared to the negative control, as shown in FIG. 1B. Increased expression of this receptor at least partly explains the enhanced IL-15 signaling in the CRL5 edited cells, as CD122 is a receptor for both IL-2 and IL-15.

To assess the efficiency of the CRISPR-mediated knockouts, Western blotting was performed on protein lysates from the primary human NK cells edited with the indicated sgRNA. As shown in FIG. 1C, the Western blotting results generally demonstrated an efficient knockout for the indicated sgRNA (e.g., knocking out ARIH2 using a CRISPR system resulted in reduced protein expression of ARIH2, knocking out UBE2F using a CRISPR system resulted in reduced protein expression of UBE2F, etc.). Unexpectedly, when CUL5 was ablated in the NK cells, there was a concomitant reduction in the protein expression of CUL5 and RNF7.

Subsequently, the cytotoxic capacity of the CRL5 complex-ablated NK cells was assessed using a serial challenge assay with primary human NK cells edited with the indicated sgRNA and K562 cells as target cells. The assay was conducted under low IL-15 conditions (1 ng/mL IL-15) either in the absence or presence of the immunosuppressive factor adenosine (NECA), as shown in FIG. 1D (top panel and lower panel, respectively). Superior killing of the K562 target cells by the CRL5 complex-ablated NK cells became evident in the fourth challenge (“R4”) where the negative control NK cells underwent functional exhaustion. FIG. 1E shows a summary of the results of FIG. 1D presented as a percentage of increased killing compared to the negative control NK cells. CD107 is known to be a marker of NK cell degranulation and is considered to be a functional marker for NK cell activity. FIG. 1F shows the level of CD107 during the first round of the serial challenge assay. Increased levels of CD107 were exhibited in the CRL5 complex-ablated NK cells compared to the negative control, corroborating activation of the CRL5 complex-ablated NK cells.

To assess functional activation of NK cells following CRL5 complex ablation, cell culture supernatants were collected during the serial challenge assay and subjected to a multiplex cytokine assay. FIG. 1G shows the results of the cytokine release assay. Briefly, knocking out UBE2F in the primary human NK cells resulted in a significant increase in TNF-α secretion, and knocking out UBE2F, CUL5, DCUN1D3, or RNF7 resulted in a significant decrease in IL-6 secretion compared to the negative control.

### C. Example 3—CRL5 Complex-Ablated CAR-N K Cells Vs. Target Cells Engineered to Express BCMA

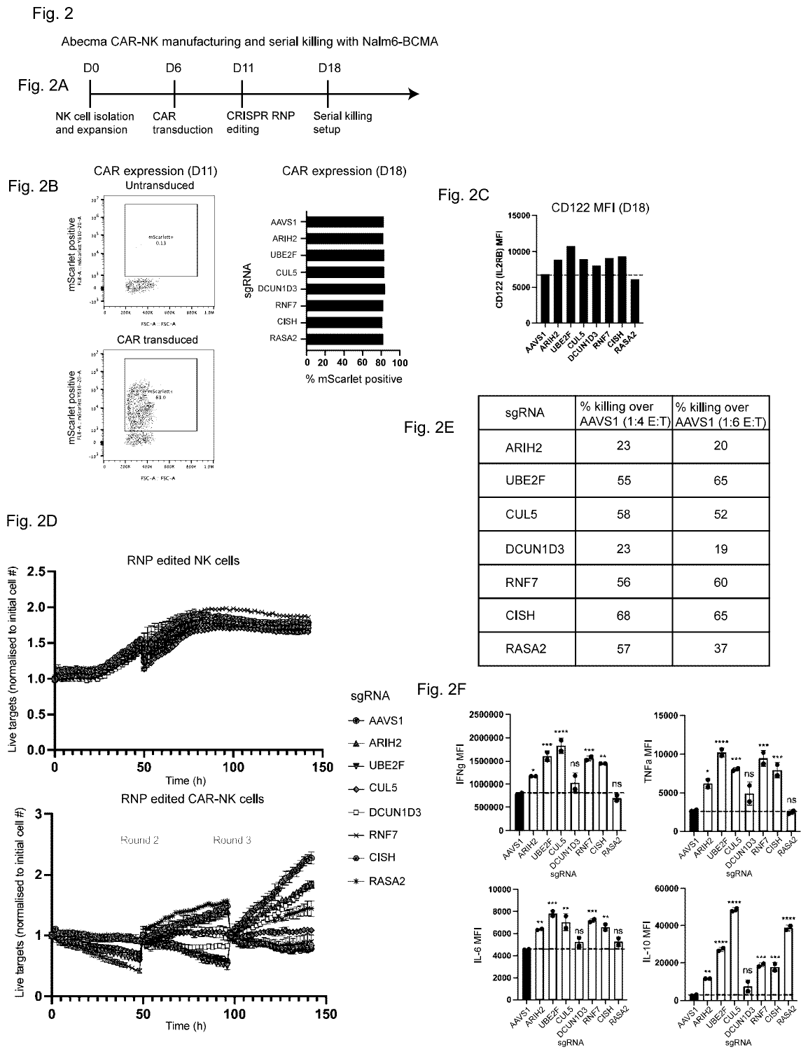

In an effort to further improve the cytotoxic capacity of the CRL5 complex-ablated NK cells, NK cells were transduced with an ABECMA CAR construct prior to CRISPR-mediated inhibition of a select component of the CRL5 complex (ARIH2, UBE2F, CUL5, DCUN1D3, or RNF7), one of two positive control genes (CISH or RASA2), or a negative control gene (AAVS1), as shown in FIG. 2.

FIG. 2A shows a schematic describing the manufacturing of NK cells modified using an ABECMA CAR construct followed by editing of one component of the CRL5 complex, CISH, RASA2, or the negative control gene AAVS1 using a CRISPR system. FIG. 2B shows representative FACS plots displaying the percentage of CAR transduction, as measured by mScarlet fluorescence, in the primary NK cells before and after CRISPR RNP editing (top panel and bottom panel, respectively). High CAR expression was maintained over time and was not affected by the RNP editing. As shown in FIG. 2C, elevated CD122 expression was observed in the CRL5 complex-ablated CAR-NK cells, similar to the CRL5 complex-ablated NK cells in Example 2.

The effect of CRL5-ablated NK cells and CRL5-ablated CAR-NK cells against target cells (Nalm6) that were designed to express BCMA (Nalm6-BCMA) was assessed using a serial challenge assay performed on an Incucyte, as shown in FIG. 2D (top panel and bottom panel, respectively). The results of the serial challenge assay are summarized in FIG. 2E as a percentage of increased killing compared to the negative control. NK cells edited with the indicated sgRNA were plated with Nalm6-BCMA cells at an E:T ratio of 1:4 or 1:6 (E:T ratio of 1:4 not shown in FIG. 2D). The NK cells that were not transfected with the ABECMA CAR construct did not show natural cytotoxicity against the target cells, whereas some of the CAR-NK cells exhibited varying degrees of cytotoxicity against the target cells. By the third round of the serial challenge assay, the negative control cells had exhausted their proliferative capacity and stopped killing the target cells, whereas the CRL5 complex-ablated CAR-NK cells continued to exhibit cytotoxicity against the target cells. The efficiency of increased killing varied between the different CRL5 complex-ablated targets, with CUL5 being the most effective CRL5 complex edit at an E:T ratio of 1:4 and UBE2F being the most effective CRL5 complex edit at an E:T ratio of 1:6.

To assess functional activation of CAR-NK cells following CRL5 complex ablation, cell culture supernatants were collected during the serial challenge assay and subjected to a multiplex cytokine assay. FIG. 2F shows the results of the cytokine release assay. Briefly, knocking out ARIH2, UBE2F, CUL5, or RNF7 in the CAR-NK cells resulted in a significant increase in IFN-γ, TNF-α, IL-6, and IL-10 secretion compared to the negative control.

### D. Example 4—CRL5 Complex-Ablated CA R-N K Cells Vs. Target Cells Exhibiting Endogenous Expression of BCMA

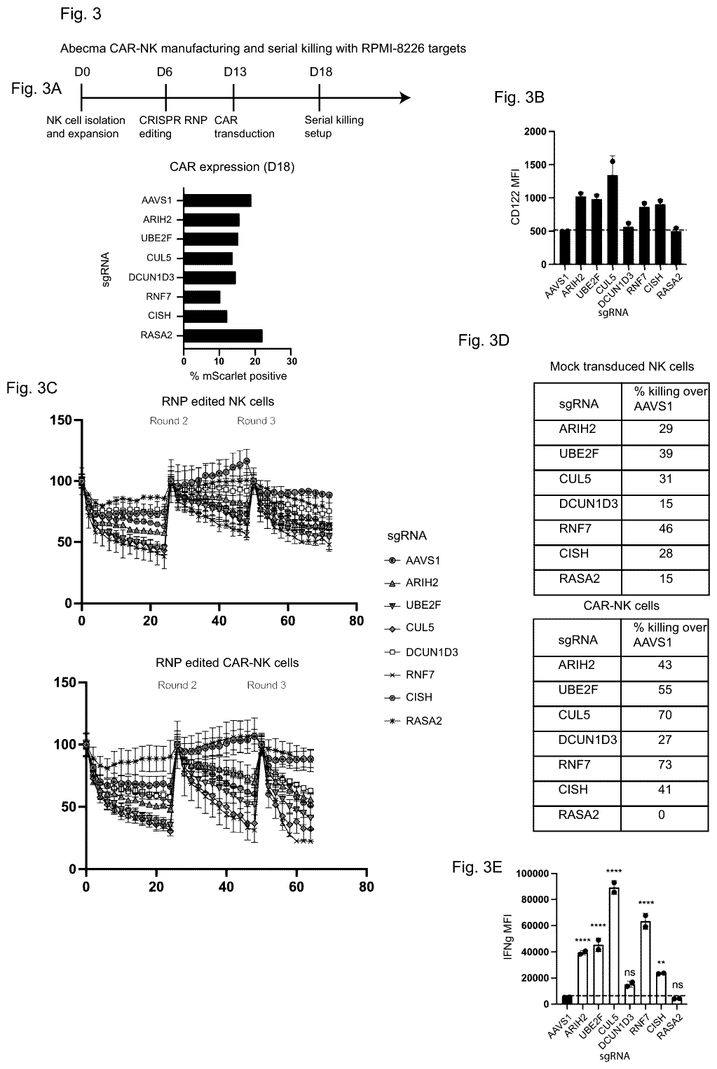

Subsequently, the effect of CRL5-ablated NK cells and CRL5-ablated CAR-NK cells against target cells (RPMI-8226) exhibiting endogenous expression of BCMA was assessed, as shown in FIG. 3.

FIG. 3A shows a schematic describing the manufacturing of NK cells modified using an ABECMA CAR construct followed by editing of one component of the CRL5 complex, CISH, RASA2, or the negative control gene AAVS1 using a CRISPR system. As shown in FIG. 3B, elevated CD122 expression was observed in the CRL5 complex-ablated CAR-NK cells, similar to the CRL5 complex-ablated NK cells in Example 2.

The effect of CRL5-ablated NK cells and CRL5-ablated CAR NK cells against target cells (RPMI-8226) that exhibit endogenous expression of BCMA was assessed using a serial challenge assay performed on an Incucyte, as shown in FIG. 3C (top panel and bottom panel, respectively). The results of the serial challenge assay are summarized in FIG. 3D as a percentage of increased killing compared to the negative control. NK cells edited with the indicated sgRNA were plated with RPMI-8266 cells at an E:T ratio of 1:2 (target cell numbers normalized based on the CAR expression). The NK cells that were not transfected with the ABECMA CAR construct exhibited some natural cytotoxicity against the target cells. Ablation of the CRL5 complex resulted in an increase in target cell killing compared to the negative control. Target cell killing was more pronounced in the CRL5 complex-ablated CAR-NK cells. In the mock transduced NK cells, targeting of RNF7 using the CRISPR system resulted in the largest percent increase in target cell killing compared to the negative control. In the CAR-NK cells, targeting of RNF7 using the CRISPR system also resulted in the largest percent increase in target cell killing compared to the negative control, with CUL5 ablation exhibiting only a slightly lower increased percent killing compared to RNF7.

To assess functional activation of CAR-NK cells following CRL5 complex ablation, cell culture supernatants were collected during the serial challenge assay and IFN-γ was measured using AlphaLISA human IFN-γ detection kit. FIG. 3E shows the results of the IFN-γ AlphaLISA assay. Briefly, knocking out ARIH2, UBE2F, CUL5, or RNF7 in the CAR-NK cells resulted in a significant increase in IFN-γ secretion compared to the negative control.

### E. Example 5—Screening and Identification of Genetic Targets (CRL5 Complex-Independent Genes)

Current ACTs can have limited proliferative capacity and be susceptible to varying degrees of immune suppression. As a result of such deficiencies, the cytotoxic capacity and the ability of ACTs to stimulate the production of cytokines can be limited. In an effort to identify potential genetic targets for the generation of improved ACT products, high-throughput CRISPR screens were performed in human NK cells to interrogate different aspects of the NK cell phenotype and function.

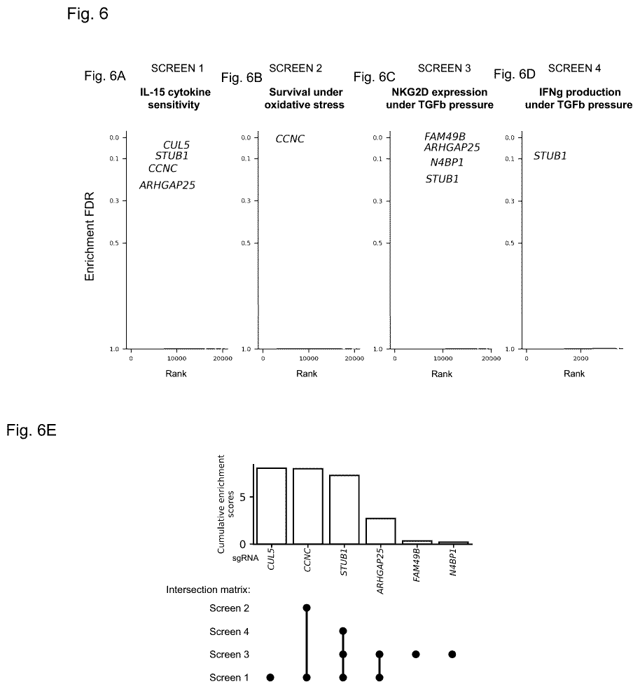

The graphs shown in FIGS. 6A-6E depict CRISPR screen results highlighting gRNA enrichment ranks and overall significance (represented as false discovery rate (FDR) values) for positive controls in each of the performed screens. Generally, a lower rank signifies a beneficial phenotype associated with a genetic ablation.

FIG. 6A depicts the results of the first high-throughput CRISPR screen (IL-15 cytokine sensitivity). Briefly, Cas9⁺ NK-92 cells carrying a sgRNA library were cultured under low IL-15 cytokine conditions (1 ng/mL) for 18 days, and surviving clones were subjected to nucleic acid extraction, sequencing, and quantification. Changes in the relative frequency of edited cell clones reflect changes in cell proliferation and/or survival, such that genes with a low rank demonstrated gRNA enrichment, and deletion of this gene was beneficial. Conversely, genes with a high rank showed gRNA dropout due to the deleterious effects of losing this gene. Guides targeting STAT5 signaling components (i.e., STAT5A, STAT5B, IL2RB, and IL2RG) showed strong dropout and correspondingly carry high ranks. Guide RNAs targeting CISH (a known negative regulator of STAT5 signaling) showed strong enrichment, reflecting the survival benefits associated with loss of this gene under low IL-15 conditions.

FIG. 6B depicts the results of the second high-throughput CRISPR screen (survival under oxidative stress). Briefly, Cas9⁺ NK-92 cells carrying a sgRNA library were cultured under optimal IL-15 cytokine conditions with the addition of Kynurenine at 400 uM for 14 days, which is known to induce oxidative stress and result in a loss of cell viability. Subsequently, clones that survived (low rank) or dropped out (high rank) of the population were subjected to nucleic acid extraction, sequencing, and quantification. Guides targeting components of the glutathione signaling pathway (i.e., ESD, GCLM, SLC7A11, and GSTP1) showed the strongest dropout.

FIG. 6C depicts the results of the third high-throughput CRISPR screen (NKG2D expression under TGF-β pressure). Briefly, Cas9⁺ NK-92 cells carrying a sgRNA library were treated with TGF-β (5 ng/mL) for 48h, and the clones were subjected to FACS sorting based on NKG2D expression (high NKG2D expression corresponding to low rank, and low NKG2D expression corresponding to high rank). The FACS sorted cells were subjected to nucleic acid extraction and sequencing. Guides targeting KLRK1 (NKG2D) and the main signaling adaptor HCST were top hits in the NKG2D low group. Guides targeting the TGF-β signaling pathway enriched in the NKG2D high group.

FIG. 6D depicts the results of the fourth high-throughput CRISPR screen (IFN-γ production under TGF-β pressure). Briefly, primary NK cells (obtained from a donor) carrying a druggable sgRNA library were edited with a Cas9 nuclease, treated with the cytokines IL-12 (2.5 ng/ml) and IL-18 (6.25 ng/ml) overnight in the presence of TGF-β (5 ng/ml) for 6h, and the clones were subjected to FACS sorting based on IFN-γ expression (gene knockouts associated high IFN-γ expression corresponding to a low rank, and knockouts associated with low IFN-γ expression corresponding to high rank). The FACS sorted cells were subjected to nucleic acid extraction and sequencing. Guides targeted against IFN-γ and IL-12 receptor signaling components were top hits in the IFN-γ low group. Guides targeting TGF-β receptor and intracellular signal transduction components were enriched in the IFN-γ high group.

FIG. 6E depicts the cumulative enrichment score for indicated gene candidates across all screens. Generally, a high cumulative enrichment score reflects a low rank in a particular screen and/or a low rank across multiple screens. FIG. 6E further depicts an intersection matrix below the bar graph indicating if a gene scored as a significant low rank hit in a screen. Analysis of the data from the aforementioned high-throughput CRISPR screens resulted in the identification of various potential targets orthogonal to the CRL5 complex, as well as CUL5 (a representative member of the CRL5 complex). Briefly, genetic ablation of CUL5 resulted in a significant low rank with respect to screen 1. Genetic ablation of CCNC resulted in a significant low rank with respect to screens 1 and 2. Genetic ablation of STUB1 resulted in a significant low rank with respect to screens 1, 3, and 4. Genetic ablation of ARHGAP25 resulted in a significant low rank with respect to screens 1 and 3. Genetic ablation of FAM49B or N4BP1 both resulted in a significant low rank with respect to screen 3.

### F. Example 6—Initial Hit Validation

In an effort to validate the results identified in the high throughput CRISPR screens (see, e.g., Example 5), NK cells derived from human donors were subjected to CRISPR-mediated ablation of select targets (as described in further detail below; see, e.g., Example 7).

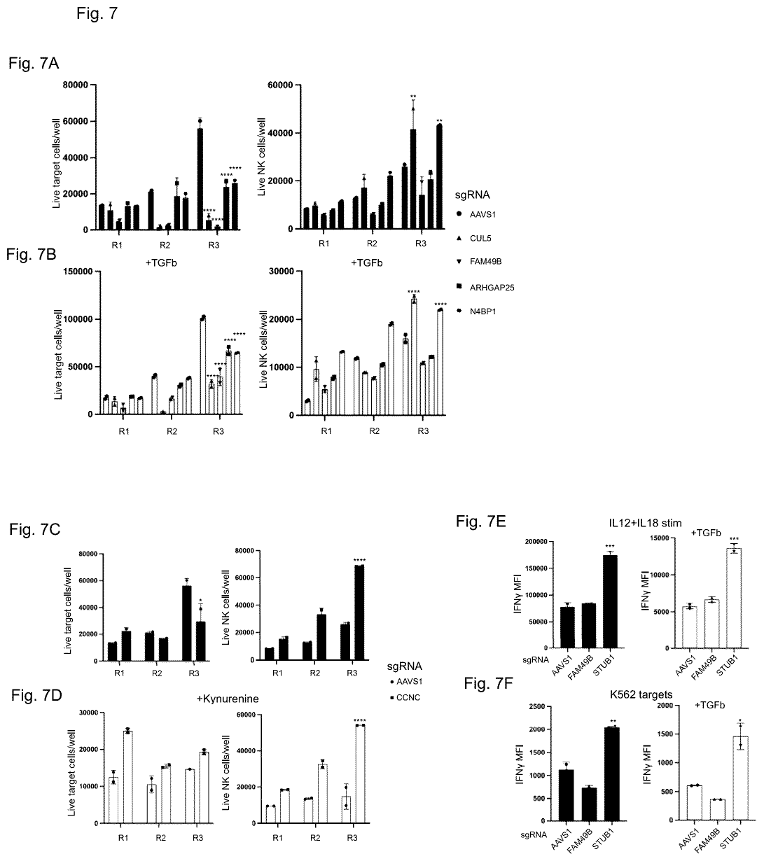

As shown in FIGS. 7A and 7B, hits identified in the cytotoxicity screen (NKG2D expression) in the context of TGF-β suppression were tested in a serial challenge assay and assessed by FACS. Both live target cells and live NK cells were quantified in each well. By round 3 (R3) of the assays, negative control NK cells (sgRNA directed to AAVS1) underwent functional exhaustion, whereas NK cells edited with guides against CUL5 (positive control) or one of the targets identified in the CRISPR screens (FAM49B, ARHGAP25, and N4BP1) continued killing target cells in the absence or presence of TGF-β (5 ng/mL), as shown in FIG. 7A and FIG. 7B, respectively. N4BP1 single-edited NK cells, as well as CUL5 single-edited NK cells, demonstrated an increase in NK cell proliferation during killing.

As shown in FIG. 7C, NK cell editing against CCNC modestly increased NK cell killing in the serial challenge assays, but it enabled significant NK cell proliferation during killing. As shown in FIG. 7D, CCNC editing did not increase the killing capacity of NK cells in the presence of Kynurenine (400 uM). However, cellular survival and proliferation were maintained.

As shown in FIGS. 7E and 7F, NK cell editing against STUB1 significantly increased the capacity of NK cells to produce IFN-γ in response to stimulation from the cytokines IL-12 (2.5 ng/ml) and IL-18 (6.25 ng/ml), as shown in FIG. 7E, or during co-culturing with K562 target cells, as shown in FIG. 7F. This effect was observed in the presence or absence of TGF-β (5 ng/mL). In comparison, NK cell editing against FAM49B did not induce this effect, consistent with the results observed in the primary screening data (see, e.g., Example 5).

### G. Example 7—Generating Cells Wherein Genes Orthogonal to the CRL5 Complex are ablated and exemplary assays for assessing the effects of the genetic modifications

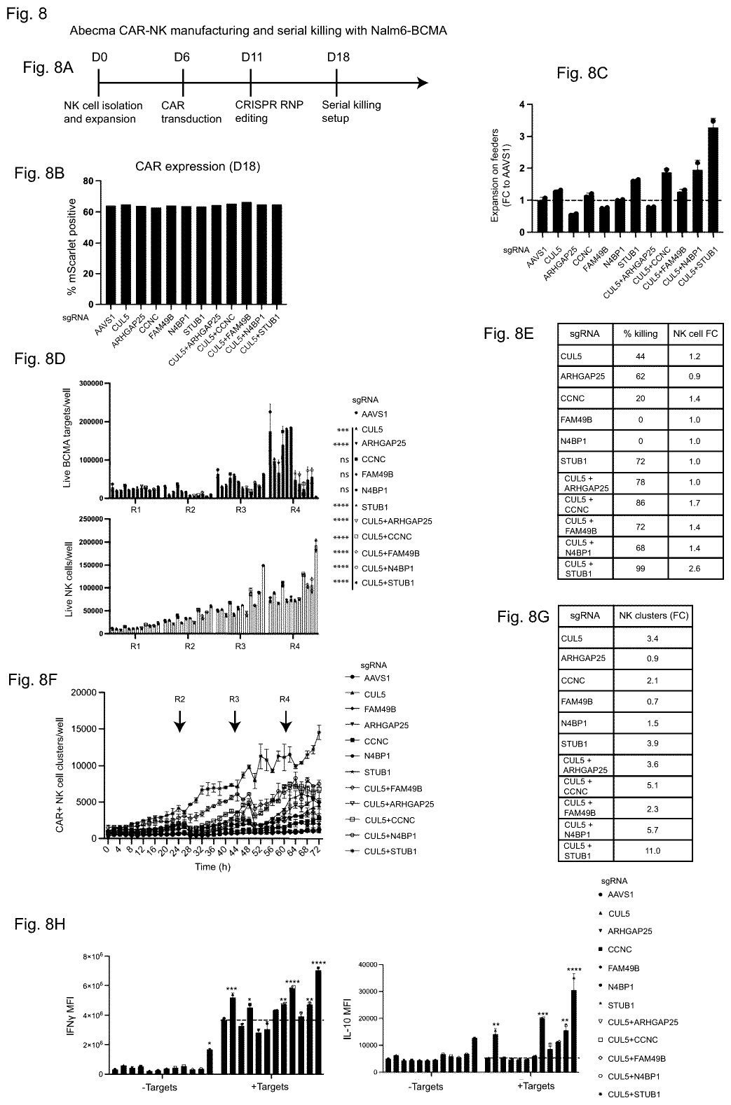

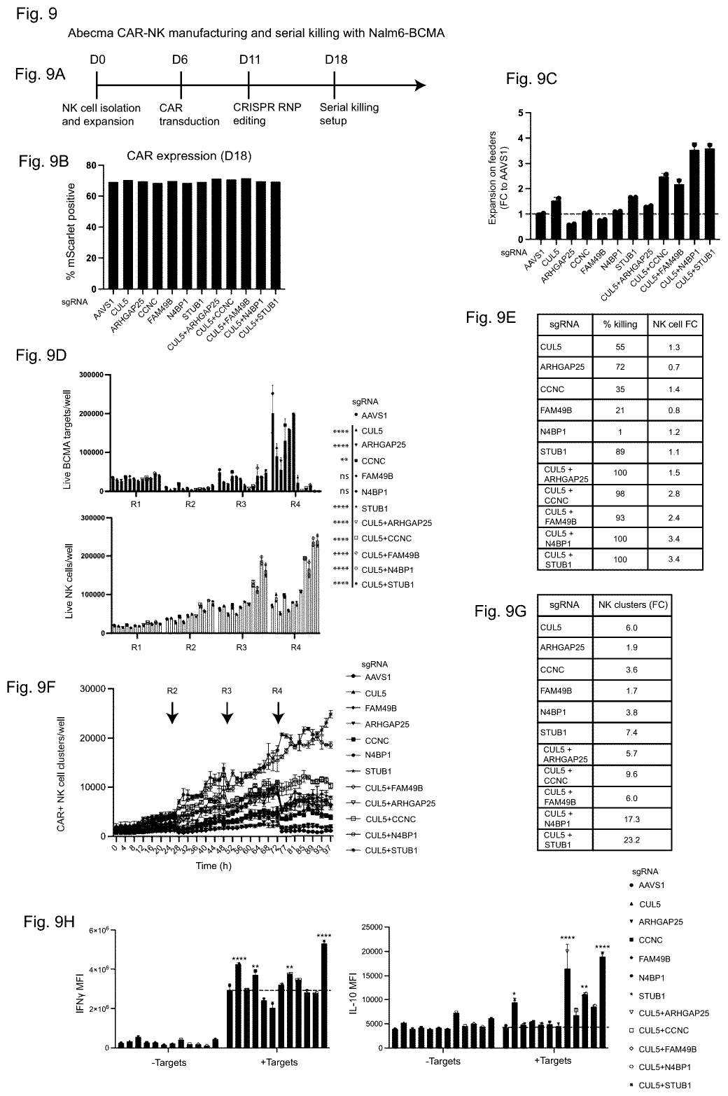

Based on the results described in Examples 5 and 6, it was hypothesized that inhibition of one or more genes orthogonal to the CRL5 complex (alone, or in combination with CRL5) in hematopoietic cells could result in the production of an ACT product with an increased proliferative capacity that was resistant to immune suppression and capable of stimulating the production of cytokines. In order to assess the effects of CRL5 complex-independent gene ablation, primary human NK cells were edited using a CRISPR system to inhibit expression or activity of (a) a CRL5 complex independent gene (ARHGAP25, CCNC, FAM49B, N4BP1, STUB1), (b) a representative member of the CRL5 complex (i.e., CUL5) (c) a CRL5 complex-independent gene in combination with CUL5 (CUL5/ARHGAP25, CUL5/CCNC, CUL5/FAM49B, CUL5/N4BP1, CUL5/STUB1), or (d) a negative control gene (AAVS1), as shown in FIGS. 8 and 9.

Methods used to generate the genetically modified hematopoietic cells and downstream assays to assess the effects of inhibiting genes independent of the CRL5 complex (alone, or in combination with a representative member of the CRL5 complex) are described below.

1. Primary Human NK Cell Isolation from PBMC Donors:

Human blood samples were obtained and subjected to centrifugation to retrieve the buffy coat fraction of the blood. Subsequently, primary human NK cells were isolated from peripheral blood mononuclear cells (PBMCs) in the buffy coat fraction using a Human NK cell isolation kit (Miltenyi Biotec). The isolated cells were subsequently cultured in NK MACs medium (Miltenyi Biotec) supplemented with 10% horse serum (HS) and 5 ng/mL IL-15, and were initially expanded using K562-41BBL-mIL-21 feeder cells. After 7 days of expansion, the NK cells were prepared for various assays, as described herein.

### 2. Primary Human T-Cell Isolation and Expansion:

T-cells were activated and expanded from PBMC donors in RPMI-1640 containing 10% fetal calf serum (FCS), 1× GlutaMAX, 1% pen./strep., 0.1% β-mercaptoethanol, 25 mM HEPES, 1% non-essential amino acids, and 1% sodium pyruvate in the presence of 32 ng/mL soluble OKT3 and 600 U/mL IL-2 for 48 h before RNP editing and TCR/CAR transduction. Vδ2⁺ T-cells were expanded from human PBMC donors in RPMI-1640 containing 10% fetal calf serum (FCS), 1× GlutaMAX, 1% pen./strep., 0.1% β-mercaptoethanol, 25 mM HEPES, 1% non-essential amino acids, and 1% sodium pyruvate in the presence of 5 μM Zoledronate and 100 U/mL IL-2 for 5 days before CAR transduction and RNP editing.

### 3. Viral Production and Lentiviral Transduction:

HEK293T cells were transfected with an ABECMA CAR-mScarlet expression construct or 1G4 TCR expression construct, as well as gag/pol and envelope plasmids, using Lipofectamine 3000. Viral supernatant was filtered (0.45 μM) and concentrated using ultracentrifugation (14,208g for 120 minutes). Concentrated virus was aliquoted and stored at −80° C. Each viral batch was titrated using mScarlet expression in the HEK293T cells to determine the viral titer (transduction units/mL of viral stock).

Following the 7-day expansion period described above, NK cells were seeded in Opti-MEM supplemented with 0.1% heat-inactivated fetal calf serum (FCS), 20 ng/mL IL-15, and 10 g/mL vectofusin (Miltenyi Biotec) in a 48-well plate with or without the packaged ABECMA CAR virus at a multiplicity of infection (MOI) of 5. The plate was centrifuged at 1200g for 60 minutes at 32° C. and allowed to incubate overnight. Following the incubation period, the cells were harvested, washed, and reseeded in standard NK cell culture medium in a G-Rex 48-well plate. To generate 1G4-TCR NK cells, expanded NK cells were double transduced with the 1G4 TCR lentivirus and a lentivirus encoding for CD3 complex as described above.

Activated T-cells were transduced with the packaged Abecma CAR or 1G4 TCR lentivirus using RetroNectin-coated plates and spininfection for 1 h at 1200 g at 32° C. Expanded γδ-T-cells were transduced with the packaged Abecma CAR lentivirus using 10 μg/mL vectofusin and spininfection for 1 h at 1200 g at 32° C.

### 4. CRISPR Ribonucleoprotein Editing:

CRISPR ribonucleoprotein (RNP) complexes were assembled using a purified Cas9 protein (61 μM) and sgRNA (100 μM) against the genes of interest in an electroporation buffer. The complex was added directly into the resuspended NK cells (1×10⁶ cells per reaction), which were subsequently electroporated using the CM137 pulse code (Amaxa, Lonza). Following electroporation, the cells were co-cultured with K562-41BBL-mIL-21 feeder cells for another 7 days to allow the cells to recover and expand. T-cells were electroporated using the same code and protocol for RNP editing.

### 5. Flow Cytometry:

NK cells were stained for 20-30 minutes at room temperature for select surface marker expression. Cells were then analyzed directly on a CytoFLEX S (Beckman Coulter; equipped with a 405 nm laser, a 488 nm laser, a 561 nm laser, and a 638 nm laser). Data were analyzed using FlowJo software (Versions 9.9.6 and 10.5.3; BD Bioscience). The following antibodies were used: CD56 (A700), CD56 (BV421), CD122-APC (Beckman Coulter); and LIVE/DEAD Fixable Near-IR Dead Cell Stain kit (Invitrogen). For T-cell staining, the following antibodies were used: CD3 BV650, CD122-PE, CD8 BV605, and CD4 FITC. For γδ-T-cell staining, the following antibodies were used: CD3 BV650, and Vδ2 TCR BV421.

### 6. Proliferation Assay:

NK cell proliferation was measured using an EdU uptake assay. RNP-edited NK cells previously cultured either in high (5 ng/mL) or low (1 ng/mL) IL-15 containing medium were exposed to EdU labeling (2 μM final concentration) overnight. Following the incubation period, the cells were collected and the incorporated EdU was detected using the Click-IT reaction and FACS analysis according to the manufacturer's instructions. Statistics were calculated using one-way analysis of variance (ANOVA) with Dunnett's multiple comparison correction (ns indicates not significant, *P<0.05, **P<0.01, ***P<0.001, ****P<0.0001).

For quantifying EdU incorporation in T-cells, the same protocol was followed as above but T-cells were cultured in RPMI+10% FCS and 600 U/mL IL-2.

### 7. Serial Challenge Assay:

CAR NK cells were co-cultured with targets cells (Nalm6-BCMA-GFP) at different effector:target (E:T) ratios in a 96-well format. Culture medium was supplemented with a low IL-15 concentration (1 ng/mL) with or without the inclusion of the immunosuppressive factor adenosine (NECA; 300 μM final concentration). Fresh target cells were added every 24-48 hours to allow for serial rounds of killing, up to four rounds in total. In FACS experiments, after every round of target challenge, leftover GFP positive target cell, as well as viable NK cell numbers, were enumerated using counting beads. In the live-cell imaging experiments (Incucyte), GFP-positive targets and CAR-mScarlet positive NK cell clusters were quantified using automated Incucyte software continuously for up to 4 days. For immunosuppression experiments, 5 ng/mL TGF-β was added in the co-culture assay. Statistics were calculated using one-way or two-way ANOVA with Dunnett multiple comparison correction (ns indicates not significant, *P<0.05, **P<0.01, ***P<0.001, ****P<0.0001).

The same protocol was followed for 1G4 TCR NK cells, Abecma CAR-T-cells, and Abecma CAR γδ-T-cells. Adenosine (NECA) was used at 300 μM final concentration.

### 8. Cytokine Release Assay:

Supernatant was collected from the abovementioned co-culture assays following the first or second round of target cell killing. Supernatant was aliquoted and frozen at −80° C. IFN-γ, TNF-α, IL-6, and IL-10 cytokines were detected using the Human LEGENDplex Th1 panel with samples assayed in duplicate by flow cytometry (5-plex, BioLegend). In some assays, IFN-γ was measured using AlphaLISA human IFN-γ detection kit (PerkinElmer). Statistics were calculated using one-way ANOVA with Dunnett multiple comparison correction (ns indicates not significant, *P<0.05, **P<0.01, ***P<0.001, ****P<0.0001). For in vivo experiments, supernatants from digested tumors were collected and the cytokine levels were measured using LEGENDplex Th1 panel as described above.

### H. Example 8—CRL5 Complex-Independent Gene Ablated CAR-NK Cells Vs. Target Cells Engineered to Express BCMA (Donor 1)

CAR-NK cells in which a gene independent of the CRL5 complex was ablated (alone, or in combination with a representative member of the CRL5 complex (CUL5)), or a negative control gene (AAVS1), were generated as described above (Example 7) using primary NK cells obtained from a first donor. After the CRL5 complex-independent gene ablated NK cells were generated, various experiments were performed to assess the effects of the genetic modifications.

FIG. 8A shows a schematic describing the production of NK cells modified using an ABECMA CAR construct followed by editing of: (1) a CRL5 complex independent gene (ARHGAP25, CCNC, FAM49B, N4BP1, STUB1), (2) a representative member of the CRL5 complex (i.e., CUL5) (3) a CRL5 complex-independent gene in combination with CUL5 (CUL5/ARHGAP25, CUL5/CCNC, CUL5/FAM49B, CUL5/N4BP1, CUL5/STUB1), or (4) a negative control gene (AAVS1), prior to performing a serial killing assay. FIG. 8B shows representative FACS plots displaying the percentage of CAR transduction, as measured by mScarlet fluorescence, in the CAR-NK cells after CRISPR RNP editing. High CAR expression was maintained over time and was not affected by the RNP editing.

To assess the effect of the genetic ablations on the proliferative capacity of CAR-NK cells, CAR-NK cells were co-cultured with feeder cells over a 6-day period. A quantification of CAR-NK cell expansion is shown in FIG. 8C, expressed as fold change (FC) difference in comparison to the negative control (AAVS1). Briefly, ablation of CUL5, CCNC, STUB1, CUL5/CCNC, CUL5/FAM49B, CUL5/N4BP1, and CUL5/STUB1 resulted in an increase in CAR-NK cell expansion on feeder cells compared to the negative control.

Subsequently, the cytotoxic capacity of the genetically modified CAR-NK cells was assessed using a serial challenge assay with the edited CAR-NK cells against Nalm6-BCMA-GFP target cells. Cells were plated at an effector:target (E:T) ratio of 1:8. FIG. 8D shows graphs depicting the amount of live BCMA targets per well (top panel) and the amount of live CAR-NK cells per well (bottom panel) for the four rounds (R1, R2, R3, and R4) of the serial challenge assay. FIG. 8E shows a summary of the results of FIG. 8D (R4) presented as (1) a percentage of increased killing compared to the negative control, and (2) CAR-NK cell fold-change compared to the negative control. Although CAR-NK cells potently killed BCMA expressing targets, by the fourth round of challenges (R4), negative control cells underwent functional exhaustion and stopped killing, whereas several single-edited (i.e., genetic ablation of one gene orthogonal to the CRL5 complex) and double-edited (i.e., genetic ablation of one gene orthogonal to the CRL5 complex in combination with genetic ablation of CUL5) cells continued to eliminate the targets with various degrees of efficiency.

FIG. 8F shows a quantification of live-cell imaging of the CAR-NK cell clustering (represented as CAR+NK cell clusters per well) while killing target cells during the serial challenge assay using an Incucyte. FIG. 8G shows a summary of the results of FIG. 8F (R4) presented as fold change in NK cell clusters per well at the end point of the serial challenge assay on the Incucyte. Several single-edited and all double-edited CAR-NK cells resulted in an increase in NK clusters compared to the negative control (ranging from 1.5-fold to 11.0-fold).

To assess functional activation of NK cells following ablation of genes orthogonal to the CRL5 complex (alone, or in combination with CUL5), cell culture supernatants were collected during the serial challenge assay (R1) and expression of select cytokines was measured using AlphaLISA human IFN-γ detection kit or a LEGENDplex TH1 panel, as shown in FIG. 8H. Several single-edited and double-edited CAR-NK cells resulted in an increase in IFN-γ secretion compared to the negative control. Several double-edited CAR-NK cells resulted in an increase in IL-10 secretion compared to the negative control.

### L. Example 9—CRL5 Complex-Independent Gene Ablated CAR-NK Cells Vs. Target Cells Engineered to Express BCMA (Donor 2)

In an effort to corroborate the effects observed following ablation of genes independent of the CRL5 complex (alone, or in combination with CUL5), the experiments described in Example 8 were repeated using CAR-NK cells generated as described above (Example 7) using primary NK cells obtained from a second donor.

FIG. 9A shows a schematic describing the production of NK cells modified using an ABECMA CAR construct followed by editing of. (1) a CRL5 complex independent gene (ARHGAP25, CCNC, FAM49B, N4BP1, STUB1), (2) a representative member of the CRL5 complex (i.e., CUL5) (3) a CRL5 complex-independent gene in combination with CUL5 (CUL5/ARHGAP25, CUL5/CCNC, CUL5/FAM49B, CUL5/N4BP1, CUL5/STUB1), or (4) a negative control gene (AAVS1), prior to performing a serial killing assay. FIG. 9B shows representative FACS plots displaying the percentage of CAR transduction, as measured by mScarlet fluorescence, in the CAR-NK cells after CRISPR RNP editing. High CAR expression was maintained over time and was not affected by the RNP editing.

To assess the effect of the genetic ablations on the proliferative capacity of CAR-NK cells, CAR-NK cells were co-cultured with feeder cells over a 6-day period. A quantification of CAR-NK cell expansion is shown in FIG. 9C, expressed as fold change (FC) difference in comparison to the negative control (AAVS1). Briefly, ablation of CUL5, N4BP1, STUB1, CUL5/ARHGAP25, CUL5/CCNC, CUL5/FAM49B, CUL5/N4BP1, and CUL5/STUB1 resulted in an increase in CAR-NK cell expansion on feeder cells compared to the negative control.

Subsequently, the cytotoxic capacity of the genetically modified CAR-NK cells was assessed using a serial challenge assay with the edited CAR-NK cells against Nalm6-BCMA-GFP target cells. Cells were plated at an E:T ratio of 1:8. FIG. 9D shows graphs depicting the amount of live BCMA targets per well (top panel) and the amount of live CAR-NK cells per well (bottom panel) for the four rounds (R1, R2, R3, and R4) of the serial challenge assay. FIG. 9E shows a summary of the results of FIG. 9D (R4) presented as (1) a percentage of increased killing compared to the negative control, and (2) CAR-NK cell fold-change compared to the negative control. Although CAR-NK cells potently killed BCMA expressing targets, by the fourth round of challenges (R4), negative control cells underwent functional exhaustion and stopped killing, whereas several single-edited (i.e., genetic ablation of one gene orthogonal to the CRL5 complex) and double-edited (i.e., genetic ablation of one gene orthogonal to the CRL5 complex in combination with genetic ablation of CUL5) cells continued to eliminate the targets with various degrees of efficiency.

FIG. 9F shows a quantification of live-cell imaging of the CAR-NK cell clustering (represented as CAR⁺ NK cell clusters per well) while killing target cells during the serial challenge assay using an Incucyte. FIG. 9G shows a summary of the results of FIG. 9F (R4) presented as fold change in NK cell clusters per well at the end point of the serial challenge assay on the Incucyte. All single-edited and all double-edited CAR-NK cells resulted in an increase in NK clusters compared to the negative control (ranging from 1.7-fold to 23.2-fold).

To assess functional activation of NK cells following ablation of genes orthogonal to the CRL5 complex (alone, or in combination with CUL5), cell culture supernatants were collected during the serial challenge assay (R1) and expression of select cytokines was measured using AlphaLISA human IFN-γ detection kit or a LEGENDplex TH1 panel, as shown in FIG. 9H. Several single-edited and double-edited CAR-NK cells resulted in an increase in IFN-γ secretion compared to the negative control. Several double-edited CAR-NK cells resulted in an increase in IL-10 secretion compared to the negative control.

### J. Example 10—CAR-NK Cell Validation Assays Under Immunosuppressive Conditions

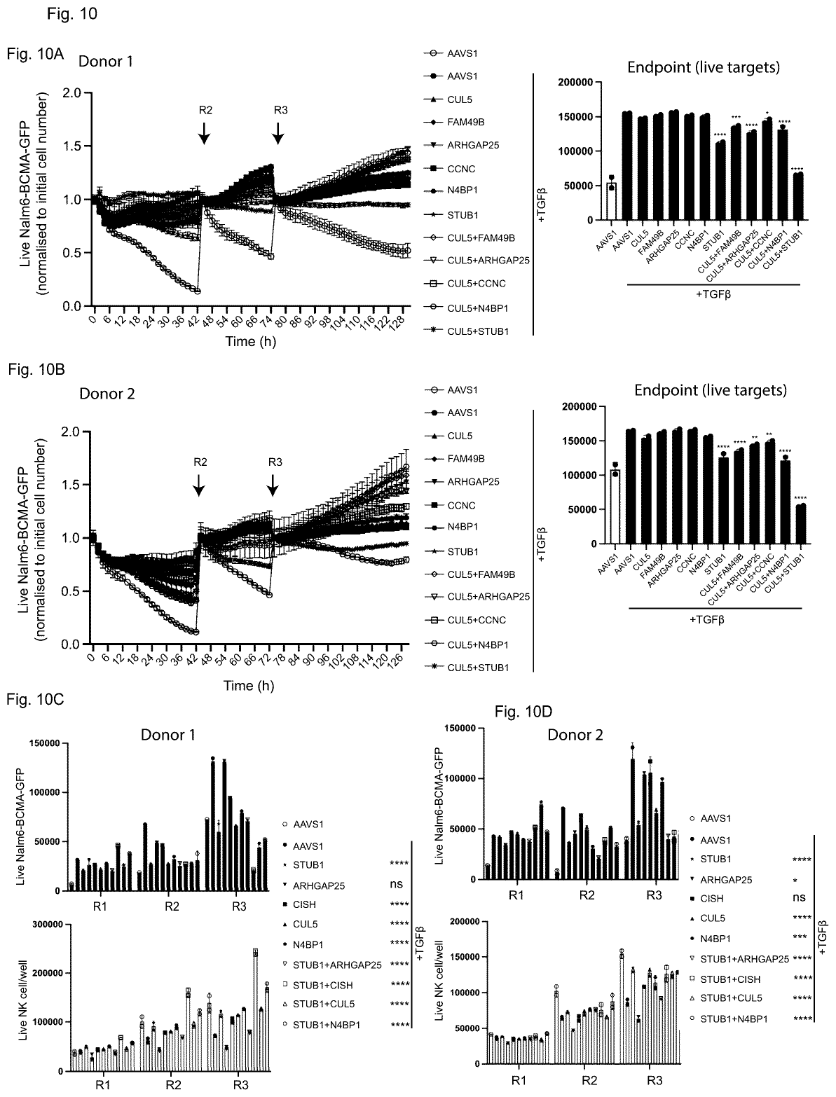

In an effort to investigate the effects observed following ablation of genes independent of the CRL5 complex (alone, or in combination with a CRL5 complex gene) under immunosuppressive conditions, serial challenge assays were performed and assessed using a live-cell imaging system (Incucyte; FIGS. 10A and 10B) or FACS (FIGS. 10C and 10D). Single-edited and double-edited CAR-NK cells were generated from Donor 1 and Donor 2, as described above (see, e.g., Examples 7-9). For the single-edited CAR-NK cells, one of the following genes was ablated using a CRISPR system: AAVS1 (negative control), CUL5 (positive control), ARHGAP25, CCNC, FAM49B, N4BP1, or STUB1, as shown in FIGS. 10A and 10B; or AAVS1, STUB1, ARHGAP25, CISH, CUL5, or N4BP1, as shown in FIGS. 10C and 10D. For the double-edited CAR-NK cells, one of the following combinations of genes was ablated using a CRISPR system: CUL5/FAM49B, CUL5/ARHGAP25, CUL5/CCNC, CUL5/N4BP1, or CUL5/STUB1, as shown in Figs. bOA and 10B; or STUB1/ARHGAP25, STUB1/CISH, STUB1/CUL5, or STUB1/N4BP1, as shown in FIGS. 10C and 10D.

Serial challenge assays were performed as described above using single- or double-edited CAR-NK cells in co-culture with Nalm6-BCMA-GFP target cells (1:8 E:T ratio) in the presence of TGF-β (5 ng/mL). Data for CAR-NK cells derived from Donor 1 is shown in Figs. bOA and 10C, and data for CAR-NK cells derived from Donor 2 is shown in Figs. bOB and 10D. As is evidenced from the kinetics graphs shown in FIG. 10, TGF-β induces significant immunosuppression of CAR-NK cells as early as the first round of target challenge, with complete loss of functionality by round 3 (R3) in the negative control. The bar graphs accompanying FIGS. 10A and 10B show absolute number of live target cells per well at assay endpoint. Similarly, FIGS. 10C and 10D show live target cell counts and NK cell counts per well. Some knockout combinations enabled CAR-NK cells to remain functional in the presence of (i) high target cell burden, and (ii) high levels of TGF-β.

### K. Example 11

Additional experiments were performed to further validate the results described in Examples 1-10 using different cellular contexts. Generally, cells were established, and assays were performed, as described in Examples 1 and 7.

### 1. 1G4-TCR T-Cells:

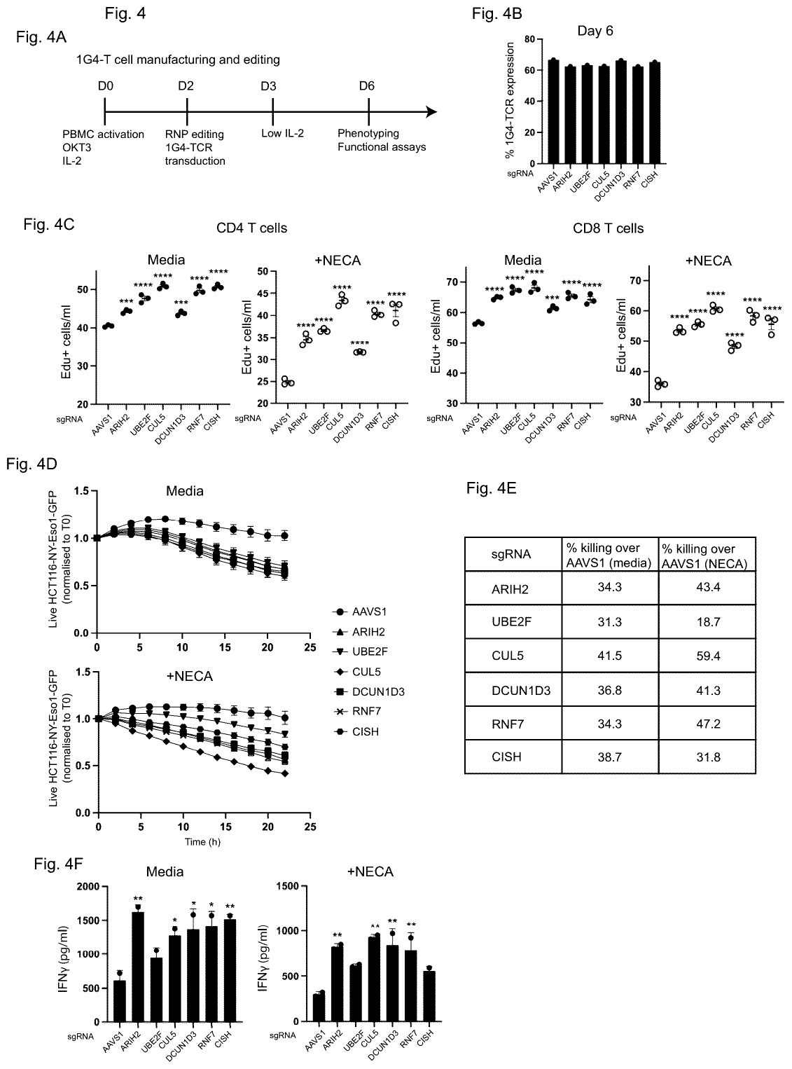

CRL5 complex-ablated T-cells transduced with the 1G4-TCR expression construct were generated (as shown in FIG. 4A) and expression of the exogenous TCR was confirmed (as shown in FIG. 4B). Subsequently, proliferation of edited CD4 and CD8 T-cells was assessed in the presence or absence of the immunosuppressive factor adenosine. Increased EdU uptake by T-cells edited for the CRL5 complex was shown under both experimental settings as compared to the AAVS1 control group, with a more pronounced effect exhibited under immunosuppressive conditions, as shown in FIG. 4C. Further, a short-term killing assay was performed using the edited 1G4 T-cells and HCT116-NY-Eso1-GFP target cells in the presence or absence of adenosine (as shown in FIG. 4D). Similar to the results of the EdU uptake experiment, T-cells edited for the CRL5 complex showed an increased capacity for killing target cells under both experimental settings as compared to the AAVS1 control group, as shown in FIG. 4E. Still further, IFNγ release was assessed during the short-term killing assay. As shown in FIG. 4F, the CRL5 edited 1G4 T-cells released more IFNγ compared to the AAVS1 control group.

### 2 Abecma CAR-T-Cells:

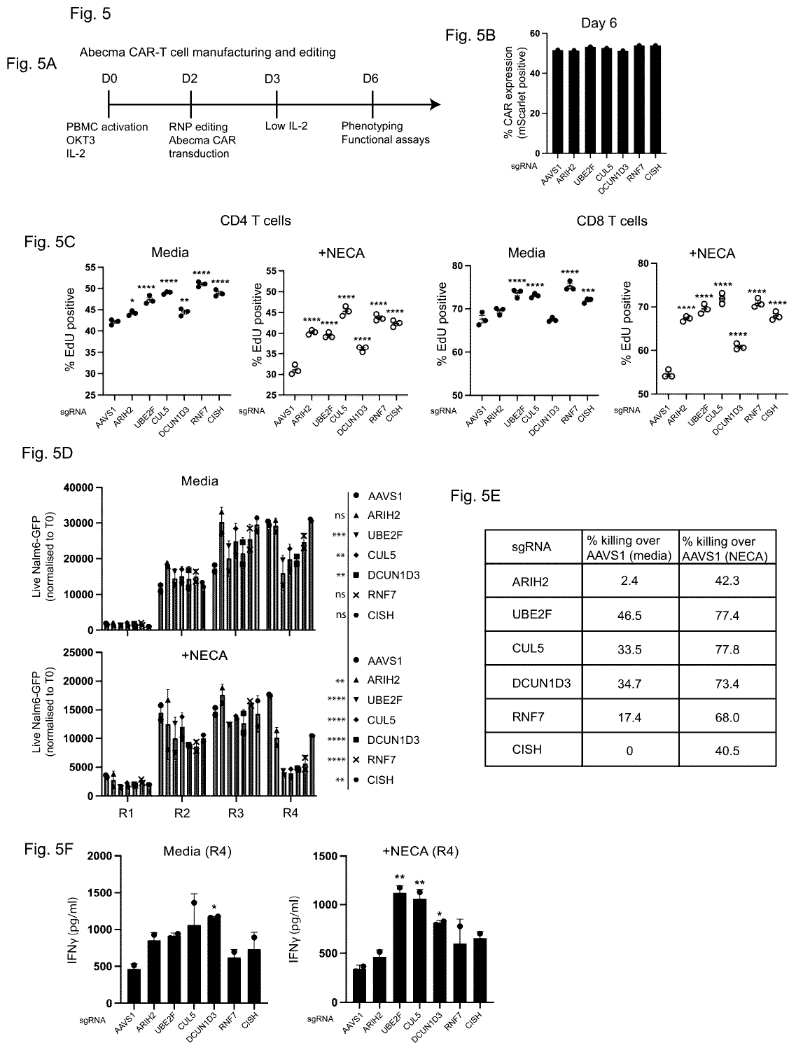

CRL5 complex-ablated T-cells transduced with the Abecma CAR construct were generated (as shown in FIG. 5A) and expression of the CAR construct was confirmed (as shown in FIG. 5B). Subsequently, proliferation of edited CD4 and CD8 T-cells was assessed in the presence or absence of the immunosuppressive factor adenosine. Increased EdU uptake by T-cells edited for the CRL5 complex was shown under both experimental settings as compared to the AAVS1 control group, with a more pronounced effect exhibited under immunosuppressive conditions, as shown in FIG. 5C. Further, a serial challenge assay (serial killing assay) was performed using the edited CAR-T-cells and Nalm6-BCMA-GFP target cells in the presence or absence of adenosine (as shown in FIG. 5D). Similar to the results of the EdU uptake experiment, T-cells edited for the CRL5 complex showed an increased capacity for killing target cells under both experimental settings as compared to the AAVS1 control group, even at the last round (R4) of the serial challenge assay, as shown in FIG. 5E. Still further, IFNγ release was assessed during the last round (R4) of the serial challenge assay. As shown in FIG. 5F, the CRL5 edited CAR-T-cells released more IFNγ compared to the AAVS1 control group.

### 3. 1G4-TCR NK Cells:

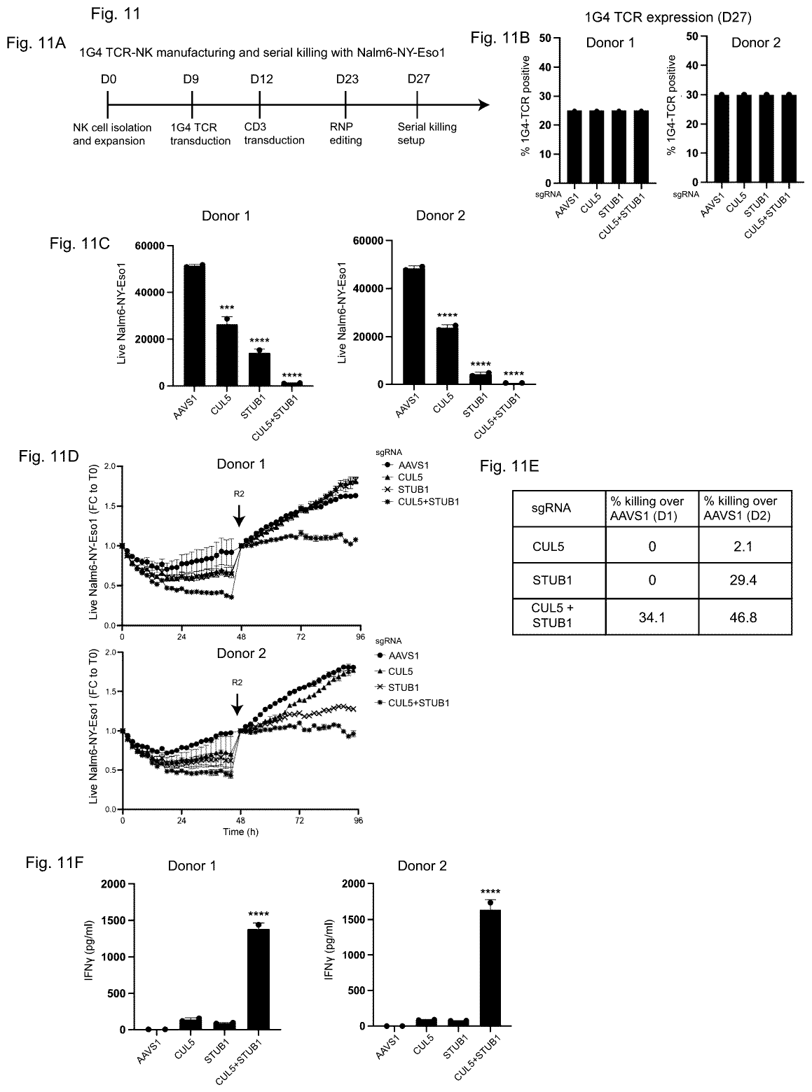

NK cells transduced with the 1G4-TCR expression construct were generated using NK cells derived from one of two donors (as shown in FIG. 11A). Following transduction with the 1G4-TCR expression construct, NK cells were subjected to RNP editing of: (i) AAVS1, (ii) CUL5 (alone), (iii) STUB1 (alone), or (iv) CUL5⁺ STUB1. Expression of the exogenous TCR was confirmed, as shown in FIG. 111B. Subsequently, a short-term killing assay (24 h; Incucyte) was performed using the edited 1G4 NK cells and Nalm6-NY-Eso1-GFP target cells. As shown in FIG. 11C, quantification of the short-term killing assay demonstrated that the genetic edits (CUL5, STUB1, or CUL5⁺ STUB1) resulted in an increased killing capacity of the targets cells compared to the AAVS1 control group. Interestingly, the combination of genetic modifications to CUL5 (a representative member of the CRL5 complex) and STUB1 (a gene orthogonal to the CRL5 complex) exhibited the largest increase in % killing over the control group. Further, a serial challenge assay was performed (R1 and R2) using the edited 1G4 NK cells and Nalm6-NY-Eso1-GFP target cells, as shown in FIG. 11D. Similar to the results of the short-term killing assay, the combination of genetic edits to CUL5⁺ STUB1 resulted in an increased killing capacity for the NK cells derived from both donors, as shown in FIG. 11E. Still further, IFNγ release was assessed during the short-term killing assay. Surprisingly, the combination of genetic modifications to CUL5 and STUB1 exhibited a dramatic increase in IFNγ release compared to the AAVS1 control group (as shown in FIG. 11F).

### 4. Abecma-CAR γδ-T-Cells:

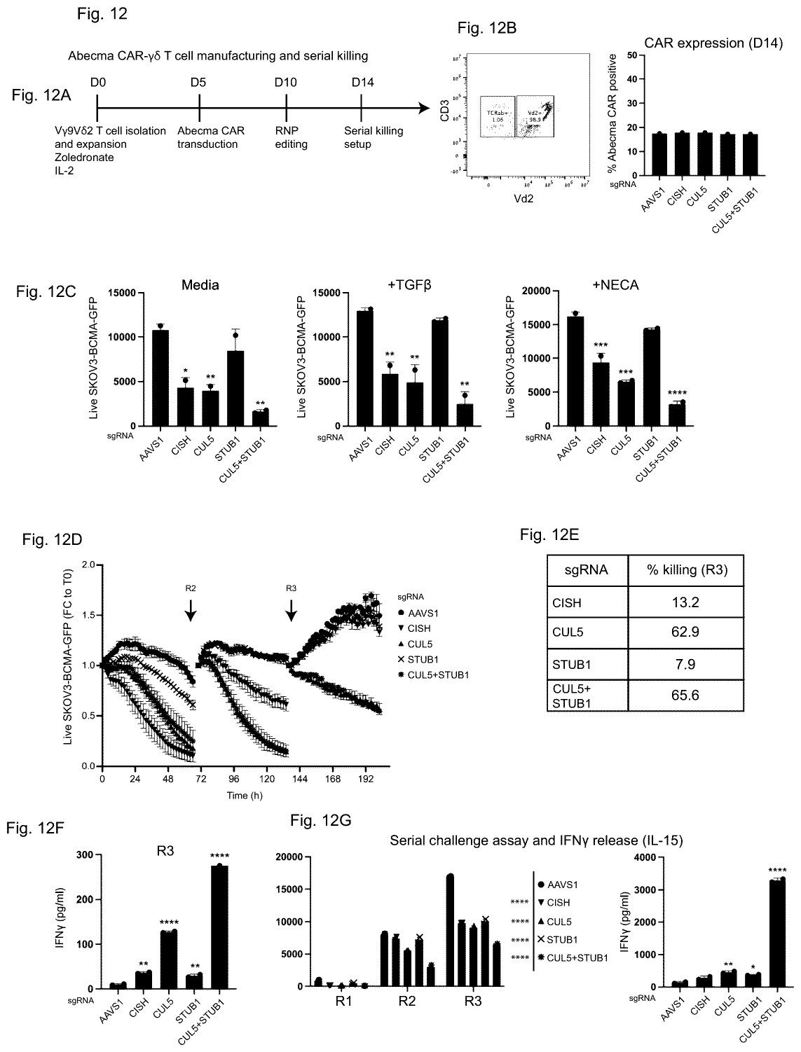

γδ-T-cells transduced with the Abecma CAR construct were generated as shown in FIG. 12A. Following transduction with the Abecma CAR construct, γδ-T-cells were subjected to RNP editing of: (i) AAVS1, (ii) CUL5 (alone), (iii) STUB1 (alone), (iv) CUL5⁺ STUB1, or (v) CISH. Purity of the γδ-T-cells and expression of the CAR construct were confirmed, as shown in FIG. 12B. Subsequently, a short-term killing assay (24 h; Incucyte) was performed using the edited γδ-T-cells and SKOV3-BCMA-GFP target cells in the presence or absence of an immunosuppressive factor (TGF-0 or adenosine). As shown in FIG. 12C, the short-term killing assay demonstrated that the combination of genetic edits to CUL5⁺ STUB1 resulted in a pronounced increased killing capacity of the target cells compared to the AAVS1 control group under all three experimental conditions. Further, a serial challenge assay was performed (R1, R2, and R3) using the edited γδ-T-cells and SKOV3-BCMA-GFP target cells, as shown in FIG. 12D. Similar to the results of the short-term killing assay, the combination of genetic edits to CUL5⁺ STUB1 resulted in a pronounced increased killing capacity of the target cells during all round of the serial challenge assay (see, e.g., FIG. 12D and FIG. 12E). Still further, IFNγ release was assessed during the third round (R3) of the serial challenge assay. Again, the combination of genetic edits to CUL5⁺ STUB1 resulted in the largest amount of IFNγ production (as shown in FIG. 12F). Subsequently, another serial challenge assay was performed using the edited γδ-T-cells and SKOV3-BCMA-GFP target cells similar to the experimental design shown in FIG. 12A except that the γδ-T-cells were expanded in the presence of the cytokine IL-15 instead of the cytokine IL-2. Under these conditions, the combination of genetic edits to CUL5⁺ STUB1 provided a clear advantage in serial killing and IFNγ release compared to the AAVS1 control group and single-edited groups, as shown in FIG. 12G.

### L. Example 12—CAR-NK Cell Adoptive Transfer

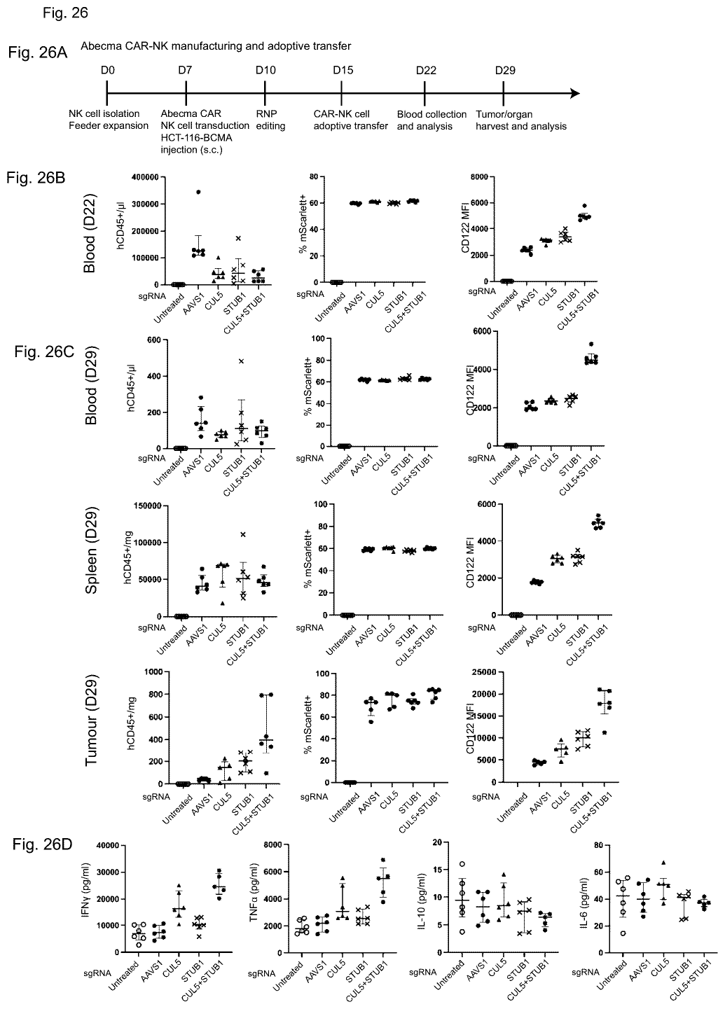

NK cells were isolated, expanded, transduced with the Abecma CAR construct, and edited with indicated sgRNA (i.e., AAVS1 (control), CUL5 (alone), STUB1 (alone), or CUL5⁺ STUB1 (“combination group”)), as shown in FIG. 26A. Approximately 10×10⁶ of the edited Abecma CAR-NK cells were adoptively transferred into the NSG mice inoculated with the BCMA-expressing HCT-116 tumor cells 15 days post-isolation (D15). NK cells were supported by injection of human rIL-15 via 2 μg intraperitoneal injections once every three days. Cheek bleeds were performed 7 days after adoptive transfer (D22). Further, tumor samples, blood, and the spleen were obtained 14 days after adoptive transfer (D29). CAR-NK cell persistence, CAR-mScarlet, and CD122 expression were measured in each of the collected samples, and release of select cytokines ( ) was measured in the supernatants obtained by digesting the tumors with a Collagenase IV (1 mg/mL)/DNase (0.1 mg/mL) digestion cocktail.

As shown in FIG. 26B, CAR transduction was maintained in vivo, and all edited groups exhibited an increase in CD122 expression compared to the AAVS1 controls seven days after adoptive transfer, with the combination group showing the largest increase. Edited CAR-NK cell numbers were decreased compared to the AAVS1 control, especially in the combination group, potentially indicating an increased recruitment into the tumors. Similarly, 14 days after adoptive transfer, increased CD122 expression was maintained in the tumor microenvironment (TME) and peripherally, as shown in FIG. 26C. mScarlet expression was increased in all groups in the TME compared to the periphery, indicating CAR engagement and CAR-driven proliferation of CAR-NK cells. Further, persistence of CAR-NK cells was increased compared to the AAVS1 control within the tumors. As shown in FIG. 26D, release of IFNγ and TNFα were significantly increased, especially within the combination group, compared to the AAVS1 control, whereas IL-10 and IL-6 remained unchanged.

What is claimed is:

1. A genetically modified hematopoietic cell, comprising:
(A) one or more genetic modifications that inhibit expression or activity of polypeptide products encoded by one or more CRL5 complex-independent genes, wherein:
(i) the one or more CRL5 complex-independent genes are selected from a group comprising: (a) STUB1, (b) CCNC, (c) FAM49B, (d) N4BP1, (e) ARHGAP25, or
(f) any combination thereof; and
(ii) expression or activity of the one or more polypeptide products is inhibited by at least about 50% compared to a control hematopoietic cell; and
(B) one or more genetic modifications that inhibit expression or activity of polypeptide products encoded by one or more CRL5 complex genes, wherein:
(i) the one or more CRL5 complex genes are selected from a group comprising:
(a) CUL5, (b) ARIH2, (c) DCUN1D3, (d) RNF7, (e) UBE2F, (f) CISH, or (g) any combination thereof; and
(ii) expression or activity of the one or more polypeptide products is inhibited by at least about 50% compared to a control hematopoietic cell.

2. The genetically modified hematopoietic cell of claim 1, wherein the genetic modifications inactivate (i) the one or more CRL5 complex-independent genes, and/or (ii) the one or more CRL5 complex-independent genes.

3. The genetically modified hematopoietic cell according to any one of claims 1 or 2, wherein:
(1) the one or more CRL5 complex-independent genes are inhibited or inactivated using a first genetic modification system selected from a group comprising: (i) a clustered, regularly interspaced, short palindromic repeats (CRISPR) system, (ii) a transcription activator-like effector nuclease (TALEN) system, (iii) a zinc finger nuclease (ZFN) system, (iv) a meganuclease system, (v) a short hairpin RNA (shRNA) system, (vi) a small interfering RNA (siRNA) system, (vii) a microRNA (miRNA) system, or (viii) an antisense RNA system; and
(2) the one or more CRL5 complex genes are inhibited or inactivated using a second genetic modification system selected from a group comprising: (i) a clustered, regularly interspaced, short palindromic repeats (CRISPR) system, (ii) a transcription activator-like effector nuclease (TALEN) system, (iii) a zinc finger nuclease (ZFN) system, (iv) a meganuclease system, (v) a short hairpin RNA (shRNA) system, (vi) a small interfering RNA (siRNA) system, (vii) a microRNA (miRNA) system, or (viii) an antisense RNA system.

4. The genetically modified hematopoietic cell according to any one of claims 1-3, wherein the genetically modified hematopoietic cell comprises a natural killer (NK) cell.

5. The genetically modified hematopoietic cell of claim 4, wherein the NK cell comprises a CAR-NK cell.

6. The genetically modified hematopoietic cell according to any one of claims 1-5, wherein the genetic modifications to (i) the one or more CRL5 complex-independent genes, and (ii) the one or more CRL5 complex genes improve proliferation of the genetically modified hematopoietic cell by about 125% to about 150%, about 250% to about 300%, about 400% to about 500%, about 850% to about 900%, about 1600% to about 1650%, or about 2200% to about 2250% compared to proliferation of a control hematopoietic cell, as determined by a first assay.

7. The genetically modified hematopoietic cell according to any one of claims 1-6, wherein the genetic modifications to (i) the one or more CRL5 complex-independent genes, and (ii) the one or more CRL5 complex genes improve proliferation of the genetically modified hematopoietic cell by about 1.25-fold to about 1.50-fold, about 2.5-fold to about 3.0-fold, about 4.0-fold to about 5.0-fold, about 8.5-fold to about 9.0-fold, about 16.0-fold to about 16.5-fold, or about 22.0-fold to about 22.5-fold compared to proliferation of a control hematopoietic cell, as determined by a first assay.

8. The genetically modified hematopoietic cell according to any one of claims 6 or 7, wherein the first assay comprises: (i) a quantification of cells and/or cell clusters using a cell imaging system (e.g., Incucyte), (ii) a 5-ethynyl-2′-deoxyuridine (EdU) incorporation assay, (iii) a bromodeoxyuridine (BrdU) incorporation assay, (iv) a tetrazolium salt reduction assay, (v) an ATP assay, or (vi) a Ki-67 staining assay.

9. The genetically modified hematopoietic cell according to any one of claims 1-8, wherein the genetic modifications to (i) the one or more CRL5 complex-independent genes, and (ii) the one or more CRL5 complex genes improve resistance to immune suppression of the genetically modified hematopoietic cell by about 6.00% to about 9.00%, about 9.00% to about 15.0%, about 15.0% to about 21.0%, about 21.0% to about 27.0%, about 45.0% to about 48.0%, about 54.0% to about 69.0%, about 60.0% to about 70.0%, or about 81.0% to about 84.0% compared to resistance to immune suppression of a control hematopoietic cell, as determined by a second assay.

10. The genetically modified hematopoietic cell according to any one of claims 1-9, wherein the genetic modifications to (i) the one or more CRL5 complex-independent genes, and (ii) the one or more CRL5 complex genes improve resistance to immune suppression of the genetically modified hematopoietic cell by about 0.06-fold to about 0.09-fold, about 0.09-fold to about 0.15-fold, about 0.15-fold to about 0.21-fold, about 0.21-fold to about 0.27-fold, about 0.45-fold to about 0.48-fold, about 0.54-fold to about 0.69-fold, about 0.60-fold to about 0.70-fold, or about 0.81-fold to about 0.84-fold compared to immune suppression of a control hematopoietic cell, as determined by a second assay.

11. The genetically modified hematopoietic cell according to any one of claims 9 or 10, wherein the second assay comprises (i) a serial challenge assay, or (ii) a cytokine release assay.

12. The genetically modified hematopoietic cell according to any one of claims 1-11, wherein the genetic modifications to (i) the one or more CRL5 complex-independent genes, and (ii) the one or more CRL5 complex genes improve the cytotoxic capacity of the genetically modified hematopoietic cell by about 67.0% to about 72.0%, about 78.0% to about 87.0%, or about 90.0% to about 100.0% compared to cytotoxic capacity of a control hematopoietic cell, as determined by a third assay.

13. The genetically modified hematopoietic cell according to any one of claims 1-12, wherein the genetic modifications to (i) the one or more CRL5 complex-independent genes, and (ii) the one or more CRL5 complex genes improve the cytotoxic capacity of the genetically modified hematopoietic cell by about 0.67-fold to about 0.72-fold, about 0.78-fold to about 0.87-fold, or about 0.9-fold to about 1.0-fold compared to cytotoxic capacity of a control hematopoietic cell, as determined by a third assay.

14. The genetically modified hematopoietic cell according to any one of claims 12 or 13, wherein the third assay comprises: (i) a quantification of cells and/or cell clusters using a cell imaging system (e.g., Incucyte), (ii) a serial challenge assay, (iii) an enzymatic cytotoxicity assay, (iv) an apoptosis assay, (v) an autophagy assay, or (vi) a cell viability assay.

15. The genetically modified hematopoietic cell according to any one of claims 1-14, wherein the genetic modifications to (i) the one or more CRL5 complex-independent genes, and (ii) the one or more CRL5 complex genes improve the killing efficiency of cancer cells by the genetically modified hematopoietic cell by about 67.0% to about 72.0%, about 78.0% to about 87.0%, or about 90.0% to about 100.0% compared to the killing efficiency of cancer cells by a control hematopoietic cell, as determined by a fourth assay.

16. The genetically modified hematopoietic cell according to any one of claims 1-15, wherein the genetic modifications to (i) the one or more CRL5 complex-independent genes, and (ii) the one or more CRL5 complex genes improve the killing efficiency of cancer cells by the genetically modified hematopoietic cell by about 0.67-fold to about 0.72-fold, about 0.78-fold to about 0.87-fold, or about 0.9-fold to about 1.0-fold compared to the killing efficiency of cancer cells by a control hematopoietic cell, as determined by a fourth assay.

17. The genetically modified hematopoietic cell according to any one of claims 15 or 16, wherein the fourth assay comprises: (i) a quantification of cells and/or cell clusters using a cell imaging system (e.g., Incucyte), (ii) a serial challenge assay, (iii) an enzymatic cytotoxicity assay, (iv) an apoptosis assay, (v) an autophagy assay, or (vi) a cell viability assay.

18. The genetically modified hematopoietic cell according to any one of claims 1-17, wherein the genetic modifications to (i) the one or more CRL5 complex-independent genes, and (ii) the one or more CRL5 complex genes improve capability to stimulate production of one or more cytokines by the genetically modified hematopoietic cell by about 5.0% to about 10.0%, about 15.0% to about 20.0%, about 25.0% to about 30.0%, about 55.0% to about 60.0%, about 70.0% to about 75.0%, about 80.0% to about 85.0%, about 90.0% to about 100.0%, about 120.0% to about 125.0%, about 160.0% to about 165.0%, about 205.0% to about 210.0%, about 280.0% to about 300.0%, about 340.0% to about 350.0%, or about 505.0% to about 510.0% compared to capability to stimulate production of one or more cytokines of a control hematopoietic cell, as determined by a fifth assay.

19. The genetically modified hematopoietic cell according to any one of claims 1-18, wherein the genetic modifications to (i) the one or more CRL5 complex-independent genes, and (ii) the one or more CRL5 complex genes improve capability to stimulate production of one or more cytokines by the genetically modified hematopoietic cell by about 0.05-fold to about 0.10-fold, about 0.15-fold to about 0.20-fold, about 0.25-fold to about 0.30-fold, about 0.55-fold to about 0.60-fold, about 0.70-fold to about 0.75-fold, about 0.80-fold to about 0.85-fold, about 0.90-fold to about 1.00-fold, about 1.20-fold to about 1.25-fold, about 1.60-fold to about 1.65-fold, about 2.05-fold to about 2.10-fold, about 2.80-fold to about 3.00-fold, about 3.40-fold to about 3.50-fold, or about 5.05-fold to about 5.10-fold compared to capability to stimulate production of one or more cytokines of a control hematopoietic cell, as determined by a fifth assay.

20. The genetically modified hematopoietic cell according to any one of claims 18 or 19, wherein the one or more cytokines are selected from the group consisting of: (i) granulocyte colony-stimulating factor (G-CSF), (ii) granulocyte-macrophage colony-stimulating factor (GM-CSF), (iii) interferon alpha (IFN-α), (iv) interferon gamma (IFN-γ), (v) interleukin-1 beta (IL-13), (vi) IL-2, (vii) IL-4, (viii) IL-5, (ix) IL-6, (x) IL-8, (xi) IL-10, (xii) interleukin-12 p70 (IL-12 p70), (xiii) IL-13, (xiv) IL-17A, (xv) IL-18, (xvi) interferon gamma-induced protein 10 (IP-10), (xvii) monocyte chemoattractant protein-1 (MCP-1), (xviii) macrophage inflammatory protein-1 alpha (MIP-1α), (xix) MIP-1 beta (MIP-1β), (xx) tumor necrosis factor alpha (TNF-α), (xxi) TNF-β, and (xxii) any combination thereof.

21. The genetically modified hematopoietic cell according to any one of claims 18-20, wherein the fifth assay comprises: (i) a ProcartaPlex multiplex cytokine assay, (ii) a Human LEGENDplex TH1 panel, (iii) an AlphaLISA assay, (iv) an intracellular cytokine stain assay, (v) a Luminex bead-based cytokine release assay, (vi) a MACS cytokine secretion assay, (vii) an ELISA, or (viii) an ELISpot assay.

22. The genetically modified hematopoietic cell according to any one of claims 1-21, wherein the control hematopoietic cell comprises a wild-type NK cell.

23. A method of generating a genetically modified cell population for treatment of a subject that has cancer, the method comprising:
(a) obtaining a plurality of hematopoietic cells;
(b) inhibiting expression of one or more CRL5 complex-independent genes, wherein the one or more CRL5 complex-independent genes is selected from a group comprising: (i) STUB1, (ii) CCNC, (iii) FAM49B, (iv) N4BP1, (v) ARHGAP25, and (vi) any combination thereof;
(c) inhibiting expression of one or more CRL5 complex genes, wherein the one or more CRL5 complex genes is selected from a group comprising: (i) CUL5, (ii) ARIH2, (iii) DCUN1D3, (iv) RNF7, (v) UBE2F, (vi) CISH, or (vii) any combination thereof;
(d) selecting hematopoietic cells in which the one or more CRL5 complex-independent genes and the one or more CRL5 complex genes are inhibited; and
(e) expanding the selected hematopoietic cells ex vivo, thereby generating a genetically modified cell population for treatment of a subject that has cancer.

24. The method of claim 23, wherein the plurality of hematopoietic cells is derived from (i) the subject that has cancer, (ii) a donor that does not have cancer, or (iii) a cell line.

25. The method according to claim 24, wherein the cell line is selected from the group consisting of: (i) an induced pluripotent stem cell (iPSC) line, and (ii) a hematopoietic stem cell (HSC) line.

26. A method of treating cancer, comprising:
administering a population of cells comprising a genetically modified hematopoietic cell according to any one of claims 1-22 to a subject that has cancer.

27. The method of claim 26, wherein the population of cells is derived from (i) the subject that has cancer, (ii) a donor that does not have cancer, or (iii) a cell line.

28. The method of claim 27, wherein the cell line is selected from the group consisting of: (i) an induced pluripotent stem cell (iPSC) line, and (ii) a hematopoietic stem cell (HSC) line.

29. The method of any one of claims 26-28, wherein the population of cells comprises about 100,000 (one hundred thousand) cells to about 100,000,000,000 (one hundred billion) cells.

30. The method of any one of claims 26-29, wherein prior to the administering, the subject has received a myeloablative conditioning regimen, a nonmyeloablative conditioning regimen, or a reduced intensity conditioning regimen.

31. A genetically modified hematopoietic cell, comprising:
a genetic modification to one or more CRL5 complex genes that inhibits expression or activity of the polypeptide encoded by the one or more CRL5 complex genes, wherein expression or activity of the polypeptide product is inhibited by at least about 50% compared to a control hematopoietic cell.

32. The genetically modified hematopoietic cell of claim 31, wherein the one or more CRL5 complex genes are selected from the group consisting of: (i) CUL5, (ii) ARIH2, (iii) DCUN1D3, (iv) RNF7, (v) UBE2F, and (vi) any combination thereof.

33. The genetically modified hematopoietic cell according to any one of claims 31 or 32, wherein the genetic modification of the one or more CRL5 complex genes inactivates the one or more CRL5 complex genes.

34. The genetically modified hematopoietic cell according to any one of claims 31-33, wherein the one or more CRL5 complex genes are inhibited using: (i) a clustered, regularly interspaced, short palindromic repeats (CRISPR) system, (ii) a transcription activator-like effector nuclease (TALEN) system, (iii) a zinc finger nuclease (ZFN) system, (iv) a meganuclease system, (v) a short hairpin RNA (shRNA) system, (vi) a small interfering RNA (siRNA) system, (vii) a microRNA (miRNA) system, or (viii) an antisense RNA system.

35. The genetically modified hematopoietic cell according to any one of claims 31-34, wherein the genetically modified hematopoietic cell is a cell selected from the group consisting of: (i) a T-cell, (ii) a natural killer (NK) cell, and (iii) a tumor-infiltrating lymphocyte (TIL).

36. The genetically modified hematopoietic cell of claim 35, wherein the T-cell comprises a chimeric antigen receptor-(CAR-) T-cell or a T-cell receptor-(TCR-) T-cell.

37. The genetically modified hematopoietic cell of claim 35, wherein the NK cell comprises a CAR-NK cell or a TCR-NK cell.

38. The genetically modified hematopoietic cell according to any one of claims 31-37, wherein the genetic modification to one or more CRL5 complex genes improves proliferation of the modified hematopoietic cell by about 10% to about 100% compared to proliferation of a control hematopoietic cell, as determined by a first assay.

39. The genetically modified hematopoietic cell according to any one of claims 31-38, wherein the genetic modification to one or more CRL5 complex genes improves proliferation of the modified hematopoietic cell by about 1.0-fold to about 15.0-fold compared to proliferation of a control hematopoietic cell, as determined by a first assay.

40. The genetically modified hematopoietic cell according to any one of claims 38 or 39, wherein the first assay comprises: (i) a 5-ethynyl-2′-deoxyuridine (EdU) incorporation assay, (ii) a bromodeoxyuridine (BrdU) incorporation assay, (iii) a tetrazolium salt reduction assay, (iv) an ATP assay, or (v) a Ki-67 staining assay.

41. The genetically modified hematopoietic cell according to any one of claims 31-40, wherein the genetic modification to one or more CRL5 complex genes improves resistance to immune suppression by about 10% to about 100% compared to resistance to immune suppression of a control hematopoietic cell, as determined by a second assay.

42. The genetically modified hematopoietic cell according to any one of claims 31-41, wherein the genetic modification to one or more CRL5 complex genes improves resistance to immune suppression by about 1.0-fold to about 10.0-fold compared to immune suppression of a control hematopoietic cell, as determined by a second assay.

43. The genetically modified hematopoietic cell according to any one of claims 41 or 42, wherein the second assay comprises (i) a serial challenge assay, or (ii) a cytokine release assay.

44. The genetically modified hematopoietic cell according to any one of claims 31-43, wherein the genetic modification to one or more CRL5 complex genes improves the cytotoxic capacity by about 10% to about 100% compared to cytotoxic capacity of a control hematopoietic cell, as determined by a third assay.

45. The genetically modified hematopoietic cell according to any one of claims 31-44, wherein the genetic modification to one or more CRL5 complex genes improves the cytotoxic capacity by about 0.5-fold to about 10.0-fold compared to cytotoxic capacity of a control hematopoietic cell, as determined by a third assay.

46. The genetically modified hematopoietic cell according to any one of claims 44 or 45, wherein the third assay comprises: (i) a serial challenge assay, (ii) an enzymatic cytotoxicity assay, (iii) an apoptosis assay, (iv) an autophagy assay, or (v) a cell viability assay.

47. The genetically modified hematopoietic cell according to any one of claims 31-46, wherein the genetic modification to one or more CRL5 complex genes improves the killing efficiency of cancer cells by the genetically modified hematopoietic cells by about 10% to about 100% compared to the killing efficiency of cancer cells by a control hematopoietic cell, as determined by a fourth assay.

48. The genetically modified hematopoietic cell according to any one of claims 31-47, wherein the genetic modification to one or more CRL5 complex genes improves the killing efficiency of cancer cells by the genetically modified hematopoietic cells by about 0.5-fold to about 10.0-fold compared to the killing efficiency of cancer cells by a control hematopoietic cell, as determined by a fourth assay.

49. The genetically modified hematopoietic cell according to any one of claims 47 or 48, wherein the fourth assay comprises: (i) a serial challenge assay, (ii) an enzymatic cytotoxicity assay, (iii) an apoptosis assay, (iv) an autophagy assay, or (v) a cell viability assay.

50. The genetically modified hematopoietic cell according to any one of claims 31-49, wherein the genetic modification to one or more CRL5 complex genes improves capability to stimulate production of one or more cytokines by about 10% to about 100% compared to capability to stimulate production of one or more cytokines of a control hematopoietic cell, as determined by a fifth assay.

51. The genetically modified hematopoietic cell according to any one of claims 31-50, wherein the genetic modification to one or more CRL5 complex genes improves capability to stimulate production of one or more cytokines by about 0.5-fold to about 20.0-fold compared to capability to stimulate production of one or more cytokines of a control hematopoietic cell, as determined by a fifth assay.

52. The genetically modified hematopoietic cell according to any one of claims 50 or 51, wherein the one or more cytokines are selected from the group consisting of: (i) granulocyte colony-stimulating factor (G-CSF), (ii) granulocyte-macrophage colony-stimulating factor (GM-CSF), (iii) interferon alpha (IFN-α), (iv) interferon gamma (IFN-γ), (v) interleukin-1 beta (IL-13), (vi) IL-2, (vii) IL-4, (viii) IL-5, (ix) IL-6, (x) IL-8, (xi) IL-10, (xii) interleukin-12 p70 (IL-12 p70), (xiii) IL-13, (xiv) IL-17A, (xv) IL-18, (xvi) interferon gamma-induced protein 10 (IP-10), (xvii) monocyte chemoattractant protein-1 (MCP-1), (xviii) macrophage inflammatory protein-1 alpha (MIP-la), (xix) MIP-1 beta (MIP-1β), (xx) tumor necrosis factor alpha (TNF-α), (xxi) TNF-β, and (xxii) any combination thereof.

53. The genetically modified hematopoietic cell according to any one of claims 50-52, wherein the fifth assay comprises: (i) a ProcartaPlex multiplex cytokine assay, (ii) a Human LEGENDplex TH1 panel, (iii) an AlphaLISA assay, (iv) an intracellular cytokine stain assay, (v) a Luminex bead-based cytokine release assay, (vi) a MACS cytokine secretion assay, (vii) an ELISA, or (viii) an ELISpot assay.

54. The genetically modified hematopoietic cell according to any one of claims 31-53, wherein the control hematopoietic cell is selected from the group consisting of: (i) a wild-type T-cell, (ii) a wild-type NK cell, and (iii) a wild-type TIL.

55. A method of generating a genetically modified cell population for treatment of a subject that has cancer, the method comprising:
(a) obtaining a plurality of hematopoietic cells;
(b) inhibiting expression of one or more CRL5 complex genes, wherein the one or more CRL5 complex genes is selected from the group consisting of: (i) CUL5, (ii) ARIH2, (iii) DCUN1D3, (iv) RNF7, (v) UBE2F, and (vi) any combination thereof;
(c) selecting hematopoietic cells in which the one or more CRL5 complex genes are inhibited; and
(d) expanding the selected hematopoietic cells ex vivo, thereby generating a genetically modified cell population for treatment of a subject that has cancer.

56. The method of claim 55, wherein the plurality of hematopoietic cells is derived from (i) the subject that has cancer, (ii) a donor that does not have cancer, or (iii) a cell line.

57. The method of claim 56, wherein the cell line is selected from the group consisting of: (i) an induced pluripotent stem cell (iPSC) line, and (ii) a hematopoietic stem cell (HSC) line.

58. A method of treating cancer, comprising:
administering a population of cells comprising a genetically modified hematopoietic cell according to any one of claims 31-54 to a subject that has cancer.

59. The method of claim 58, wherein the population of cells is derived from (i) the subject that has cancer, (ii) a donor that does not have cancer, or (iii) a cell line.

60. The method of claim 59, wherein the cell line is selected from the group consisting of: (i) an induced pluripotent stem cell (iPSC) line, and (ii) a hematopoietic stem cell (HSC) line.

61. The method of any one of claims 58-60, wherein the population of cells comprises about 100,000 (one hundred thousand) cells to about 100,000,000,000 (one hundred billion) cells.

62. The method of any one of claims 58-61, wherein prior to the administering, the subject has received a myeloablative conditioning regimen, a nonmyeloablative conditioning regimen, or a reduced intensity conditioning regimen.

63. A genetically modified hematopoietic cell, comprising:
one or more genetic modifications that inhibit expression or activity of polypeptide products encoded by one or more CRL5 complex-independent genes, wherein:
(i) the one or more CRL5 complex-independent genes are selected from a group comprising: (a) STUB1, (b) CCNC, (c) FAM49B, (d) N4BP1, (e) ARHGAP25, or
(f) any combination thereof; and
(ii) expression or activity of the one or more polypeptide products is inhibited by at least about 50% compared to a control hematopoietic cell.

64. The genetically modified hematopoietic cell of claim 63, wherein the one or more genetic modifications of the one or more CRL5 complex-independent genes inactivates the one or more CRL5 complex-independent genes.

65. The genetically modified hematopoietic cell according to any one of claims 63 or 64, wherein the one or more CRL5 complex-independent genes are inhibited using: (i) a clustered, regularly interspaced, short palindromic repeats (CRISPR) system, (ii) a transcription activator-like effector nuclease (TALEN) system, (iii) a zinc finger nuclease (ZFN) system, (iv) a meganuclease system, (v) a short hairpin RNA (shRNA) system, (vi) a small interfering RNA (siRNA) system, (vii) a microRNA (miRNA) system, or (viii) an antisense RNA system.

66. The genetically modified hematopoietic cell according to any one of claims 63-65, wherein the genetically modified hematopoietic cell comprises a natural killer (NK) cell.

67. The genetically modified hematopoietic cell of claim 66, wherein the NK cell comprises a CAR-NK cell.

68. The genetically modified hematopoietic cell according to any one of claims 63-67, wherein the one or more genetic modifications to one or more CRL5 complex-independent genes improves proliferation of the genetically modified hematopoietic cell by about 50% to about 75%, about 75% to about 100%, about 250% to about 300%, or about 625% to about 650% compared to proliferation of a control hematopoietic cell, as determined by a first assay.

69. The genetically modified hematopoietic cell according to any one of claims 63-68, wherein the one or more genetic modifications to one or more CRL5 complex-independent genes improves proliferation of the genetically modified hematopoietic cell by about 0.5-fold to about 0.75-fold, about 0.75-fold to about 1.0-fold, about 2.5-fold to about 3.0-fold, or about 6.25-fold to about 6.50-fold compared to proliferation of a control hematopoietic cell, as determined by a first assay.

70. The genetically modified hematopoietic cell according to any one of claims 68 or 69, wherein the first assay comprises: (i) a quantification of cells and/or cell clusters using a cell imaging system (e.g., Incucyte), (ii) a 5-ethynyl-2′-deoxyuridine (EdU) incorporation assay, (iii) a bromodeoxyuridine (BrdU) incorporation assay, (iv) a tetrazolium salt reduction assay, (v) an ATP assay, or (vi) a Ki-67 staining assay.

71. The genetically modified hematopoietic cell according to any one of claims 63-70, wherein the one or more genetic modifications to one or more CRL5 complex-independent genes improves resistance to immune suppression of the genetically modified hematopoietic cell by about 1.5% to about 6.0%, about 21.0% to about 24.0%, about 27.0% to about 30.0%, about 42.0% to about 45.0%, or about 53.0% to about 55.0% compared to resistance to immune suppression of a control hematopoietic cell, as determined by a second assay.

72. The genetically modified hematopoietic cell according to any one of claims 63-71, wherein the one or more genetic modifications to one or more CRL5 complex-independent genes improves resistance to immune suppression of the genetically modified hematopoietic cell by about 0.015-fold to about 0.060-fold, about 0.21-fold to about 0.24-fold, about 0.27-fold to about 0.30-fold, about 0.42-fold to about 0.45-fold, or about 0.53-fold to about 0.55-fold compared to immune suppression of a control hematopoietic cell, as determined by a second assay.

73. The genetically modified hematopoietic cell according to any one of claims 71 or 72, wherein the second assay comprises (i) a serial challenge assay, or (ii) a cytokine release assay.

74. The genetically modified hematopoietic cell according to any one of claims 63-73, wherein the one or more genetic modifications to one or more CRL5 complex-independent genes improves the cytotoxic capacity of the genetically modified hematopoietic cell by about 18.0% to about 24.0%, about 60.0% to about 63.0%, about 72.0% to about 75.0%, or about 87% to about 90% compared to cytotoxic capacity of a control hematopoietic cell, as determined by a third assay.

75. The genetically modified hematopoietic cell according to any one of claims 63-74, wherein the one or more genetic modifications to one or more CRL5 complex-independent genes improves the cytotoxic capacity of the genetically modified hematopoietic cell by about 0.18-fold to about 0.24-fold, about 0.60-fold to about 0.63-fold, about 0.72-fold to about 0.75-fold, or about 0.87-fold to about 0.90-fold compared to cytotoxic capacity of a control hematopoietic cell, as determined by a third assay.

76. The genetically modified hematopoietic cell according to any one of claims 74 or 75, wherein the third assay comprises: (i) a quantification of cells and/or cell clusters using a cell imaging system (e.g., Incucyte), (ii) a serial challenge assay, (iii) an enzymatic cytotoxicity assay, (iv) an apoptosis assay, (v) an autophagy assay, or (vi) a cell viability assay.

77. The genetically modified hematopoietic cell according to any one of claims 63-76, wherein the one or more genetic modifications to one or more CRL5 complex-independent genes improves the killing efficiency of cancer cells by the genetically modified hematopoietic cell by about 18.0% to about 24.0%, about 60.0% to about 63.0%, about 72.0% to about 75.0%, or about 87.0% to about 90.0% compared to the killing efficiency of cancer cells by a control hematopoietic cell, as determined by a fourth assay.

78. The genetically modified hematopoietic cell according to any one of claims 63-77, wherein the one or more genetic modifications to one or more CRL5 complex-independent genes improves the killing efficiency of cancer cells by the genetically modified hematopoietic cell by about 0.18-fold to about 0.24-fold, about 0.60-fold to about 0.63-fold, about 0.72-fold to about 0.75-fold, or about 0.87-fold to about 0.90-fold compared to the killing efficiency of cancer cells by a control hematopoietic cell, as determined by a fourth assay.

79. The genetically modified hematopoietic cell according to any one of claims 77 or 78, wherein the fourth assay comprises: (i) a quantification of cells and/or cell clusters using a cell imaging system (e.g., Incucyte), (ii) a serial challenge assay, (iii) an enzymatic cytotoxicity assay, (iv) an apoptosis assay, (v) an autophagy assay, or (vi) a cell viability assay.

80. The genetically modified hematopoietic cell according to any one of claims 63-79, wherein the one or more genetic modifications to one or more CRL5 complex-independent genes improves capability to stimulate production of one or more cytokines by the genetically modified hematopoietic cell by about 5.0% to about 10.0%, about 10.0% to about 15.0%, about 15.0% to about 20.0%, about 20.0% to about 25.0%, or about 25.0% to about 30.0% compared to capability to stimulate production of one or more cytokines of a control hematopoietic cell, as determined by a fifth assay.

81. The genetically modified hematopoietic cell according to any one of claims 63-80, wherein the one or more genetic modifications to one or more CRL5 complex-independent genes improves capability to stimulate production of one or more cytokines by the genetically modified hematopoietic cell by about 0.05-fold to about 0.10-fold, about 0.10-fold to about 0.15-fold, about 0.15-fold to about 0.20-fold, about 0.20-fold to about 0.25-fold, or about 0.25-fold to about 0.30-fold compared to capability to stimulate production of one or more cytokines of a control hematopoietic cell, as determined by a fifth assay.

82. The genetically modified hematopoietic cell according to any one of claims 80 or 81, wherein the one or more cytokines are selected from the group consisting of: (i) granulocyte colony-stimulating factor (G-CSF), (ii) granulocyte-macrophage colony-stimulating factor (GM-CSF), (iii) interferon alpha (IFN-α), (iv) interferon gamma (IFN-γ), (v) interleukin-1 beta (IL-13), (vi) IL-2, (vii) IL-4, (viii) IL-5, (ix) IL-6, (x) IL-8, (xi) IL-10, (xii) interleukin-12 p70 (IL-12 p70), (xiii) IL-13, (xiv) IL-17A, (xv) IL-18, (xvi) interferon gamma-induced protein 10 (IP-10), (xvii) monocyte chemoattractant protein-1 (MCP-1), (xviii) macrophage inflammatory protein-1 alpha (MIP-1α), (xix) MIP-1 beta (MIP-1β), (xx) tumor necrosis factor alpha (TNF-α), (xxi) TNF-β, and (xxii) any combination thereof.

83. The genetically modified hematopoietic cell according to any one of claims 80-82, wherein the fifth assay comprises: (i) a ProcartaPlex multiplex cytokine assay, (ii) a Human LEGENDplex TH1 panel, (iii) an AlphaLISA assay, (iv) an intracellular cytokine stain assay, (v) a Luminex bead-based cytokine release assay, (vi) a MACS cytokine secretion assay, (vii) an ELISA, or (viii) an ELISpot assay.

84. The genetically modified hematopoietic cell according to any one of claims 63-83, wherein the control hematopoietic cell comprises a wild-type NK cell.

85. A method of generating a genetically modified cell population for treatment of a subject that has cancer, the method comprising:
(a) obtaining a plurality of hematopoietic cells;
(b) inhibiting expression of one or more CRL5 complex-independent genes, wherein the one or more CRL5 complex genes is selected from a group comprising: (i) STUB1, (ii) CCNC, (iii) FAM49B, (iv) N4BP1, (v) ARHGAP25, or (vi) any combination thereof;
(c) selecting hematopoietic cells in which the one or more CRL5 complex-independent genes are inhibited; and
(d) expanding the selected hematopoietic cells ex vivo, thereby generating a genetically modified cell population for treatment of a subject that has cancer.

86. The method of claim 85, wherein the plurality of hematopoietic cells is derived from (i) the subject that has cancer, (ii) a donor that does not have cancer, or (iii) a cell line.

87. The method according to claim 86, wherein the cell line is selected from the group consisting of: (i) an induced pluripotent stem cell (iPSC) line, and (ii) a hematopoietic stem cell (HSC) line.

88. A method of treating cancer, comprising:
administering a population of cells comprising a genetically modified hematopoietic cell according to any one of claims 63-84 to a subject that has cancer.

89. The method of claim 88, wherein the population of cells is derived from (i) the subject that has cancer, (ii) a donor that does not have cancer, or (iii) a cell line.

90. The method of claim 89, wherein the cell line is selected from the group consisting of: (i) an induced pluripotent stem cell (iPSC) line, and (ii) a hematopoietic stem cell (HSC) line.

91. The method of any one of claims 88-90, wherein the population of cells comprises about 100,000 (one hundred thousand) cells to about 100,000,000,000 (one hundred billion) cells.

92. The method of any one of claims 88-91, wherein prior to the administering, the subject has received a myeloablative conditioning regimen, a nonmyeloablative conditioning regimen, or a reduced intensity conditioning regimen.
